(19) United States
(12) Patent Application Publication
(10) Pub. No.: US 2023/0036794 A1
(43) Pub. Date: Feb. 2, 2023

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Dong Min KANG, Suwon-si (KR); Jiyun KWON, Suwon-si (KR); Byungku KIM, Suwon-si (KR); Junmo PARK, Suwon-si (KR); Hansol SEO, Suwon-si (KR); Jin Sook KIM, Suwon-si (KR); Namheon LEE, Suwon-si (KR); Kipo JANG, Suwon-si (KR); Sung-Hyun JUNG, Suwon-si (KR); Ho Kuk JUNG, Suwon-si (KR); Youngkyoung JO, Suwon-si (KR); Bowon CHOI, Suwon-si (KR)

(21) Appl. No.: 17/845,288

(22) Filed: Jun. 21, 2022

(30) Foreign Application Priority Data
Jun. 21, 2021 (KR) 10-2021-0080242
Jun. 20, 2022 (KR) 10-2022-0075165

(51) Int. Cl. H01L 51/00; C07D 333/50; C09K 11/06; C07D 409/12; C07D 307/77; C07D 409/04; C07D 405/04

(52) U.S. Cl. CPC H01L 51/0061; C07D 333/50; C09K 11/06; H01L 51/006; C07D 409/12; C07D 307/77; C07D 409/04; C07D 405/04; H01L 51/0067; H01L 51/0073; H01L 51/0052; H01L 51/5016

(57) ABSTRACT

A compound for an organic optoelectronic device, a composition for an organic optoelectronic device, an organic optoelectronic device, and a display device, the compound being represented by Chemical Formula 1:

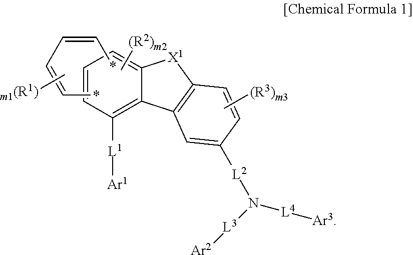

### CROSS-REFERENCE TO RELATED APPLICATION

This application claims priority to and the benefit of Korean Patent Application No. 10-2021-0080242, filed in the Korean Intellectual Property Office on Jun. 21, 2021, and Korean Patent Application No. 10-2022-0075165, filed in the Korean Intellectual Property Office on Jun. 20, 2022, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., organic optoelectronic diode) is a device capable of converting electrical energy and optical energy to each other.

Organic optoelectronic devices may be divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photo conductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### SUMMARY

The embodiments may be realized by providing a compound for an organic optoelectronic device, the compound being represented by Chemical Formula 1:

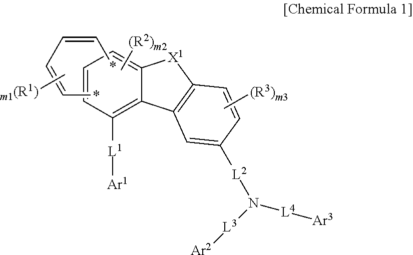

wherein, in Chemical Formula 1, X¹ is O or S, L¹ to L⁴ are each independently a single bond or a substituted or unsubstituted C6 to C30 arylene group, R¹ to R³ are each independently hydrogen, deuterium, or a substituted or unsubstituted C1 to C10 alkyl group, Ar¹ to Ar³ are each independently a substituted or unsubstituted C6 to C30 aryl group or a substituted or unsubstituted C2 to C30 heterocyclic group, m1 an integer of 1 to 4, m2 is 1, m3 an integer of 1 to 3, and * is a linking carbon.

The embodiments may be realized by providing a composition for an organic optoelectronic device, the composition including a first compound; and a second compound, wherein the first compound is the compound for an organic optoelectronic device according to an embodiment, and the second compound is represented by Chemical Formula 2:

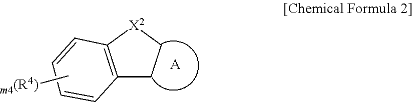

in Chemical Formula 2, X² is O, S, N-L^{a}-R^{a}, CR^{b}R^{c}, or SiR^{d}R^{e}, L^{a} is a single bond or a substituted or unsubstituted C6 to C12 arylene group, R^{a}, R^{b}, R^{c}, R^{d}, R^{e}, and R⁴ are each independently hydrogen, deuterium, a substituted or unsubstituted C1 to C30 alkyl group, a substituted or unsubstituted C6 to C30 aryl group, or a substituted or unsubstituted C2 to C30 heterocyclic group, m4 is an integer 1 to 4, and A is a ring of Group III,

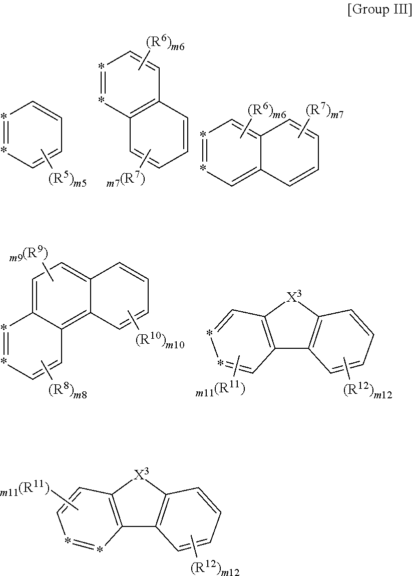

in Group III, * is a linking carbon, X³ is O or S, R⁵ to R¹² are each independently hydrogen, deuterium, a substituted or unsubstituted C1 to C30 alkyl group, a substituted or unsubstituted C6 to C30 aryl group, or a substituted or unsubstituted C2 to C30 heterocyclic group, m5, m7, m10, and m12 are each independently an integer of 1 to 4, m6, m8, m9, and m11 are each independently 1 or 2, and at least one of Ra and R⁴ to R¹² is a group represented by Chemical Formula a,

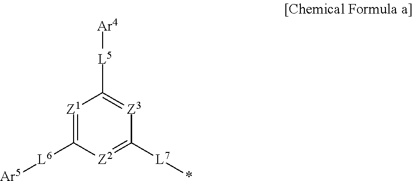

in Chemical Formula a, Z¹ to Z³ are each independently N or CR^{f}, at least two of Z¹ to Z³ are N, R^{f} is hydrogen, deuterium, a substituted or unsubstituted C1 to C10 alkyl group, or a substituted or unsubstituted C6 to C20 aryl group, L⁵ to L⁷ are each independently a single bond or a substituted or unsubstituted C6 to C30 arylene group, Ar⁴ and Ar⁵ are each independently a substituted or unsubstituted C6 to C30 aryl group or a substituted or unsubstituted C2 to C30 heterocyclic group, and * is a linking point.

The embodiments may be realized by providing an organic optoelectronic device including an anode and a cathode facing each other, and at least one organic layer between the anode and the cathode, wherein the at least one organic layer includes the compound for an organic optoelectronic device according to an embodiment.

The embodiments may be realized by providing an organic optoelectronic device including an anode and a cathode facing each other, at least one organic layer between the anode and the cathode, wherein the at least one organic layer includes the composition for an organic optoelectronic device according to an embodiment.

The embodiments may be realized by providing a display device including the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view illustrating an organic light emitting diode according to embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples, “substituted” refers to replacement of at least one hydrogen of a substituent or a compound by deuterium, a C1 to C20 alkyl group, a C6 to C30 aryl group, or a cyano group. In addition, in specific examples, “substituted” refers to replacement of at least one hydrogen of a substituent or a compound by deuterium, a C1 to C5 alkyl group, a C6 to C18 aryl group, or a cyano group. In addition, in specific examples, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

“Unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example a phenyl group, a naphthyl group, and the like, a group in which two or more hydrocarbon aromatic moieties may be linked by a sigma bond, for example a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted benzophenanthrenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof, but is not limited thereto.

More specifically, the substituted or unsubstituted C2 to C30 heterocyclic group may be a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group, a substituted or unsubstituted benzofuranofluorenyl group, a substituted or unsubstituted benzothiophenefluorenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a compound for an organic optoelectronic device according to an embodiment is described.

The compound for an organic optoelectronic device according to an embodiment may be represented by, e.g., Chemical Formula 1.

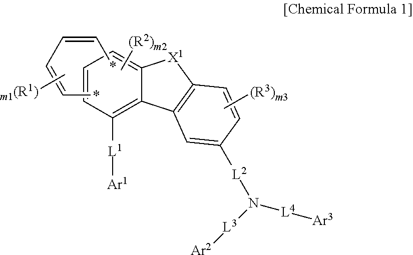

In Chemical Formula 1, X¹ may be, e.g., O or S. As used herein, the term “or” is not an exclusive term, e.g., “A or B” would include A, B, or A and B.

L¹ to L⁴ may each independently be or include, e.g., a single bond or a substituted or unsubstituted C6 to C30 arylene group.

R¹ to R³ may each independently be or include, e.g., hydrogen, deuterium or a substituted or unsubstituted C1 to C10 alkyl group.

Ar¹ to Ar³ may each independently be or include, e.g., a substituted or unsubstituted C6 to C30 aryl group or a substituted or unsubstituted C2 to C30 heterocyclic group.

m1 may be, e.g., an integer of 1 to 4.

m2 may be, e.g., 1.

m3 may be, e.g., an integer of 1 to 3.

* is a linking point or linking carbon. As used herein, the term “linking carbon” refers to a shared carbon at which fused rings are linked. For example, the *s may be adjacent carbons of the R²-containing ring to form a fused ring structure.

The compound represented by Chemical Formula 1 may have a more stable T1 energy level and excellent thermal stability by having a benzon[b]naphtho[2,1-d]furan or benzo[b]naphtho[1,2-d]furan backbone to help realize long life-span characteristics. The compound may have more stable HOMO energy as a dopant by including a substituted or unsubstituted amine group at the 2nd position, and a substituted or unsubstituted C6 to C30 aryl group or a substituted or unsubstituted C2 to C30 heterocyclic group at the 7th position and thus may have improved hole-transporting characteristics of the luminous efficiency to help exhibit high luminous efficiency and life-span characteristics. By introducing a substituent at the 7th position, the HOMO energy may be further stabilized, and Tg (glass transition temperature) may be improved by creating a structure having steric hindrance, thereby improving device processability.

In an implementation, the compound represented by Chemical Formula 1 may be represented by, e.g., Chemical Formula 1A or Chemical Formula 1B.

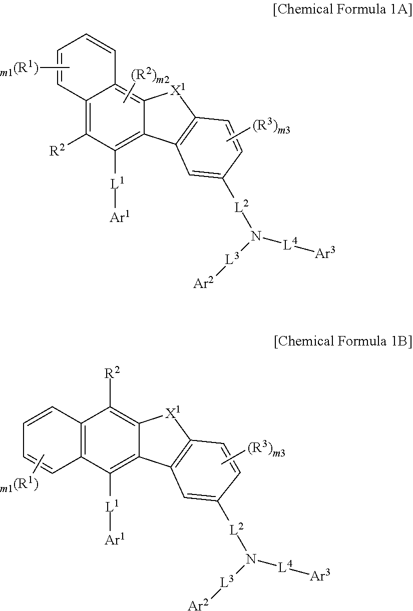

In Chemical Formula 1A and Chemical Formula 1B, X¹, L¹ to L⁴, Ar¹ to Ar³, R¹ to R³, m1, and m3 may be the same as those described above.

In an implementation, Ar¹ may be, e.g., a substituted or unsubstituted phenyl group, a substituted or unsubstituted biphenyl group, a substituted or unsubstituted naphthyl group, a substituted or unsubstituted anthracenyl group, a substituted or unsubstituted phenanthrenyl group, a substituted or unsubstituted chrysenyl group, a substituted or unsubstituted benzophenanthrenyl group, a substituted or unsubstituted triphenylene group, a substituted or unsubstituted fluorenyl group, a substituted or unsubstituted dibenzosilolyl group, a substituted or unsubstituted dibenzofuranyl group, a substituted or unsubstituted dibenzothiophenyl group, or a substituted or unsubstituted carbazolyl group.

In an implementation, L¹ may be, e.g., a single bond or a substituted or unsubstituted phenyl group.

In an implementation, the moiety -L¹-Ar¹ may be, e.g., a moiety of Group I.

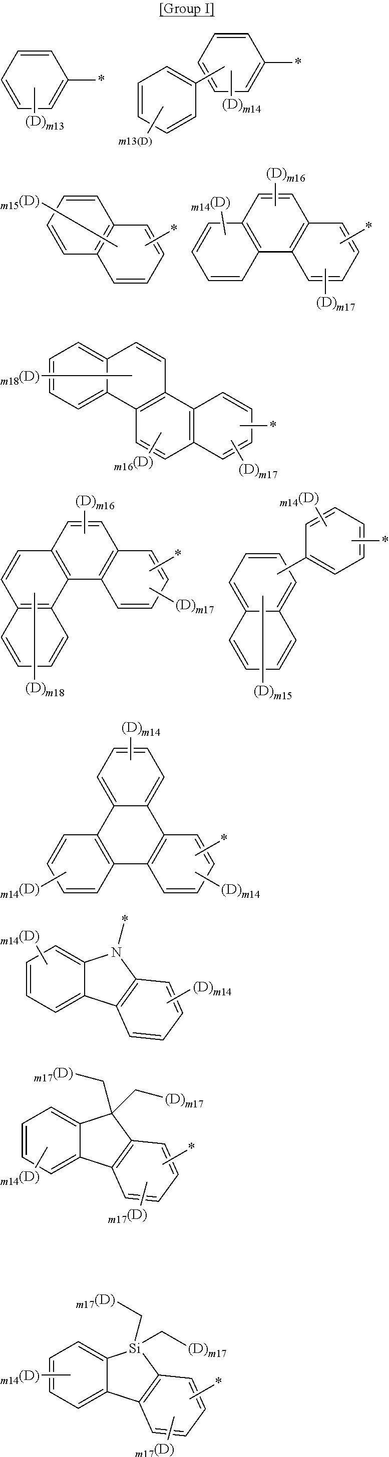

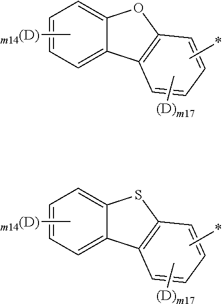

In Group I,

D is deuterium,

m13 may be, e.g., an integer of 0 to 5,

m14 may be, e.g., an integer of 0 to 4,

m15 may be, e.g., an integer of 0 to 7,

m16 may be, e.g., an integer of 0 to 2,

m17 may be, e.g., an integer of 0 to 3,

m18 may be, e.g., an integer of 0 to 6, and

* is a linking point.

In the definitions of m13 to m18, 0 means that all hydrogen atoms are not substituted with deuterium and remain as hydrogen atoms, e.g., “unsubstituted”.

In an implementation, Ar² and Ar³ may each independently be, e.g., a substituted or unsubstituted phenyl group, a substituted or unsubstituted biphenyl group, a substituted or unsubstituted naphthyl group, a substituted or unsubstituted phenanthrenyl group, a substituted or unsubstituted chrysenyl group, a substituted or unsubstituted fluorenyl group, a substituted or unsubstituted benzofluorenyl group, a substituted or unsubstituted benzophenanthrenyl group, a substituted or unsubstituted dibenzofuranyl group, a substituted or unsubstituted dibenzothiophenyl group, a substituted or unsubstituted dibenzosilolyl group, a substituted or unsubstituted benzonaphthofuranyl group, a substituted or unsubstituted benzonaphthothiophenyl group, a substituted or unsubstituted benzoxazolyl group, or a substituted or unsubstituted phenanthrooxazolyl group.

In an implementation, L³ and L⁴ may each independently be, e.g., a single bond, a substituted or unsubstituted phenylene group, or a substituted or unsubstituted naphthylene group.

In an implementation, moieties -L³-Ar² and -L⁴-Ar³ may each independently be, e.g., a moiety of Group II.

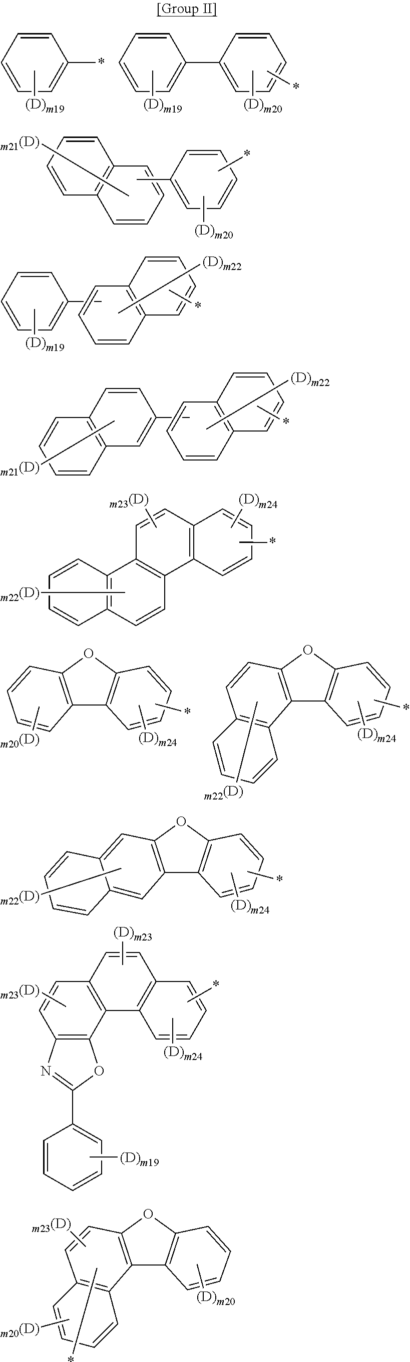

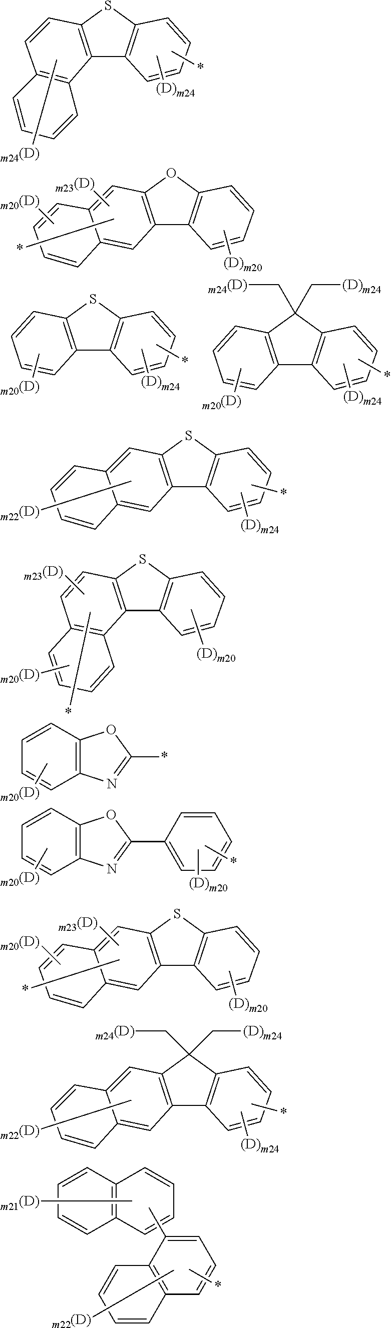

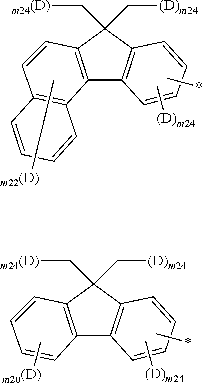

In Group II,

D is deuterium,

m19 may be, e.g., an integer of 0 to 5,

m20 may be, e.g., an integer of 0 to 4,

m21 may be, e.g., an integer of 0 to 7,

m22 may be, e.g., an integer of 0 to 6,

m23 may be, e.g., an integer of 0 to 2,

m24 may be, e.g., an integer of 0 to 3, and

* is a linking point.

In the definitions of m19 to m24, 0 means that all hydrogen atoms are not substituted with deuterium and remain as hydrogen atoms, e.g., “unsubstituted”.

In an implementation, R¹ to R³ may each independently be, e.g., hydrogen, deuterium, or a substituted or unsubstituted C1 to C5 alkyl group.

In an implementation, R¹ to R³ may each independently be, e.g., hydrogen or deuterium.

In an implementation, Ar¹ may be, e.g., a substituted or unsubstituted phenyl group, a substituted or unsubstituted naphthyl group, a substituted or unsubstituted dibenzofuranyl group, or a substituted or unsubstituted dibenzothiophenyl group.

In an implementation, Ar² and Ar³ may each independently be, e.g., a substituted or unsubstituted phenyl group, a substituted or unsubstituted biphenyl group, a substituted or unsubstituted naphthyl group, a substituted or unsubstituted fluorenyl group, a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group.

In an implementation, the compound represented by Chemical Formula 1 may be, e.g., a compound of Group 1.

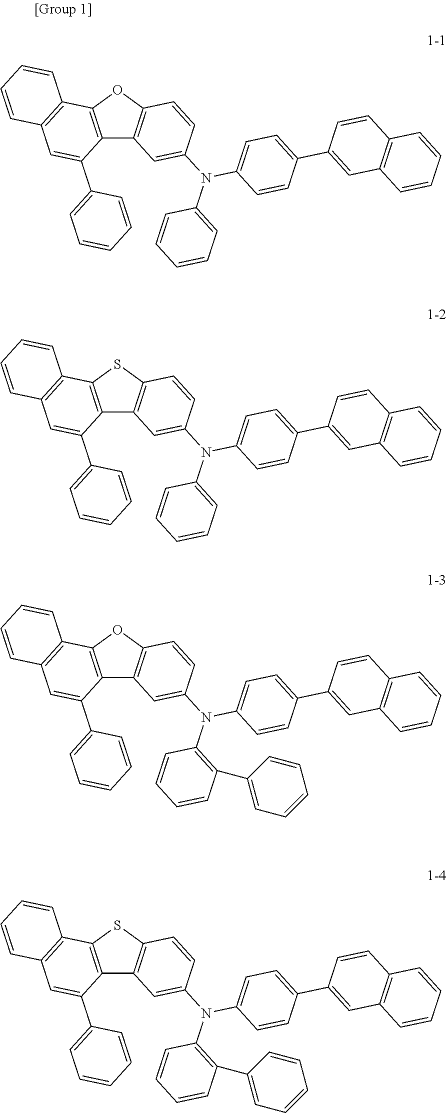 [SMILES: c1ccc(-c2cc3ccccc3c3oc4ccc(N(c5ccccc5)c5ccc(-c6ccc7ccccc7c6)cc5)cc4c23)cc1 ; c1ccc(-c2cc3ccccc3c3sc4ccc(N(c5ccccc5)c5ccc(-c6ccc7ccccc7c6)cc5)cc4c23)cc1 ; c1ccc(-c2ccccc2N(c2ccc(-c3ccc4ccccc4c3)cc2)c2ccc3oc4c5ccccc5cc(-c5ccccc5)c4c3c2)cc1 ; c1ccc(-c2ccccc2N(c2ccc(-c3ccc4ccccc4c3)cc2)c2ccc3sc4c5ccccc5cc(-c5ccccc5)c4c3c2)cc1]

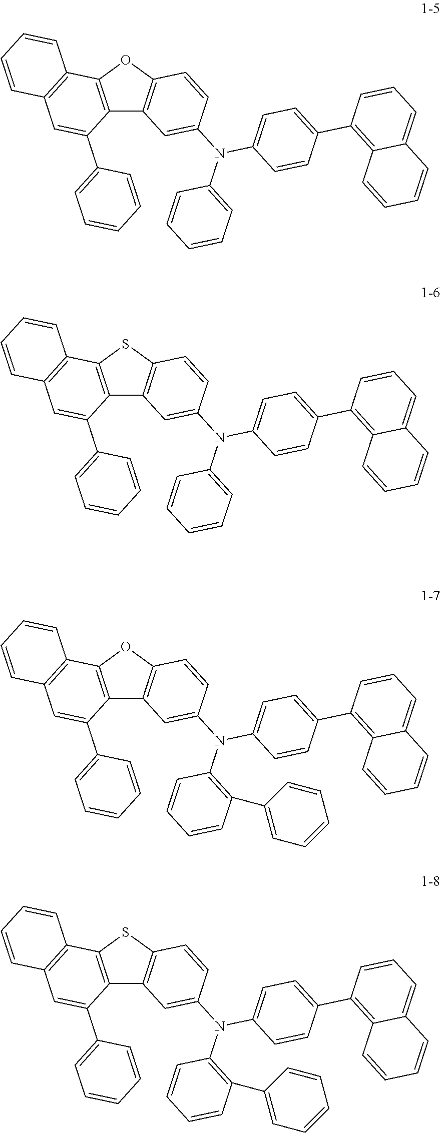 [SMILES: c1ccc(-c2cc3ccccc3c3oc4ccc(N(c5ccccc5)c5ccc(-c6cccc7ccccc67)cc5)cc4c23)cc1 ; c1ccc(-c2cc3ccccc3c3sc4ccc(N(c5ccccc5)c5ccc(-c6cccc7ccccc67)cc5)cc4c23)cc1 ; c1ccc(-c2ccccc2N(c2ccc(-c3cccc4ccccc34)cc2)c2ccc3oc4c5ccccc5cc(-c5ccccc5)c4c3c2)cc1 ; c1ccc(-c2ccccc2N(c2ccc(-c3cccc4ccccc34)cc2)c2ccc3sc4c5ccccc5cc(-c5ccccc5)c4c3c2)cc1]

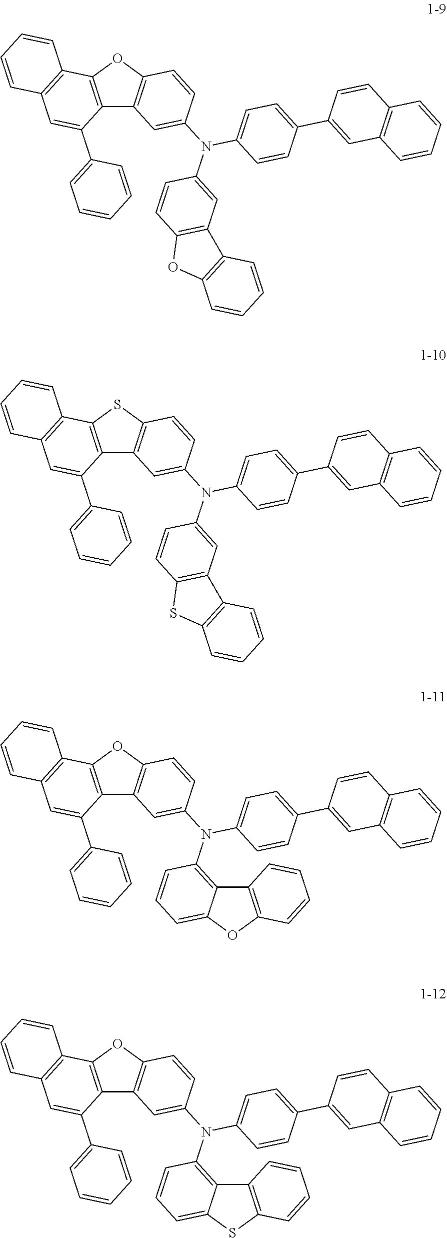 [SMILES: c1ccc(-c2cc3ccccc3c3oc4ccc(N(c5ccc(-c6ccc7ccccc7c6)cc5)c5ccc6oc7ccccc7c6c5)cc4c23)cc1 ; c1ccc(-c2cc3ccccc3c3sc4ccc(N(c5ccc(-c6ccc7ccccc7c6)cc5)c5ccc6sc7ccccc7c6c5)cc4c23)cc1 ; c1ccc(-c2cc3ccccc3c3oc4ccc(N(c5ccc(-c6ccc7ccccc7c6)cc5)c5cccc6oc7ccccc7c56)cc4c23)cc1 ; c1ccc(-c2cc3ccccc3c3oc4ccc(N(c5ccc(-c6ccc7ccccc7c6)cc5)c5cccc6sc7ccccc7c56)cc4c23)cc1]

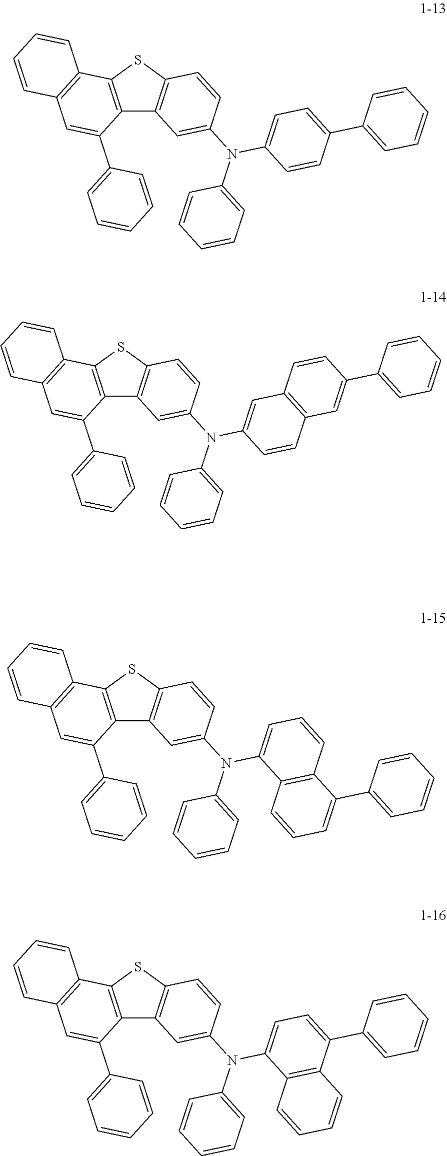 [SMILES: c1ccc(-c2ccc(N(c3ccccc3)c3ccc4sc5c6ccccc6cc(-c6ccccc6)c5c4c3)cc2)cc1 ; c1ccc(-c2ccc3cc(N(c4ccccc4)c4ccc5sc6c7ccccc7cc(-c7ccccc7)c6c5c4)ccc3c2)cc1 ; c1ccc(-c2cccc3c(N(c4ccccc4)c4ccc5sc6c7ccccc7cc(-c7ccccc7)c6c5c4)cccc23)cc1 ; c1ccc(-c2ccc(N(c3ccccc3)c3ccc4sc5c6ccccc6cc(-c6ccccc6)c5c4c3)c3ccccc23)cc1]

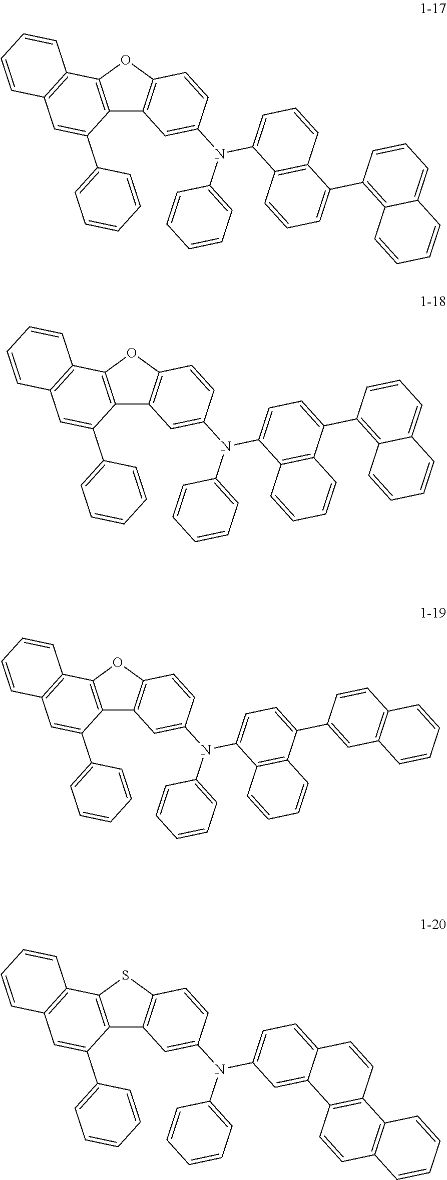 [SMILES: c1ccc(-c2cc3ccccc3c3oc4ccc(N(c5ccccc5)c5cccc6c(-c7cccc8ccccc78)cccc56)cc4c23)cc1 ; c1ccc(-c2cc3ccccc3c3oc4ccc(N(c5ccccc5)c5ccc(-c6cccc7ccccc67)c6ccccc56)cc4c23)cc1 ; c1ccc(-c2cc3ccccc3c3oc4ccc(N(c5ccccc5)c5ccc(-c6ccc7ccccc7c6)c6ccccc56)cc4c23)cc1 ; c1ccc(-c2cc3ccccc3c3sc4ccc(N(c5ccccc5)c5ccc6ccc7c8ccccc8ccc7c6c5)cc4c23)cc1]

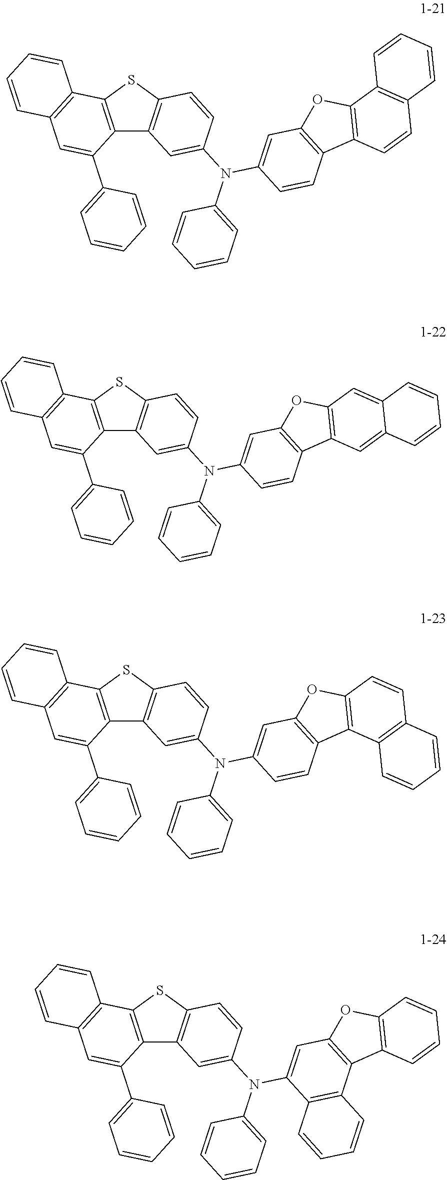 [SMILES: c1ccc(-c2cc3ccccc3c3sc4ccc(N(c5ccccc5)c5ccc6c(c5)oc5c7ccccc7ccc65)cc4c23)cc1 ; c1ccc(-c2cc3ccccc3c3sc4ccc(N(c5ccccc5)c5ccc6c(c5)oc5cc7ccccc7cc56)cc4c23)cc1 ; c1ccc(-c2cc3ccccc3c3sc4ccc(N(c5ccccc5)c5ccc6c(c5)oc5ccc7ccccc7c56)cc4c23)cc1 ; c1ccc(-c2cc3ccccc3c3sc4ccc(N(c5ccccc5)c5cc6oc7ccccc7c6c6ccccc56)cc4c23)cc1]

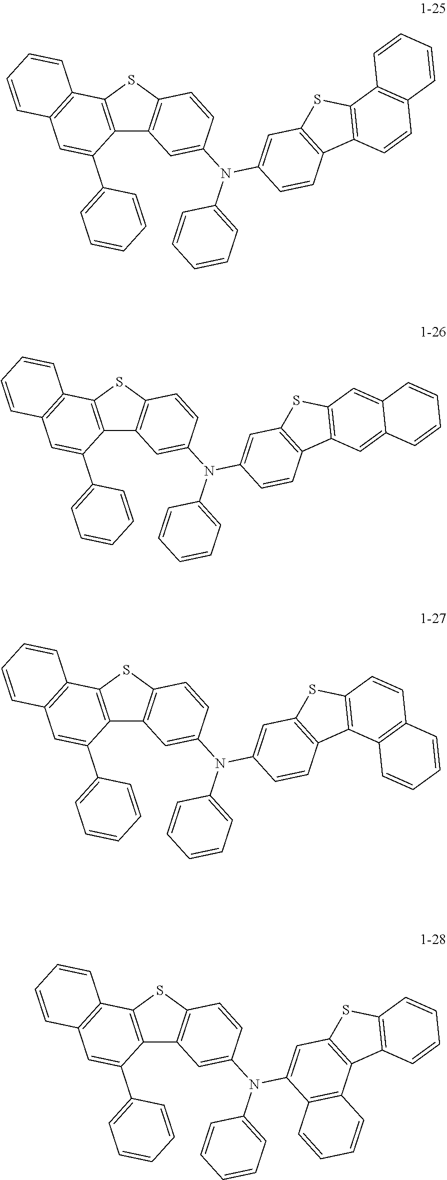 [SMILES: c1ccc(-c2cc3ccccc3c3sc4ccc(N(c5ccccc5)c5ccc6c(c5)sc5c7ccccc7ccc65)cc4c23)cc1 ; c1ccc(-c2cc3ccccc3c3sc4ccc(N(c5ccccc5)c5ccc6c(c5)sc5cc7ccccc7cc56)cc4c23)cc1 ; c1ccc(-c2cc3ccccc3c3sc4ccc(N(c5ccccc5)c5ccc6c(c5)sc5ccc7ccccc7c56)cc4c23)cc1 ; c1ccc(-c2cc3ccccc3c3sc4ccc(N(c5ccccc5)c5cc6sc7ccccc7c6c6ccccc56)cc4c23)cc1]

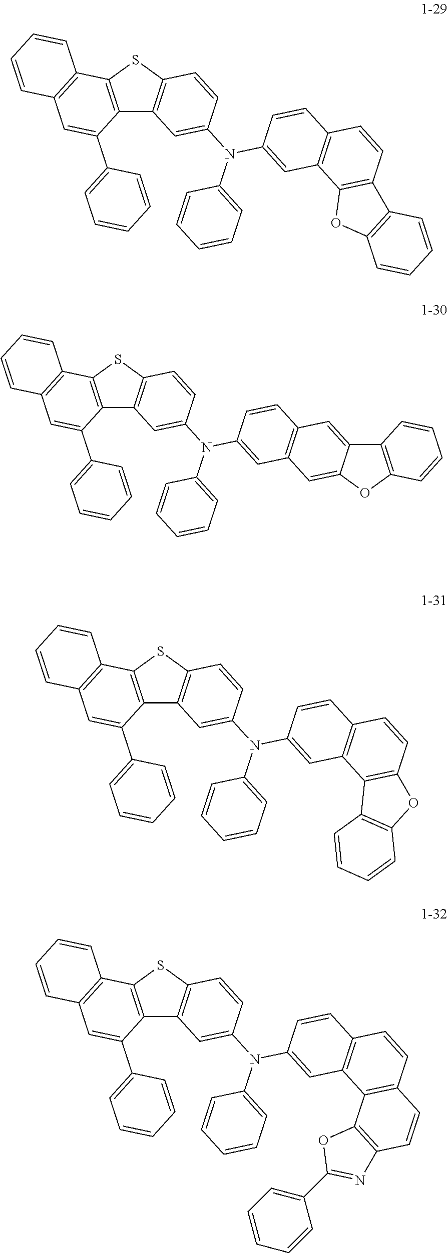 [SMILES: c1ccc(-c2cc3ccccc3c3sc4ccc(N(c5ccccc5)c5ccc6ccc7c8ccccc8oc7c6c5)cc4c23)cc1 ; c1ccc(-c2cc3ccccc3c3sc4ccc(N(c5ccccc5)c5ccc6cc7c(cc6c5)oc5ccccc57)cc4c23)cc1 ; c1ccc(-c2cc3ccccc3c3sc4ccc(N(c5ccccc5)c5ccc6ccc7oc8ccccc8c7c6c5)cc4c23)cc1 ; c1ccc(-c2nc3ccc4ccc5ccc(N(c6ccccc6)c6ccc7sc8c9ccccc9cc(-c9ccccc9)c8c7c6)cc5c4c3o2)cc1]

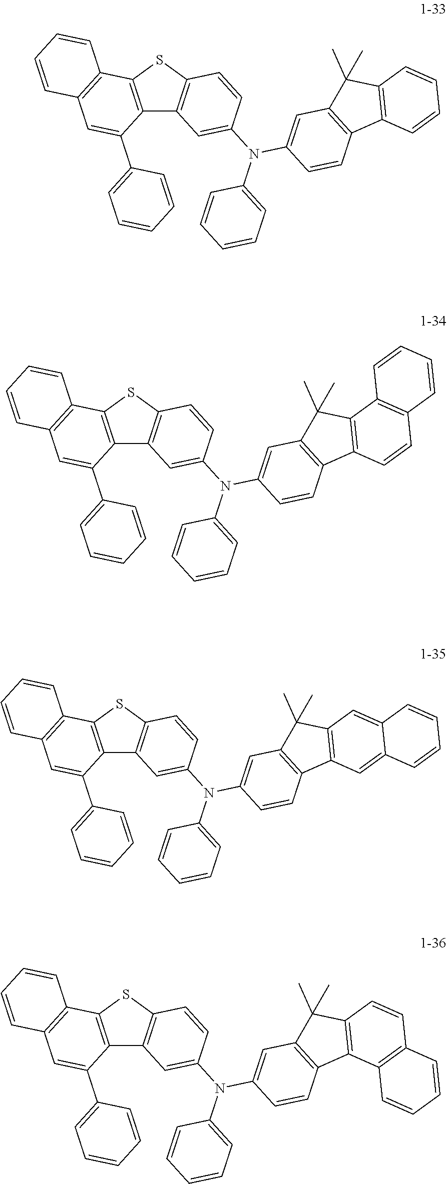 [SMILES: CC1(C)c2ccccc2-c2ccc(N(c3ccccc3)c3ccc4sc5c6ccccc6cc(-c6ccccc6)c5c4c3)cc21 ; CC1(C)c2cc(N(c3ccccc3)c3ccc4sc5c6ccccc6cc(-c6ccccc6)c5c4c3)ccc2-c2ccc3ccccc3c21 ; CC1(C)c2cc(N(c3ccccc3)c3ccc4sc5c6ccccc6cc(-c6ccccc6)c5c4c3)ccc2-c2cc3ccccc3cc21 ; CC1(C)c2cc(N(c3ccccc3)c3ccc4sc5c6ccccc6cc(-c6ccccc6)c5c4c3)ccc2-c2c1ccc1ccccc21]

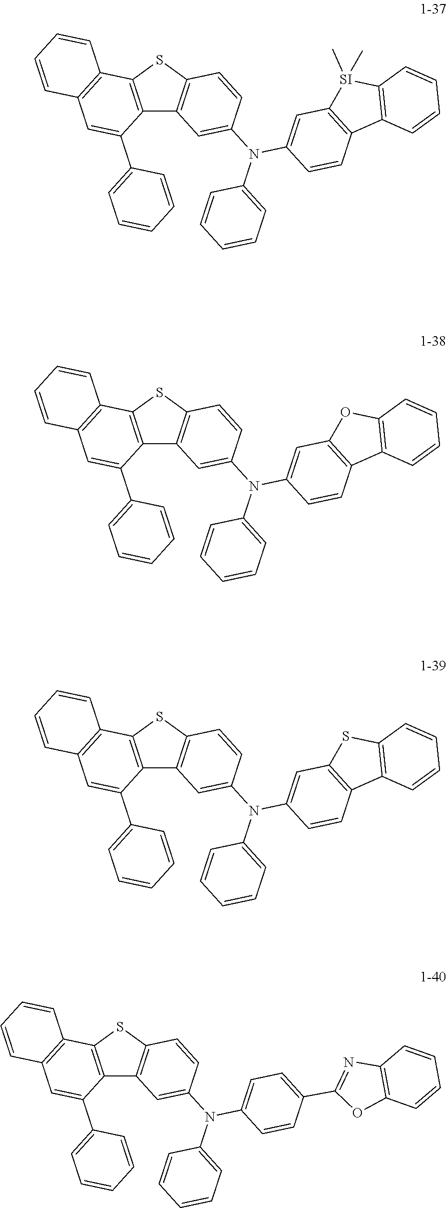

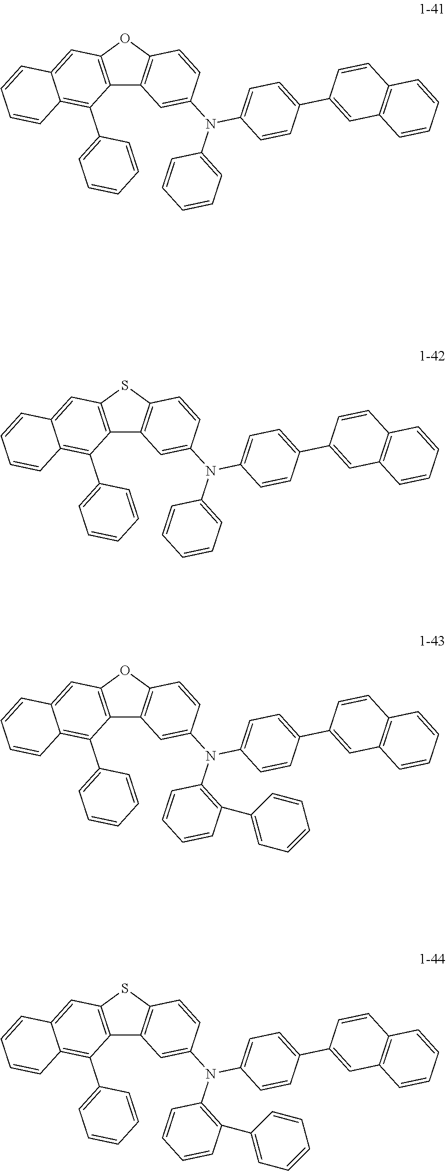 [SMILES: c1ccc(-c2c3ccccc3cc3oc4ccc(N(c5ccccc5)c5ccc(-c6ccc7ccccc7c6)cc5)cc4c23)cc1 ; c1ccc(-c2c3ccccc3cc3sc4ccc(N(c5ccccc5)c5ccc(-c6ccc7ccccc7c6)cc5)cc4c23)cc1 ; c1ccc(-c2ccccc2N(c2ccc(-c3ccc4ccccc4c3)cc2)c2ccc3oc4cc5ccccc5c(-c5ccccc5)c4c3c2)cc1 ; c1ccc(-c2ccccc2N(c2ccc(-c3ccc4ccccc4c3)cc2)c2ccc3sc4cc5ccccc5c(-c5ccccc5)c4c3c2)cc1]

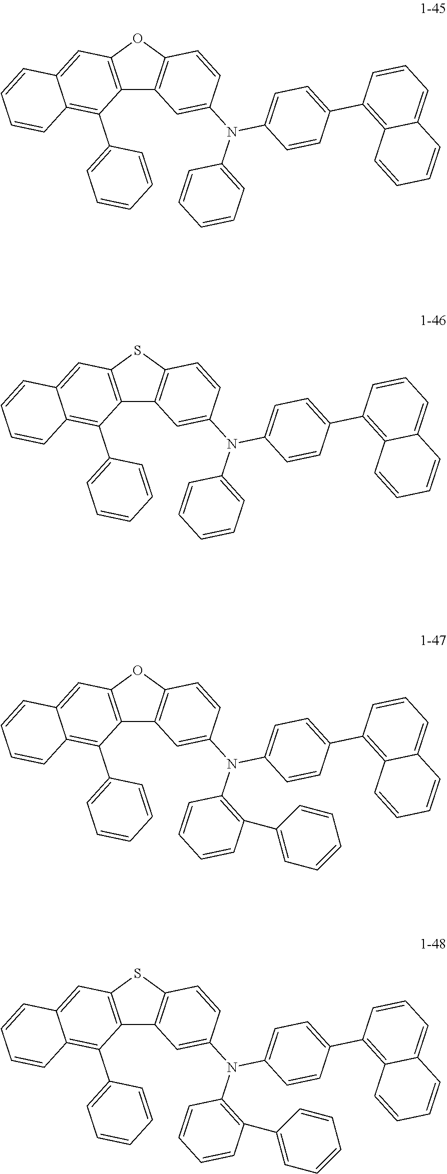 [SMILES: c1ccc(-c2c3ccccc3cc3oc4ccc(N(c5ccccc5)c5ccc(-c6cccc7ccccc67)cc5)cc4c23)cc1 ; c1ccc(-c2c3ccccc3cc3sc4ccc(N(c5ccccc5)c5ccc(-c6cccc7ccccc67)cc5)cc4c23)cc1 ; c1ccc(-c2ccccc2N(c2ccc(-c3cccc4ccccc34)cc2)c2ccc3oc4cc5ccccc5c(-c5ccccc5)c4c3c2)cc1 ; c1ccc(-c2ccccc2N(c2ccc(-c3cccc4ccccc34)cc2)c2ccc3sc4cc5ccccc5c(-c5ccccc5)c4c3c2)cc1]

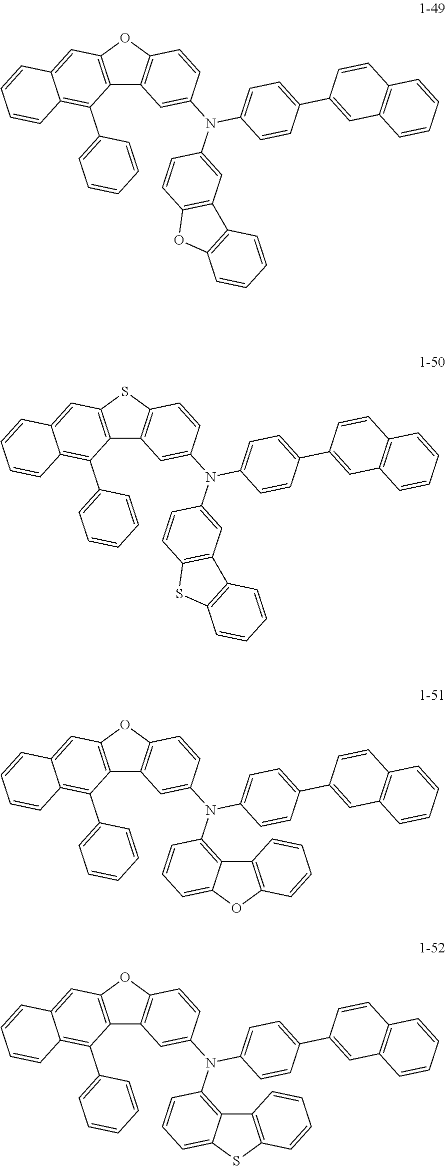 [SMILES: c1ccc(-c2c3ccccc3cc3oc4ccc(N(c5ccc(-c6ccc7ccccc7c6)cc5)c5ccc6oc7ccccc7c6c5)cc4c23)cc1 ; c1ccc(-c2c3ccccc3cc3sc4ccc(N(c5ccc(-c6ccc7ccccc7c6)cc5)c5ccc6sc7ccccc7c6c5)cc4c23)cc1 ; c1ccc(-c2c3ccccc3cc3oc4ccc(N(c5ccc(-c6ccc7ccccc7c6)cc5)c5cccc6oc7ccccc7c56)cc4c23)cc1 ; c1ccc(-c2c3ccccc3cc3oc4ccc(N(c5ccc(-c6ccc7ccccc7c6)cc5)c5cccc6sc7ccccc7c56)cc4c23)cc1]

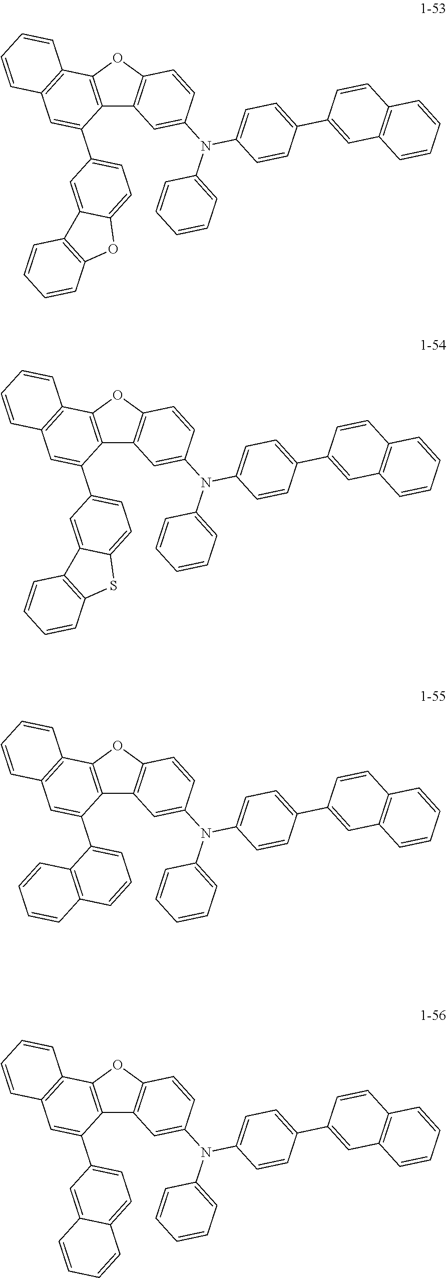 [SMILES: c1ccc(N(c2ccc(-c3ccc4ccccc4c3)cc2)c2ccc3oc4c5ccccc5cc(-c5ccc6oc7ccccc7c6c5)c4c3c2)cc1 ; c1ccc(N(c2ccc(-c3ccc4ccccc4c3)cc2)c2ccc3oc4c5ccccc5cc(-c5ccc6sc7ccccc7c6c5)c4c3c2)cc1 ; c1ccc(N(c2ccc(-c3ccc4ccccc4c3)cc2)c2ccc3oc4c5ccccc5cc(-c5cccc6ccccc56)c4c3c2)cc1 ; c1ccc(N(c2ccc(-c3ccc4ccccc4c3)cc2)c2ccc3oc4c5ccccc5cc(-c5ccc6ccccc6c5)c4c3c2)cc1]

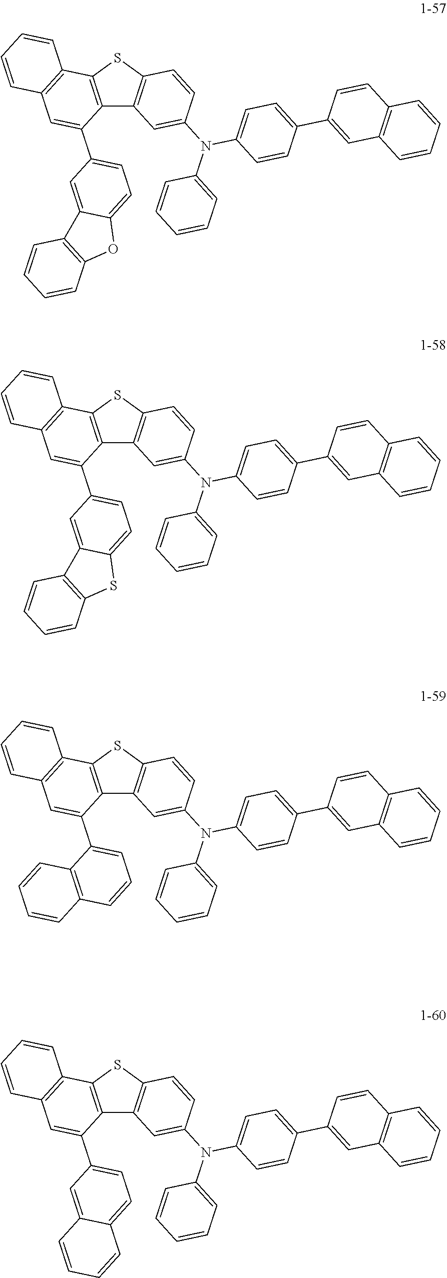 [SMILES: c1ccc(N(c2ccc(-c3ccc4ccccc4c3)cc2)c2ccc3sc4c5ccccc5cc(-c5ccc6oc7ccccc7c6c5)c4c3c2)cc1 ; c1ccc(N(c2ccc(-c3ccc4ccccc4c3)cc2)c2ccc3sc4c5ccccc5cc(-c5ccc6sc7ccccc7c6c5)c4c3c2)cc1 ; c1ccc(N(c2ccc(-c3ccc4ccccc4c3)cc2)c2ccc3sc4c5ccccc5cc(-c5cccc6ccccc56)c4c3c2)cc1 ; c1ccc(N(c2ccc(-c3ccc4ccccc4c3)cc2)c2ccc3sc4c5ccccc5cc(-c5ccc6ccccc6c5)c4c3c2)cc1]

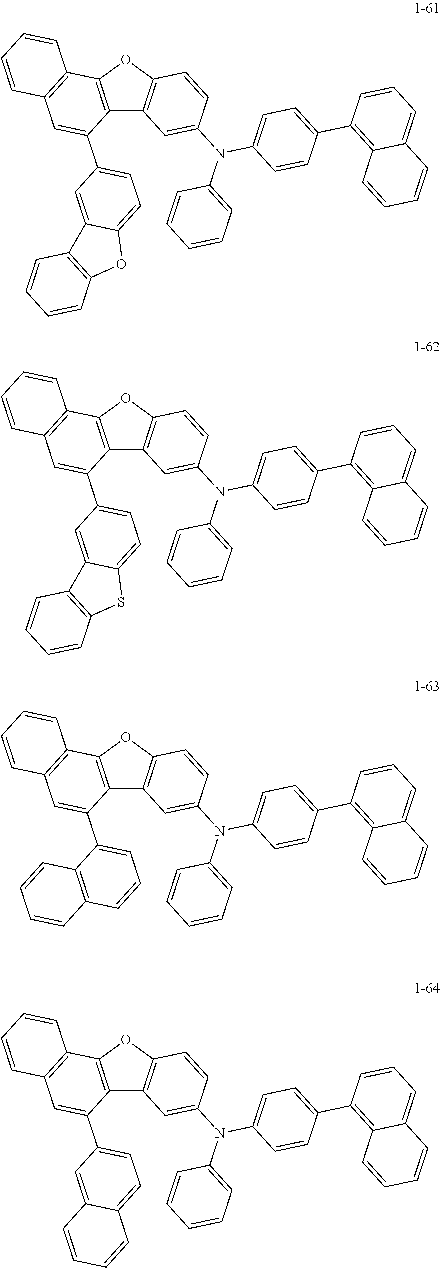 [SMILES: c1ccc(N(c2ccc(-c3cccc4ccccc34)cc2)c2ccc3oc4c5ccccc5cc(-c5ccc6oc7ccccc7c6c5)c4c3c2)cc1 ; c1ccc(N(c2ccc(-c3cccc4ccccc34)cc2)c2ccc3oc4c5ccccc5cc(-c5ccc6sc7ccccc7c6c5)c4c3c2)cc1 ; c1ccc(N(c2ccc(-c3cccc4ccccc34)cc2)c2ccc3oc4c5ccccc5cc(-c5cccc6ccccc56)c4c3c2)cc1 ; c1ccc(N(c2ccc(-c3cccc4ccccc34)cc2)c2ccc3oc4c5ccccc5cc(-c5ccc6ccccc6c5)c4c3c2)cc1]

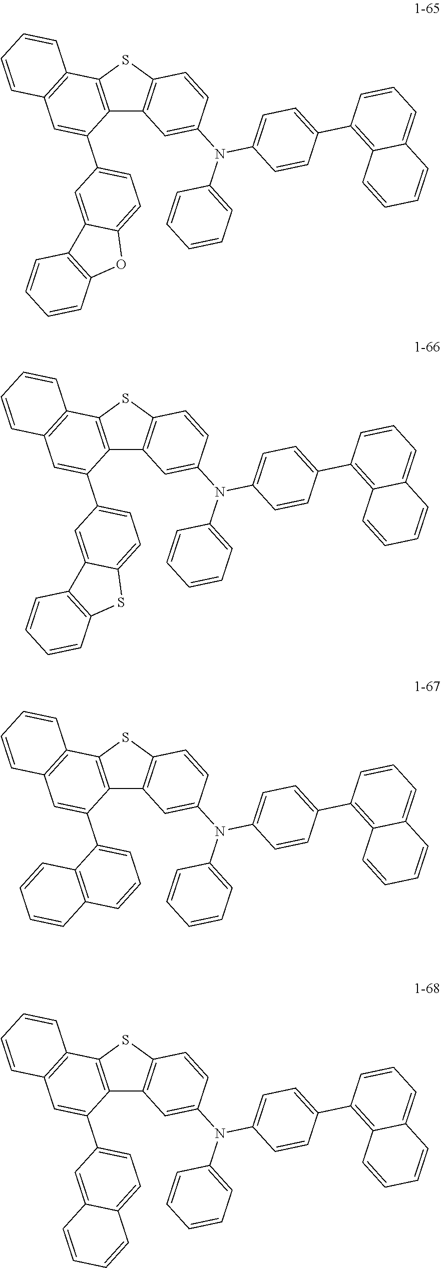 [SMILES: c1ccc(N(c2ccc(-c3cccc4ccccc34)cc2)c2ccc3sc4c5ccccc5cc(-c5ccc6oc7ccccc7c6c5)c4c3c2)cc1 ; c1ccc(N(c2ccc(-c3cccc4ccccc34)cc2)c2ccc3sc4c5ccccc5cc(-c5ccc6sc7ccccc7c6c5)c4c3c2)cc1 ; c1ccc(N(c2ccc(-c3cccc4ccccc34)cc2)c2ccc3sc4c5ccccc5cc(-c5cccc6ccccc56)c4c3c2)cc1 ; c1ccc(N(c2ccc(-c3cccc4ccccc34)cc2)c2ccc3sc4c5ccccc5cc(-c5ccc6ccccc6c5)c4c3c2)cc1]

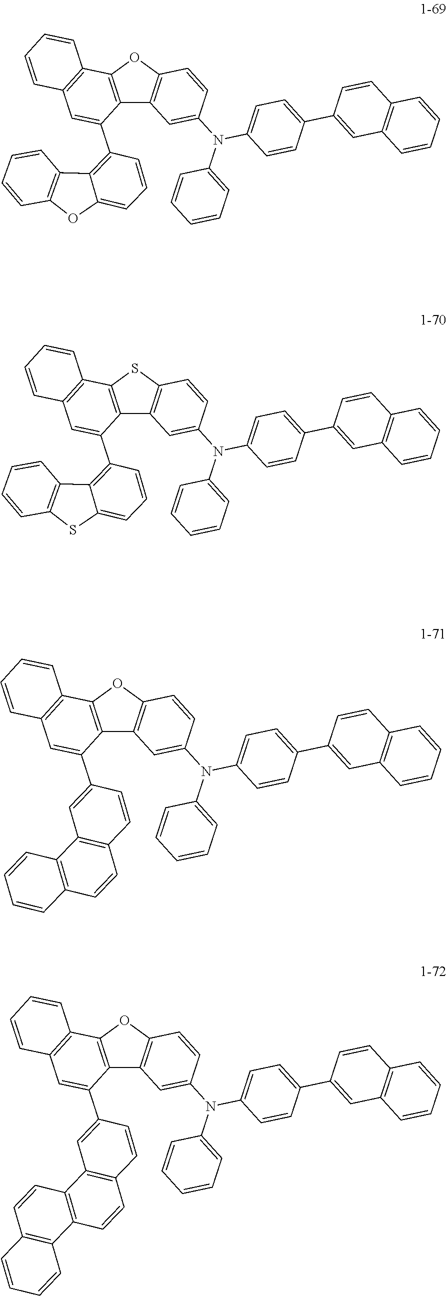 [SMILES: c1ccc(N(c2ccc(-c3ccc4ccccc4c3)cc2)c2ccc3oc4c5ccccc5cc(-c5cccc6oc7ccccc7c56)c4c3c2)cc1 ; c1ccc(N(c2ccc(-c3ccc4ccccc4c3)cc2)c2ccc3sc4c5ccccc5cc(-c5cccc6sc7ccccc7c56)c4c3c2)cc1 ; c1ccc(N(c2ccc(-c3ccc4ccccc4c3)cc2)c2ccc3oc4c5ccccc5cc(-c5ccc6ccc7ccccc7c6c5)c4c3c2)cc1 ; c1ccc(N(c2ccc(-c3ccc4ccccc4c3)cc2)c2ccc3oc4c5ccccc5cc(-c5ccc6ccc7c8ccccc8ccc7c6c5)c4c3c2)cc1]

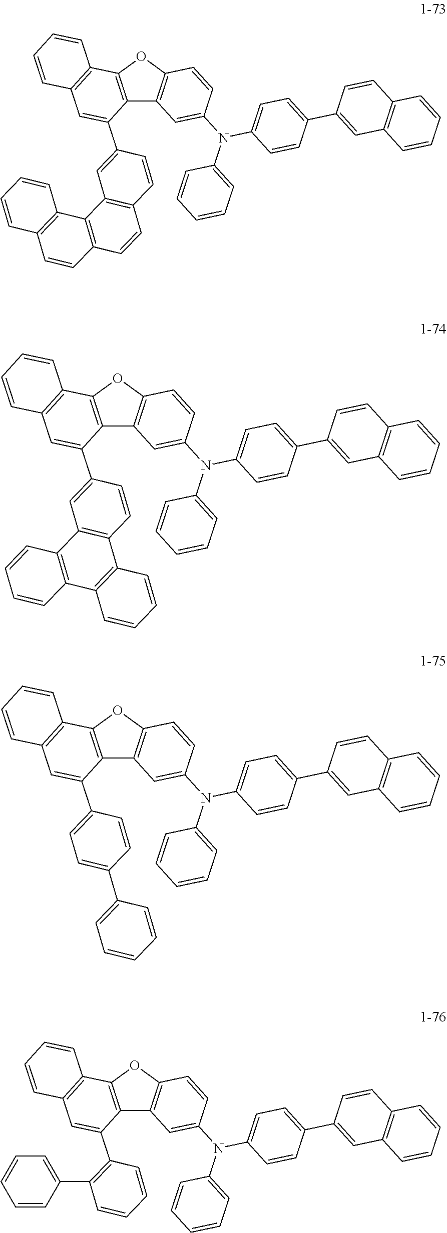 [SMILES: c1ccc(N(c2ccc(-c3ccc4ccccc4c3)cc2)c2ccc3oc4c5ccccc5cc(-c5ccc6ccc7ccc8ccccc8c7c6c5)c4c3c2)cc1 ; c1ccc(N(c2ccc(-c3ccc4ccccc4c3)cc2)c2ccc3oc4c5ccccc5cc(-c5ccc6c7ccccc7c7ccccc7c6c5)c4c3c2)cc1 ; c1ccc(-c2ccc(-c3cc4ccccc4c4oc5ccc(N(c6ccccc6)c6ccc(-c7ccc8ccccc8c7)cc6)cc5c34)cc2)cc1 ; c1ccc(-c2ccccc2-c2cc3ccccc3c3oc4ccc(N(c5ccccc5)c5ccc(-c6ccc7ccccc7c6)cc5)cc4c23)cc1]

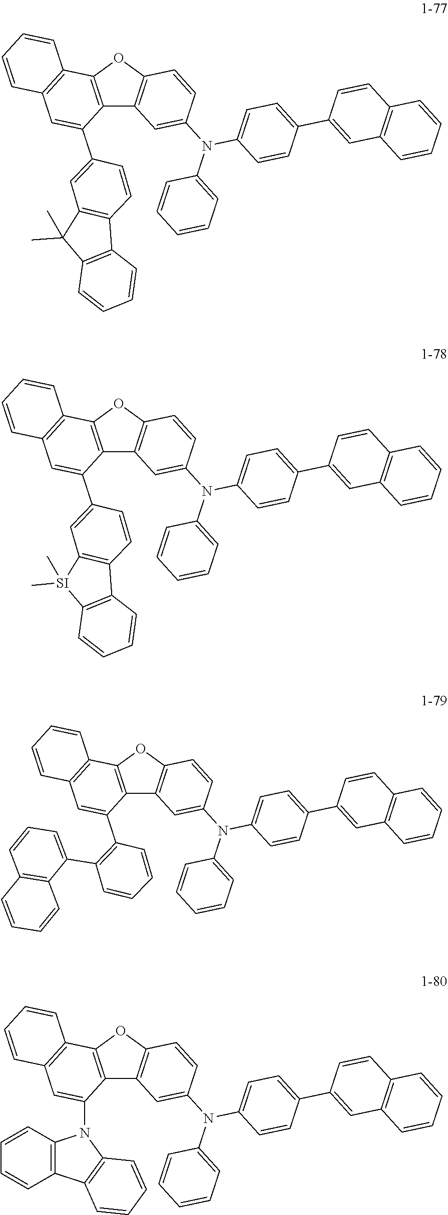

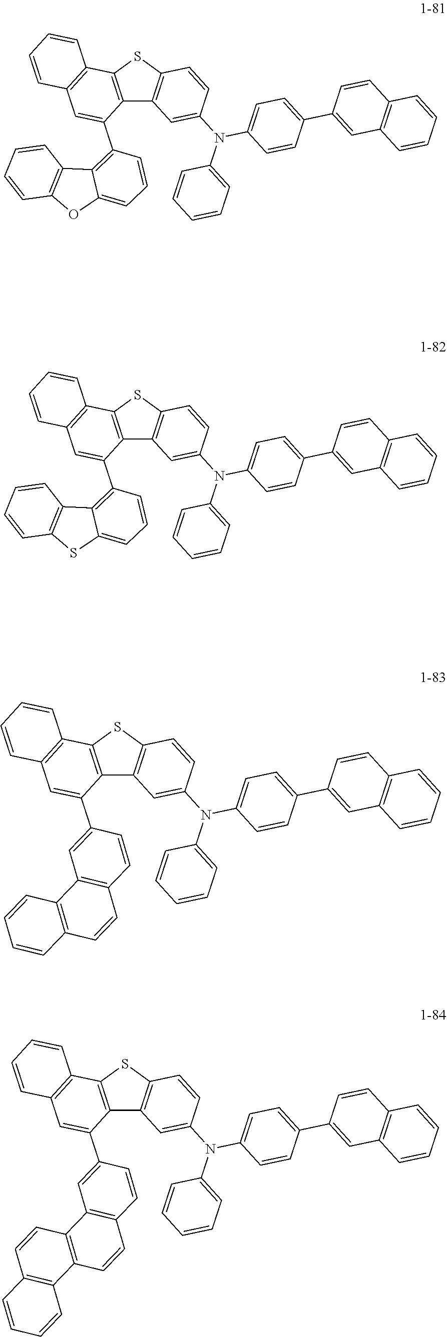 [SMILES: c1ccc(N(c2ccc(-c3ccc4ccccc4c3)cc2)c2ccc3sc4c5ccccc5cc(-c5cccc6oc7ccccc7c56)c4c3c2)cc1 ; c1ccc(N(c2ccc(-c3ccc4ccccc4c3)cc2)c2ccc3sc4c5ccccc5cc(-c5cccc6sc7ccccc7c56)c4c3c2)cc1 ; c1ccc(N(c2ccc(-c3ccc4ccccc4c3)cc2)c2ccc3sc4c5ccccc5cc(-c5ccc6ccc7ccccc7c6c5)c4c3c2)cc1 ; c1ccc(N(c2ccc(-c3ccc4ccccc4c3)cc2)c2ccc3sc4c5ccccc5cc(-c5ccc6ccc7c8ccccc8ccc7c6c5)c4c3c2)cc1]

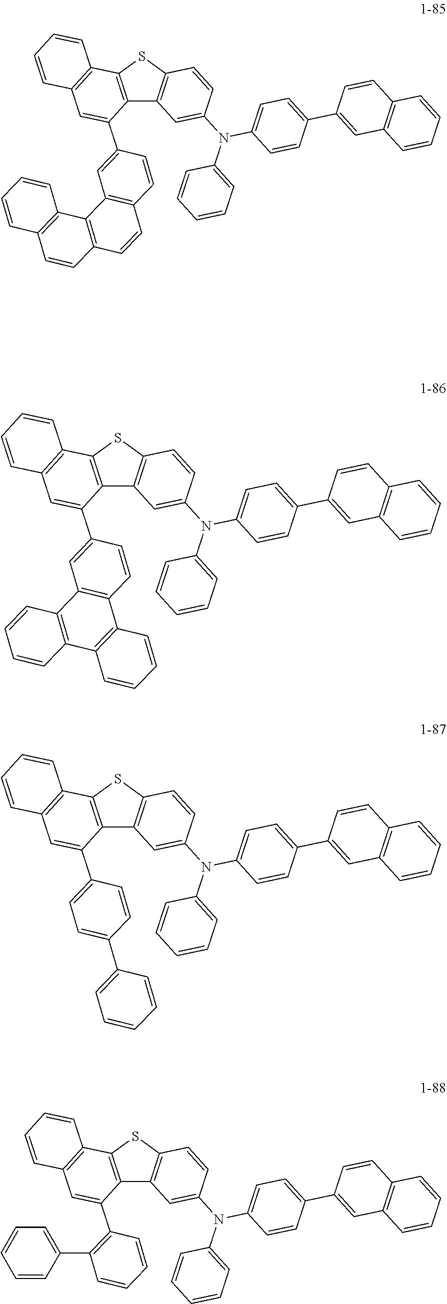 [SMILES: c1ccc(N(c2ccc(-c3ccc4ccccc4c3)cc2)c2ccc3sc4c5ccccc5cc(-c5ccc6ccc7ccc8ccccc8c7c6c5)c4c3c2)cc1 ; c1ccc(N(c2ccc(-c3ccc4ccccc4c3)cc2)c2ccc3sc4c5ccccc5cc(-c5ccc6c7ccccc7c7ccccc7c6c5)c4c3c2)cc1 ; c1ccc(-c2ccc(-c3cc4ccccc4c4sc5ccc(N(c6ccccc6)c6ccc(-c7ccc8ccccc8c7)cc6)cc5c34)cc2)cc1 ; c1ccc(-c2ccccc2-c2cc3ccccc3c3sc4ccc(N(c5ccccc5)c5ccc(-c6ccc7ccccc7c6)cc5)cc4c23)cc1]

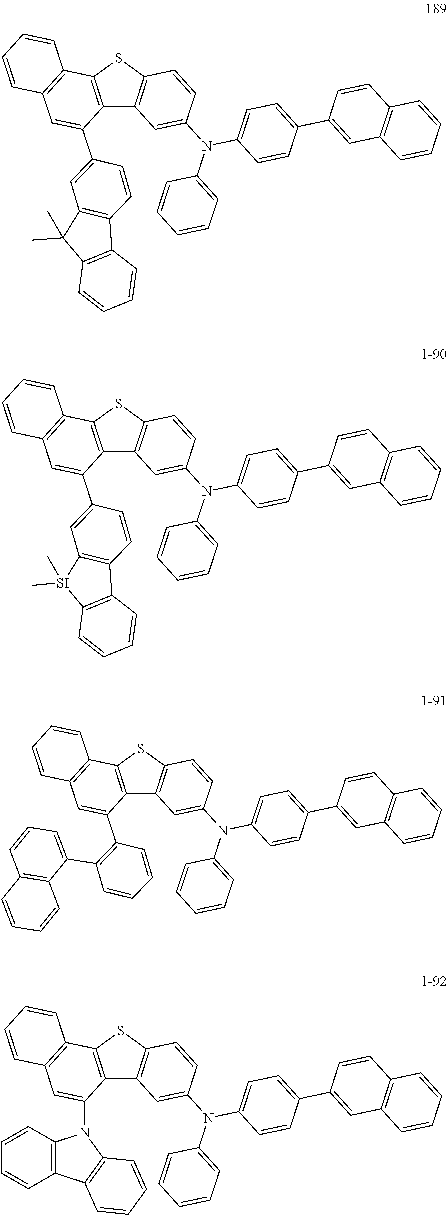

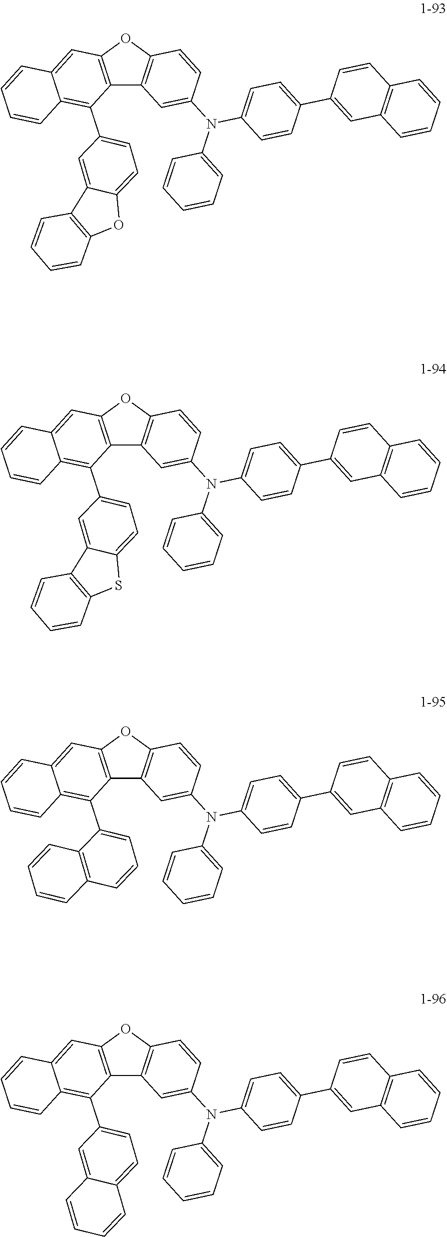 [SMILES: c1ccc(N(c2ccc(-c3ccc4ccccc4c3)cc2)c2ccc3oc4cc5ccccc5c(-c5ccc6oc7ccccc7c6c5)c4c3c2)cc1 ; c1ccc(N(c2ccc(-c3ccc4ccccc4c3)cc2)c2ccc3oc4cc5ccccc5c(-c5ccc6sc7ccccc7c6c5)c4c3c2)cc1 ; c1ccc(N(c2ccc(-c3ccc4ccccc4c3)cc2)c2ccc3oc4cc5ccccc5c(-c5cccc6ccccc56)c4c3c2)cc1 ; c1ccc(N(c2ccc(-c3ccc4ccccc4c3)cc2)c2ccc3oc4cc5ccccc5c(-c5ccc6ccccc6c5)c4c3c2)cc1]

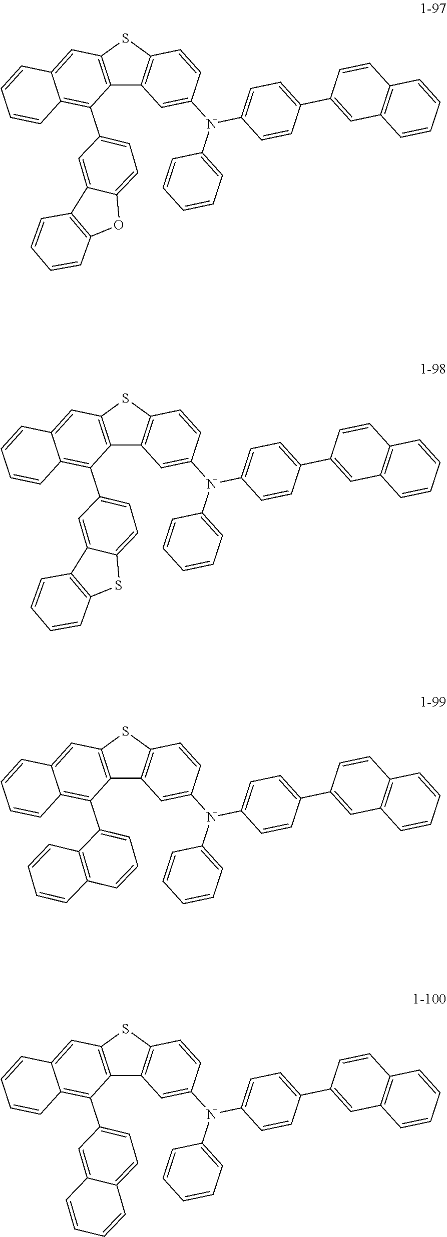 [SMILES: c1ccc(N(c2ccc(-c3ccc4ccccc4c3)cc2)c2ccc3sc4cc5ccccc5c(-c5ccc6oc7ccccc7c6c5)c4c3c2)cc1 ; c1ccc(N(c2ccc(-c3ccc4ccccc4c3)cc2)c2ccc3sc4cc5ccccc5c(-c5ccc6sc7ccccc7c6c5)c4c3c2)cc1 ; c1ccc(N(c2ccc(-c3ccc4ccccc4c3)cc2)c2ccc3sc4cc5ccccc5c(-c5cccc6ccccc56)c4c3c2)cc1 ; c1ccc(N(c2ccc(-c3ccc4ccccc4c3)cc2)c2ccc3sc4cc5ccccc5c(-c5ccc6ccccc6c5)c4c3c2)cc1]

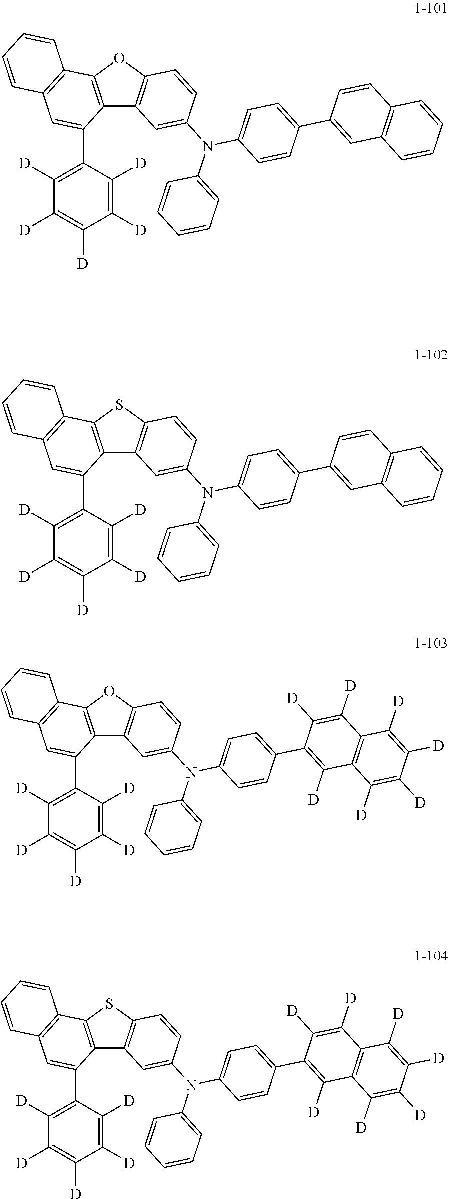 [SMILES: [2H]c1c([2H])c([2H])c(-c2cc3ccccc3c3oc4ccc(N(c5ccccc5)c5ccc(-c6ccc7ccccc7c6)cc5)cc4c23)c([2H])c1[2H] ; [2H]c1c([2H])c([2H])c(-c2cc3ccccc3c3sc4ccc(N(c5ccccc5)c5ccc(-c6ccc7ccccc7c6)cc5)cc4c23)c([2H])c1[2H] ; [2H]c1c([2H])c([2H])c(-c2cc3ccccc3c3oc4ccc(N(c5ccccc5)c5ccc(-c6c([2H])c([2H])c7c([2H])c([2H])c([2H])c([2H])c7c6[2H])cc5)cc4c23)c([2H])c1[2H] ; [2H]c1c([2H])c([2H])c(-c2cc3ccccc3c3sc4ccc(N(c5ccccc5)c5ccc(-c6c([2H])c([2H])c7c([2H])c([2H])c([2H])c([2H])c7c6[2H])cc5)cc4c23)c([2H])c1[2H]]

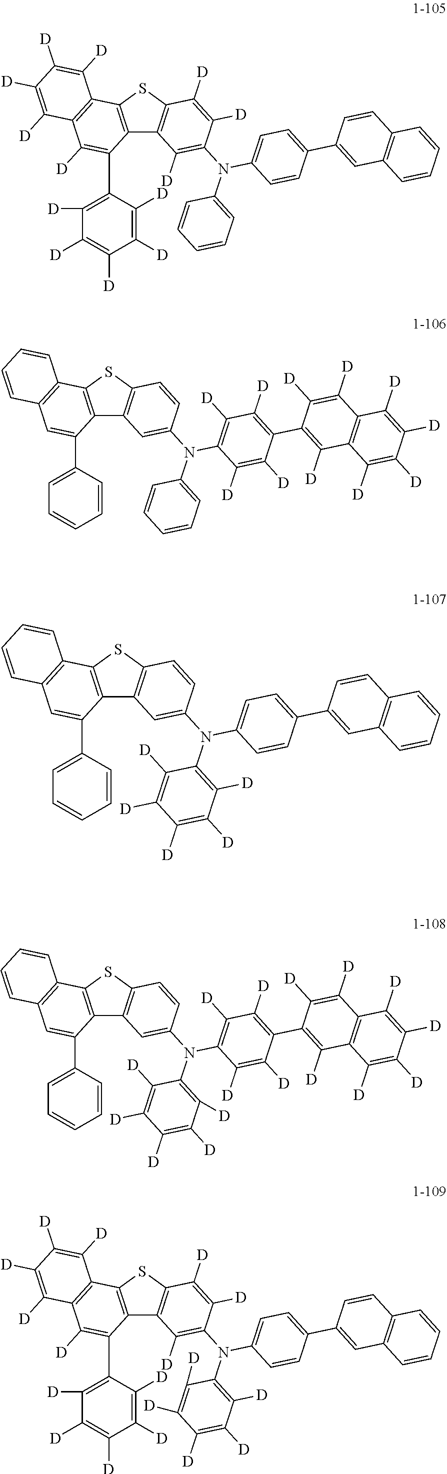

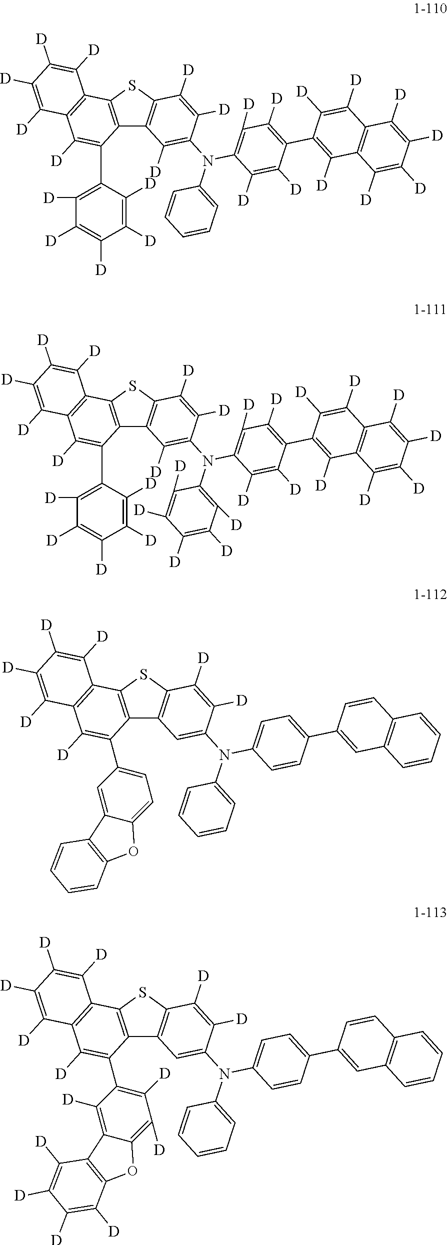

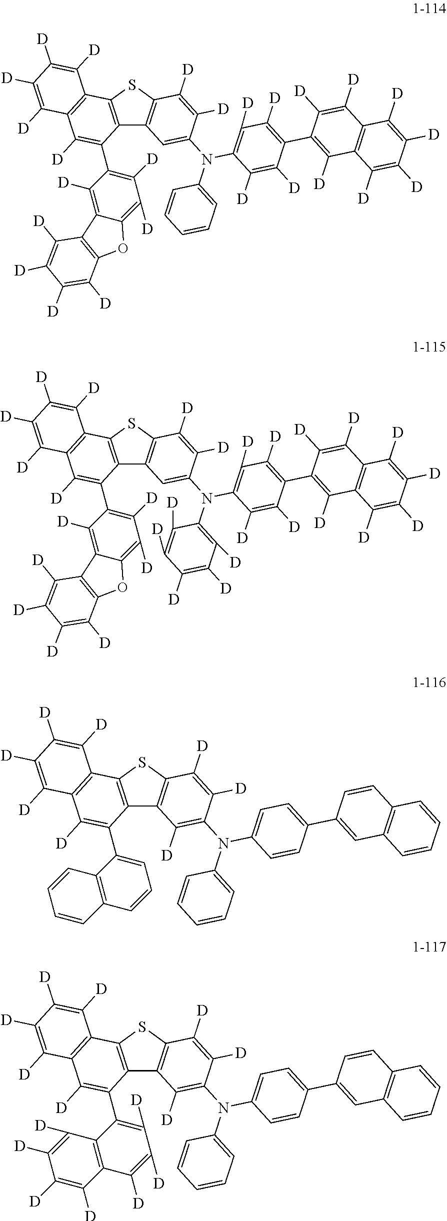

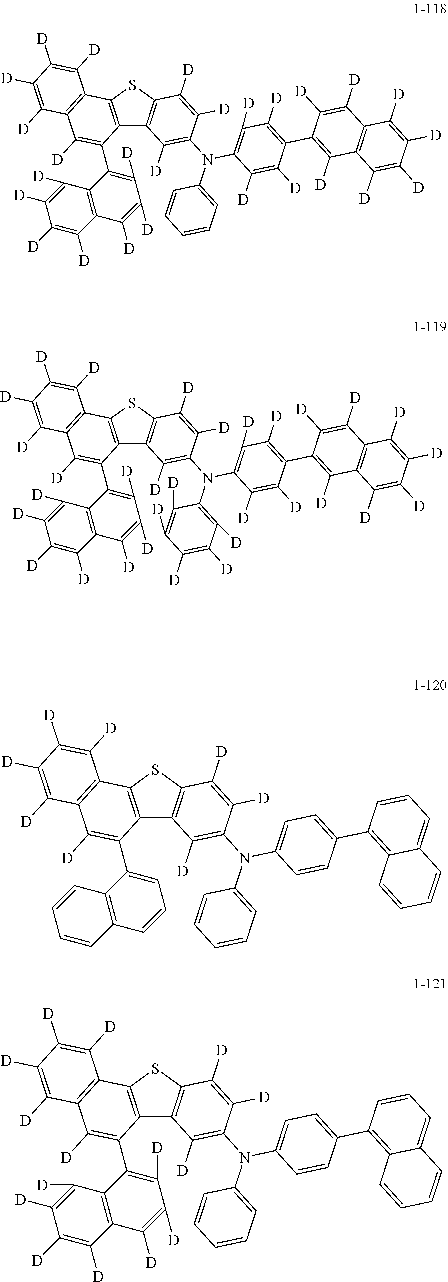

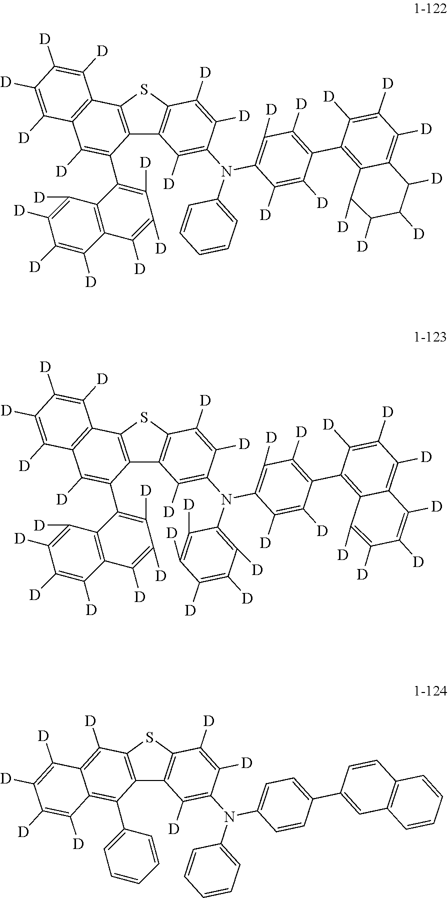

In an implementation, Chemical Formula 1 may be represented by, e.g., Chemical Formula 1A.

A composition for an organic optoelectronic device according to another embodiment may include, e.g., a first compound, and a second compound, wherein the first compound may be the aforementioned compound for an organic optoelectronic device and the second compound may be a compound for an organic optoelectronic device represented by Chemical Formula 2.

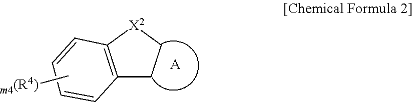

In Chemical Formula 2, X² may be, e.g., O, S, N-L^{a}-R^{a}, CR^{b}R^{c}, or SiR^{d}R^{e}.

L^{a} may be or may include, e.g., a single bond or a substituted or unsubstituted C6 to C12 arylene group.

R^{a}, R^{b}, R^{c}, R^{d}, R^{e}, and R⁴ may each independently be or include, e.g., hydrogen, deuterium, a substituted or unsubstituted C1 to C30 alkyl group, a substituted or unsubstituted C6 to C30 aryl group, or a substituted or unsubstituted C2 to C30 heterocyclic group.

m4 may be, e.g., an integer of 1 to 4.

A may be, e.g., a ring of Group III.

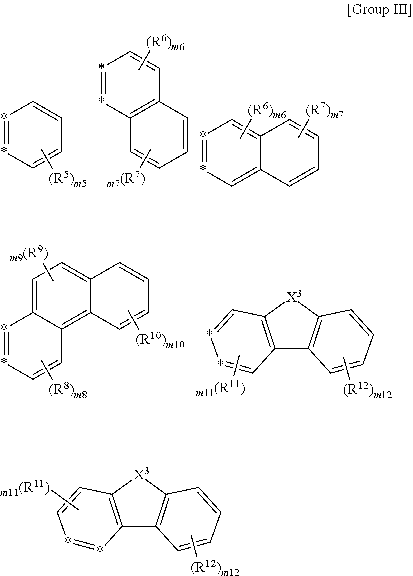

In Group III, * is a linking point.

X³ may be, e.g., O or S.

R⁵ to R¹² may each independently be or include, e.g., hydrogen, deuterium, a substituted or unsubstituted C1 to C30 alkyl group, a substituted or unsubstituted C6 to C30 aryl group, or a substituted or unsubstituted C2 to C30 heterocyclic group.

m5, m7, m10, and m12 may each independently be, e.g., an integer of 1 to 4.

m6, m8, m9, and m11 may each independently be, e.g., 1 or 2.

In an implementation, at least one of R^{a} and R⁴ to R¹² may be, e.g., a group represented by Chemical Formula a.

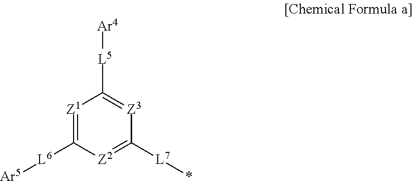

In Chemical Formula a, Z¹ to Z³ may each independently be, e.g., N or CR^{f}. In an implementation, at least two of Z¹ to Z³ may be N.

R^{f} may be or may include, e.g., hydrogen, deuterium, a substituted or unsubstituted C1 to C10 alkyl group, or a substituted or unsubstituted C6 to C20 aryl group.

L⁵ to L⁷ may each independently be or include, e.g., a single bond or a substituted or unsubstituted C6 to C30 arylene group.

Ar⁴ and Ar⁵ may each independently be or include, e.g., a substituted or unsubstituted C6 to C30 aryl group, or a substituted or unsubstituted C2 to C30 heterocyclic group.

* is a linking point.

The second compound may have, e.g., a structure substituted with a nitrogen-containing 6-membered ring.

The second compound may effectively extend an LUMO energy band by being substituted with a nitrogen-containing 6-membered ring, and when used in the light emitting layer together with the aforementioned first compound, a balance of holes and electrons may be increased to help improve luminous efficiency and life-span characteristics of a device including the same, and to help lower a driving voltage.

In an implementation, ring A of the second compound may be a ring of Group II, and the second compound may be, e.g., represented by one of Chemical Formula 2-I to Chemical Formula 2-X.

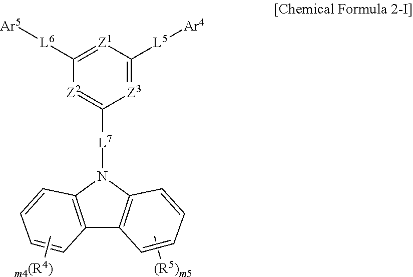

In Chemical Formula 2-I, Z¹ to Z³, R⁴, R⁵, L⁵ to L⁷, Ar⁴, Ar⁵, m4, and m5 may be defined the same as those described above.

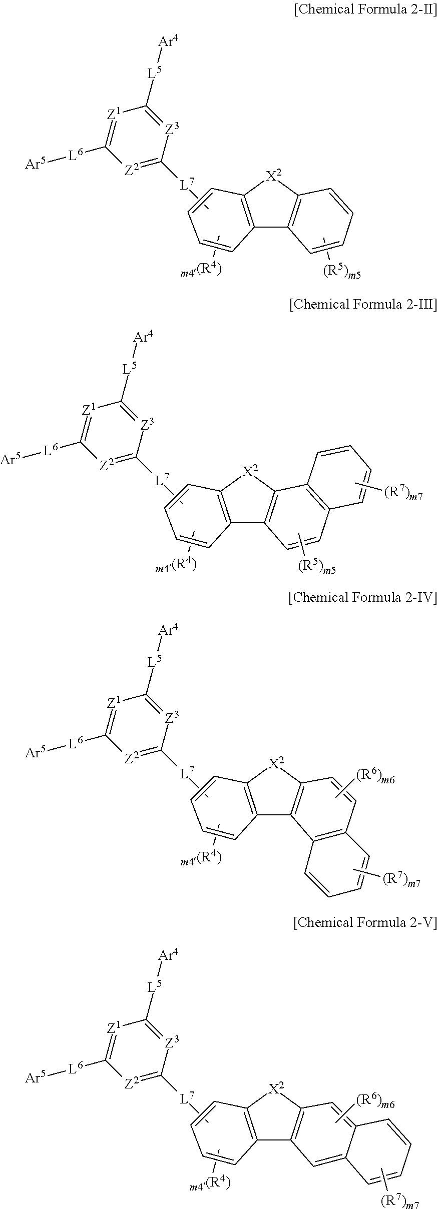

In Chemical Formula 2-II to Chemical Formula 2-V, X², Z¹ to Z³, R⁴ to R⁷, L⁵ to L⁷, Ar⁴, Ar⁵, and m5 to m7 may be defined the same as those described above.

m4′ may be, e.g., an integer of 1 to 3.

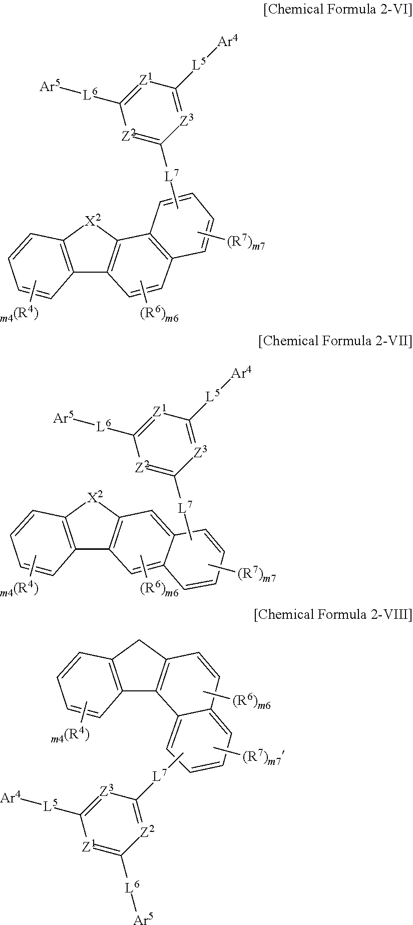

In Chemical Formula 2-VI to Chemical Formula 2-VIII, X², Z¹ to Z³, R⁴, R⁶, R⁷, L⁵ to L⁷, Ar⁴, Ar⁵, m4, and m6 may be defined the same as those described above.

m7′ may be, e.g., an integer of 1 to 3.

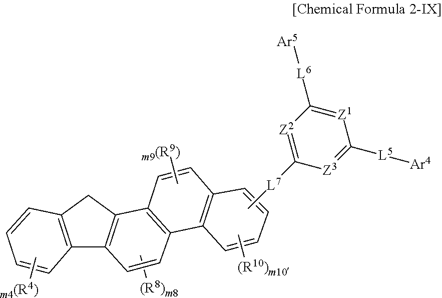

In Chemical Formula 2-IX, X², Z¹ to Z³, R⁴, R⁸ to R¹⁰, L⁵ to L⁷, Ar⁴, Ar⁵, m4, m8, and m9 may be defined the same as those described above.

m10′ may be, e.g., an integer of 1 to 3.

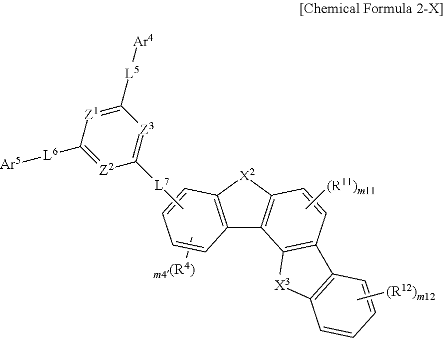

In Chemical Formula 2-X, X², X³, Z¹ to Z³, R⁴, R¹¹, R¹², L⁵ to L⁷, Ar⁴, Ar⁵, m11, and m12 may be defined the same as those described above.

m4′ may be, e.g., an integer of 1 to 3.

In an implementation, the second compound may be represented by, e.g., Chemical Formula 2-II, Chemical Formula 2-III, or Chemical Formula 2-VI.

In an implementation, the second compound may be represented by, e.g., Chemical Formula 2-II-3, Chemical Formula 2-III-1, Chemical Formula 2-VI-1, or Chemical Formula 2-VI-3.

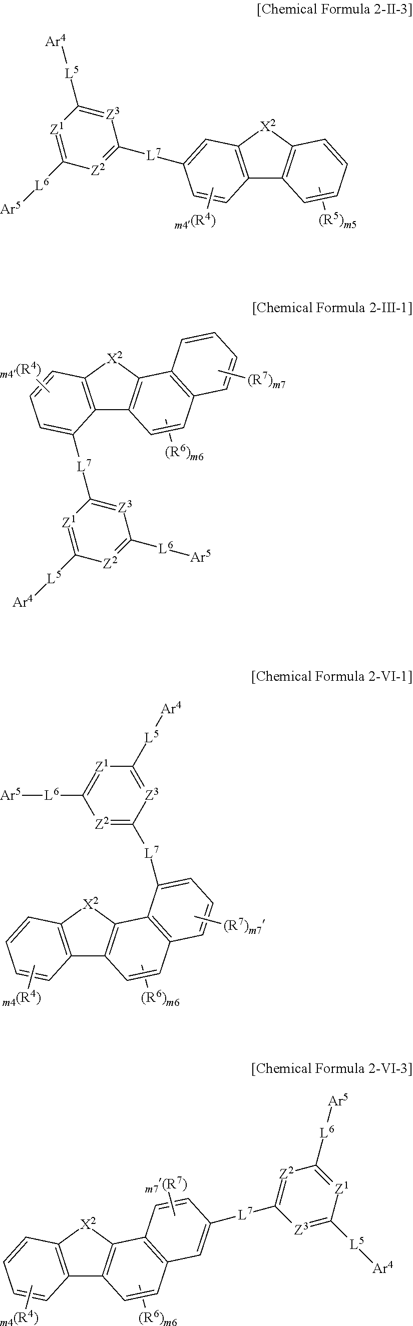

In Chemical Formula 2-II-3, Chemical Formula 2-III-1, Chemical Formula 2-VI-1, and Chemical Formula 2-VI-3, X², Z¹ to Z³, R⁴ to R⁷, L⁵ to L⁷, Ar⁴, Ar⁵, m4 to m7, m4′, and m7′ may be defined the same as those described above.

In an implementation, Ar⁴ and Ar⁵ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylene group, a substituted or unsubstituted carbazolyl group, a substituted or unsubstituted dibenzofuranyl group, a substituted or unsubstituted dibenzothiophenyl group, or a substituted or unsubstituted dibenzosilolyl group.

In an implementation, Ar⁴ and Ar⁵ may each independently be, e.g., a substituted or unsubstituted phenyl group, a substituted or unsubstituted biphenyl group, or a substituted or unsubstituted naphthyl group.

In an implementation, L⁵ to L⁷ may each independently be, e.g., a single bond, a substituted or unsubstituted phenylene group or a substituted or unsubstituted biphenylene group.

In an implementation, L⁵ may be, e.g., a single bond or a substituted or unsubstituted phenylene group. In an implementation, L⁶ and L⁷ may each independently be, e.g., a single bond, a substituted or unsubstituted phenylene group, or a substituted or unsubstituted biphenylene group.

In an implementation, L⁵ may be a single bond and L⁶ and L⁷ may each independently be a single bond or a substituted or unsubstituted phenylene group.

In an implementation, R⁴ to R¹² may each independently be, e.g., hydrogen, deuterium, a substituted or unsubstituted C1 to C10 alkyl group, a substituted or unsubstituted C6 to C12 aryl group, or a substituted or unsubstituted C2 to C18 heterocyclic group.

In an implementation, R⁴ to R¹² may each independently be, e.g., hydrogen, deuterium, a substituted or unsubstituted phenyl group, or a substituted or unsubstituted naphthyl group.

In an implementation, X² may be, e.g., O, S, CR^{b}R^{c}, or SiR^{d}R^{e}, in which R^{b}, R^{c}, R^{d}, and Re may each independently be, e.g., a substituted or unsubstituted C1 to C10 alkyl group or a substituted or unsubstituted C6 to C20 aryl group.

In an implementation, R^{b}, R^{c}, R^{d}, and Re may each independently be, e.g., a substituted or unsubstituted methyl group, a substituted or unsubstituted phenyl group, or a substituted or unsubstituted biphenyl group.

In an implementation, the second compound may be, e.g., a compound of Group 2.

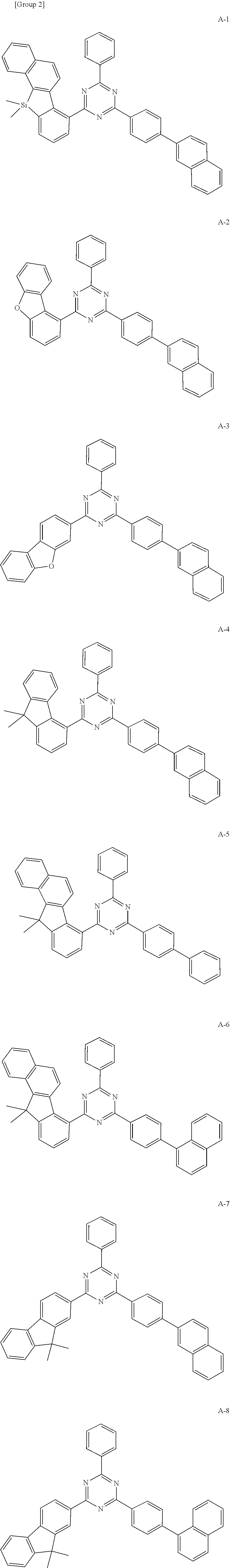

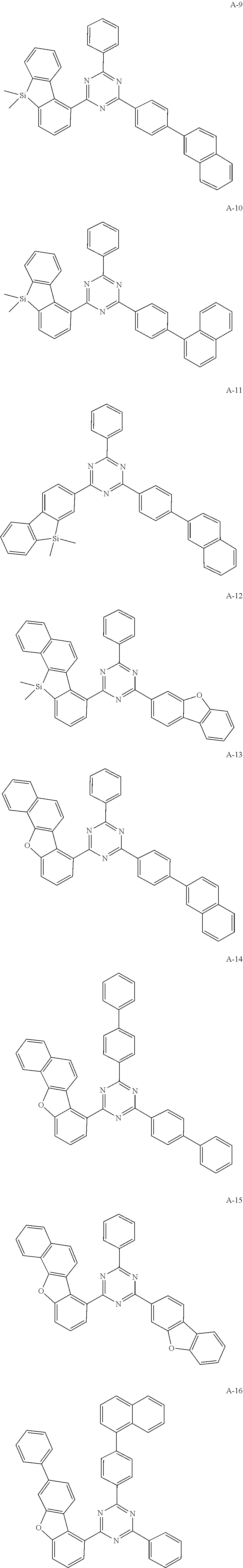

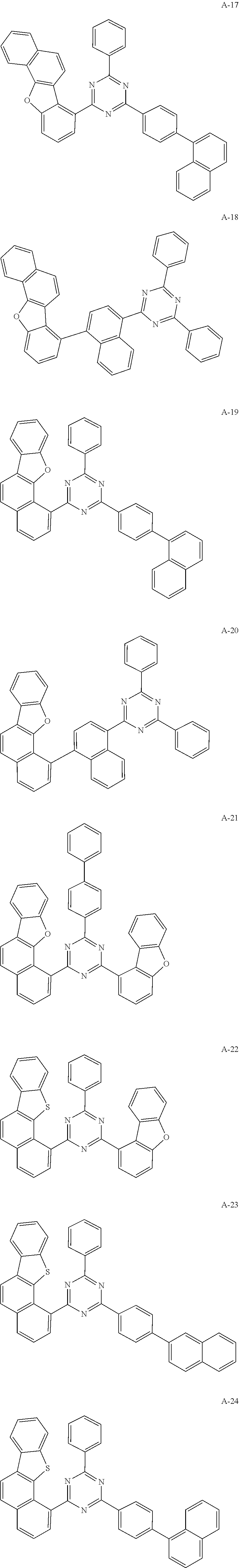

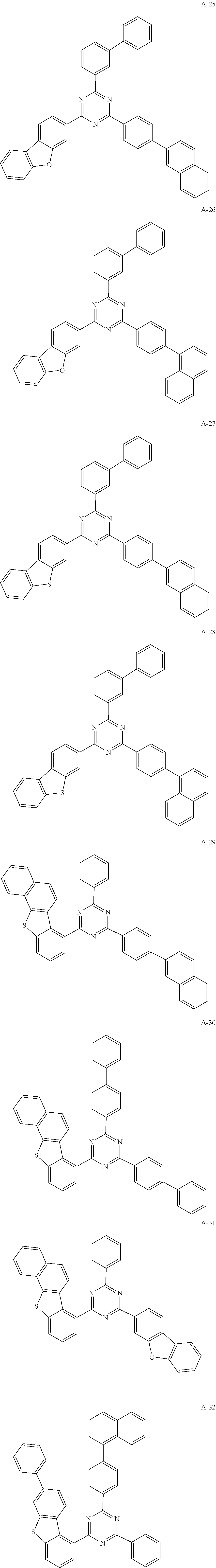

[chemical structure]

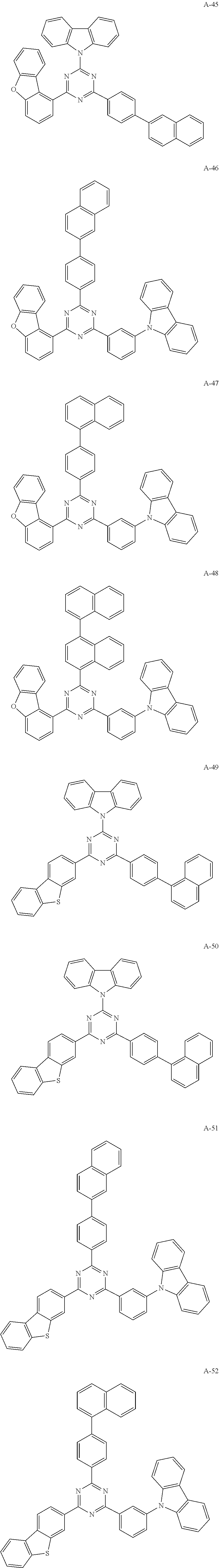

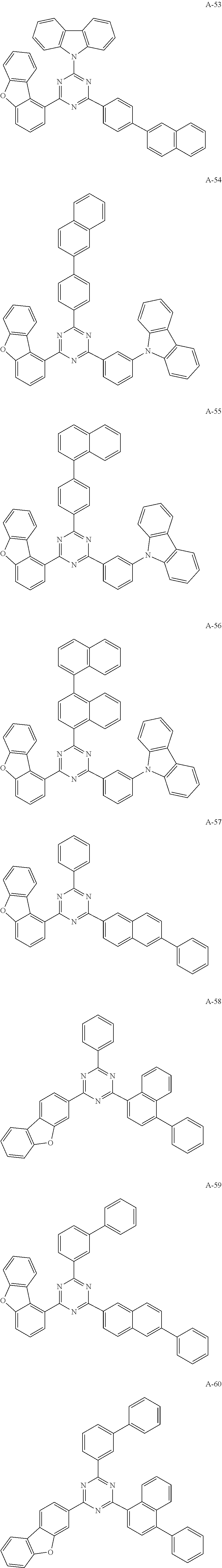

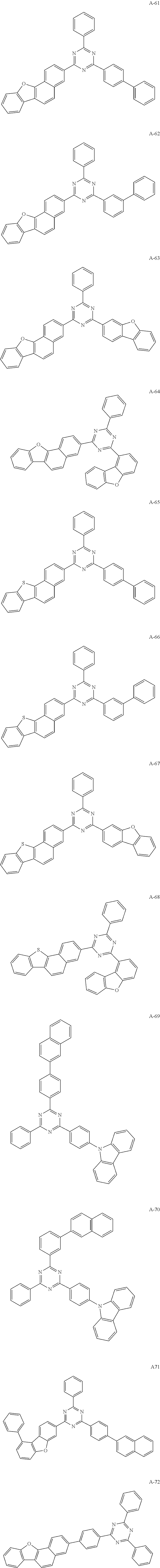

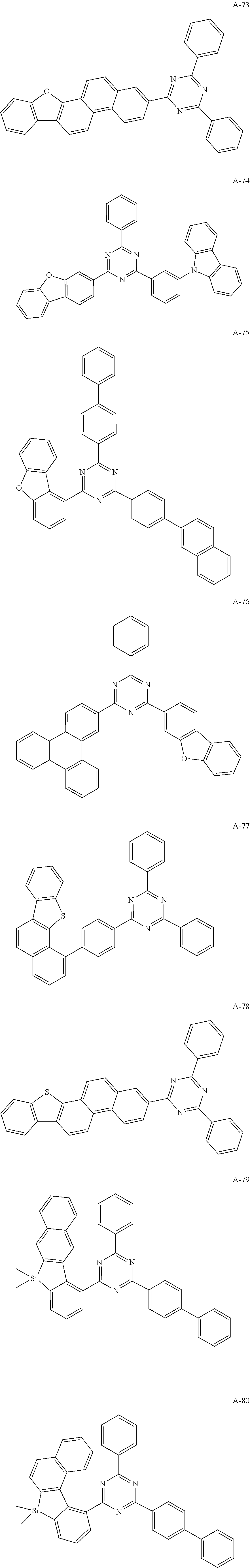

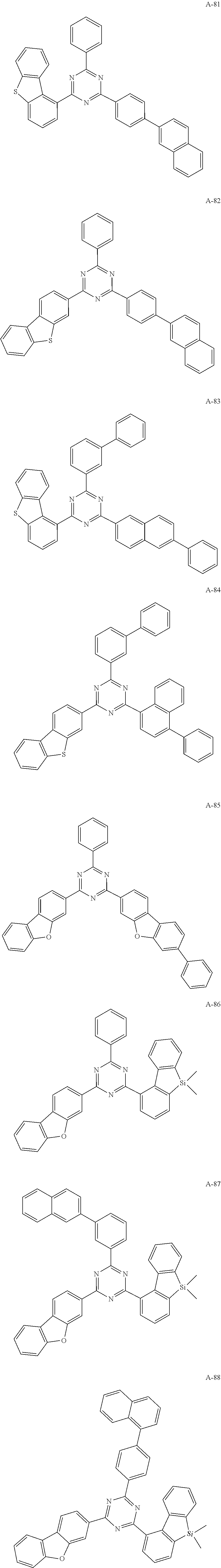

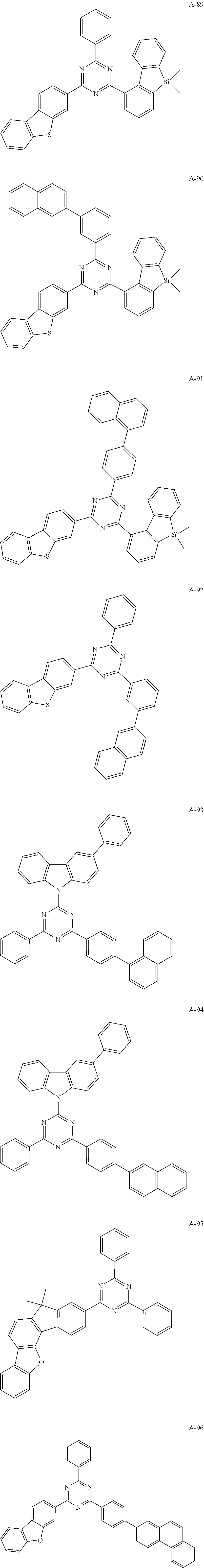

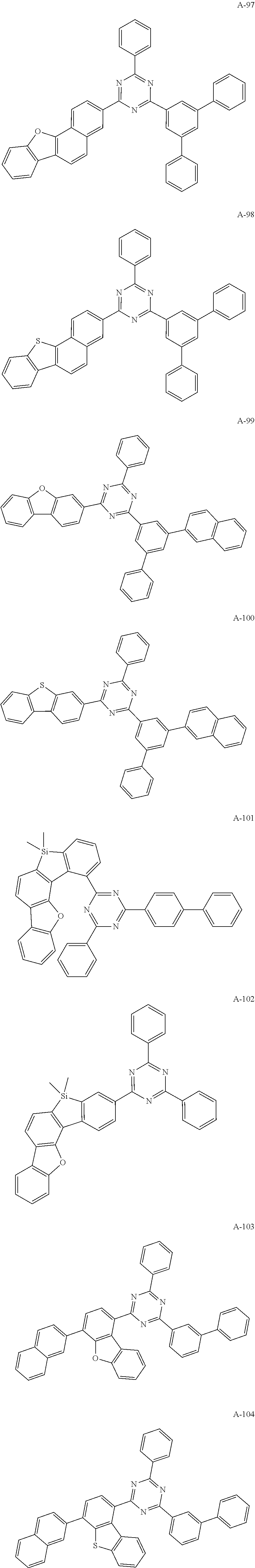

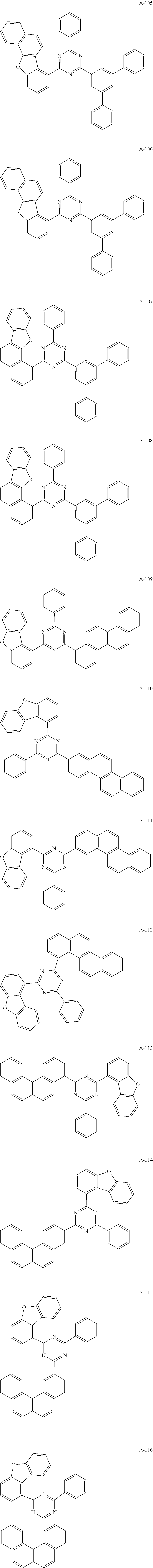

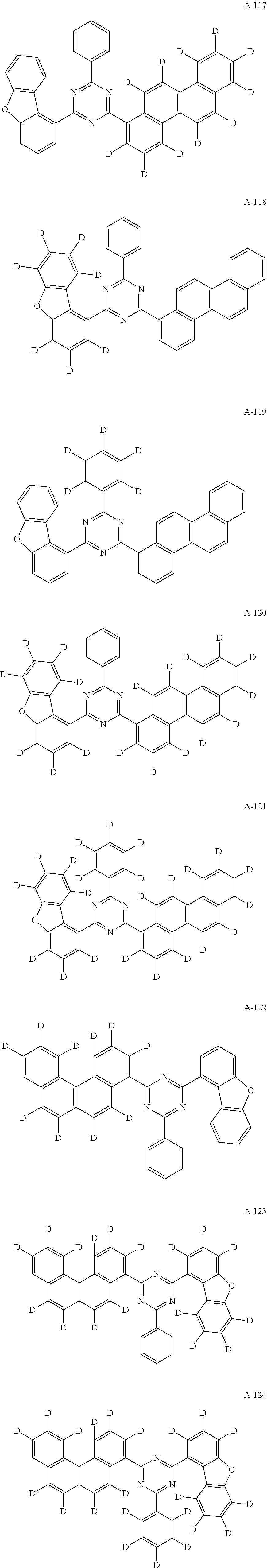

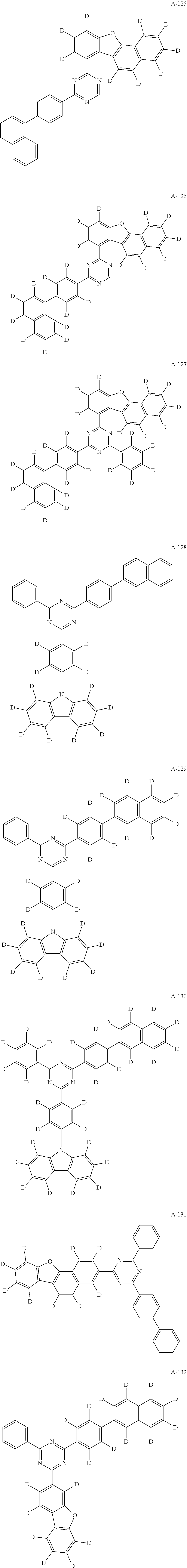

In an implementation, the composition for an organic optoelectronic device may include, e.g., the first compound represented by Chemical Formula 1 A or Chemical Formula 1B and the second compound represented by one of Chemical Formula 2-II-3, Chemical Formula 2-III-1, and Chemical Formula 2-VI-3.

The first compound and the second compound may be included (e.g., mixed) in a weight ratio of about 1:99 to about 99:1. Within the range, a desirable weight ratio may be adjusted using an electron transport capability of the first compound and a hole transport capability of the second compound to realize bipolar characteristics and thus to improve efficiency and life-span. Within the range, they may be, e.g., included in a weight ratio of about 10:90 to about 90:10, or about 20:80 to about 80:20, e.g., a weight ratio of about 20:80 to about 70:30, about 20:80 to about 60:40, and about 30:70 to about 60:40. In an implementation, they may be included in a weight ratio of about 40:60, about 50:50, or about 60:40.

One or more compounds may be further included in addition to the aforementioned first and second compounds.

The aforementioned compound for an organic optoelectronic device or composition for an organic optoelectronic device may be a composition that further includes a dopant.

The dopant may be, e.g., a phosphorescent dopant, such as a red, green, or blue phosphorescent dopant, and may be, e.g., a red or green phosphorescent dopant.

The dopant is a material mixed with the compound or composition for an organic optoelectronic device in a small amount to cause light emission and may be generally a material such as a metal complex that emits light by multiple excitation into a triplet or more. The dopant may be, e.g., an inorganic, organic, or organic-inorganic compound, and one or more types thereof may be used.

Examples of the dopant may include a phosphorescent dopant and examples of the phosphorescent dopant may include an organic metal compound including Ir, Pt, Os, Ti, Zr, Hf, Eu, Tb, Tm, Fe, Co, Ni, Ru, Rh, Pd, or a combination thereof. In an implementation, the phosphorescent dopant may be, e.g., a compound represented by Chemical Formula Z.

L⁸MX⁴ [Chemical Formula Z]

In Chemical Formula Z, M may be a metal, and L⁸ and X⁴ may each independently be ligands forming a complex compound with M.

M may be, e.g., Ir, Pt, Os, Ti, Zr, Hf, Eu, Tb, Tm, Fe, Co, Ni, Ru, Rh, Pd, or a combination thereof, and L⁸ and X⁴ may be, e.g., a bidentate ligand.

In an implementation, the ligands represented by L⁸ and X⁴ may be ligands of Group A.

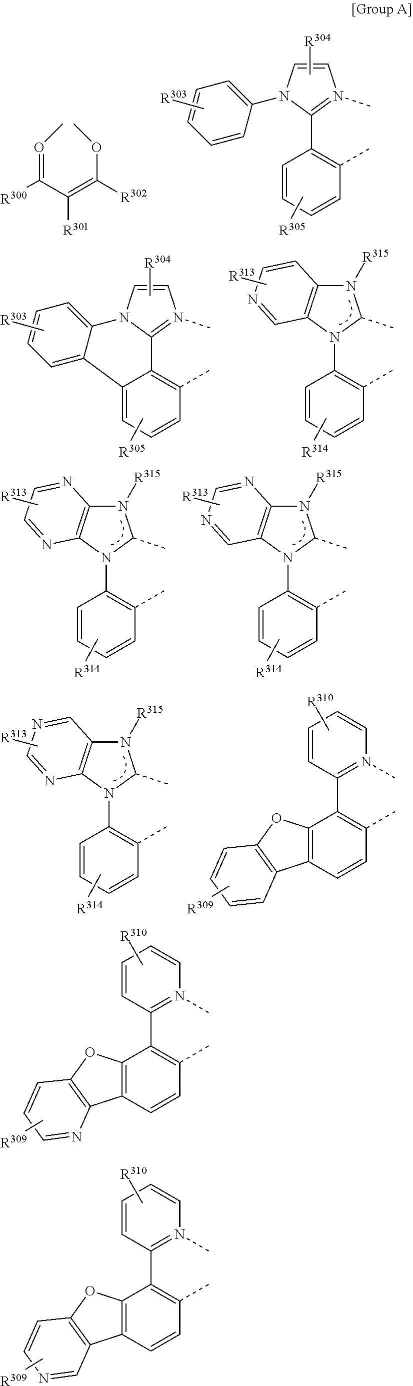

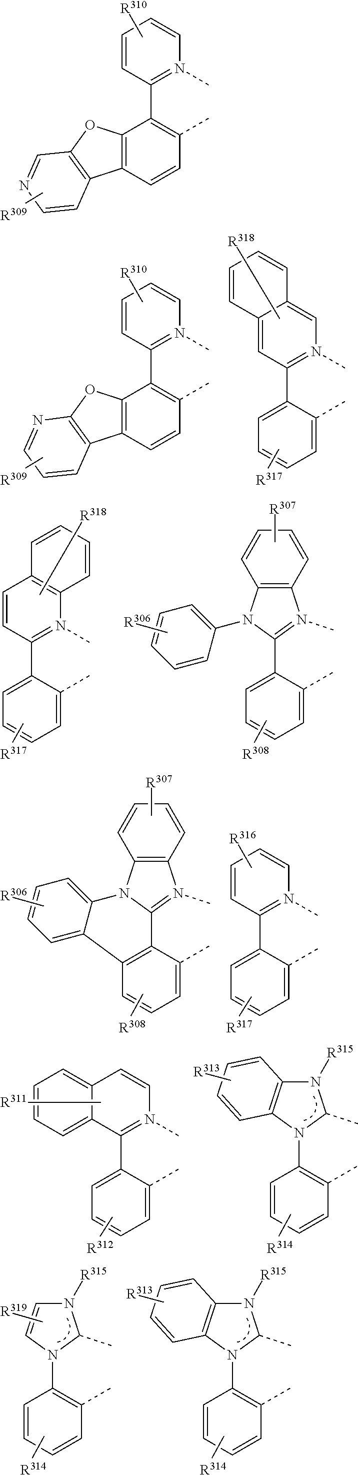

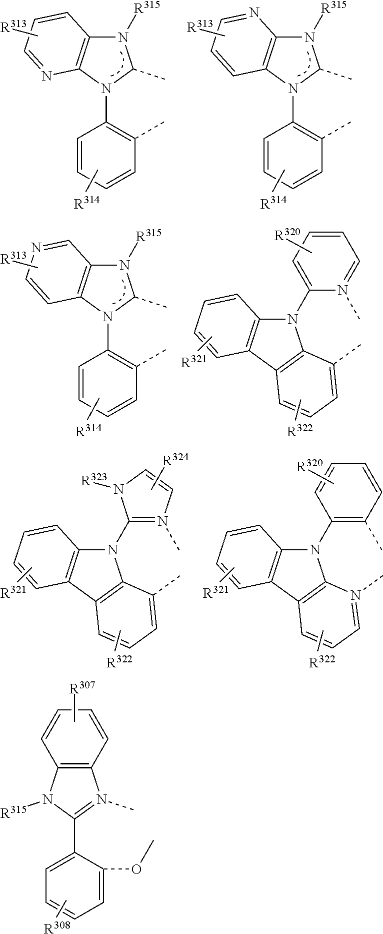

In Group A,

R³⁰⁰ to R³⁰² may each independently be, e.g., hydrogen, deuterium, a C1 to C30 alkyl group that is substituted or unsubstituted with a halogen, a C6 to C30 aryl group that is substituted or unsubstituted with a C1 to C30 alkyl, or a halogen, and

R³⁰³ to R³²⁴ may each independently be, e.g., hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

In an implementation, it may include a dopant represented by Chemical Formula V.

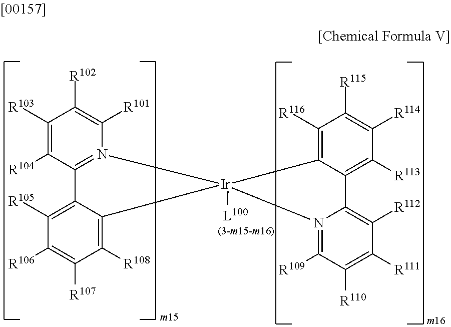

In Chemical Formula V,

R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ may each independently be, e.g., a C1 to C6 alkyl group,

at least one of R¹⁰¹ to R¹¹⁶ may be a functional group represented by Chemical Formula V-1,

L¹⁰⁰ may be a bidentate ligand of a monovalent anion, and is a ligand that coordinates to iridium through a lone pair of electrons of carbon or heteroatom,

m15 and m16 may each independently be an integer of 0 to 3, and

m15+m16 may be an integer of 1 to 3.

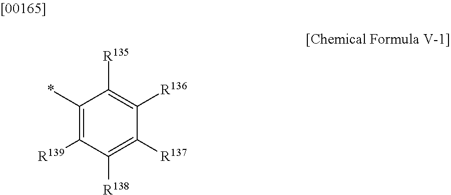

In Chemical Formula V-1,

R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ may each independently be, e.g., a C1 to C6 alkyl group, and

* is a linking site.

In an implementation, a dopant represented by Chemical Formula Z-1 may be included.

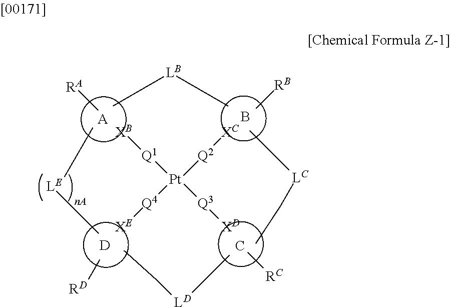

In Chemical Formula Z-1, rings A, B, C, and D may each independently be, e.g., a 5-membered or 6-membered carbocyclic or heterocyclic ring;

R^{A}, R^{B}, R^{c}, and RD may each independently be, e.g., mono-, di-, tri-, or tetra-substitution, or unsubstitution;

L^{B}, L^{C}, and L^{D} may each independently be, e.g., a direct bond, BR, NR, PR, O, S, Se, C═O, S═O, SO₂, CRR′, SiRR′, GeRR′, and a combination thereof;

when nA is 1, L^{E} may be a direct bond, BR, NR, PR, O, S, Se, C═O, S═O, SO₂, CRR′, SiRR′, GeRR′, or a combination thereof, when nA is 0, L^{E} does not exist; and

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ are optionally linked to each other to provide a ring; X^{B}, X^{C}, X^{D}, and X^{E} may each independently be, e.g., carbon and nitrogen; and Q¹, Q², Q³, and Q⁴ may each independently be, e.g., oxygen or a direct bond.

The dopant according to an embodiment may be a platinum complex, and may be, e.g., represented by Chemical Formula VI.

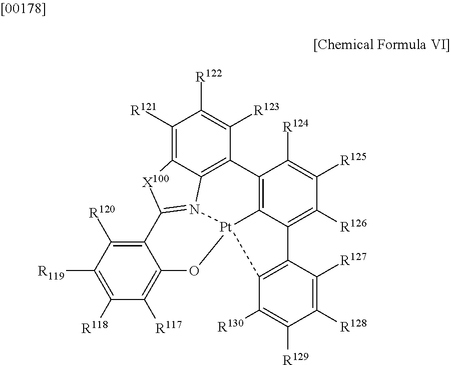

In Chemical Formula VI,

X¹⁰⁰ may be O, S, or NR¹³¹,

R¹¹⁷ to R¹³¹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ may each independently be, e.g., a C1 to C6 alkyl group, and

at least one of R¹¹⁷ to R¹³¹ may be —SiR¹³²R¹³³R¹³⁴ or a tert-butyl group.

Hereinafter, an organic optoelectronic device including the aforementioned compound for an organic optoelectronic device or composition for an organic optoelectronic device is described.

The organic optoelectronic device may be a suitable device to convert electrical energy into photoenergy and vice versa, e.g., an organic photoelectric device, an organic light emitting diode, an organic solar cell, or an organic photo-conductor drum.

Herein, an organic light emitting diode as one example of an organic optoelectronic device is described referring to the drawing.

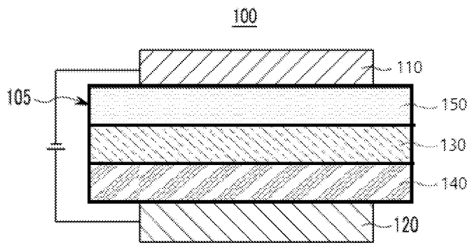

The FIG. 1s a cross-sectional view illustrating an organic light emitting diode according to an embodiment.

Referring to the FIGURE, an organic light emitting diode 100 according to an embodiment may include an anode 120 and a cathode 110 facing each other and an organic layer 105 between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, or the like or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; a multi-layer structure material such as LiF/Al, LiO₂/Al, LiF/Ca, LiF/Al, or BaF₂/Ca.

The organic layer 105 may include the aforementioned compound for an organic optoelectronic device or composition for an organic optoelectronic device.

The organic layer 105 may include a light emitting layer 130 and the light emitting layer 130 may include the aforementioned compound for an organic optoelectronic device or composition for an organic optoelectronic device.

The composition for an organic optoelectronic device further including the dopant may be, e.g., a red light emitting composition.

The light emitting layer 130 may include, e.g., the aforementioned compound for an organic optoelectronic device or composition for an organic optoelectronic device as each phosphorescent host.

The organic layer may further include a charge transport region in addition to the light emitting layer.

The charge transport region may be, e.g., a hole transport region 140.

The hole transport region 140 may further increase hole injection and/or hole mobility between the anode 120 and the light emitting layer 130 and block electrons. In an implementation, the hole transport region 140 may include a hole transport layer between the anode 120 and the light emitting layer 130, and a hole transport auxiliary layer between the light emitting layer 130 and the hole transport layer and a compound of Group B may be included in at least one of the hole transport layer and the hole transport auxiliary layer.

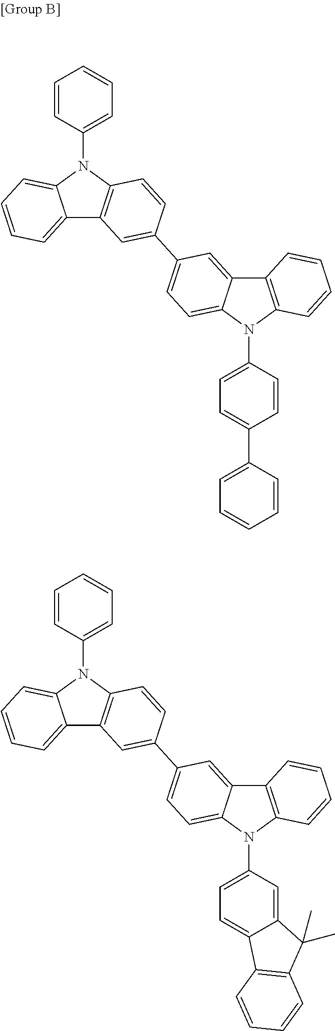 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21]

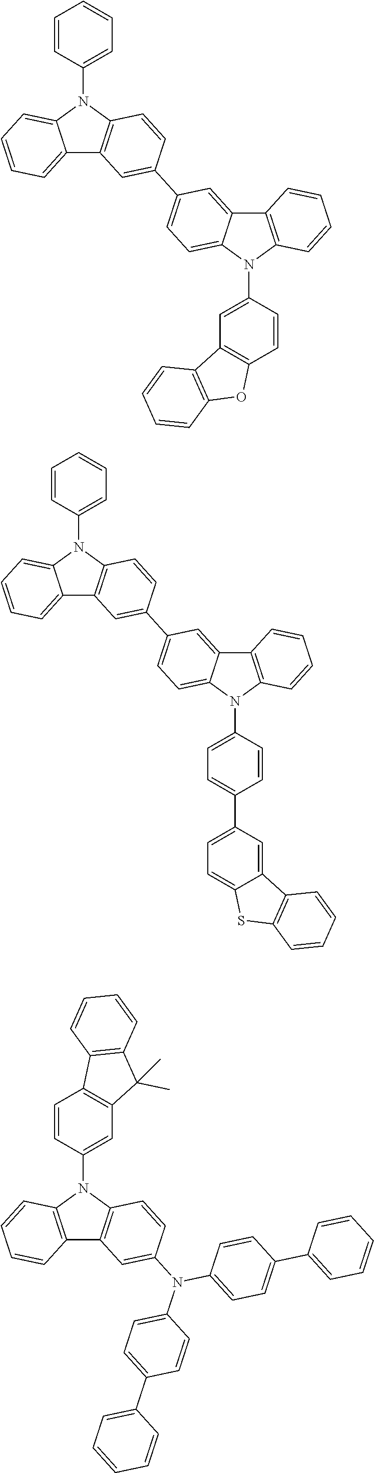 [SMILES: c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1 ; CC1(C)c2ccccc2-c2ccc(-n3c4ccccc4c4cc(N(c5ccc(-c6ccccc6)cc5)c5ccc(-c6ccccc6)cc5)ccc43)cc21]

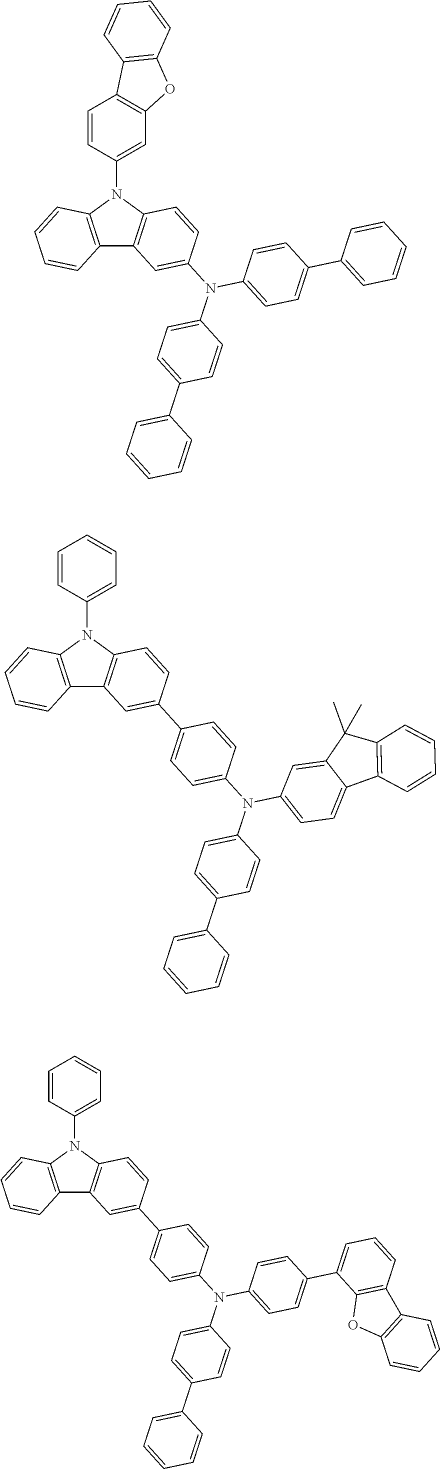 [SMILES: c1ccc(-c2ccc(N(c3ccc(-c4ccccc4)cc3)c3ccc4c(c3)c3ccccc3n4-c3ccc4c(c3)oc3ccccc34)cc2)cc1 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

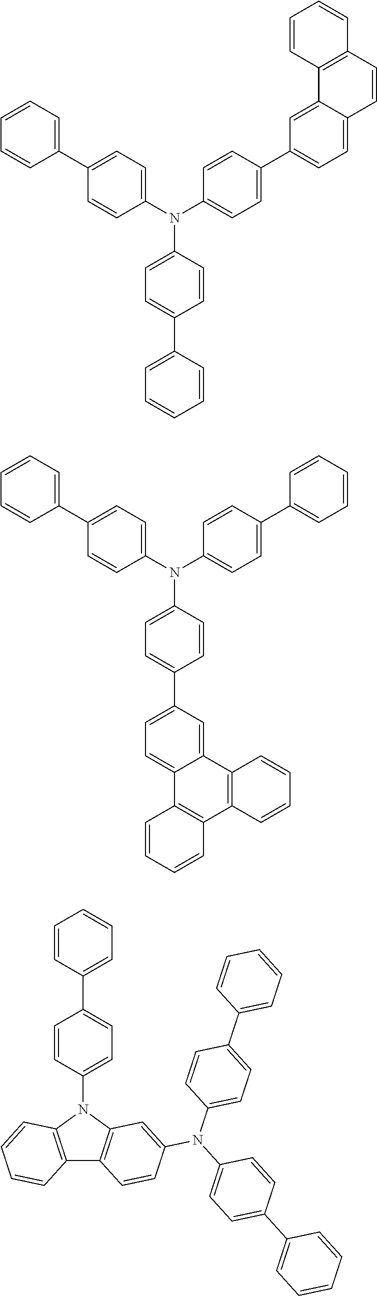 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

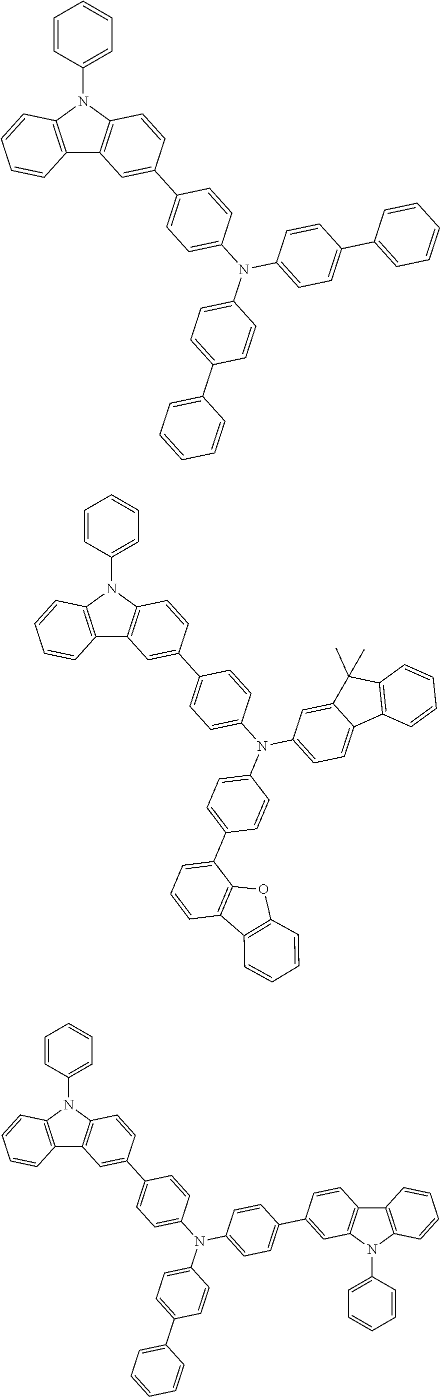 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

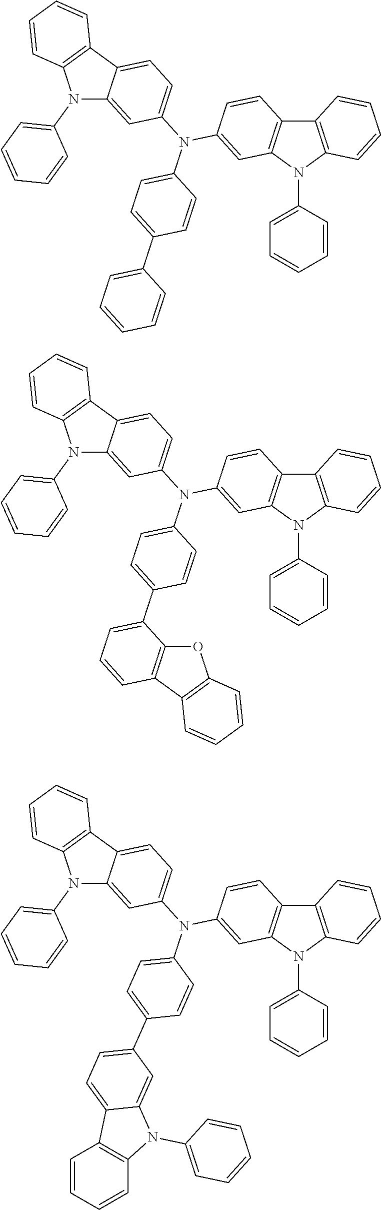 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

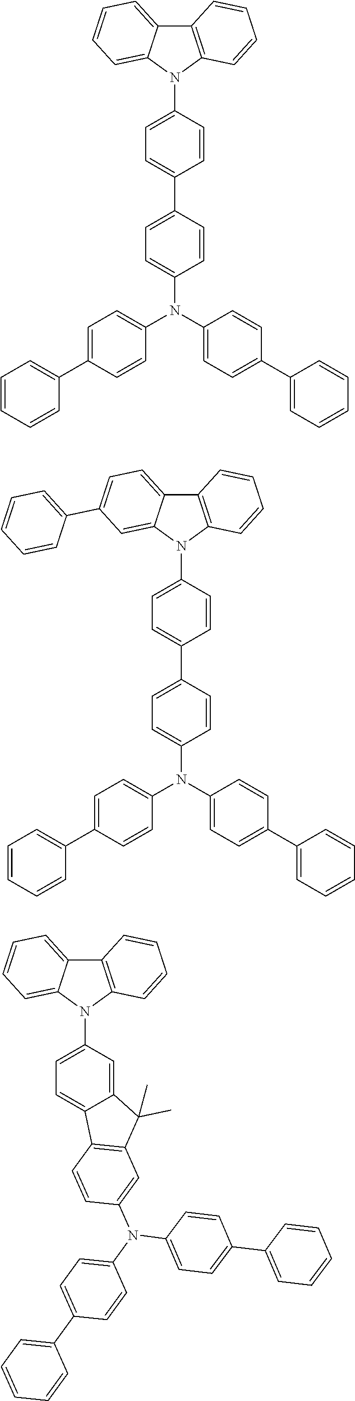

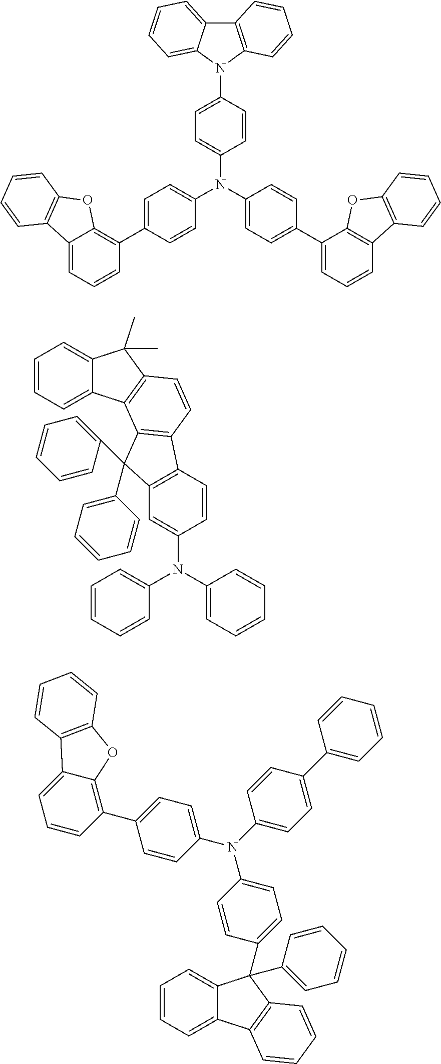 [SMILES: c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1 ; c1ccc(-c2ccc(N(c3ccc(-c4cccc5c4oc4ccccc45)cc3)c3ccc(C4(c5ccccc5)c5ccccc5-c5ccccc54)cc3)cc2)cc1]

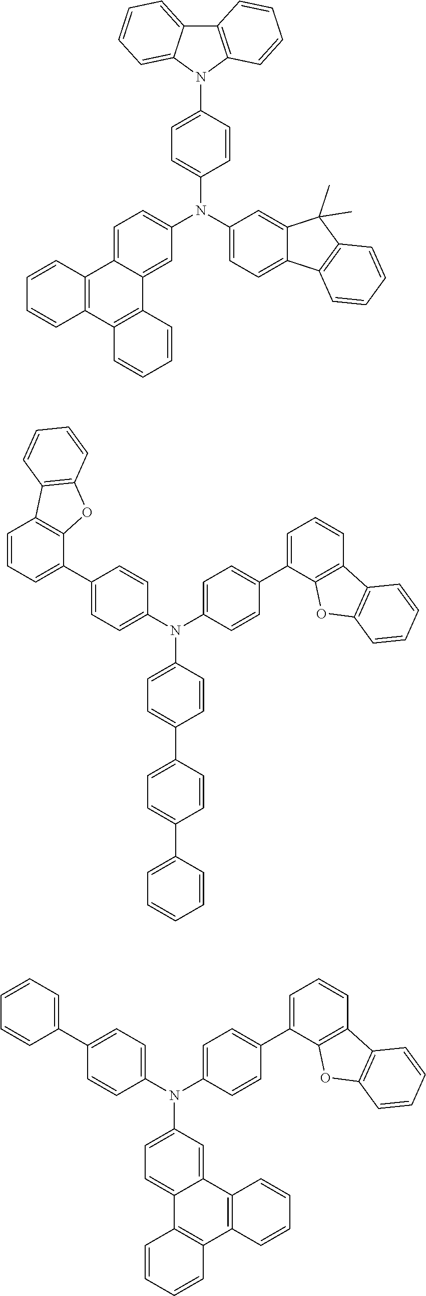 [SMILES: CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1]

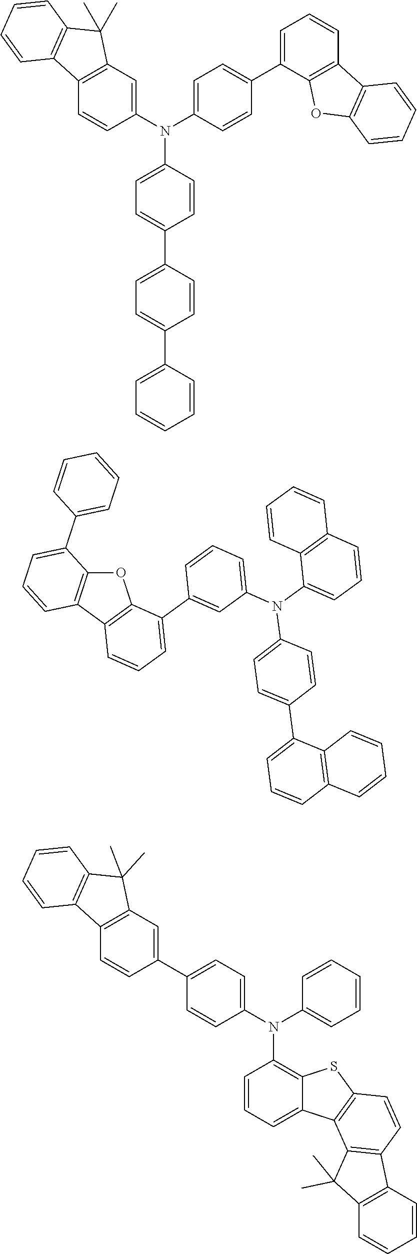 [SMILES: CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21]

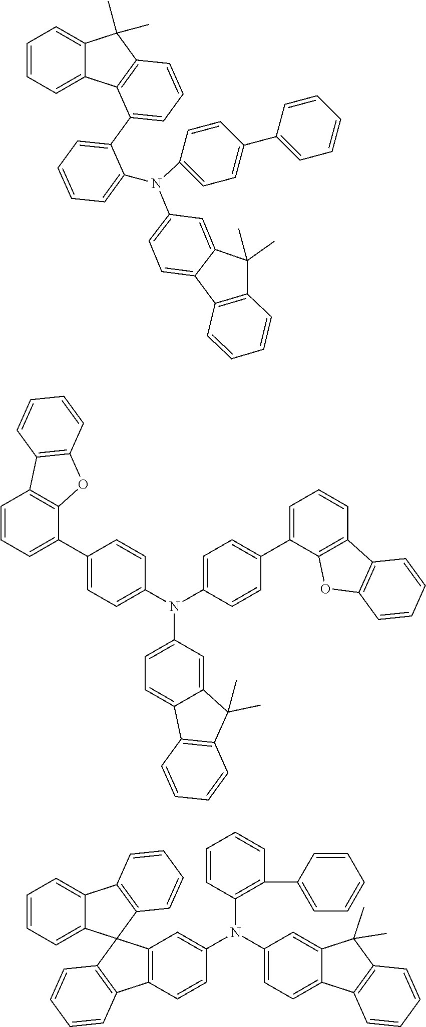

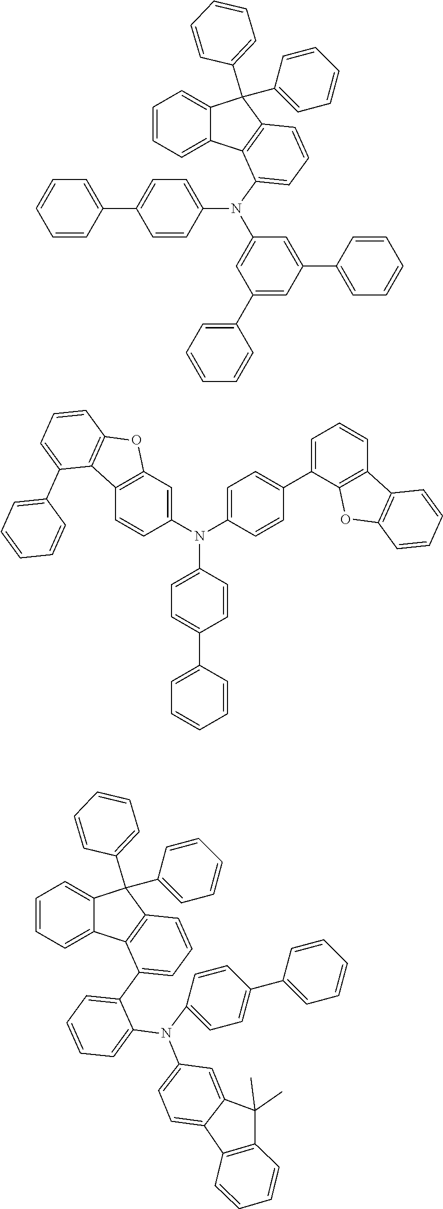 [SMILES: c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1 ; CC1(C)c2ccccc2-c2ccc(N(c3ccc(-c4ccccc4)cc3)c3ccccc3-c3cccc4c3-c3ccccc3C4(c3ccccc3)c3ccccc3)cc21]

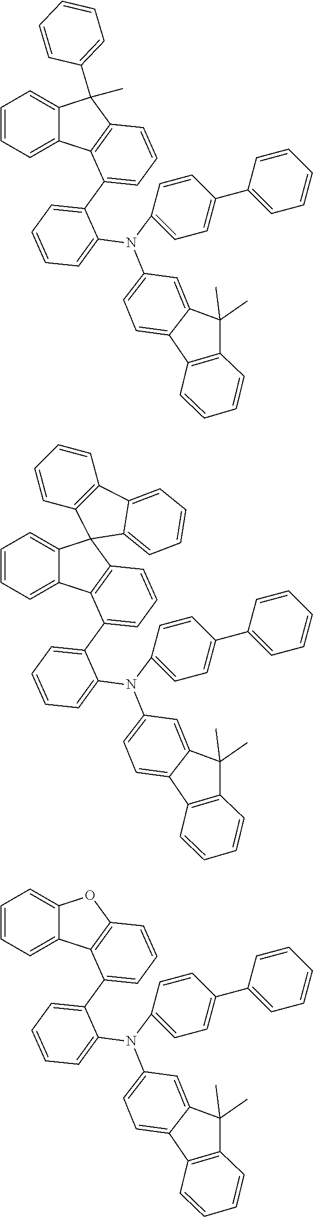 [SMILES: CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21 ; CC1(C)c2ccccc2-c2ccc(N(c3ccc(-c4ccccc4)cc3)c3ccccc3-c3cccc4oc5ccccc5c34)cc21]

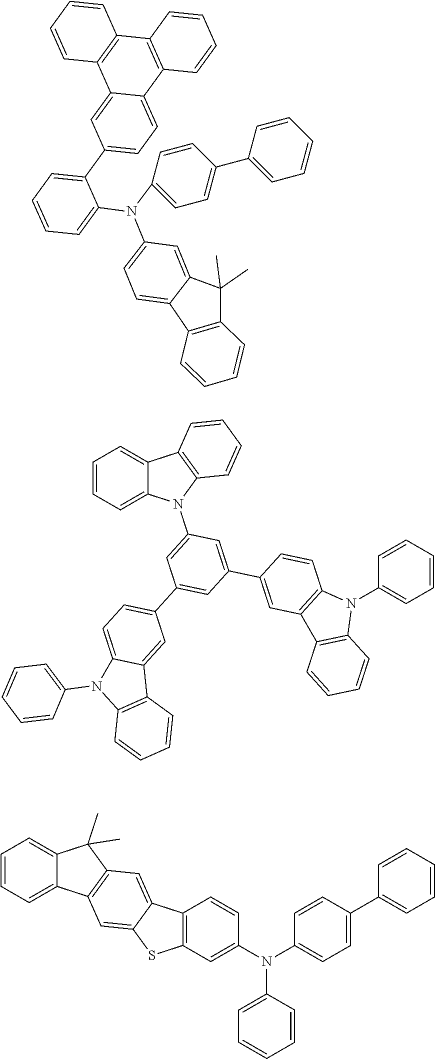

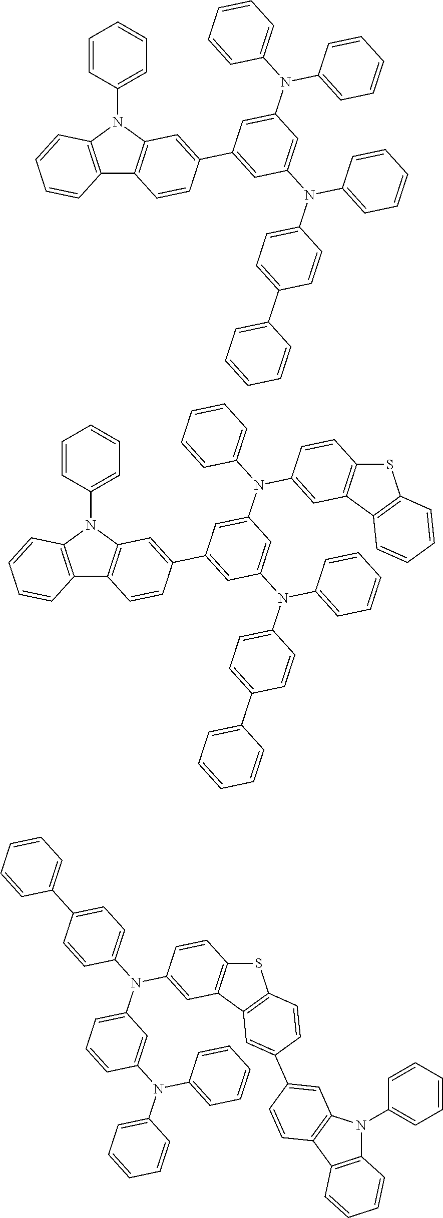

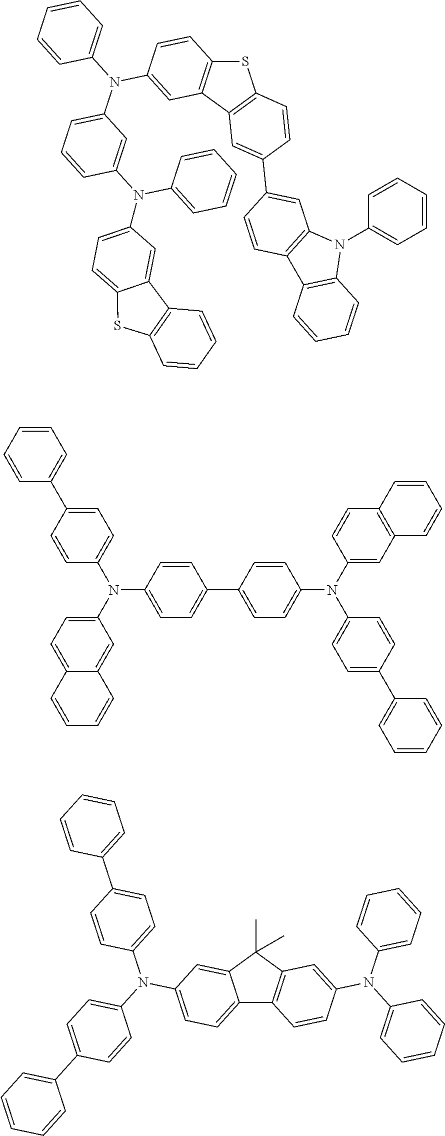 [SMILES: c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1 ; CC1(C)c2cc(N(c3ccccc3)c3ccccc3)ccc2-c2ccc(N(c3ccc(-c4ccccc4)cc3)c3ccc(-c4ccccc4)cc3)cc21]

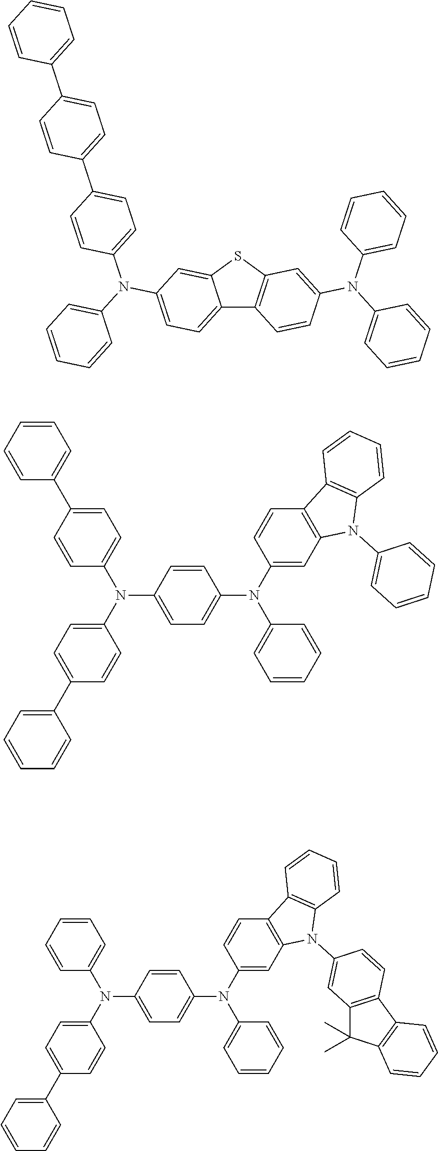 [SMILES: c1ccc(-c2ccc(-c3ccc(N(c4ccccc4)c4ccc5c(c4)sc4cc(N(c6ccccc6)c6ccccc6)ccc45)cc3)cc2)cc1 ; c1ccc(-c2ccc(N(c3ccc(-c4ccccc4)cc3)c3ccc(N(c4ccccc4)c4ccc5c6ccccc6n(-c6ccccc6)c5c4)cc3)cc2)cc1 ; CC1(C)c2ccccc2-c2ccc(-n3c4ccccc4c4ccc(N(c5ccccc5)c5ccc(N(c6ccccc6)c6ccc(-c7ccccc7)cc6)cc5)cc43)cc21]

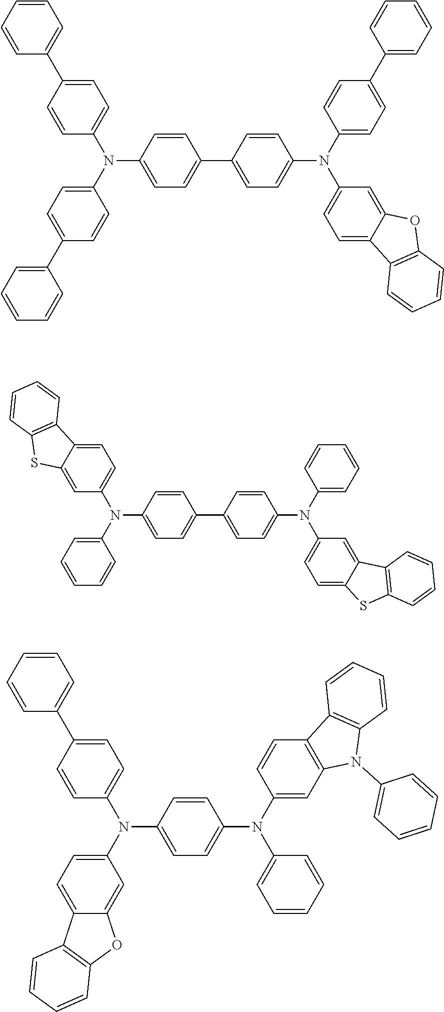 [SMILES: c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1]

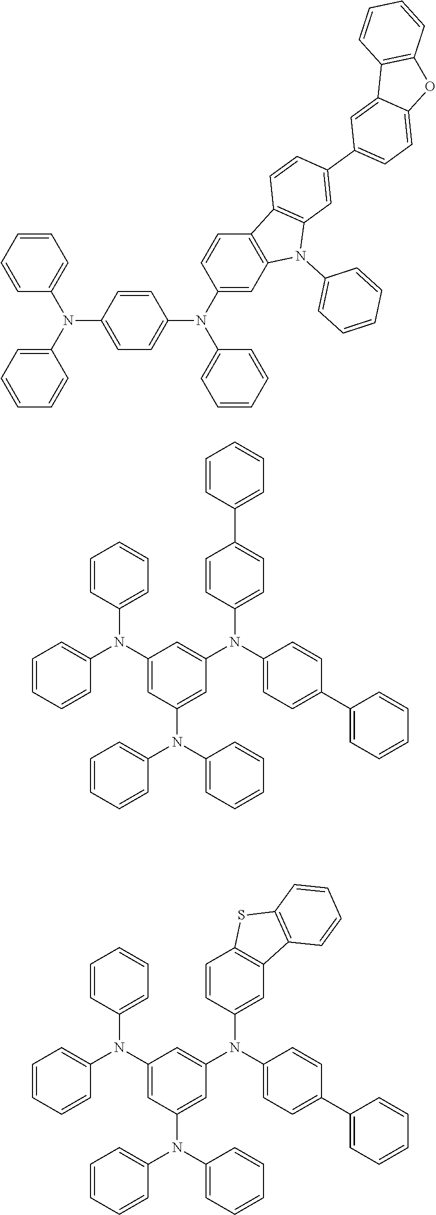

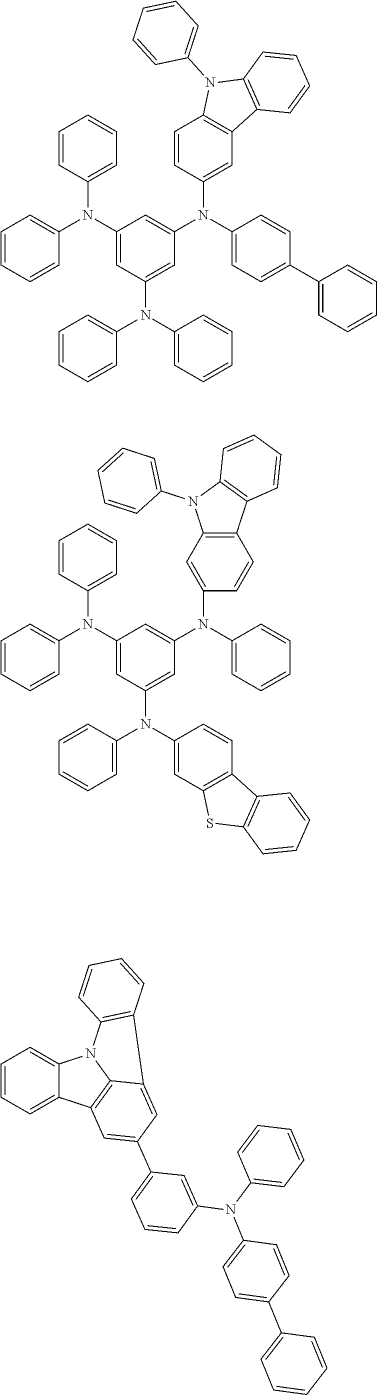 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1]

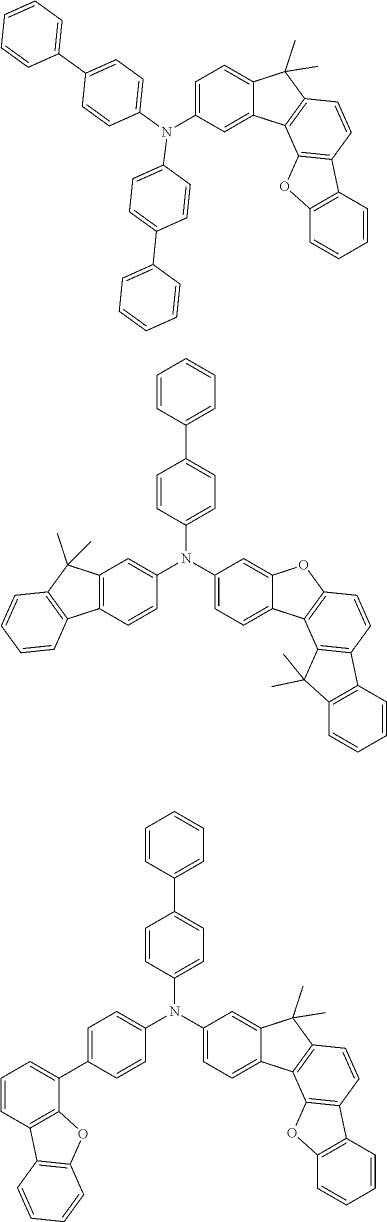

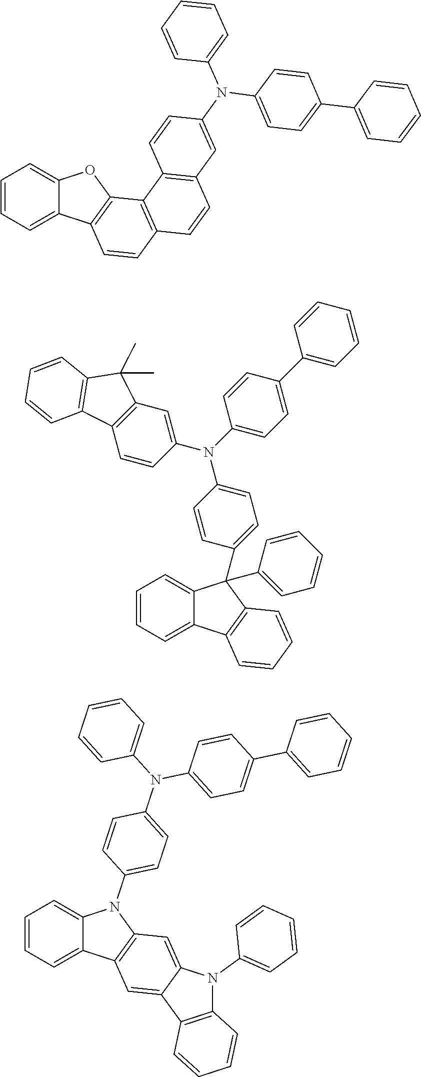 [SMILES: c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

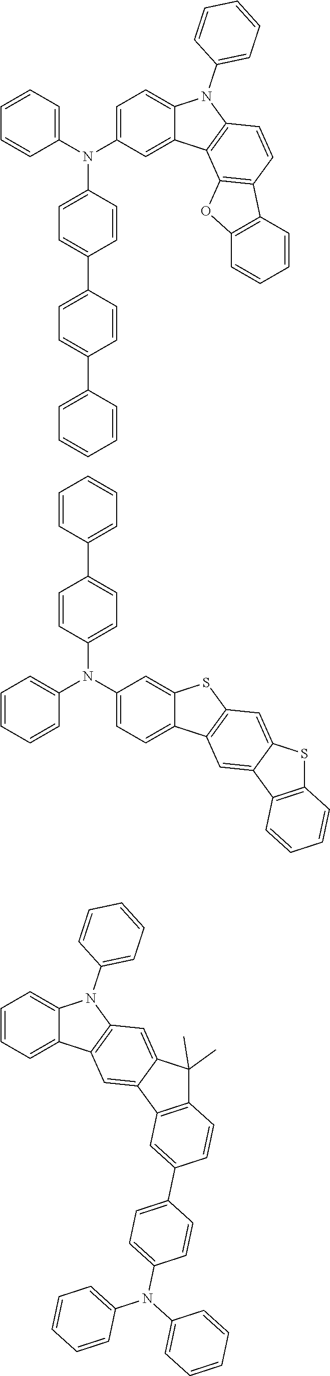

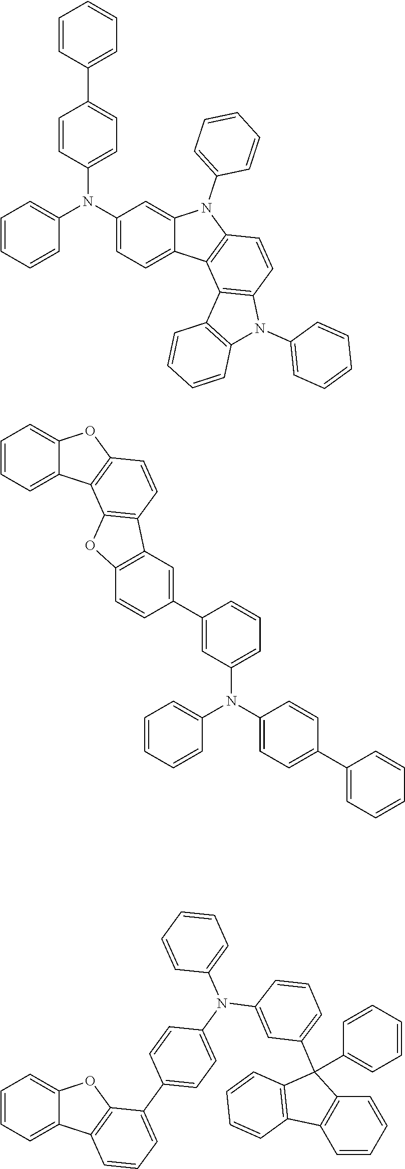 [SMILES: c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1 ; c1ccc(N(c2ccc(-c3cccc4c3oc3ccccc34)cc2)c2cccc(C3(c4ccccc4)c4ccccc4-c4ccccc43)c2)cc1]

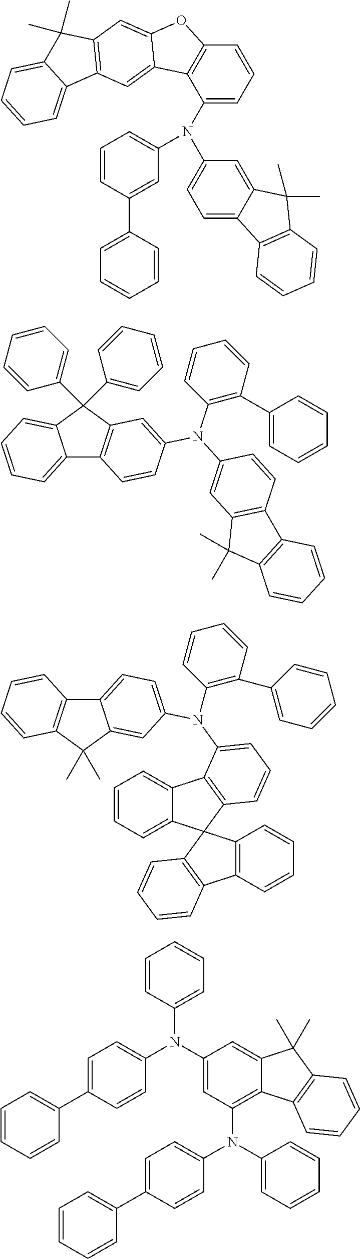

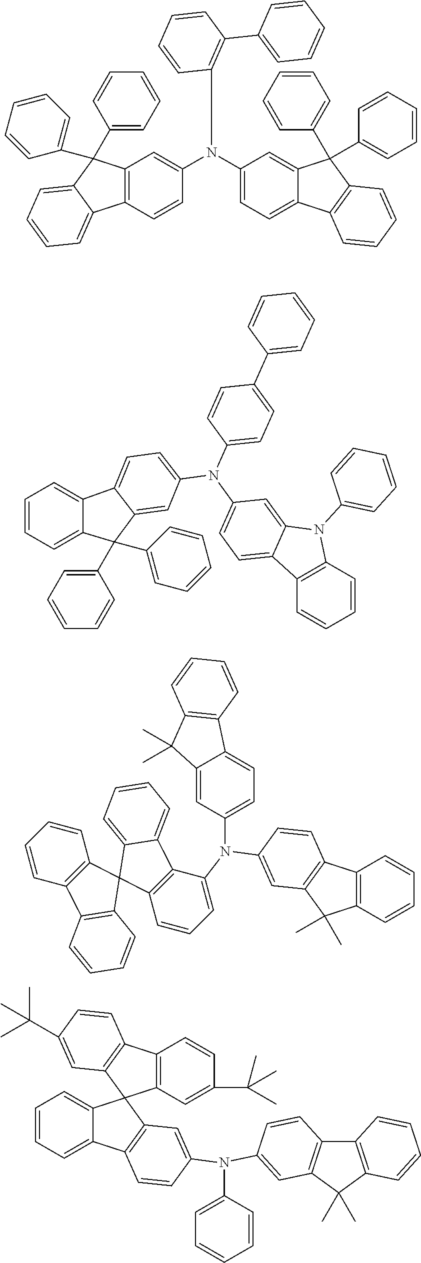

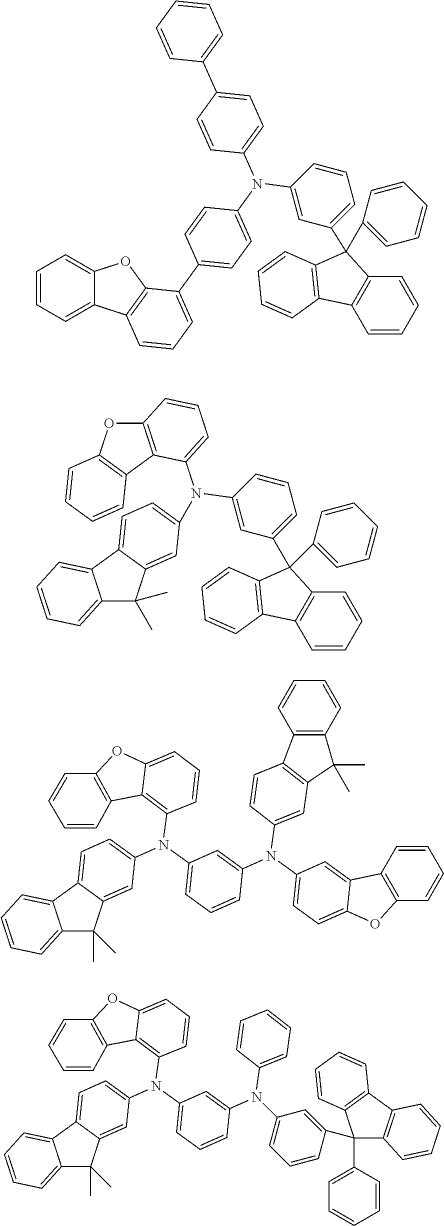

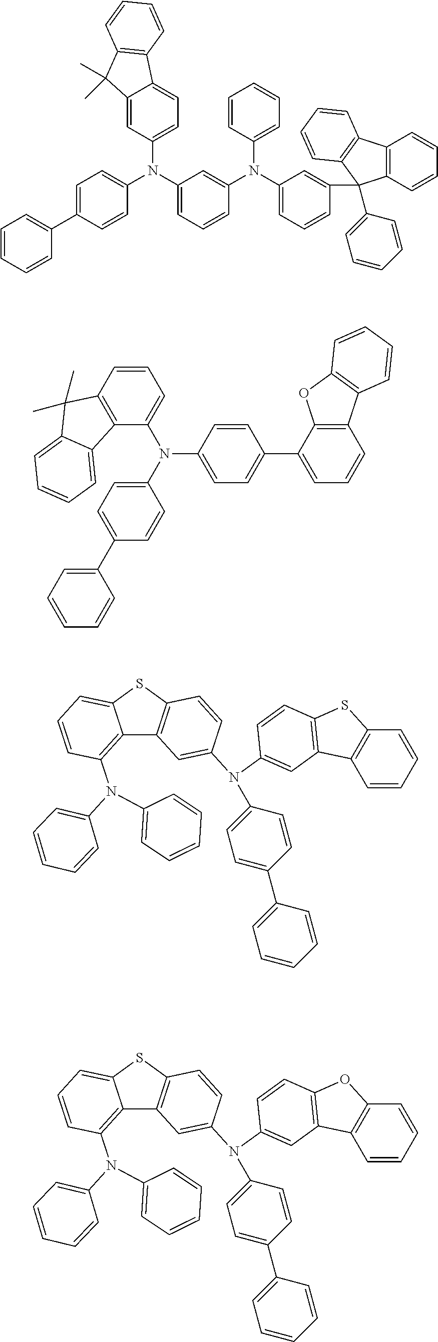 [SMILES: CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1 ; c1ccc(-c2ccc(N(c3ccc4oc5ccccc5c4c3)c3ccc4sc5cccc(N(c6ccccc6)c6ccccc6)c5c4c3)cc2)cc1]

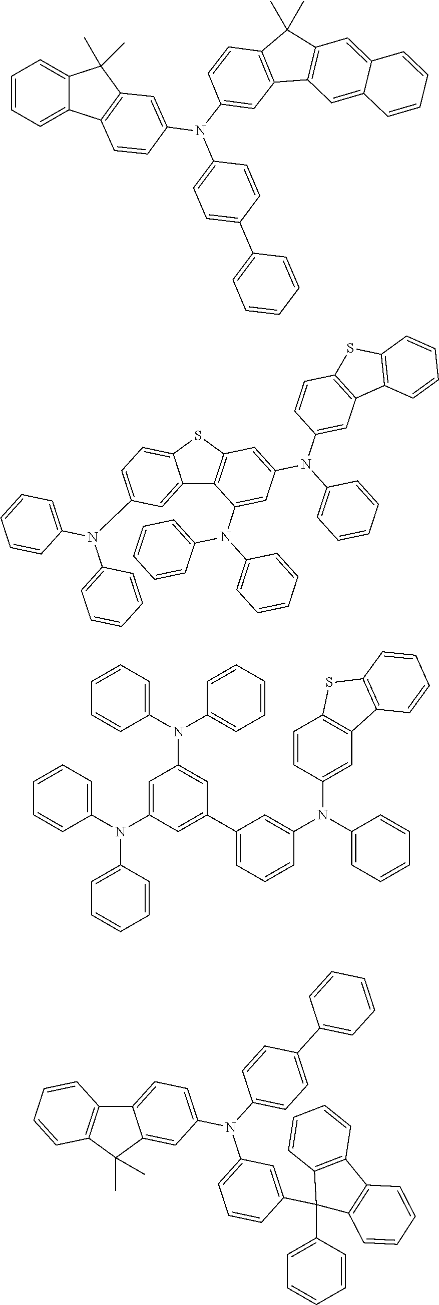

In the hole transport region, other suitable compounds may be used in addition to the compound.

In addition, the charge transport region may be, e.g., an electron transport region 150.

The electron transport region 150 may further increase electron injection and/or electron mobility and block holes between the cathode 110 and the light emitting layer 130.

In an implementation, the electron transport region 150 may include an electron transport layer between the cathode 110 and the light emitting layer 130, and an electron transport auxiliary layer between the light emitting layer 130 and the electron transport layer, and a compound of Group C may be included in at least one of the electron transport layer and the electron transport auxiliary layer.

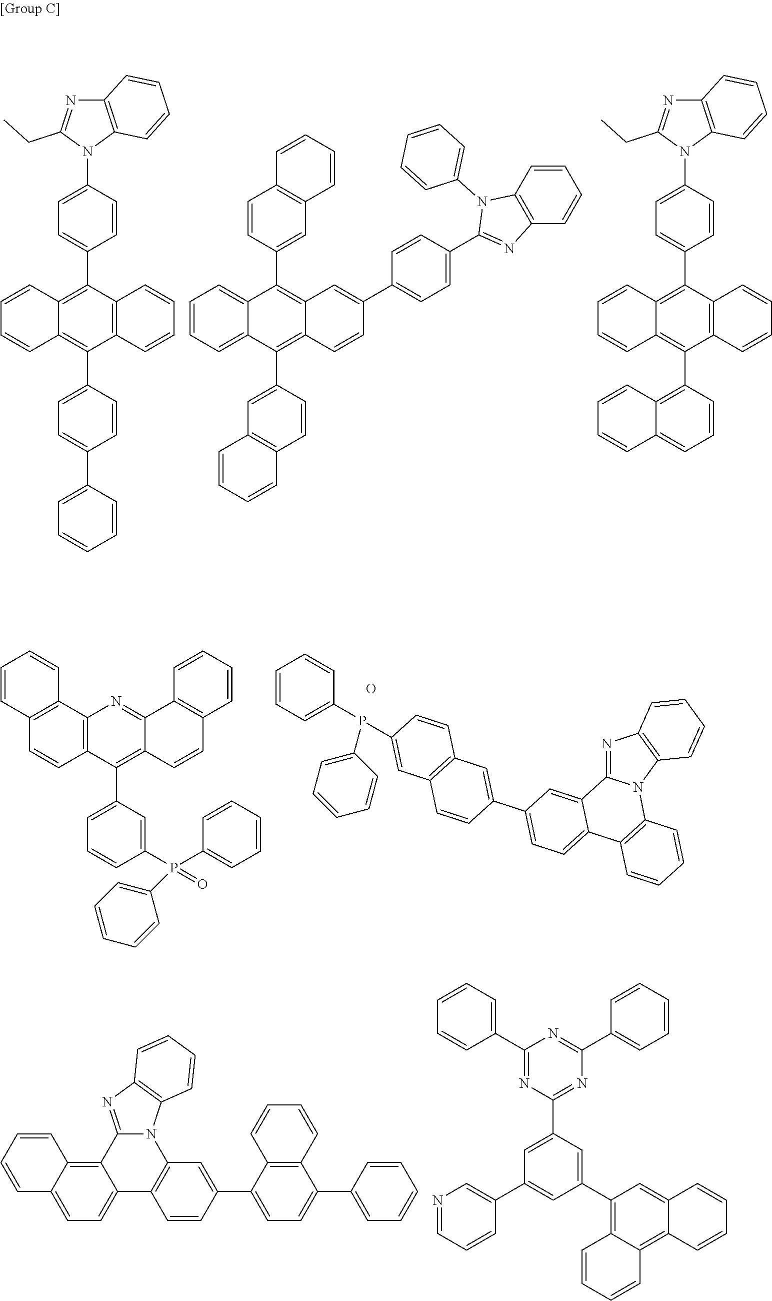

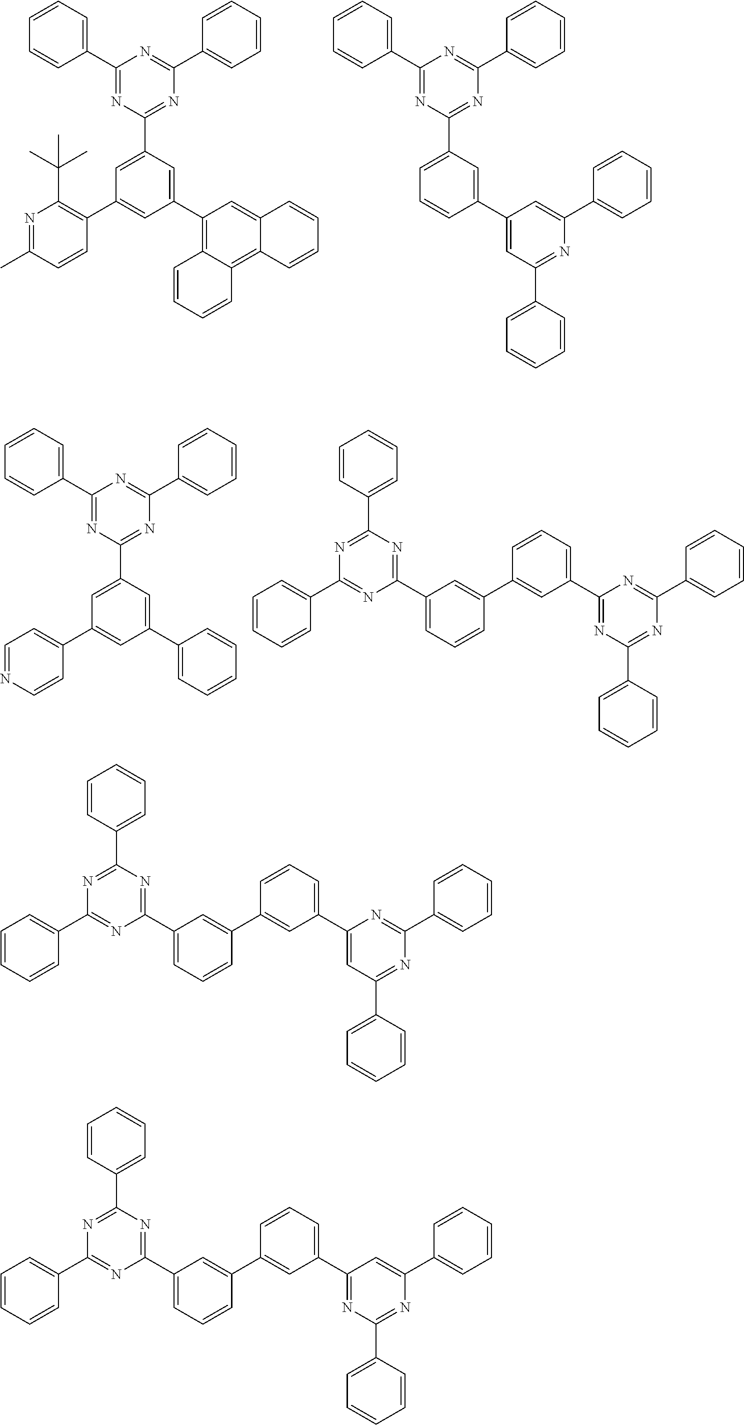

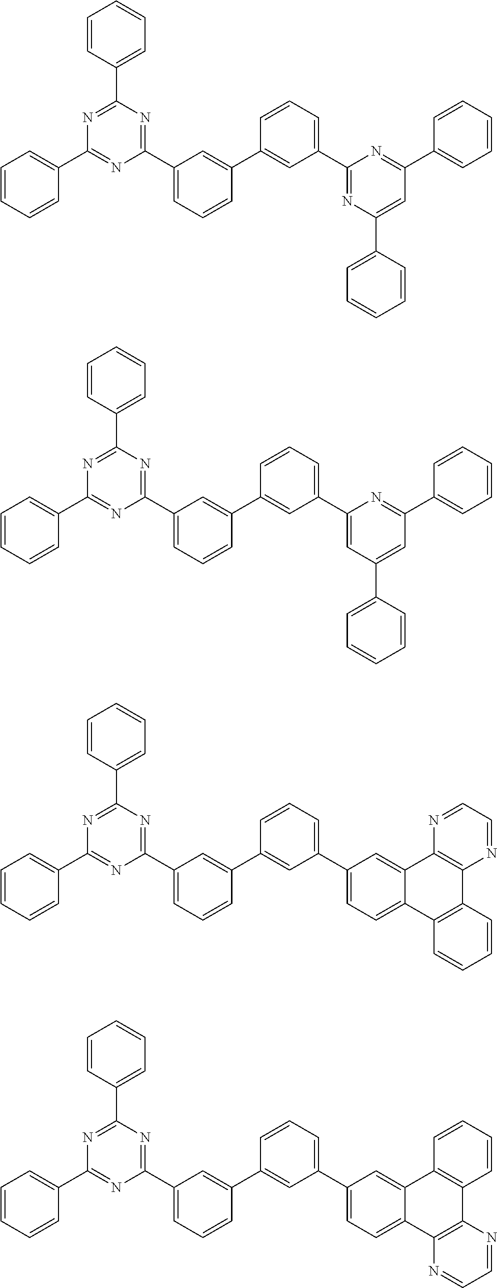 [SMILES: c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

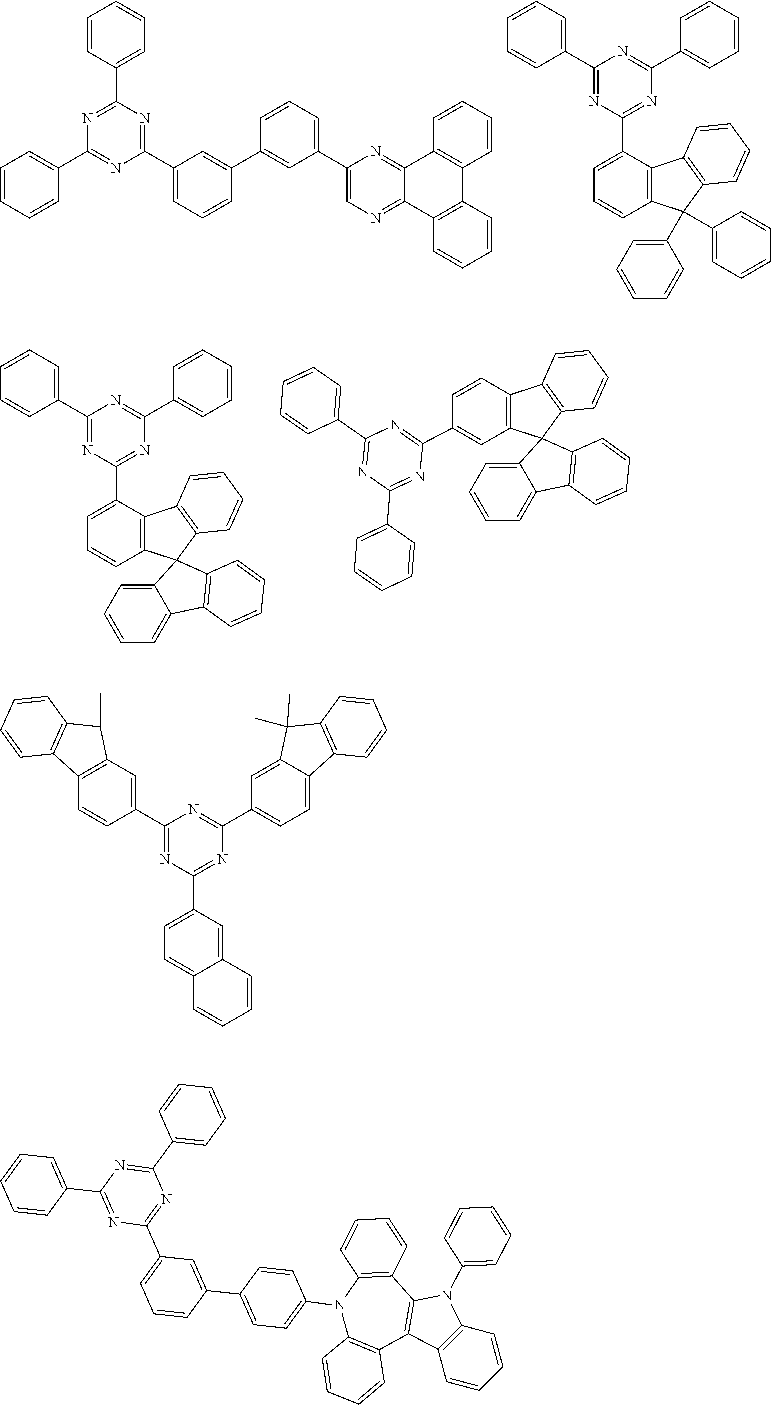 [SMILES: c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1 ; CC1c2ccccc2-c2ccc(-c3nc(-c4ccc5c(c4)C(C)(C)c4ccccc4-5)nc(-c4ccc5ccccc5c4)n3)cc21 ; c1ccc(-c2nc(-c3ccccc3)nc(-c3cccc(-c4ccc(N5c6ccccc6-c6c(n(-c7ccccc7)c7ccccc67)-c6ccccc65)cc4)c3)n2)cc1]

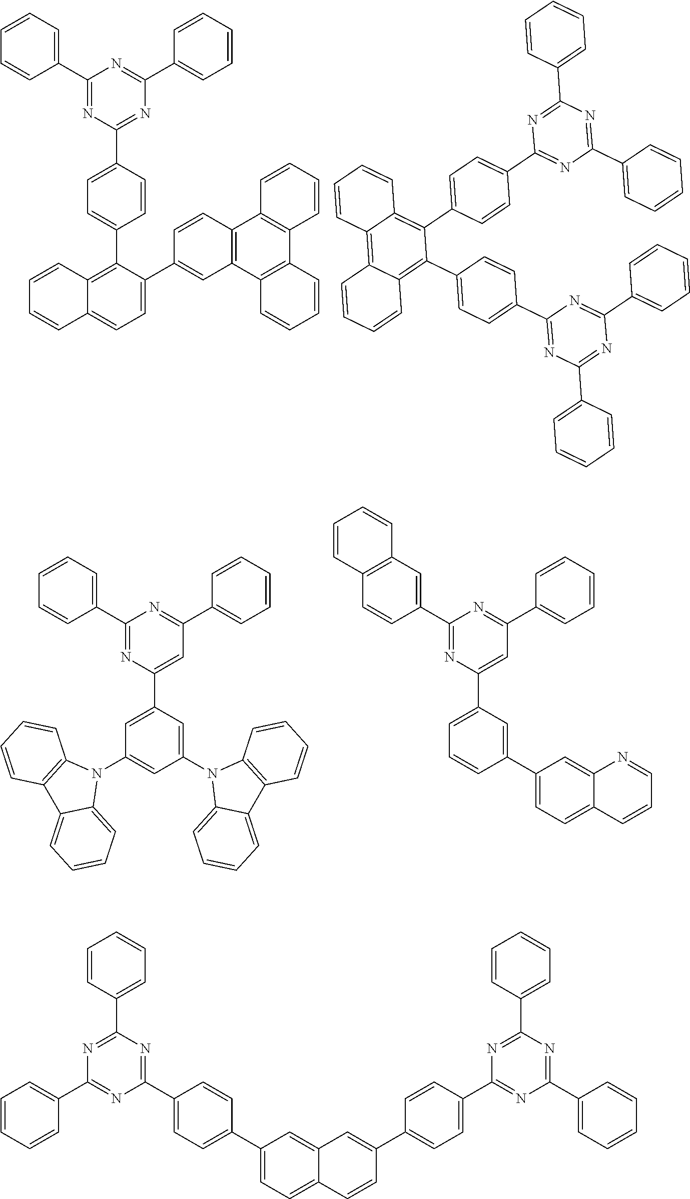

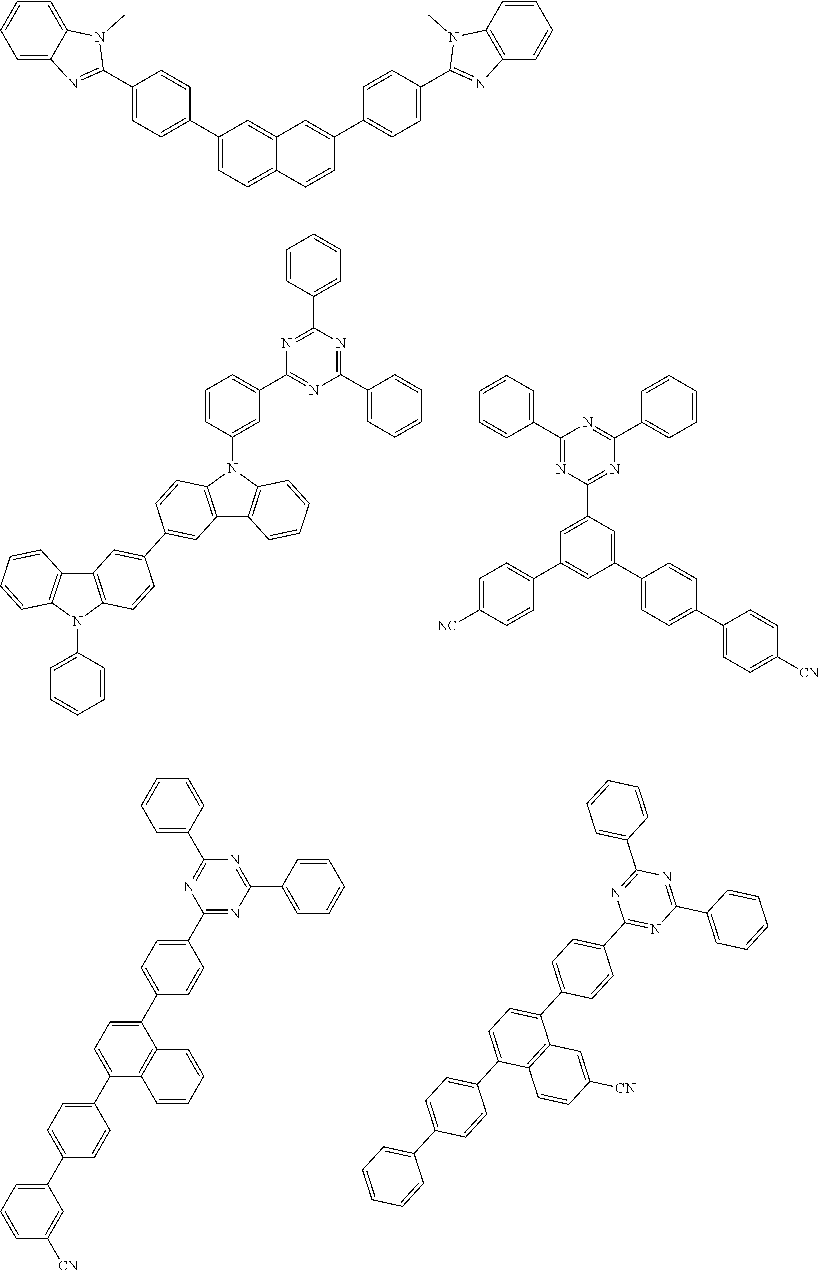 [SMILES: Cn1c(-c2ccc(-c3ccc4ccc(-c5ccc(-c6nc7ccccc7n6C)cc5)cc4c3)cc2)nc2ccccc21 ; c1ccc(-c2nc(-c3ccccc3)nc(-c3cccc(-n4c5ccccc5c5cc(-c6ccc7c(c6)c6ccccc6n7-c6ccccc6)ccc54)c3)n2)cc1 ; N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

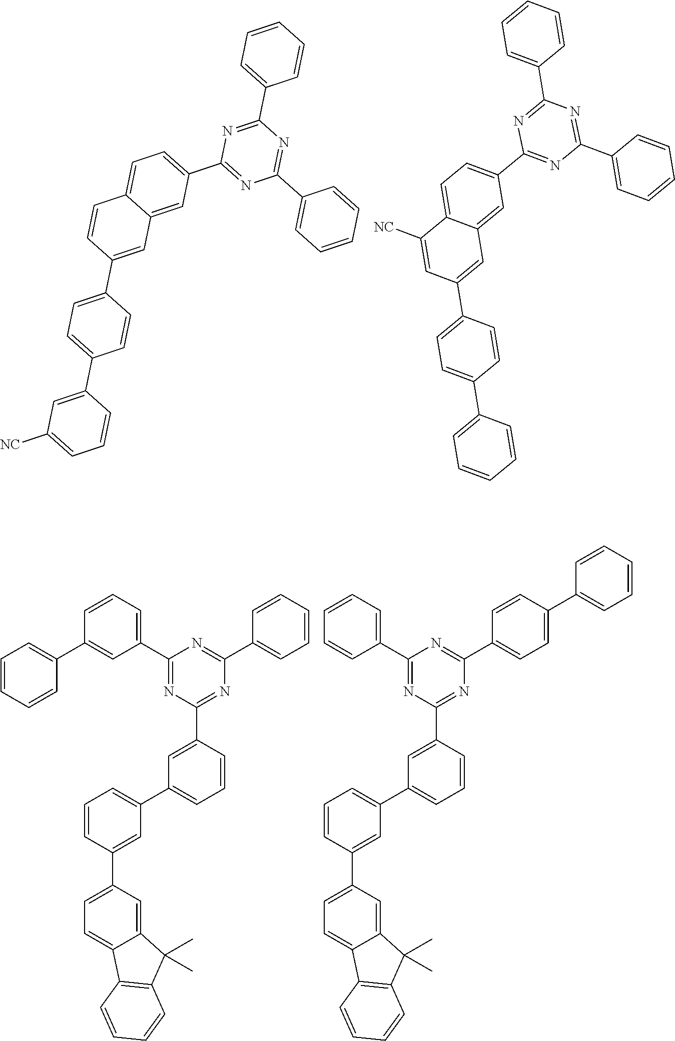

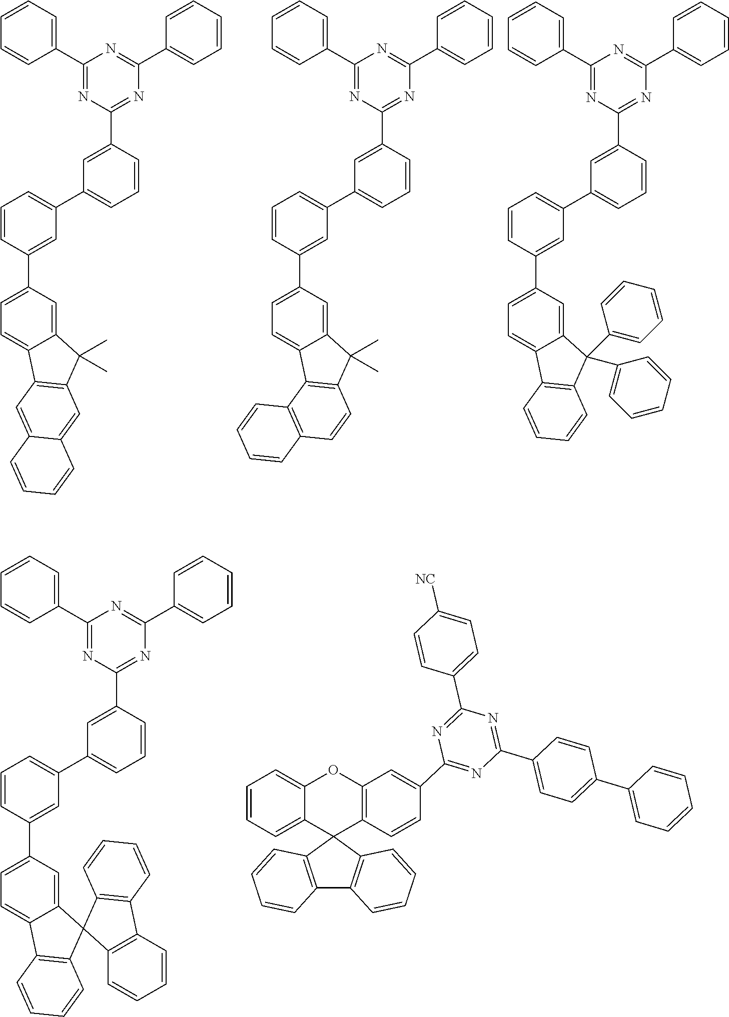

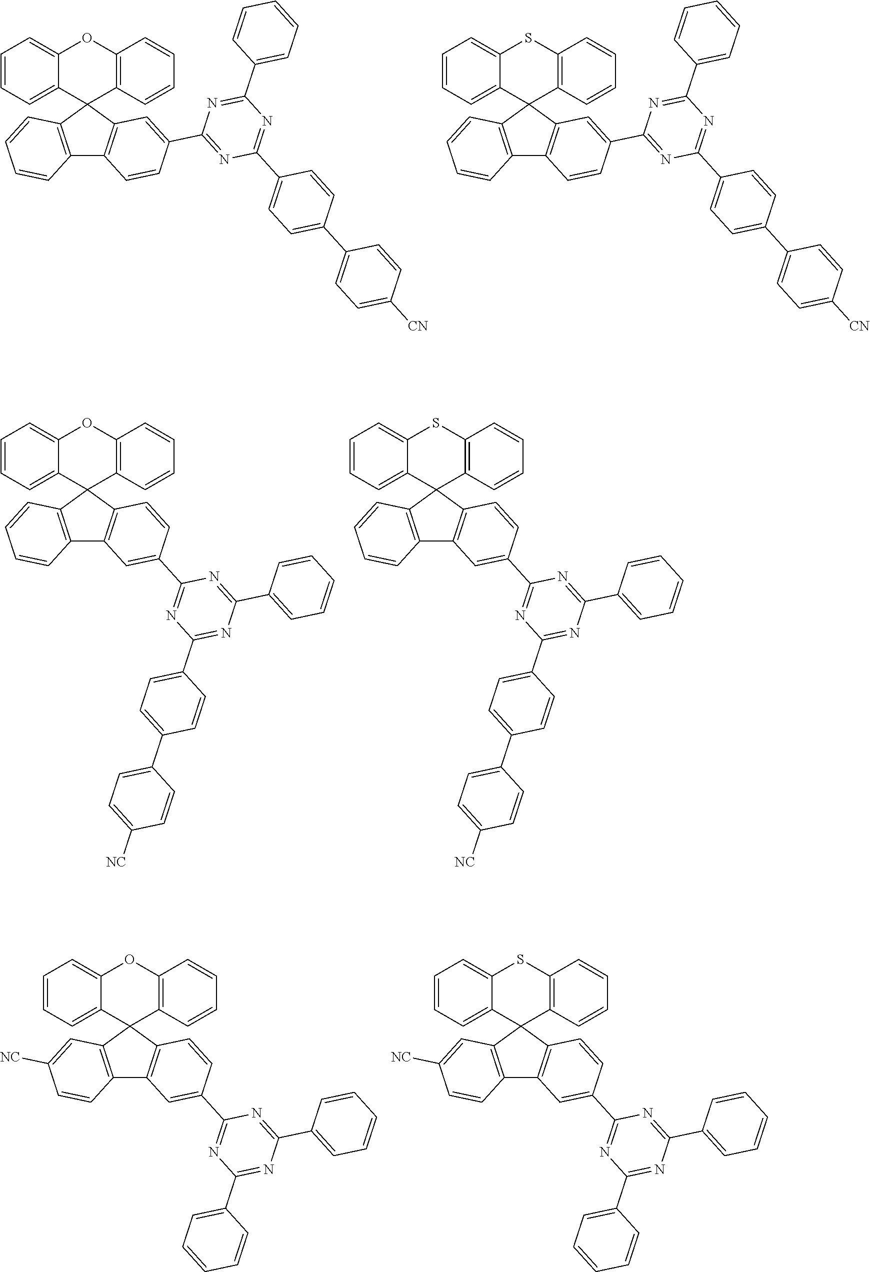

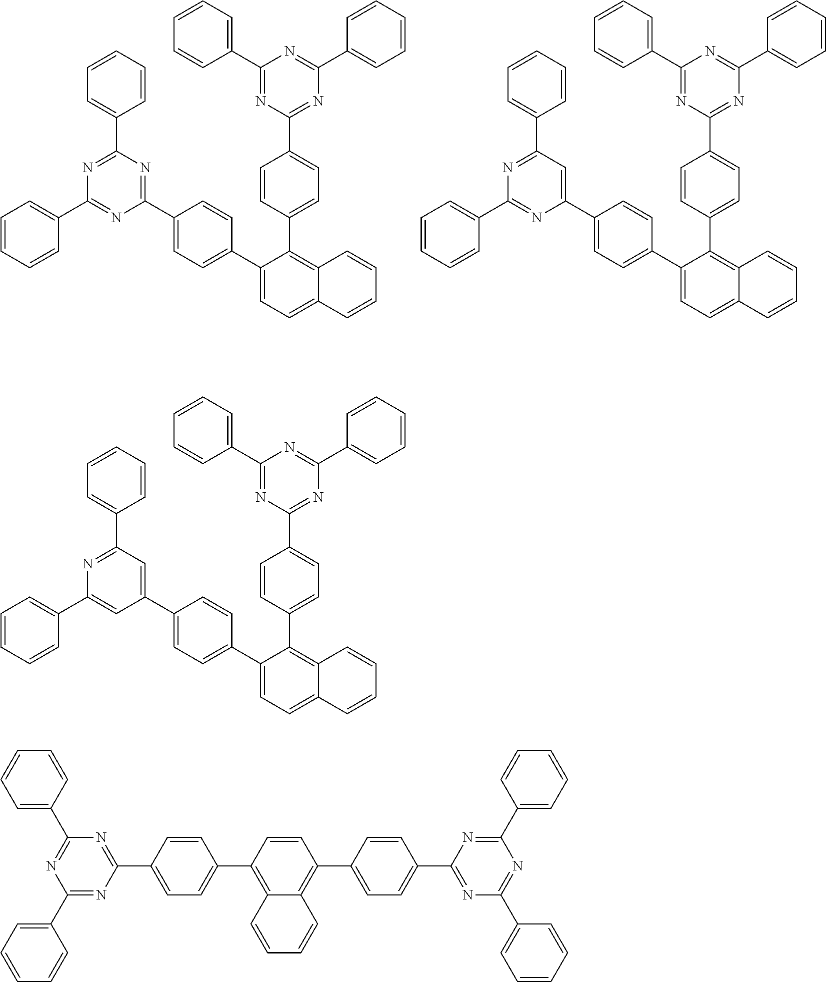 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1]

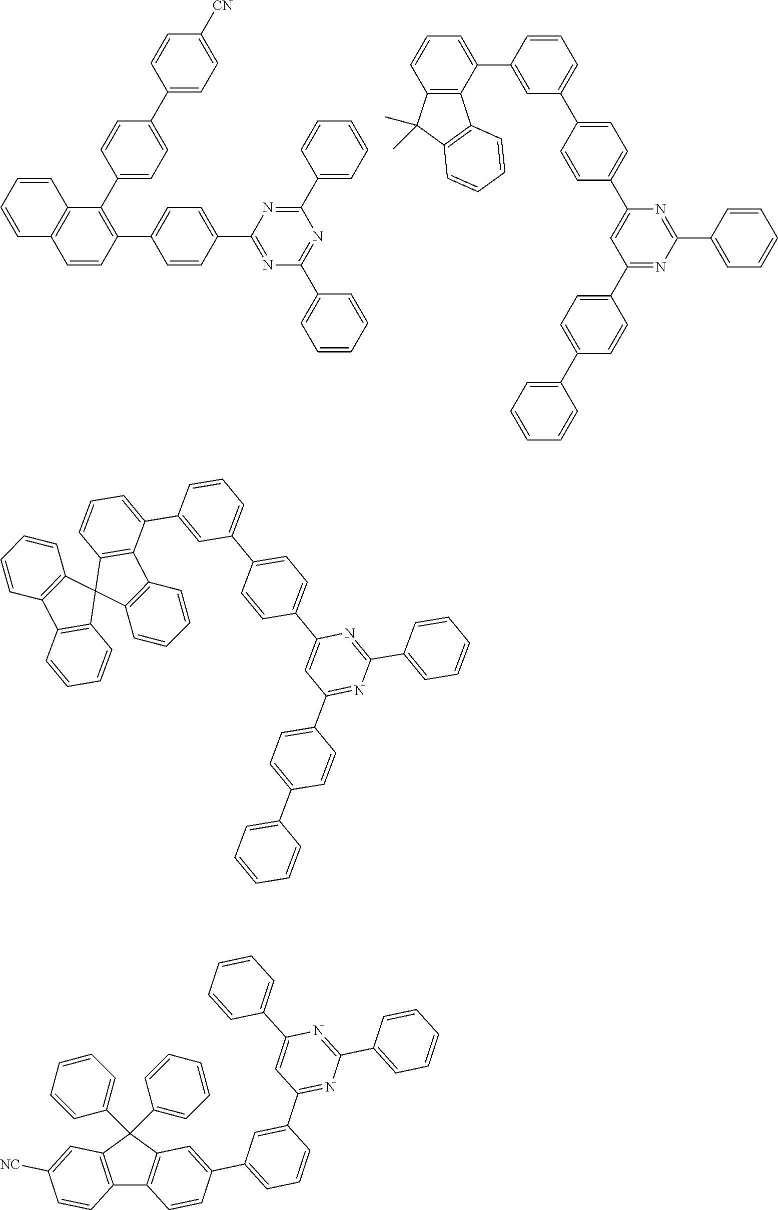

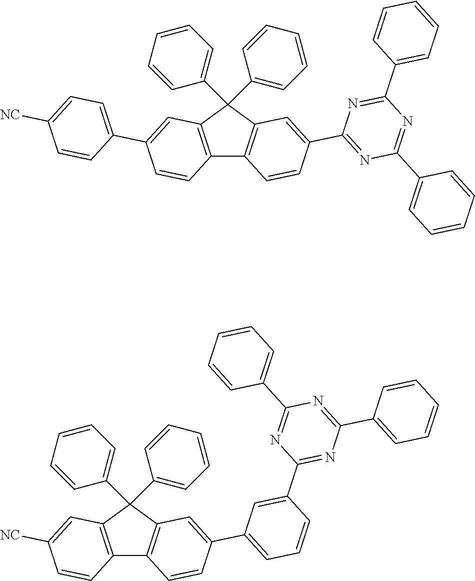 [SMILES: N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may include an organic light emitting diode including a light emitting layer as an organic layer.

Another embodiment may provide an organic light emitting diode including a light emitting layer and a hole transport region as an organic layer.

Another embodiment may provide an organic light emitting diode including a light emitting layer and an electron transport region as an organic layer.

As shown in the FIGURE, the organic light emitting diode according to the embodiment may include a hole transport region 140 and an electron transport region 150 in addition to the light emitting layer 130 as the organic layer 105.

In an implementation, the organic light emitting diode may further include an electron injection layer, a hole injection layer, or the like, in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., or Tokyo chemical industry, as far as there in no particular comment or were synthesized by suitable methods.

(Preparation of Compound for Organic Optoelectronic Device)

### Synthesis Example 1: Synthesis of Compound 1-2

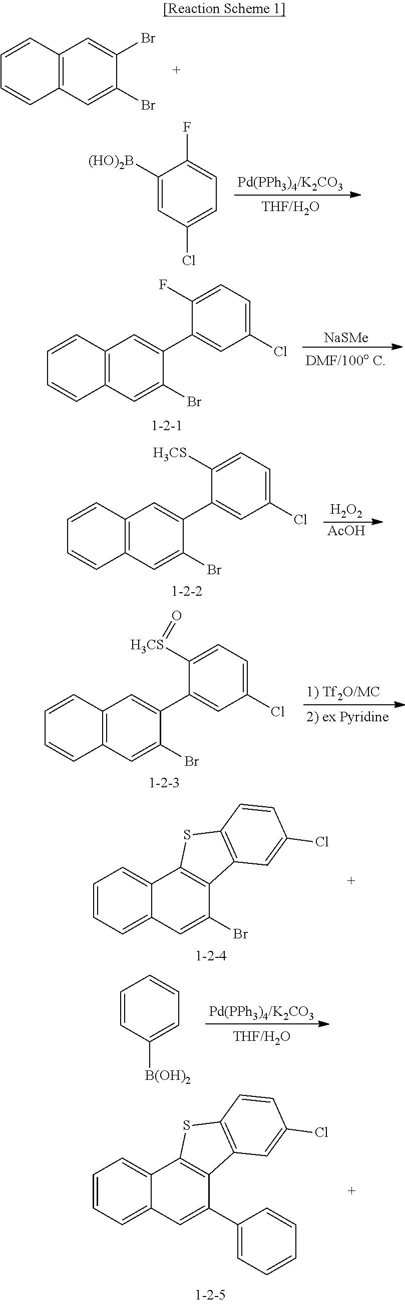

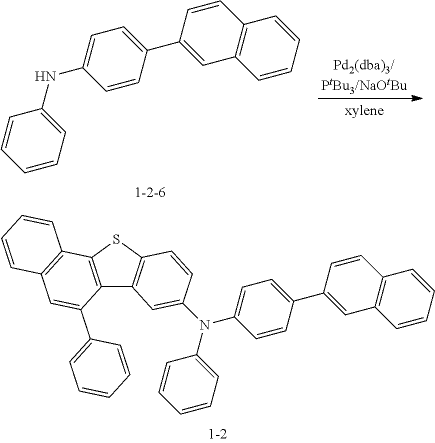

1st step: Synthesis of Intermediate 1-2-1

50.0 g (174.84 mmol) of 2,3-dibromonaphthalene, 37.26 g (192.33 mmol) of (5-chloro-2-fluorophenyl)boronic acid, 6.06 g (5.25 mmol) of Pd(PPh₃)₄, and 48.33 g (349.69 mmol) of K₂CO₃ were suspended in 800 mL of THF/400 mL of distilled water in a round-bottomed flask and then, stirred under reflux for 12 hours. When a reaction was completed, the resultant was concentrated and extracted with methylene chloride, and an organic layer therefrom was silica gel-columned, obtaining 35.0 g (Yield: 60%) of Intermediate 1-2-1.

2nd step: Synthesis of Intermediate 1-2-2

19.1 g (56.92 mmol) of Intermediate 1-2-1 and 4.03 g (57.48 mmol) of NaSCH₃ were suspended in 400 mL of DMF, and then, 20.91 g (186.34 mmol) of potassium tert-butoxide was added thereto and then, stirred at 100° C. for 12 hours. When a reaction was completed, 500 mL of distilled water was added thereto and then, extracted with EA. An organic layer therefrom was silica gel-columned, obtaining 12.0 g (Yield: 58%) of Intermediate 1-2-2.

3rd step: Synthesis of Intermediate 1-2-3

44.63 g (122.73 mmol) of Intermediate 1-2-2 was dissolved in 300 mL of acetic acid and then, slowly added to 22.6 mL (368.19 mmol) of H₂O₂ at 0° C. and then, stirred at ambient temperature for 12 hours. When a reaction was completed, after removing the solvent, the residue was extracted with EA and washed with distilled water. The solvent was concentrated and dried, obtaining 45 g (Yield: 97%) of Intermediate 1-2-3.

4th step: Synthesis of Intermediate 1-2-4

51.87 g (136.63 mmol) of Intermediate 1-2-3 was dissolved in 600 mL of methylene chloride, and 46.83 mL (273.26 mmol) of triflic anhydride was slowly added thereto at 0° C. The obtained mixture was stirred at ambient temperature for 4 hours, and 111 mL (1366.3 mmol) of pyridine was slowly added thereto at 0° C. When a reaction was completed, after removing the solvent, the residue was extracted with methylene chloride and washed with distilled water. The solvent was concentrated and then, recrystallized with toluene, obtaining 24.0 g (Yield: 51%) of Intermediate 1-2-4.

5th step: Synthesis of Intermediate 1-2-5

24.16 g (69.50 mmol) of Intermediate 1-2-4, 10.17 g (83.39 mmol) of phenylboronic acid, 2.41 g (2.08 mmol) of Pd(PPh₃)₄, and 19.21 g (138.99 mmol) of K₂CO₃ were suspended in 350 mL of THF/400 mL of distilled water and then, stirred under reflux for 12 hours. When a reaction was completed, the resultant was concentrated and extracted with methylene chloride, and an organic layer therefrom was silica gel columned, obtaining 16.7 g (Yield: 70%) of Intermediate 1-2-5.

6th step: Synthesis of Compound 1-2

16.79 g (42.52 mmol) of Intermediate 1-2-5, 15.07 g (51.02 mmol) of Intermediate 1-2-6, 6.13 g (20.19 mmol) of NaOtBu, and 3.35 g (50%, 8.29 mmol) of PtBu₃ were dissolved in 250 mL of xylene, and 2.53 g (2.76 mmol) of Pd₂(dba)₃ was added thereto and then, stirred under reflux under a nitrogen atmosphere for 12 hours. When a reaction was completed, the resultant was extracted with xylene and distilled water, and an organic layer therefrom was concentrated. The organic layer was silica gel-columned, obtaining 25.0 g (Yield: 90%) of Compound 1-2.

LC/MS calculated for: C44H29NS Exact Mass: 603.20 found for 603.89 [M+H]

### Synthesis Example 2: Synthesis of Compound 1-4

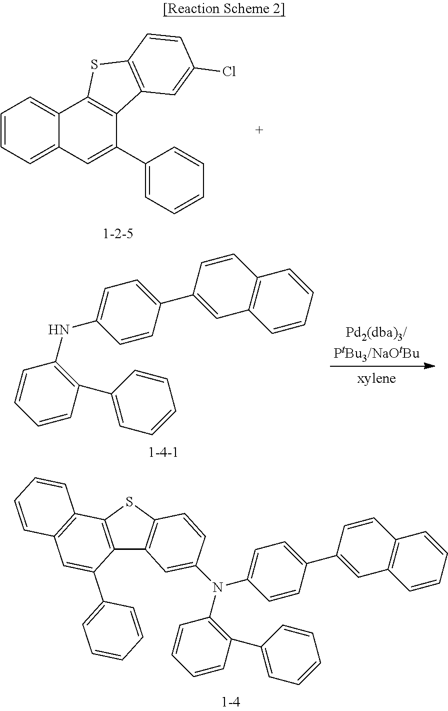 [SMILES: Clc1ccc2sc3c4ccccc4cc(-c4ccccc4)c3c2c1 ; c1ccc(-c2ccccc2Nc2ccc(-c3ccc4ccccc4c3)cc2)cc1 ; c1ccc(-c2ccccc2N(c2ccc(-c3ccc4ccccc4c3)cc2)c2ccc3sc4c5ccccc5cc(-c5ccccc5)c4c3c2)cc1]

Compound 1-4 (7.2 g, Yield: 83%) was synthesized in the same manner as the 6th step of Synthesis Example 1 except that Intermediate 1-2-5 and Intermediate 1-4-1 were used in an equivalent ratio of 1:1.2.

LC/MS calculated for: C50H33NS Exact Mass: 679.23 found for 679.76 [M+H]

### Synthesis Example 3: Synthesis of Compound 1-21

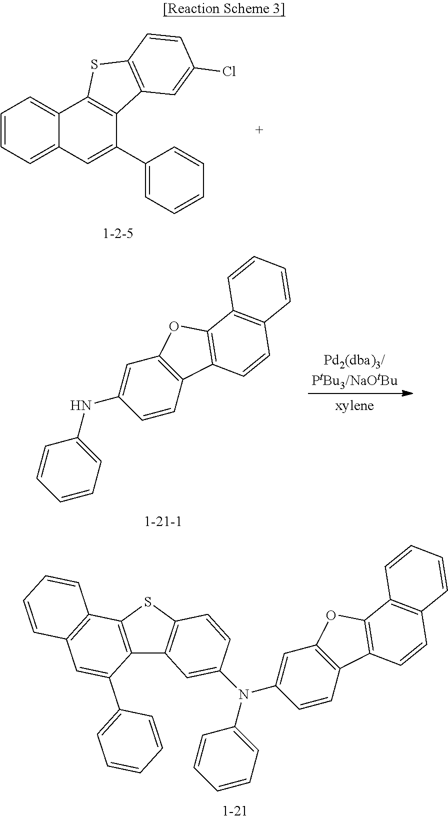 [SMILES: Clc1ccc2sc3c4ccccc4cc(-c4ccccc4)c3c2c1 ; c1ccc(Nc2ccc3c(c2)oc2c4ccccc4ccc32)cc1 ; c1ccc(-c2cc3ccccc3c3sc4ccc(N(c5ccccc5)c5ccc6c(c5)oc5c7ccccc7ccc65)cc4c23)cc1]

Compound 1-21 (6.0 g, Yield: 88%) was synthesized in the same manner as the 6th step of Synthesis Example 1 except that Intermediate 1-2-5 and Intermediate 1-21-1 were used in an equivalent ratio of 1:1.2.

LC/MS calculated for: C44H27NOS Exact Mass: 617.18 found for 617.78 [M+H]

### Synthesis Example 4: Synthesis of Compound 1-33

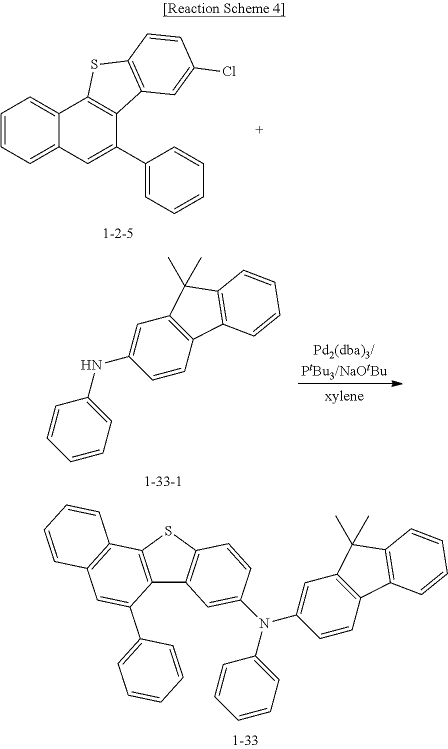

Compound 1-33 (8.0 g, Yield: 82%) was synthesized in the same manner as the 6th step of Synthesis Example 1 except that Intermediate 1-2-5 and Intermediate 1-33-1 were used in an equivalent ratio of 1:1.2.

LC/MS calculated for: C43H31NS Exact Mass: 593.22 found for 593.78 [M+H]

### Synthesis Example 5: Synthesis of Compound 1-42

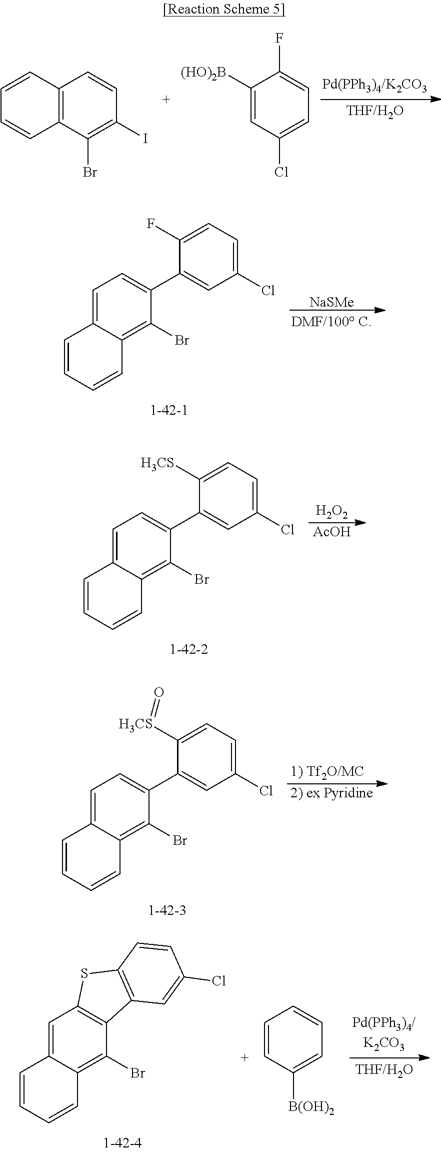

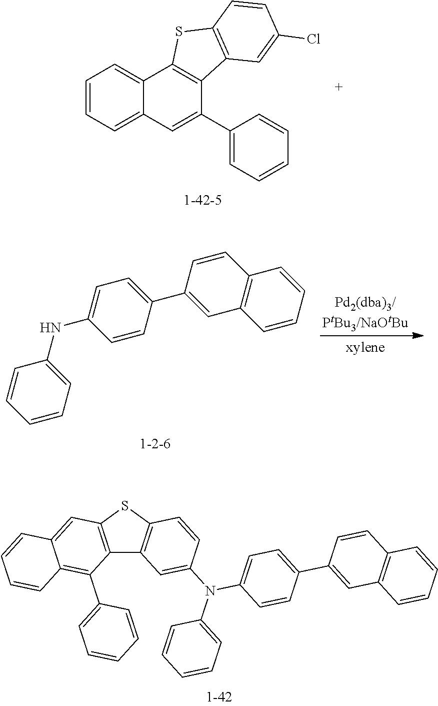 [SMILES: Clc1ccc2sc3c4ccccc4cc(-c4ccccc4)c3c2c1 ; c1ccc(Nc2ccc(-c3ccc4ccccc4c3)cc2)cc1 ; c1ccc(-c2c3ccccc3cc3sc4ccc(N(c5ccccc5)c5ccc(-c6ccc7ccccc7c6)cc5)cc4c23)cc1]

1st step: Synthesis of Intermediate 1-42-1

Intermediate 1-42-1 (42.0 g, Yield: 60%) was synthesized in the same manner as the lth step of Synthesis Example 1 except that 1-bromo-2-iodonaphthalene and (5-chloro-2-fluorophenyl)boronic acid were used in an equivalent ratio of 1:1.1.

2nd step: Synthesis of Intermediate 1-42-2

Intermediate 1-42-2 (25.0 g, Yield: 55%) was synthesized in the same manner as the 2nd step of Synthesis Example 1 except that Intermediate 1-42-1 was used.

3rd step: Synthesis of Intermediate 1-42-3

Intermediate 1-42-3 (24.0 g, Yield: 90%) was synthesized in the same manner as the 2nd step of Synthesis Example 1 except that Intermediate 1-42-2 was used.

4th step: Synthesis of Intermediate 1-42-4

Intermediate 1-42-4 (11.0 g, Yield: 51%) was synthesized in the same manner as the 4th step of Synthesis Example 1 except that Intermediate 1-42-3 was used.

5th step: Synthesis of Intermediate 1-42-5

Intermediate 1-42-5 (15.0 g, Yield: 72%) was synthesized in the same manner as the 5th step of Synthesis Example 1 except that Intermediate 1-42-4 and an intermediate of phenylboronic acid were used in an equivalent ratio of 1:1.2.

6th step: Synthesis of Compound 1-42

Compound 1-42 (8.5 g, Yield: 81%) was synthesized in the same manner as the 6th step of Synthesis Example 1 except that Intermediate 1-42-5 and Intermediate 1-2-6 were used in an equivalent ratio of 1:1.2.

LC/MS calculated for: C44H29NS Exact Mass: 603.20 found for 603.79 [M+H]

### Synthesis Example 6: Synthesis of Compound 1-55

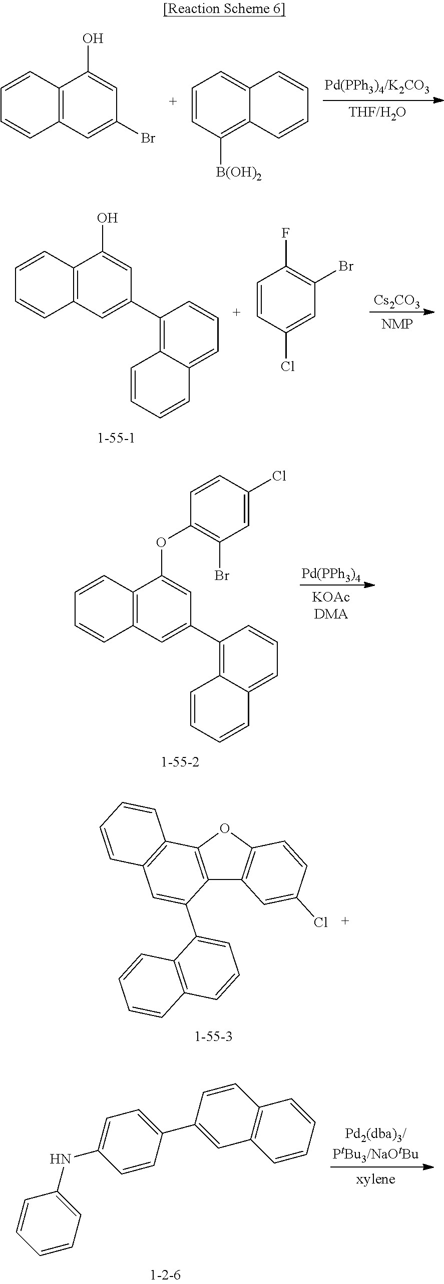

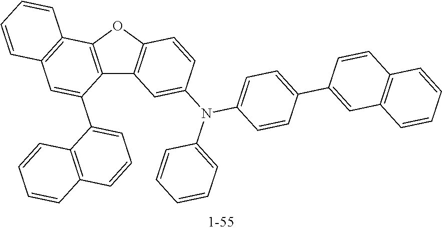 [SMILES: c1ccc(N(c2ccc(-c3ccc4ccccc4c3)cc2)c2ccc3oc4c5ccccc5cc(-c5cccc6ccccc56)c4c3c2)cc1]

1st step: Synthesis of Intermediate 1-55-1

24.16 g (108.31 mmol) of 3-bromonaphthalen-1-ol, 22.35 g (129.97 mmol) of 1-naphthaleneboronic acid, 3.75 g (3.25 mmol) of Pd(PPh₃)₄, and 29.94 g (216.61 mmol) of K₂CO₃ were suspended in 500 mL of THF/250 mL of distilled water in a round-bottomed flask and then, stirred under reflux for 12 hours. When a reaction was completed, the resultant was concentrated and extracted with methylene chloride, and an organic layer therefrom was silica gel columned, obtaining 21.0 g (Yield: 72%) of Intermediate 1-55-1.

2nd step: Synthesis of Intermediate 1-55-2

30.0 g (110.98 mmol) of Intermediate 1-55-1, 34.61 g (166.46 mmol) of 2-bromo-4-chloro-1-fluorobenzene, and 108.47 g (332.93 mmol) of Cs₂CO₃ were suspended in 100 mL of NMP and then, stirred for 12 hours at 155° C. When a reaction was completed, 500 mL of distilled water was added thereto and then, extracted with EA. An organic layer therefrom was silica gel columned, obtaining 30.0 g (Yield: 59%) of Intermediate 1-55-2.

3rd step: Synthesis of Intermediate 1-55-3

25.0 g (54.38 mmol) of Intermediate 1-55-2, 3.14 g (2.72 mmol) of Pd(PPh₃)₄, and 10.67 g (108.75 mmol) of KOAc were suspended in 200 mL of DMA and then, stirred for 12 hours at 160° C. When a reaction was completed, the resultant was concentrated and then, extracted with methylene chloride, and an organic layer therefrom was silica gel-columned, obtaining 17.0 g (Yield: 83%) of Intermediate 1-55-3.

4th step: Synthesis of Compound 1-55

Compound 1-55 (7.5 g, Yield: 76%) was synthesized in the same manner as the 6th step of Synthesis Example 1 except that Intermediate 1-55-3 and Intermediate 1-2-6 were used in an equivalent ratio of 1:1.2.

LC/MS calculated for: C48H31NO Exact Mass: 637.24 found for 637.97 [M+H]

### Synthesis Example 7: Synthesis of Compound 1-57

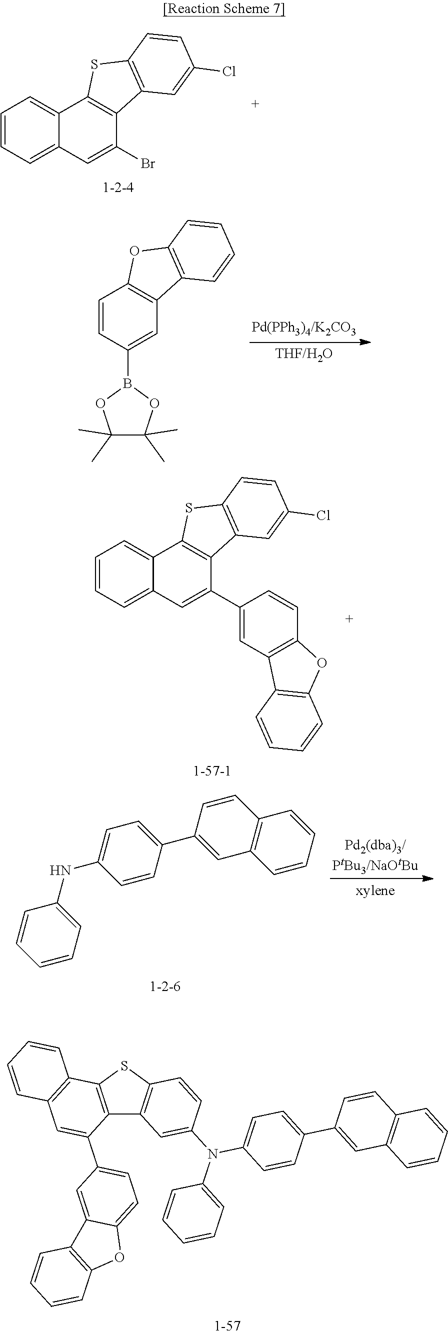 [SMILES: Clc1ccc2sc3c4ccccc4cc(Br)c3c2c1 ; CC1(C)OB(c2ccc3oc4ccccc4c3c2)OC1(C)C ; Clc1ccc2sc3c4ccccc4cc(-c4ccc5oc6ccccc6c5c4)c3c2c1 ; c1ccc(Nc2ccc(-c3ccc4ccccc4c3)cc2)cc1 ; c1ccc(N(c2ccc(-c3ccc4ccccc4c3)cc2)c2ccc3sc4c5ccccc5cc(-c5ccc6oc7ccccc7c6c5)c4c3c2)cc1]

1st step: Synthesis of Intermediate 1-57-1

Intermediate 1-57-1 (18.0 g, Yield: 78%) was synthesized in the same manner as the 5th step of Synthesis Example 1 except that Intermediate 1-2-4 and 2-(dibenzo[b,d]furan-2-yl)-4,4,5,5-tetramethyl-1,3,2-dioxaborolane were used in an equivalent ratio of 1:1.2

2nd step: Synthesis of Compound 1-57

Compound 1-57 (8.2 g, Yield: 77%) was synthesized in the same manner as the 6th step of Synthesis Example 1 except that Intermediate 1-57-1 and Intermediate 1-2-6 were used in an equivalent ratio of 1:1.2.

LC/MS calculated for: C50H31NOS Exact Mass: 693.21 found for 693.78 [M+H]

### Synthesis Example 8: Synthesis of Compound 1-59

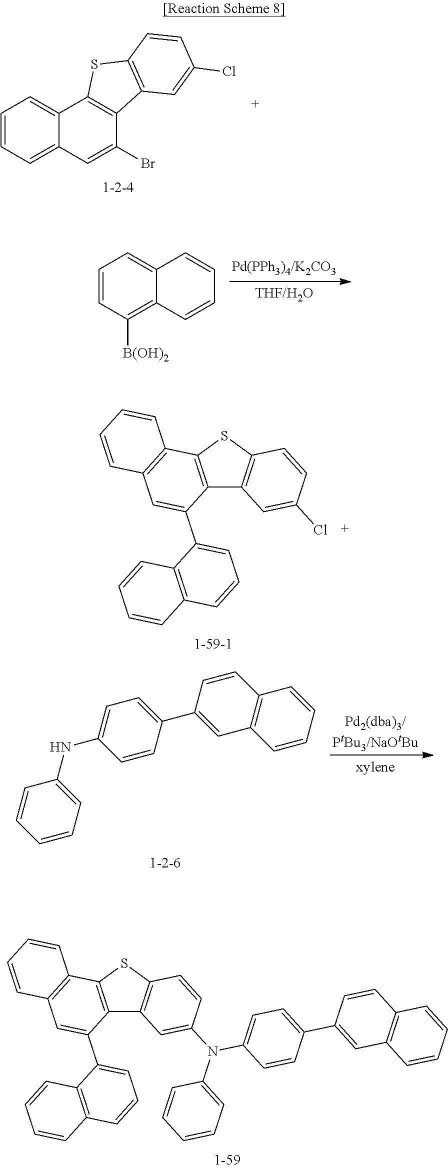 [SMILES: Clc1ccc2sc3c4ccccc4cc(Br)c3c2c1 ; OB(O)c1cccc2ccccc12 ; Clc1ccc2sc3c4ccccc4cc(-c4cccc5ccccc45)c3c2c1 ; c1ccc(Nc2ccc(-c3ccc4ccccc4c3)cc2)cc1 ; c1ccc(N(c2ccc(-c3ccc4ccccc4c3)cc2)c2ccc3sc4c5ccccc5cc(-c5cccc6ccccc56)c4c3c2)cc1]

1st step: Synthesis of Intermediate 1-59-1

Intermediate 1-59-1 (17.0 g, Yield: 81%) was synthesized in the same manner as the 5th step of Synthesis Example 1 except that Intermediate 1-2-4 and 1-naphthaleneboronic acid were used in an equivalent ratio of 1:1.2.

2nd step: Synthesis of Compound 1-59

Compound 1-59 (7.5 g, Yield: 80%) was synthesized in the same manner as the 6th step of Synthesis Example 1 except that Intermediate 1-59-1 and Intermediate 1-2-6 were used in an equivalent ratio of 1:1.2.

LC/MS calculated for: C48H31NS Exact Mass: 653.22 found for 653.88 [M+H]

### Synthesis Example 9: Synthesis of Compound 1-67

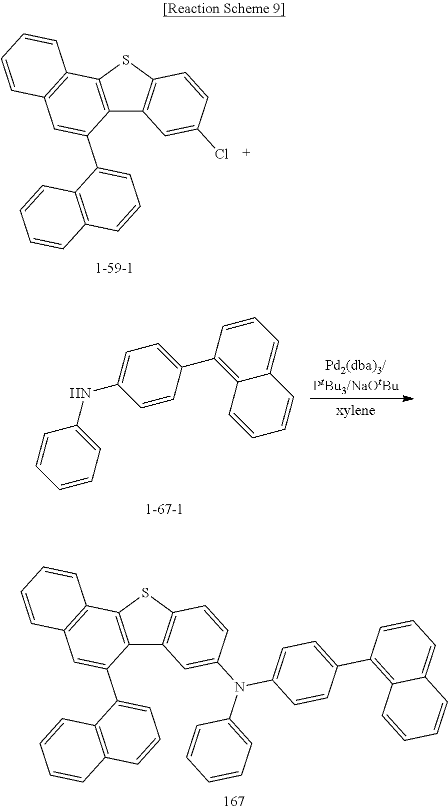 [SMILES: Clc1ccc2sc3c4ccccc4cc(-c4cccc5ccccc45)c3c2c1 ; c1ccc(Nc2ccc(-c3cccc4ccccc34)cc2)cc1 ; c1ccc(N(c2ccc(-c3cccc4ccccc34)cc2)c2ccc3sc4c5ccccc5cc(-c5cccc6ccccc56)c4c3c2)cc1]

Compound 1-67 (8.6 g, Yield: 77%) was synthesized in the same manner as the 6th step of Synthesis Example 1 except that Intermediate 1-59-1 and Intermediate 1-67-1 were used in an equivalent ratio of 1:1.2.

LC/MS calculated for: C48H31NS Exact Mass: 653.22 found for 653.90 [M+H]

### Synthesis Example 10: Synthesis of Compound 1-81

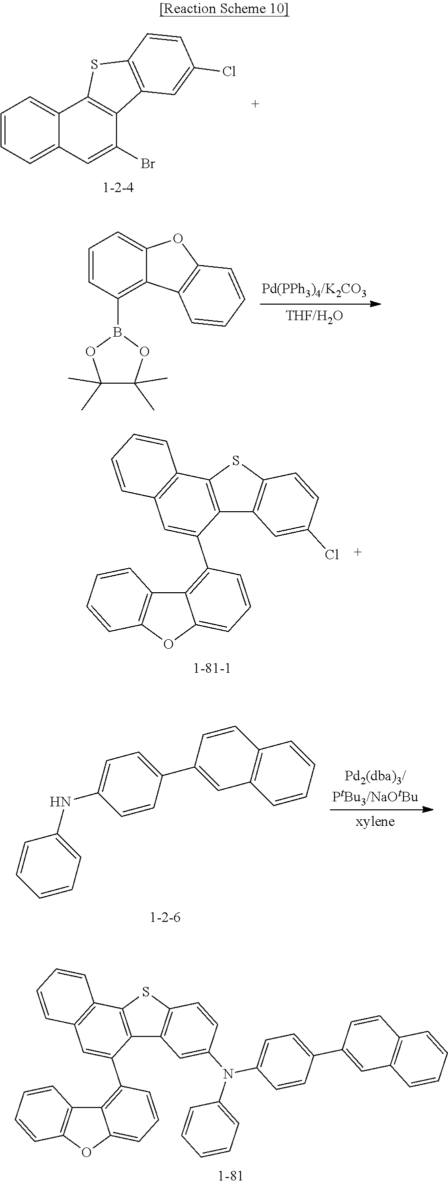

1st step: Synthesis of Intermediate 1-81-1

Intermediate 1-81-1 (26.0 g, Yield: 79%) was synthesized in the same manner as the 5th step of Synthesis Example 1 except that Intermediate 1-2-4 and 2-(dibenzo[b,d]furan-1-yl)-4,4,5,5-tetramethyl-1,3,2-dioxaborolane were used in an equivalent ratio of 1:1.2.

2nd step: Synthesis of Compound 1-81

Compound 1-81 (7.2 g, Yield: 80%) was synthesized in the same manner as the 5th step of Synthesis Example 1 except that Intermediate 1-81-1 and Intermediate 1-2-6 were used in an equivalent ratio of 1:1.2.

LC/MS calculated for: C50H31NOS Exact Mass: 693.21 found for 693.88 [M+H]

Comparative Synthesis Example 1: Synthesis of Compound Y1

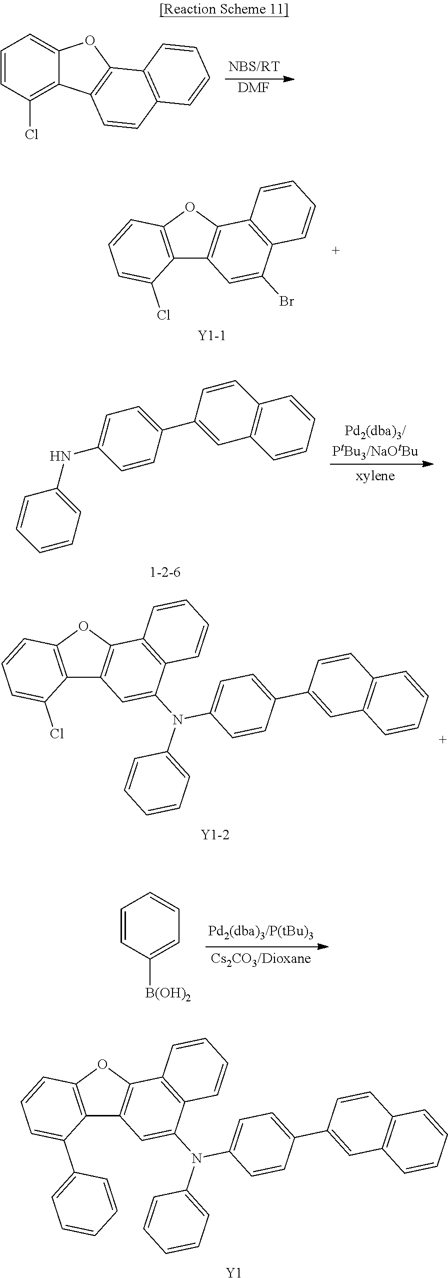

1st step: Synthesis of Intermediate Y1-1

31.47 g (124.55 mmol) of 7-chloronaphtho[1,2-b]benzofuran was dissolved in 400 mL of DMF in a round-bottomed flask, and 26.60 g (149.46 mmol) of NBS was slowly added thereto. The obtained mixture was stirred at ambient temperature for 12 hours, and 500 mL of methanol was added thereto to form a precipitate. The obtained solid was filtered with a glass filter to obtain 36.4 g (Yield: 88%) of Intermediate Y1-1.

2nd step: Synthesis of Intermediate Y1-2

16.88 g (50.91 mmol) of Intermediate Y1-1, 18.0 g (61.09 mmol) of Intermediate 1-2-6, 7.33 g (76.36 mmol) of NaOtBu, and 3.71 g (50%, 9.16 mmol) of PtBu₃ were dissolved in 250 mL of xylene, and 2.79 g (3.05 mmol) of Pd₂(dba)₃ was added thereto and then, stirred under reflux for 12 hours under a nitrogen atmosphere. When a reaction was completed, after extraction with xylene and distilled water, an organic layer therefrom was concentrated. The organic layer was silica gel-columned, obtaining 25.0 g (Yield: 90%) of Intermediate Y1-2.

3rd step: Synthesis of Compound Y1

13.93 g (25.52 mmol) of Intermediate Y1-2, 4.04 g (33.18 mmol) of phenylboronic acid, 1.16 g (1.28 mmol) of Pd₂(dba)₃, and 16.63 g (51.04 mmol) of Cs₂CO₃ were suspended in 100 mL of dioxane and then, stirred at 130° C. for 12 hours. When a reaction was completed, the resultant was concentrated and extracted with methylene chloride, and an organic layer therefrom was silica gel-columned, obtaining 10.0 g (Yield: 67%) of Compound Y1.

LC/MS calculated for: C44H29NO Exact Mass: 587.72 found for 587.98 [M+H]

Comparative Synthesis Example 2: Synthesis of Compound Y2

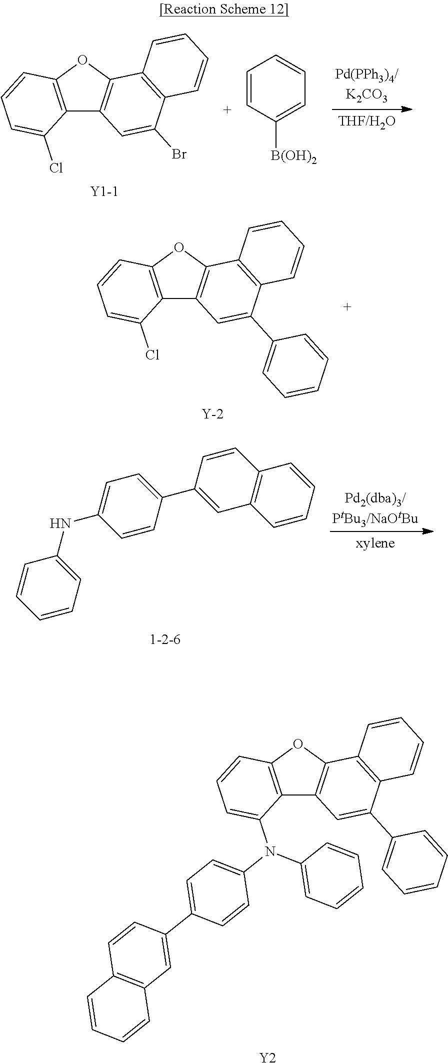

1st step: Synthesis of Intermediate Y2-1

15.00 g (45.24 mmol) of Intermediate Y1-1, 662 g (54.28 mmol) of phenylboronic acid, 1.57 g (1.36 mmol) of Pd(PPh₃)₄, and 12.50 g (90.47 mmol) of K₂CO₃ were suspended in 200 mL of THF/100 mL of distilled water in a round-bottomed flask and stirred under reflux for 12 hours. When a reaction was completed, the resultant was concentrated and extracted with methylene chloride, and an organic layer therefrom was silica gel columned, obtaining 12.5 g (Yield: 84%) of Intermediate Y2-1.

2nd step: Synthesis of Compound Y2

12.30 g (37.43 mmol) of Intermediate Y2-1, 13.27 g (44.92 mmol) of Intermediate 1-2-6, 5.39 g (56.15 mmol) of NaOtBu, and 2.73 g (50%, 2.25 mmol) of PtBu₃ were dissolved in 200 mL of xylene, and 2.06 g (2.25 mmol) of Pd₂(dba)₃ was added thereto and then, stirred under reflux for 12 hours under a nitrogen atmosphere. When a reaction was completed, after extraction with xylene and distilled water, an organic layer was concentrated. The organic layer was silica gel-columned, obtaining 18.0 g (Yield: 82%) of Compound Y2.

LC/MS calculated for: C44H29NO Exact Mass: 587.72 found for 587.96 [M+H]

### Synthesis Example 11: Synthesis of Compound A-3

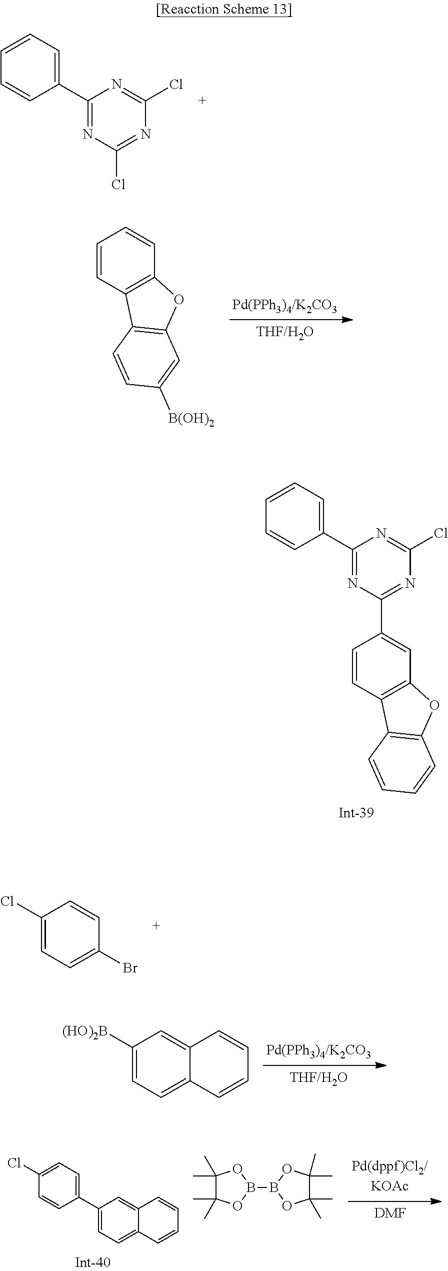

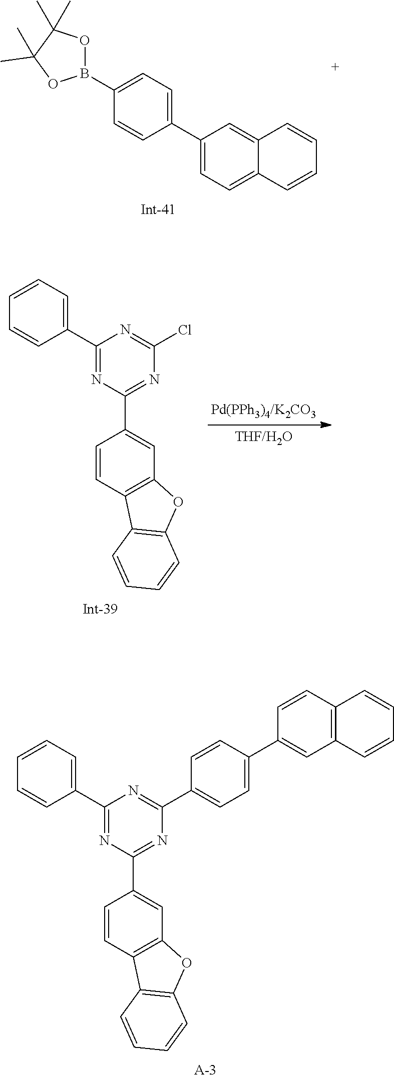

1st step: Synthesis of Intermediate Int-39

22.6 g (100 mmol) of 2,4-dichloro-6-phenyl-1,3,5-triazine was added to 200 mL of tetrahydrofuran and 100 mL of distilled water in a round-bottomed flask, 0.9 equivalent of dibenzofuran-3-boronic acid (CAS No.: 395087-89-5), 0.03 equivalent of tetrakis(triphenylphosphine)palladium, and 2 equivalent of potassium carbonate were added thereto and then, heated under reflux under a nitrogen atmosphere. After 6 hours, the reaction solution was cooled, after removing an aqueous layer, an organic layer therefrom was dried under a reduced pressure. A solid therefrom was washed with water and hexane, recrystallized with 200 mL of toluene, obtaining 21.4 g (Yield: 60%) of Intermediate Int-39.

2nd step: Synthesis of Intermediate Int-40

50.0 g (261.16 mmol) of 1-bromo-4-chloro-benzene, 44.9 g (261.16 mmol) of 2-naphthalene boronic acid, 9.1 g (7.83 mmol) of tetrakis(triphenylphosphine)palladium, and 71.2 g (522.33 mmol) of potassium carbonate were dissolved in 1,000 mL of tetrahydrofuran and 500 mL of distilled water in a round-bottomed flask and stirred under reflux under a nitrogen atmosphere. After 6 hours, the reaction solution was cooled, and after removing an aqueous layer, an organic layer therefrom was dried under a reduced pressure. The obtained solid was washed with water and hexane and recrystallized with 200 mL of toluene, obtaining 55.0 g (Yield: 88%) of Intermediate Int-40.

3rd step: Synthesis of Intermediate Int-41

100.0 g (418.92 mmol) of Intermediate Int-40 was added to 1000 mL of DMF in a round-bottomed flask, and 17.1 g (20.95 mmol) of dichlorodiphenylphosphinoferrocene palladium, 127.7 g (502.70 mmol) of bis(pinacolato)diboron, and 123.3 g (1256.76 mmol) of potassium acetate were added thereto and then, stirred under reflux for 12 hours under a nitrogen atmosphere. The reaction solution was cooled and then, added dropwise to 2 L of water to catch a solid. The obtained solid was dissolved in boiling toluene and then, filtered through silica gel, and a filtrate therefrom was concentrated. The concentrated solid was stirred with a small amount of hexane and then, filtered, obtaining 28.5 g (Yield: 70%) of Intermediate Int-41.

4th step: Synthesis of Compound A-3

10.0 g (27.95 mmol) of Intermediate Int-41, 11.1 g (33.54 mmol) of Intermediate Int-39, 1.0 g (0.84 mmol) of tetrakis(triphenylphosphine)palladium, and 7.7 g (55.90 mmol) of potassium carbonate were dissolved in 150 mL of tetrahydrofuran and 75 mL of distilled water in a round-bottomed flask and then, stirred under reflux under a nitrogen atmosphere. After 12 hours, the reaction solution was cooled, and after removing an aqueous layer, an organic layer therefrom was dried under a reduced pressure. A solid obtained therefrom was washed with water and methanol and recrystallized with 200 mL of toluene, obtaining 13.4 g (Yield: 91%) of Compound A-3.

calcd. C37H23N3O: C, 84.55; H, 4.41; N, 7.99; O, 3.04; found: C, 84.55; H, 4.41; N, 8.00; O, 3.03

### Synthesis Example 12: Synthesis of Compound A-71

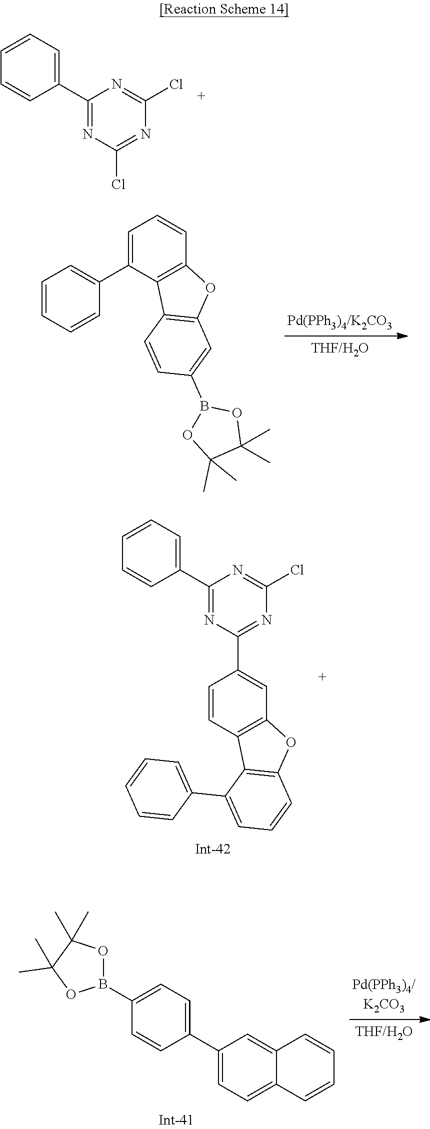

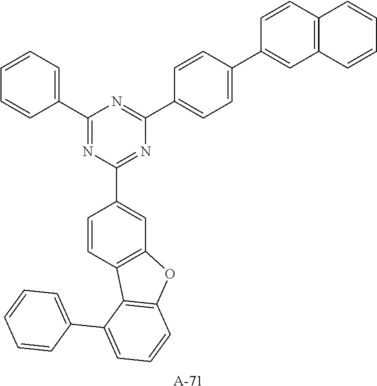

1st step: Synthesis of Intermediate Int-42

2,4-dichloro-6-phenyl-1,3,5-triazine and 1-phenyl-7-(4,4,5,5-tetramethyl-[1,3,2]dioxaborolan-2-yl)-dibenzofuran were used respectively by 1.0 equivalent to synthesize Intermediate Int-42 in the same manner as the 1^{st} step of Synthesis Example 11.

2nd step: Synthesis of Compound A-71

Intermediate Int-42 and Intermediate Int-41 were used respectively by 1.0 equivalent to synthesize Compound A-71 in the same method as the 4^{th} step of Synthesis Example 11.

calcd. C43H27N3O: C, 85.83; H, 4.52; N, 6.98; O, 2.66; found: C, 85.83; H, 4.52; N, 6.98; O, 2.66

### Synthesis Example 13: Synthesis of Compound A-61

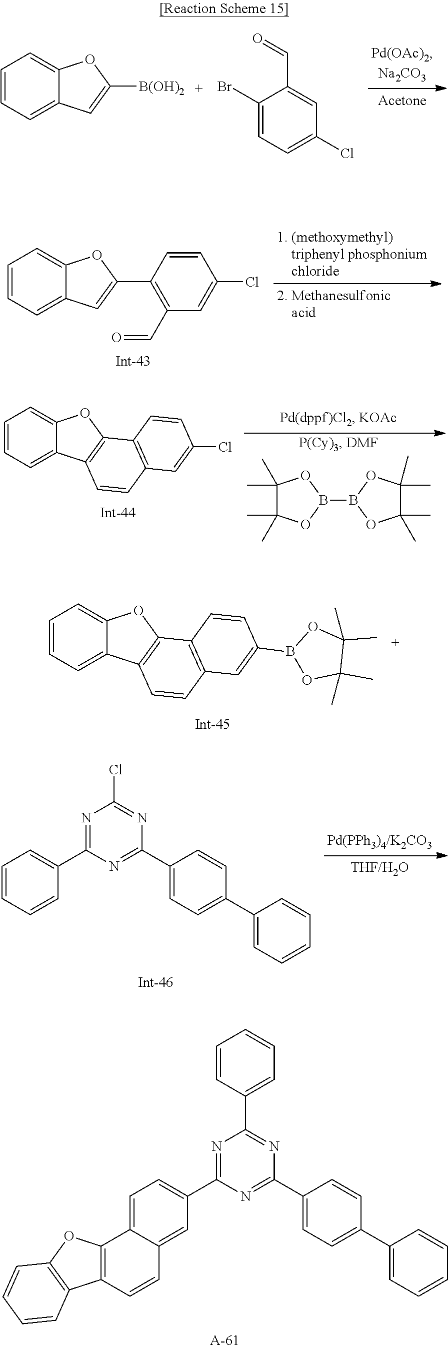

1st step: Synthesis of Intermediate Int-43

21.95 g (135.53 mmol) of 2-benzofuranylboronic acid, 26.77 g (121.98 mmol) of 2-bromo-5-chlorobenzaldehyde, 2.74 g (12.20 mmol) of Pd(OAc)₂, and 25.86 g (243.96 mmol) of Na₂CO₃ were suspended in 200 ml of acetone/220 ml of distilled water in a round-bottomed flask and then, stirred for 12 hours at ambient temperature. When a reaction was completed, the resultant was concentrated and extracted with methylene chloride, and an organic layer therefrom was silica gel-columned, obtaining 21.4 g (Yield: 68%) of Intermediate Int-43.

2nd step: Synthesis of Intermediate Int-44

20.4 g (79.47 mmol) of Intermediate Int-43 and 29.97 g (87.42 mmol) of (methoxymethyl)triphenylphosphonium chloride were suspended in 400 ml of THF, and 10.70 g (95.37 mmol) of potassium tert-butoxide was added thereto and then, stirred for 12 hours at ambient temperature. When a reaction was completed, 400 ml of distilled water was added thereto for extraction, an organic layer therefrom was concentrated and re-extracted with methylene chloride, magnesium sulfate was added to the organic layer and then, stirred for 30 minutes and filtered, and a filtrate therefrom was concentrated. Subsequently, 100 ml of methylene chloride was added to the concentrated filtrate again, and 10 ml of methanesulfonic acid was added thereto and then, stirred for 1 hour.

When a reaction was completed, a solid produced therein was filtered and dried with distilled water and methyl alcohol, obtaining 21.4 g (Yield: 65%) of Intermediate Int-44.

3rd step: Synthesis of Intermediate Int-45

12.55 g (49.66 mmol) of Intermediate Int-44, 2.43 g (2.98 mmol) of Pd(dppf)Cl₂, 15.13 g (59.60 mmol) of bis(pinacolato)diboron, 14.62 g (148.99 mmol) of KOAc, and 3.34 g (11.92 mmol) of P(Cy)₃ were suspended in 200 ml of DMF and then, stirred under reflux for 12 hours. When a reaction was completed, 200 ml of distilled water was added thereto, a solid produced therein was and filtered and extracted with methylene chloride, and an organic layer therefrom was columned with hexane:EA (4:1 v/v), obtaining 13 g (Yield: 76%) of Intermediate Int-45.

4th step: Synthesis of Compound A-61

Intermediate Int-45 and Intermediate Int-46 were used respectively by 1.0 equivalent in the same manner as the 4th step of Synthesis Example 11, synthesizing Compound A-61.

calcd. C37H23N3O: C, 84.55; H, 4.41; N, 7.99; O, 3.04; found: C, 84.55; H, 4.41; N, 7.99; O, 3.04

### Synthesis Example 14: Synthesis of Compound A-17 Synthesis

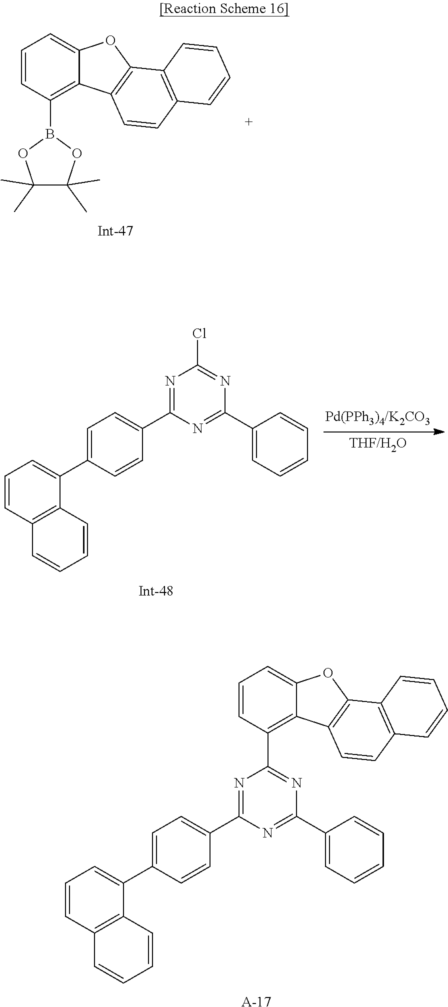 [SMILES: CC1(C)OB(c2cccc3oc4c5ccccc5ccc4c23)OC1(C)C ; Clc1nc(-c2ccccc2)nc(-c2ccc(-c3cccc4ccccc34)cc2)n1 ; c1ccc(-c2nc(-c3ccc(-c4cccc5ccccc45)cc3)nc(-c3cccc4oc5c6ccccc6ccc5c34)n2)cc1 ; "A-17"]

Intermediate Int-47 and Intermediate Int-48 were used respectively by 1.0 equivalent in the same manner as the 4th step of Synthesis Example 11, synthesizing Compound A-17.

caled. C41H25N3O: C, 85.54; H, 4.38; N, 7.30; O, 2.78; found: C, 85.53; H, 4.38; N, 7.30; O, 2.77

### Synthesis Example 15: Synthesis of Compound A-37

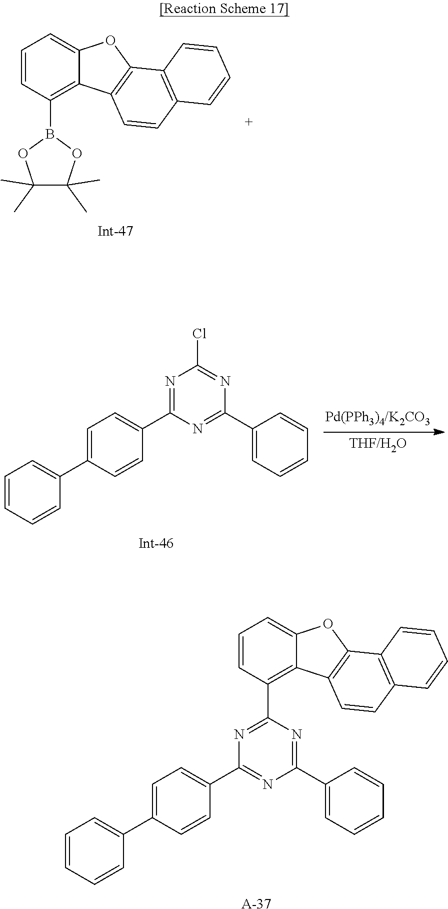 [SMILES: CC1(C)OB(c2cccc3oc4c5ccccc5ccc4c23)OC1(C)C ; Clc1nc(-c2ccccc2)nc(-c2ccc(-c3ccccc3)cc2)n1 ; c1ccc(-c2ccc(-c3nc(-c4ccccc4)nc(-c4cccc5oc6c7ccccc7ccc6c45)n3)cc2)cc1 ; "A-37"]

Intermediate Int-47 and Intermediate Int-46 were used respectively by 1.0 equivalent in the same manner as the 4th step of Synthesis Example 11, synthesizing Compound A-37.

calcd. C37H23N3O: C, 84.55; H, 4.41; N, 7.99; O, 3.04; found: C, 84.57; H, 4.40; N, 7.99; O, 3.03

Synthesis of Synthesis Examples 16 to 20

Each compound was synthesized in the same manner as the 4^{th} step of Synthesis Example 11 except that Int C shown in Table 1 was used instead of Int-41 of Synthesis Example 11, and Int D shown in Table 1 was used instead of Int-39.

TABLE 1

| Synthesis | | | | Yield | |
|---|---|---|---|---|---|
| Example | Int C | Int D | Final product | amount | Physical property data of final product |
| Synthesis | Int-49 | Int-48 | Compound A- | 8.33 g (74%) | calcd. C41H25N3S: C, 83.22; H, 4.26; N, |
| Example 16 | | | 24 | | 7.10; S, 5.42 |
| | | | | | found: C, 83.22; H, 4.26; N, 7.10; S, 5.42 |
| Synthesis | Int-50 | Int-54 | Compound A- | 6.29 g (71%) | calcd. C37H23N3S: C, 82.04; H, 4.28; N, |
| Example 17 | | | 77 | | 7.76; S, 5.92 |
| | | | | | found: C, 82.04; H, 4.28; N, 7.76; S, 5.92 |
| Synthesis | lnt-51 | Int-55 | Compound A- | 7.67 g (71%) | calcd. C41H25N3O: C, 85.54; H, 4.38; N, |
| Example 18 | | | 35 | | 7.30; O, 2.78 |
| | | | | | found: C, 85.55; H, 4.38; N, 7.29; O, 2.78 |
| Synthesis | Int-52 | Int-56 | Compound A- | 8.24 g (75%) | calcd. C39H23N3O: C, 85.23; H, 4.22; N, |
| Example 19 | | | 109 | | 7.65; O, 2.91 |
| | | | | | found: C, 85.23; H, 4.22; N, 7.65; O, 2.91 |
| Synthesis | Int-53 | Int-56 | Compound A- | 7.89 g (77%) | calcd. C39H23N3O: C, 85.23; H, 4.22; N, |
| Example 20 | | | 113 | | 7.65; O, 2.91 |
| | | | | | found: C, 85.23; H, 4.22; N, 7.65; O, 2.91 |

<Int C> 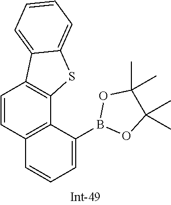 [SMILES: CC1(C)OB(c2cccc3ccc4c5ccccc5sc4c23)OC1(C)C] 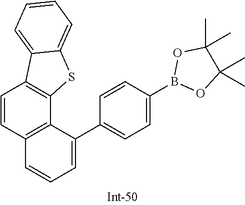 [SMILES: CC1(C)OB(c2ccc(-c3cccc4ccc5c6ccccc6sc5c34)cc2)OC1(C)C] 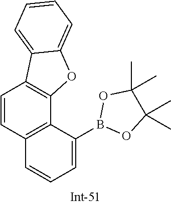 [SMILES: CC1(C)OB(c2cccc3ccc4c5ccccc5oc4c23)OC1(C)C] 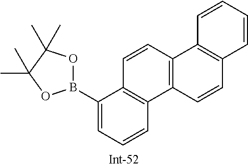 [SMILES: CC1(C)OB(c2cccc3c2ccc2c4ccccc4ccc32)OC1(C)C] 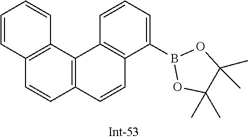 [SMILES: CC1(C)OB(c2cccc3c2ccc2ccc4ccccc4c23)OC1(C)C]
<Int D>

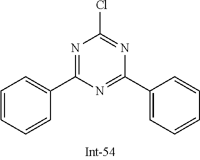 [SMILES: Clc1nc(-c2ccccc2)nc(-c2ccccc2)n1]

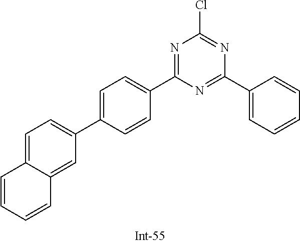 [SMILES: Clc1nc(-c2ccccc2)nc(-c2ccc(-c3ccc4ccccc4c3)cc2)n1]

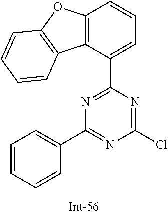 [SMILES: Clc1nc(-c2ccccc2)nc(-c2cccc3oc4ccccc4c23)n1]

(Manufacture of Organic Light Emitting Diode)

### Example 1

A glass substrate coated with ITO (Indium tin oxide) at a thickness of 1,500 Å was washed with distilled water. After washing with the distilled water, the glass substrate was ultrasonically washed with isopropyl alcohol, acetone, or methanol, a and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited on the hole transport layer to form a 1,300 Å-thick hole transport layer. On the hole transport layer, Compound B was deposited at a thickness of 700 Å to form a hole transport auxiliary layer. On the hole transport auxiliary layer, a 400 Å-thick light emitting layer was formed by using Compound 1-2 obtained in Synthesis Example 1 and doping 2 wt % of [Ir(piq)₂acac] as a dopant by vacuum-deposition. Subsequently, on the light emitting layer, Compound C was deposited at a thickness of 50 Å to form an electron transport auxiliary layer and Compound D and LiQ were simultaneously vacuum-deposited in a weight ratio of 1:1 to from a 300 Å-thick electron transport layer. On the electron transport layer, LiQ and Al were sequentially vacuum-deposited to be 15 Å-thick and 1,200 Å-thick, manufacturing an organic light emitting diode.

A structure of ITO/Compound A (3% NDP-9 doping, 100 Å)/Compound A (1,300 Å)/Compound B (700 Å)/EML [Compound 1-2 (98 wt %), Ir(piq)₂acac (2 wt %)] (400 Å) /Compound C (50 Å)/Compound D: Liq (300 Å)/LiQ (15 Å)/Al (1,200 Å).

Compound A: N-(biphenyl-4-yl)-9,9-dimethyl-N-(4-(9-phenyl-9H-carbazol-3-yl)phenyl)-9H-fluoren-2-amine

Compound B: N,N-di ([1,1′-biphenyl]-4-yl)-7,7-dimethyl-7H-fluoreno[4,3-b]benzofuran-10-amine

Compound C: 2-(3-(3-(9,9-dimethyl-9H-fluoren-2-yl)phenyl)phenyl)-4,6-diphenyl-1,3,5-triazine

Compound D: 8-(4-(4,6-di(naphthalen-2-yl)-1,3,5-triazin-2-yl)phenyl)quinoline

### Example 2

A glass substrate coated with ITO (Indium tin oxide) at a thickness of 1,500 Å was washed with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited on the hole transport layer to form a 1,300 Å-thick hole transport layer. On the hole transport layer, Compound B was deposited at a thickness of 700 Å to form a hole transport auxiliary layer. On the hole transport auxiliary layer, a 400 Å-thick light emitting layer was formed by simultaneously using Compound 1-2 of Synthesis Example 1 and Compound A-17 of Synthesis Example 14 as a host and doping 2 wt % of [Ir(piq)₂acac] as a dopant by vacuum-deposition. Compound 1-2 and Compound A-17 were used in a weight ratio of 5:5. Subsequently, on the light emitting layer, Compound C was deposited at a thickness of 50 Å to form an electron transport auxiliary layer and Compound D and LiQ was simultaneously vacuum-deposited in a weight ratio of 1:1 to from a 300 Å-thick electron transport layer. On the electron transport layer, LiQ and Al were sequentially vacuum-deposited to be 15 Å-thick and 1,200 Å-thick, manufacturing an organic light emitting diode.

A structure of ITO/Compound A (3% NDP-9 doping, 100 Å)/Compound A 1,300 Å/Compound B (700 Å)/EML [98 wt % of host (Compound 1-2: Compound A-17=5:5 (w/w)): 2 wt % of [Ir(piq)₂acac]] (400 Å)/Compound C (50 Å)/Compound D: Liq (300 Å) /LiQ (15 Å)/Al (1,200 Å).

### Examples 3 to 7 and Comparative Examples 1 and 2

Diodes of Examples 3 to 7 and Comparative Examples 1 and 2 were respectively manufactured according to the same method as Example 1 except that the host was changed as shown in Table 2.

### Examples 8 to 16 and Comparative Examples 3 and 4

Diodes according to Example 8 to 16 and Comparative Example 3 and 4 were manufactured according to the same method as Example 2 except that the host was changed into a single host, as shown in Table 3.

### Evaluations

Luminous efficiency and life-span characteristics of the organic light emitting diodes according to Examples 1 to 16 and Comparative Examples 1 to 4 were evaluated.

Specific measurement methods are as follows, and the results are shown in Tables 2 and 3.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

The luminous efficiency (cd/A) of the same current density (10 mA/cm²) was calculated using the luminance, current density, and voltage measured from the (1) and (2).

The relative values based on the luminous efficiency of Comparative Example 1 were calculated and shown in Table 2.

The relative values based on the luminous efficiency of Comparative Example 3 were calculated and shown in Table 3.

(4) Measurement of Life-span

T95 life-spans of the diodes according to Examples 1 to 16 and Comparative Examples 1 to 4 were measured as a time when their luminance decreased down to 95% relative to the initial luminance (cd/m²) after emitting light with 6,000 cd/m² as the initial luminance (cd/m²) and measuring their luminance decreases depending on a time with a Polanonix life-span measurement system.

The relative values based on the T95 life-span of Comparative Example 1 were calculated and shown in Table 2.

The relative values based on the T95 life-span of Comparative Example 3 were calculated and shown in Table 3.

TABLE 2

| | | T95 life- | Efficiency |
|---|---|---|---|
| | Host | span (%) | (%) |
| Example 1 | 1-2 | 200 | 118% |
| Example 3 | 1-42 | 220 | 120% |
| Example 4 | 1-55 | 150 | 116% |
| Example 5 | 1-57 | 220 | 122% |
| Example 6 | 1-59 | 250 | 125% |
| Comparative | Y1 | 100% | 100% |
| Example 1 | | | |
| Comparative | Y1 | 102% | 105% |
| Example 2 | | | |

TABLE 3

| | Host | | | |
|---|---|---|---|---|
| | First | Second | T95 life- | Efficiency |
| | host | host | span (%) | (%) |
| Example 2 | 1-2 | A-17 | 185 | 119 |
| Example 8 | 1-57 | | 205 | 119 |
| Example 9 | 1-59 | | 235 | 121 |
| Example 10 | 1-67 | | 220 | 122 |
| Example 11 | 1-57 | A-109 | 220 | 120 |
| Example 12 | 1-59 | | 260 | 120 |
| Example 13 | 1-67 | | 240 | 122 |
| Example 14 | 1-57 | A-113 | 218 | 117 |
| Example 15 | 1-59 | | 240 | 118 |
| Example 16 | 1-67 | | 220 | 120 |
| Comparative | Y1 | A-109 | 100% | 100% |
| Example 3 | | | | |
| Comparative | Y2 | | 115% | 102% |
| Example 4 | | | | |

Referring to Table 2, when the compounds of the Examples were applied as a host, efficiency and life-spans were improved, compared to the case where a comparative compound is applied. In addition, referring to Table 3, when a mixture of a first host and a second host according to the Examples was applied, overall efficiency and life-spans were greatly improved.

One or more embodiments may provide a compound for an organic optoelectronic device capable of implementing a high efficiency and long life-span organic optoelectronic device.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 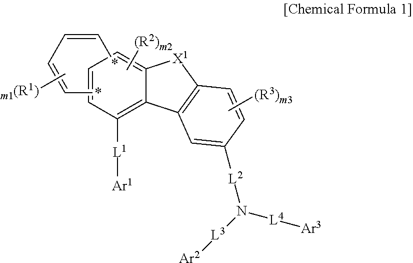
wherein, in Chemical Formula 1,
X¹ is O or S,
L¹ to L⁴ are each independently a single bond or a substituted or unsubstituted C6 to C30 arylene group,
R¹ to R³ are each independently hydrogen, deuterium, or a substituted or unsubstituted C1 to C10 alkyl group,
Ar¹ to Ar³ are each independently a substituted or unsubstituted C6 to C30 aryl group or a substituted or unsubstituted C2 to C30 heterocyclic group,
m1 an integer of 1 to 4,
m2 is 1,
m3 an integer of 1 to 3, and
* is a linking carbon.

2. The compound as claimed in claim 1, wherein:
Chemical Formula 1 is represented by Chemical Formula 1A or Chemical Formula 1B: 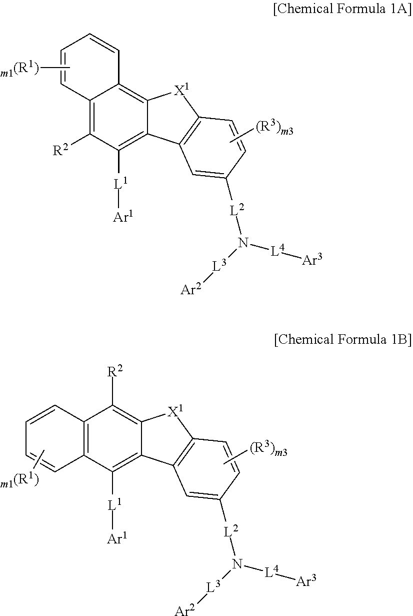
in Chemical Formula 1A and Chemical Formula 1B, X¹, L¹ to L⁴, Ar¹ to Ar³, R¹ to R³, m1, and m3 are defined the same as those of Chemical Formula 1.

3. The compound as claimed in claim 1, wherein Ar¹ is a substituted or unsubstituted phenyl group, a substituted or unsubstituted biphenyl group, a substituted or unsubstituted naphthyl group, a substituted or unsubstituted anthracenyl group, a substituted or unsubstituted phenanthrenyl group, a substituted or unsubstituted chrysenyl group, a substituted or unsubstituted benzophenanthrenyl group, a substituted or unsubstituted triphenylene group, a substituted or unsubstituted fluorenyl group, a substituted or unsubstituted dibenzosilolyl group, a substituted or unsubstituted dibenzofuranyl group, a substituted or unsubstituted dibenzothiophenyl group, or a substituted or unsubstituted carbazolyl group.

4. The compound as claimed in claim 1, wherein:
moiety -L¹-Ar¹ is a moiety of Group I, 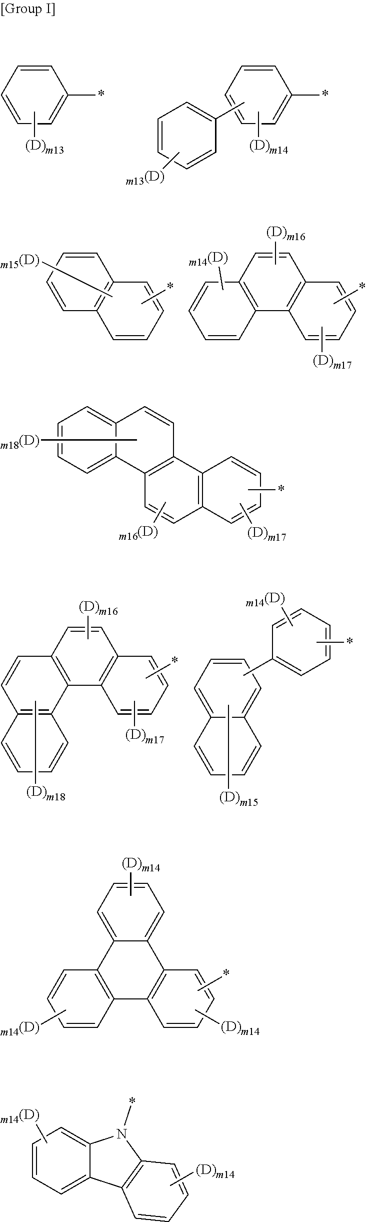 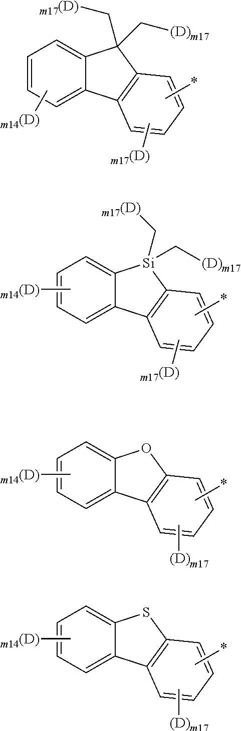
in Group I,
D is deuterium,
m13 is an integer of 0 to 5;
m14 is an integer of 0 to 4,
m15 is an integer of 0 to 7,
m16 is an integer of 0 to 2,
m17 is an integer of 0 to 3,
m18 is an integer of 0 to 6, and
* is a linking point.

5. The compound as claimed in claim 1, wherein Ar² and Ar³ are each independently a substituted or unsubstituted phenyl group, a substituted or unsubstituted biphenyl group, a substituted or unsubstituted naphthyl group, a substituted or unsubstituted phenanthrenyl group, a substituted or unsubstituted chrysenyl group, a substituted or unsubstituted fluorenyl group, a substituted or unsubstituted benzofluorenyl group, a substituted or unsubstituted benzophenanthrenyl group, a substituted or unsubstituted dibenzofuranyl group, a substituted or unsubstituted dibenzothiophenyl group, a substituted or unsubstituted dibenzosilolyl group, a substituted or unsubstituted benzonaphthofuranyl group, a substituted or unsubstituted benzonaphthothiophenyl group, a substituted or unsubstituted benzoxazolyl group, or a substituted or unsubstituted phenanthrooxazolyl group.

6. The compound as claimed in claim 1, wherein:
moieties -L³-Ar² and -L⁴-Ar³ are each independently a moiety of Group II: 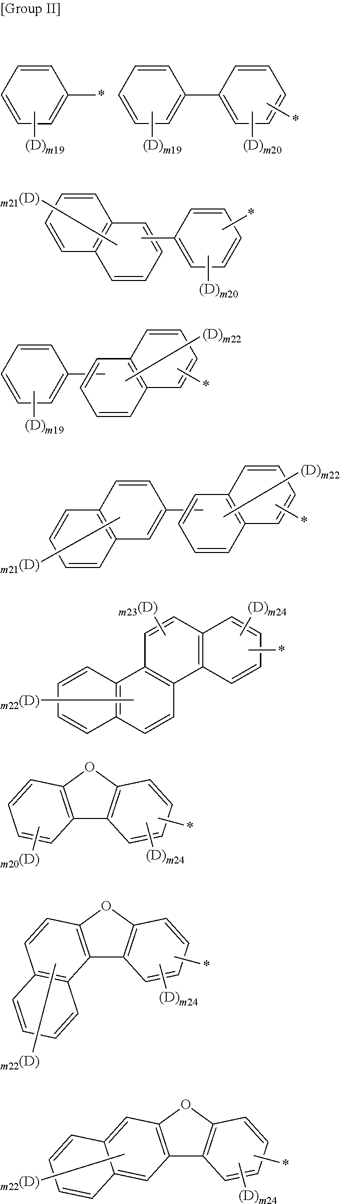 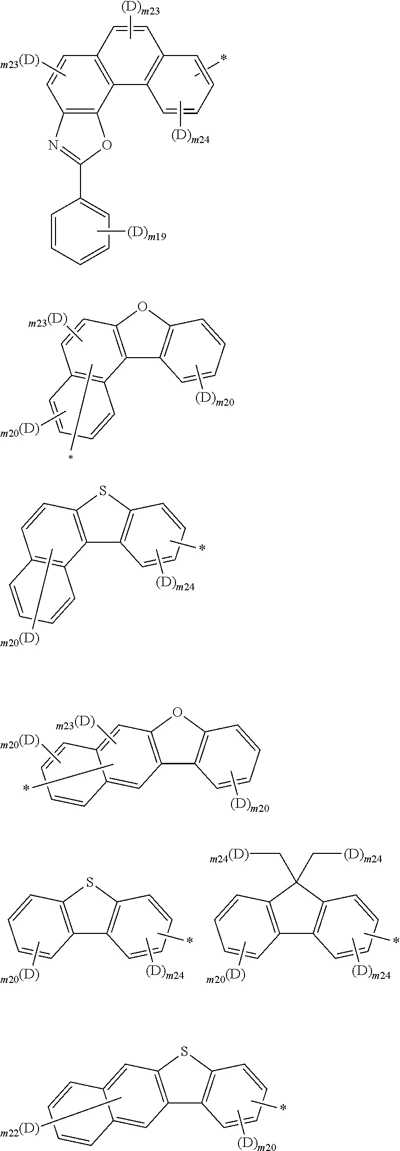 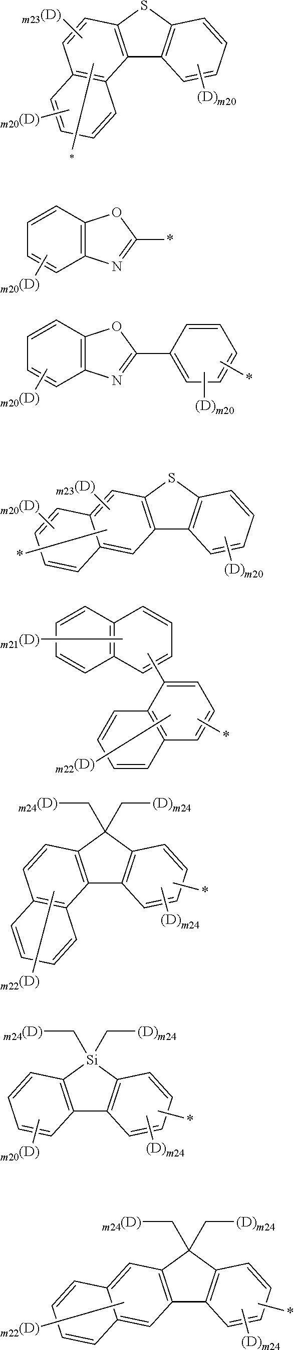
in Group II,
D is deuterium,
m19 is an integer of 0 to 5,
m20 is an integer of 0 to 4,
m21 is an integer of 0 to 7,
m22 is an integer of 0 to 6,
m23 is an integer of 0 to 2,
m24 is an integer of 0 to 3, and
* is a linking point.

7. The compound as claimed in claim 1, wherein the compound is a compound of Group 1:

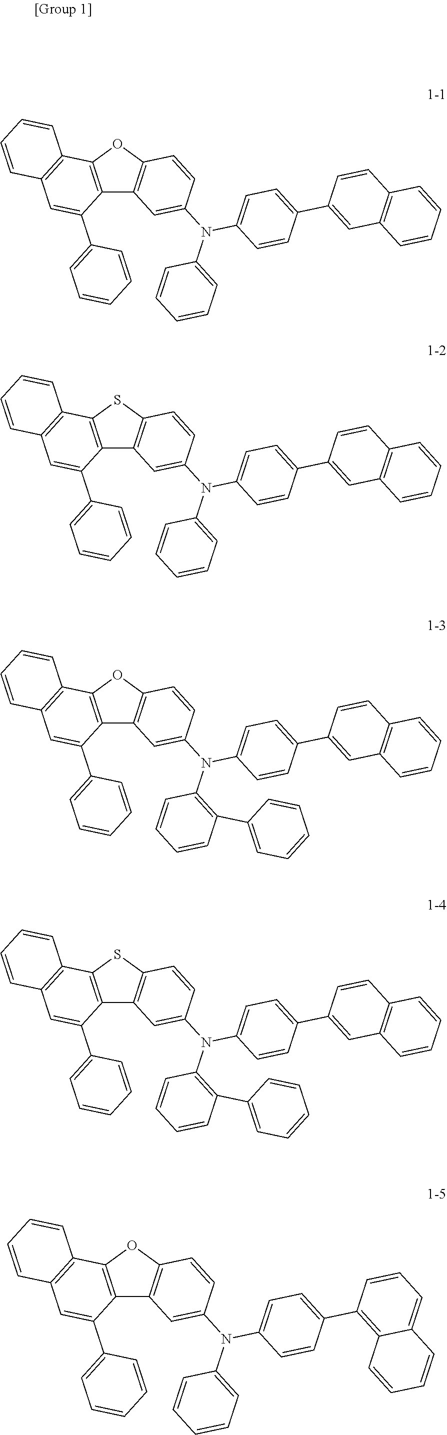 [SMILES: c1ccc(-c2cc3ccccc3c3oc4ccc(N(c5ccccc5)c5ccc(-c6ccc7ccccc7c6)cc5)cc4c23)cc1 ; c1ccc(-c2cc3ccccc3c3sc4ccc(N(c5ccccc5)c5ccc(-c6ccc7ccccc7c6)cc5)cc4c23)cc1 ; c1ccc(-c2ccccc2N(c2ccc(-c3ccc4ccccc4c3)cc2)c2ccc3oc4c5ccccc5cc(-c5ccccc5)c4c3c2)cc1 ; c1ccc(-c2ccccc2N(c2ccc(-c3ccc4ccccc4c3)cc2)c2ccc3sc4c5ccccc5cc(-c5ccccc5)c4c3c2)cc1 ; c1ccc(-c2cc3ccccc3c3oc4ccc(N(c5ccccc5)c5ccc(-c6cccc7ccccc67)cc5)cc4c23)cc1]

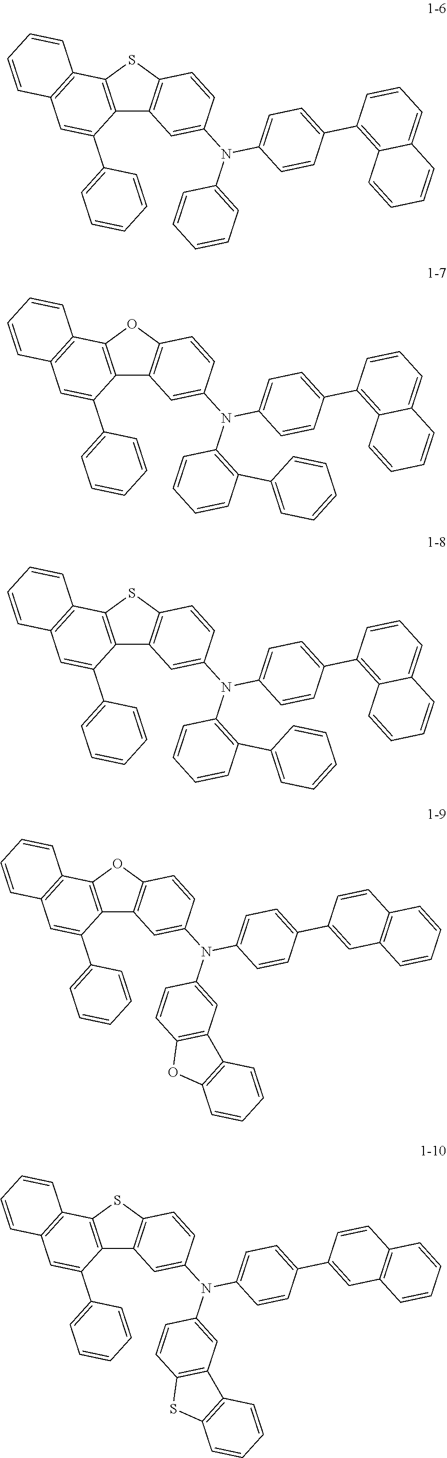 [SMILES: c1ccc(-c2cc3ccccc3c3sc4ccc(N(c5ccccc5)c5ccc(-c6cccc7ccccc67)cc5)cc4c23)cc1 ; c1ccc(-c2ccccc2N(c2ccc(-c3cccc4ccccc34)cc2)c2ccc3oc4c5ccccc5cc(-c5ccccc5)c4c3c2)cc1 ; c1ccc(-c2ccccc2N(c2ccc(-c3cccc4ccccc34)cc2)c2ccc3sc4c5ccccc5cc(-c5ccccc5)c4c3c2)cc1 ; c1ccc(-c2cc3ccccc3c3oc4ccc(N(c5ccc(-c6ccc7ccccc7c6)cc5)c5ccc6oc7ccccc7c6c5)cc4c23)cc1 ; c1ccc(-c2cc3ccccc3c3sc4ccc(N(c5ccc(-c6ccc7ccccc7c6)cc5)c5ccc6sc7ccccc7c6c5)cc4c23)cc1]

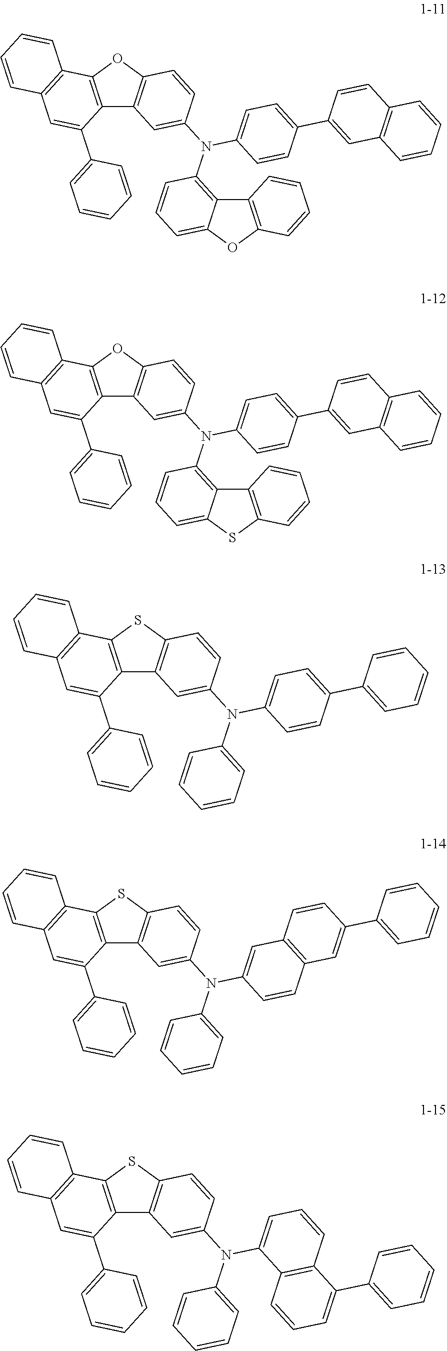 [SMILES: c1ccc(-c2cc3ccccc3c3oc4ccc(N(c5ccc(-c6ccc7ccccc7c6)cc5)c5cccc6oc7ccccc7c56)cc4c23)cc1 ; c1ccc(-c2cc3ccccc3c3oc4ccc(N(c5ccc(-c6ccc7ccccc7c6)cc5)c5cccc6sc7ccccc7c56)cc4c23)cc1 ; c1ccc(-c2ccc(N(c3ccccc3)c3ccc4sc5c6ccccc6cc(-c6ccccc6)c5c4c3)cc2)cc1 ; c1ccc(-c2ccc3cc(N(c4ccccc4)c4ccc5sc6c7ccccc7cc(-c7ccccc7)c6c5c4)ccc3c2)cc1 ; c1ccc(-c2cccc3c(N(c4ccccc4)c4ccc5sc6c7ccccc7cc(-c7ccccc7)c6c5c4)cccc23)cc1]

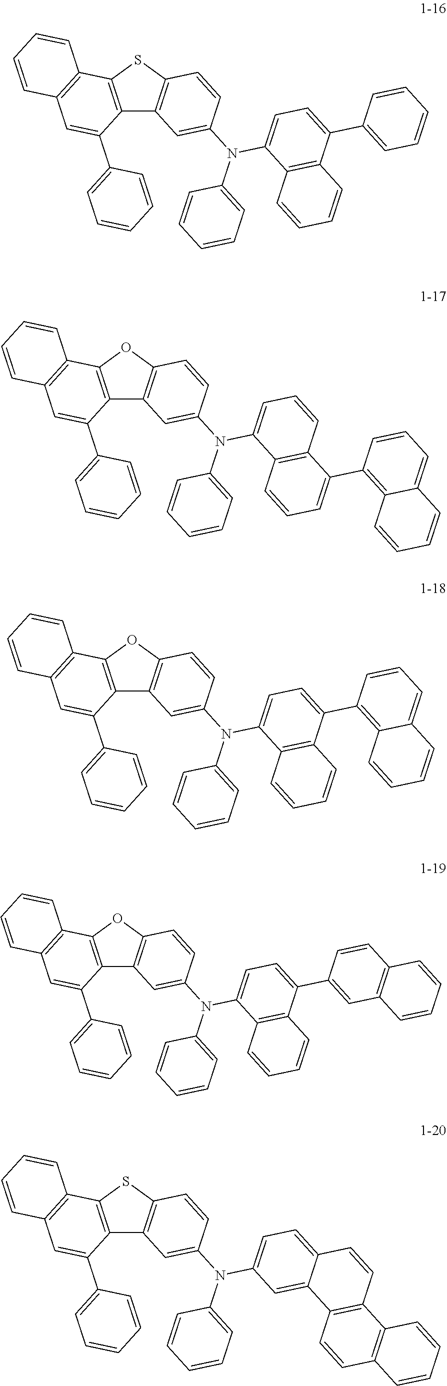 [SMILES: c1ccc(-c2ccc(N(c3ccccc3)c3ccc4sc5c6ccccc6cc(-c6ccccc6)c5c4c3)c3ccccc23)cc1 ; c1ccc(-c2cc3ccccc3c3oc4ccc(N(c5ccccc5)c5cccc6c(-c7cccc8ccccc78)cccc56)cc4c23)cc1 ; c1ccc(-c2cc3ccccc3c3oc4ccc(N(c5ccccc5)c5ccc(-c6cccc7ccccc67)c6ccccc56)cc4c23)cc1 ; c1ccc(-c2cc3ccccc3c3oc4ccc(N(c5ccccc5)c5ccc(-c6ccc7ccccc7c6)c6ccccc56)cc4c23)cc1 ; c1ccc(-c2cc3ccccc3c3sc4ccc(N(c5ccccc5)c5ccc6ccc7c8ccccc8ccc7c6c5)cc4c23)cc1]

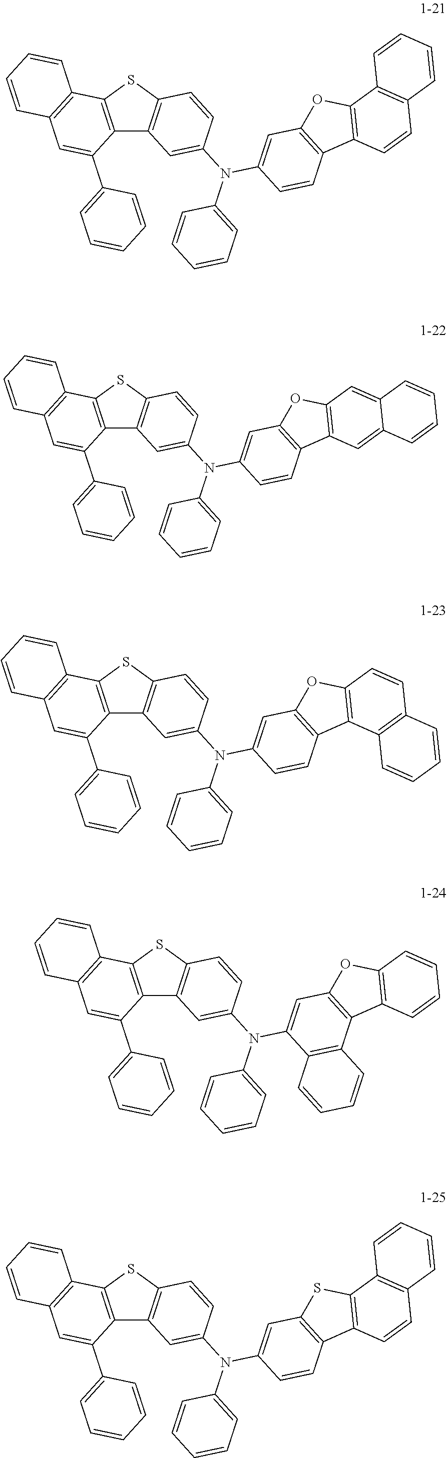 [SMILES: c1ccc(-c2cc3ccccc3c3sc4ccc(N(c5ccccc5)c5ccc6c(c5)oc5c7ccccc7ccc65)cc4c23)cc1 ; c1ccc(-c2cc3ccccc3c3sc4ccc(N(c5ccccc5)c5ccc6c(c5)oc5cc7ccccc7cc56)cc4c23)cc1 ; c1ccc(-c2cc3ccccc3c3sc4ccc(N(c5ccccc5)c5ccc6c(c5)oc5ccc7ccccc7c56)cc4c23)cc1 ; c1ccc(-c2cc3ccccc3c3sc4ccc(N(c5ccccc5)c5cc6oc7ccccc7c6c6ccccc56)cc4c23)cc1 ; c1ccc(-c2cc3ccccc3c3sc4ccc(N(c5ccccc5)c5ccc6c(c5)sc5c7ccccc7ccc65)cc4c23)cc1]

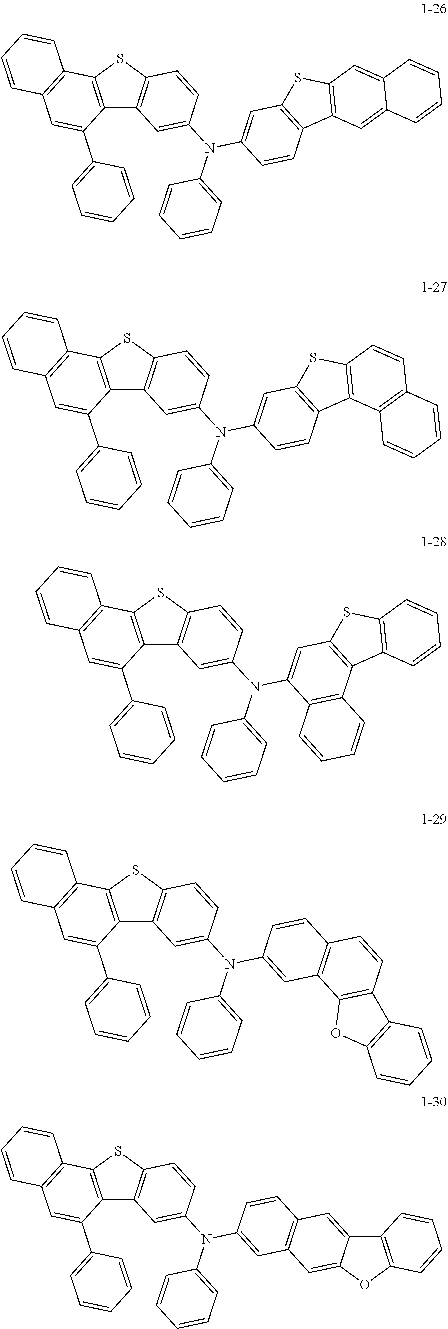 [SMILES: c1ccc(-c2cc3ccccc3c3sc4ccc(N(c5ccccc5)c5ccc6c(c5)sc5cc7ccccc7cc56)cc4c23)cc1 ; c1ccc(-c2cc3ccccc3c3sc4ccc(N(c5ccccc5)c5ccc6c(c5)sc5ccc7ccccc7c56)cc4c23)cc1 ; c1ccc(-c2cc3ccccc3c3sc4ccc(N(c5ccccc5)c5cc6sc7ccccc7c6c6ccccc56)cc4c23)cc1 ; c1ccc(-c2cc3ccccc3c3sc4ccc(N(c5ccccc5)c5ccc6ccc7c8ccccc8oc7c6c5)cc4c23)cc1 ; c1ccc(-c2cc3ccccc3c3sc4ccc(N(c5ccccc5)c5ccc6cc7c(cc6c5)oc5ccccc57)cc4c23)cc1]

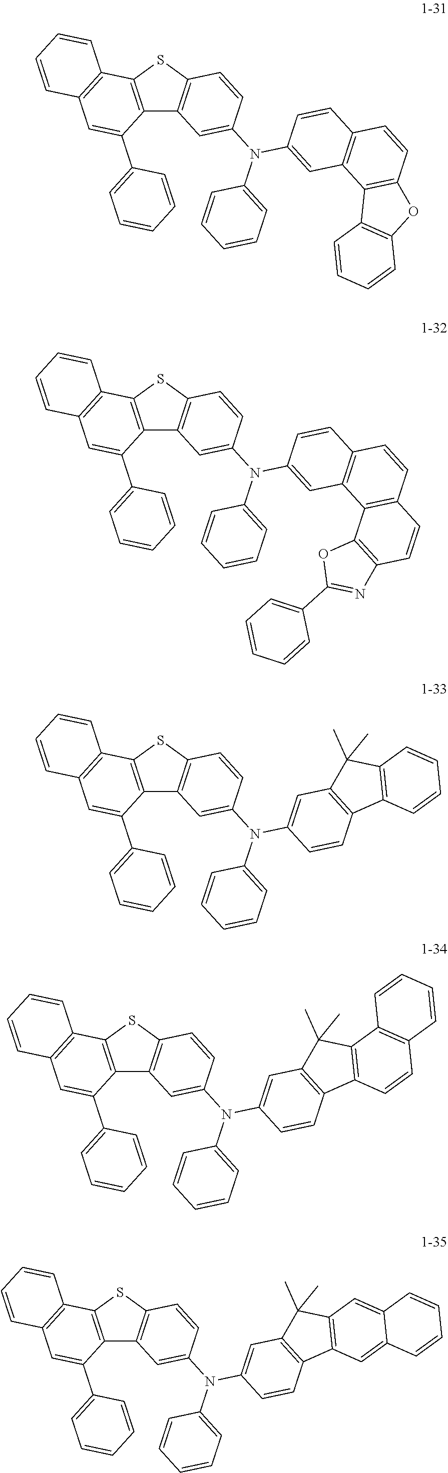 [SMILES: c1ccc(-c2cc3ccccc3c3sc4ccc(N(c5ccccc5)c5ccc6ccc7oc8ccccc8c7c6c5)cc4c23)cc1 ; c1ccc(-c2nc3ccc4ccc5ccc(N(c6ccccc6)c6ccc7sc8c9ccccc9cc(-c9ccccc9)c8c7c6)cc5c4c3o2)cc1 ; CC1(C)c2ccccc2-c2ccc(N(c3ccccc3)c3ccc4sc5c6ccccc6cc(-c6ccccc6)c5c4c3)cc21 ; CC1(C)c2cc(N(c3ccccc3)c3ccc4sc5c6ccccc6cc(-c6ccccc6)c5c4c3)ccc2-c2ccc3ccccc3c21 ; CC1(C)c2cc(N(c3ccccc3)c3ccc4sc5c6ccccc6cc(-c6ccccc6)c5c4c3)ccc2-c2cc3ccccc3cc21]

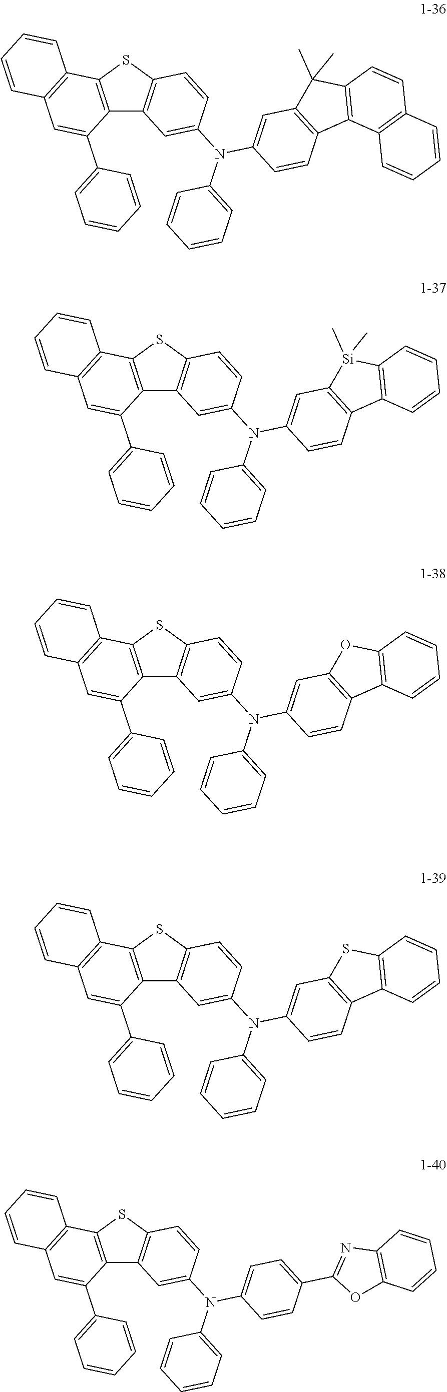 [SMILES: CC1(C)c2cc(N(c3ccccc3)c3ccc4sc5c6ccccc6cc(-c6ccccc6)c5c4c3)ccc2-c2c1ccc1ccccc21 ; C[Si]1(C)c2ccccc2-c2ccc(N(c3ccccc3)c3ccc4sc5c6ccccc6cc(-c6ccccc6)c5c4c3)cc21 ; c1ccc(-c2cc3ccccc3c3sc4ccc(N(c5ccccc5)c5ccc6c(c5)oc5ccccc56)cc4c23)cc1 ; c1ccc(-c2cc3ccccc3c3sc4ccc(N(c5ccccc5)c5ccc6c(c5)sc5ccccc56)cc4c23)cc1 ; c1ccc(-c2cc3ccccc3c3sc4ccc(N(c5ccccc5)c5ccc(-c6nc7ccccc7o6)cc5)cc4c23)cc1]

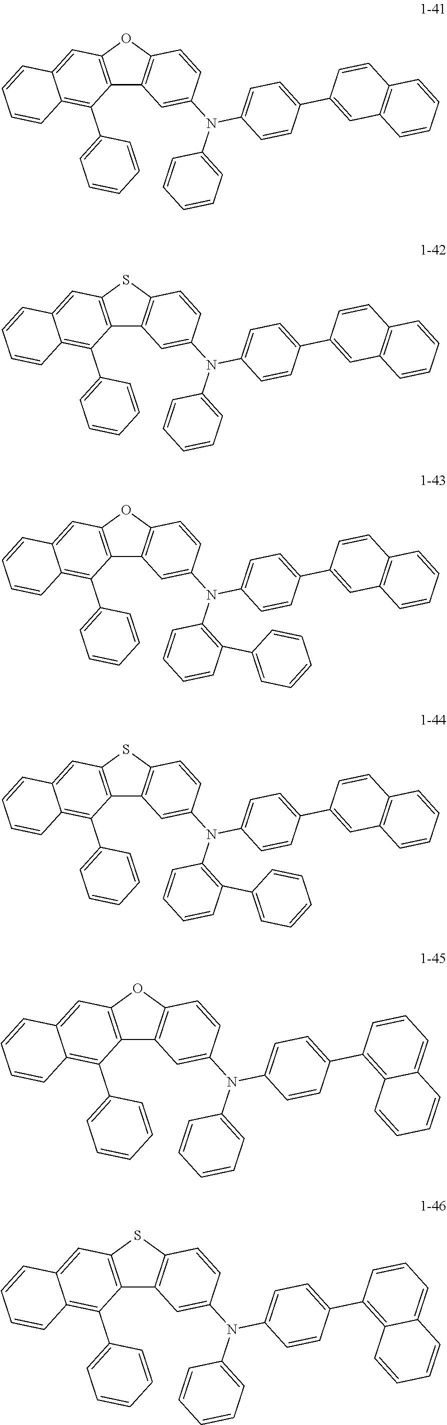 [SMILES: c1ccc(-c2c3ccccc3cc3oc4ccc(N(c5ccccc5)c5ccc(-c6ccc7ccccc7c6)cc5)cc4c23)cc1 ; c1ccc(-c2c3ccccc3cc3sc4ccc(N(c5ccccc5)c5ccc(-c6ccc7ccccc7c6)cc5)cc4c23)cc1 ; c1ccc(-c2ccccc2N(c2ccc(-c3ccc4ccccc4c3)cc2)c2ccc3oc4cc5ccccc5c(-c5ccccc5)c4c3c2)cc1 ; c1ccc(-c2ccccc2N(c2ccc(-c3ccc4ccccc4c3)cc2)c2ccc3sc4cc5ccccc5c(-c5ccccc5)c4c3c2)cc1 ; c1ccc(-c2c3ccccc3cc3oc4ccc(N(c5ccccc5)c5ccc(-c6cccc7ccccc67)cc5)cc4c23)cc1 ; c1ccc(-c2c3ccccc3cc3sc4ccc(N(c5ccccc5)c5ccc(-c6cccc7ccccc67)cc5)cc4c23)cc1]

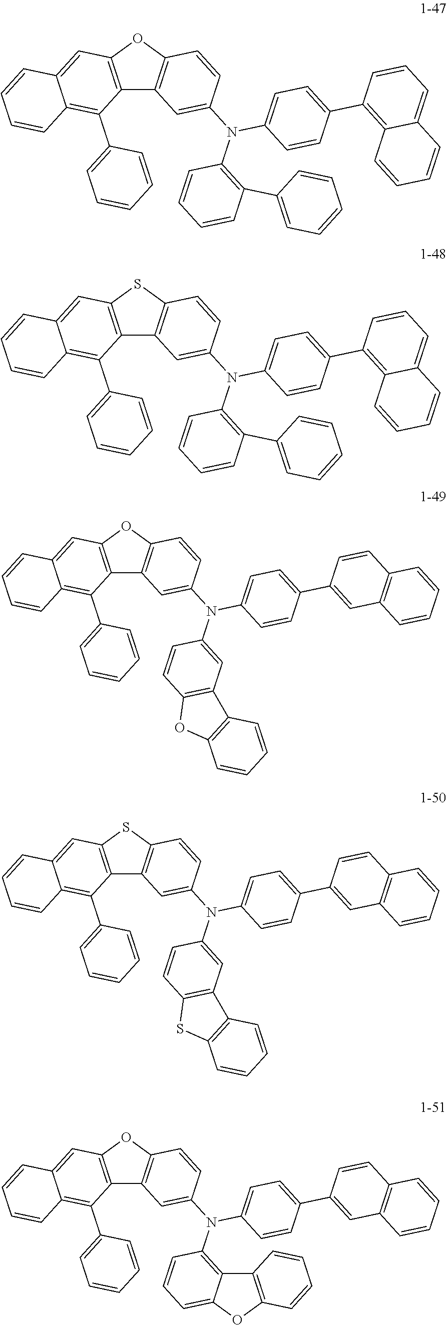 [SMILES: c1ccc(-c2ccccc2N(c2ccc(-c3cccc4ccccc34)cc2)c2ccc3oc4cc5ccccc5c(-c5ccccc5)c4c3c2)cc1 ; c1ccc(-c2ccccc2N(c2ccc(-c3cccc4ccccc34)cc2)c2ccc3sc4cc5ccccc5c(-c5ccccc5)c4c3c2)cc1 ; c1ccc(-c2c3ccccc3cc3oc4ccc(N(c5ccc(-c6ccc7ccccc7c6)cc5)c5ccc6oc7ccccc7c6c5)cc4c23)cc1 ; c1ccc(-c2c3ccccc3cc3sc4ccc(N(c5ccc(-c6ccc7ccccc7c6)cc5)c5ccc6sc7ccccc7c6c5)cc4c23)cc1 ; c1ccc(-c2c3ccccc3cc3oc4ccc(N(c5ccc(-c6ccc7ccccc7c6)cc5)c5cccc6oc7ccccc7c56)cc4c23)cc1]

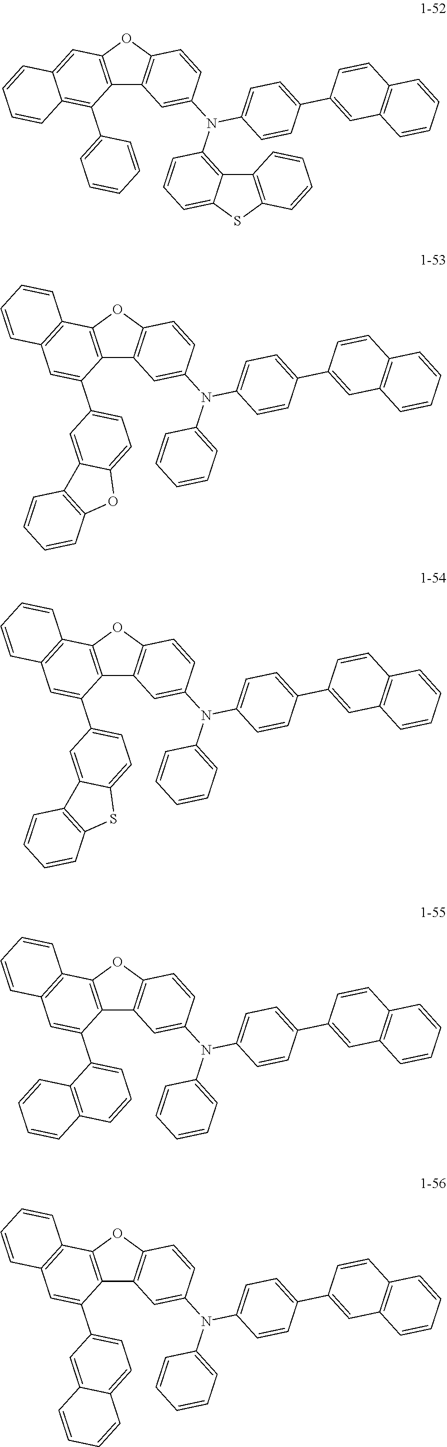 [SMILES: c1ccc(-c2c3ccccc3cc3oc4ccc(N(c5ccc(-c6ccc7ccccc7c6)cc5)c5cccc6sc7ccccc7c56)cc4c23)cc1 ; c1ccc(N(c2ccc(-c3ccc4ccccc4c3)cc2)c2ccc3oc4c5ccccc5cc(-c5ccc6oc7ccccc7c6c5)c4c3c2)cc1 ; c1ccc(N(c2ccc(-c3ccc4ccccc4c3)cc2)c2ccc3oc4c5ccccc5cc(-c5ccc6sc7ccccc7c6c5)c4c3c2)cc1 ; c1ccc(N(c2ccc(-c3ccc4ccccc4c3)cc2)c2ccc3oc4c5ccccc5cc(-c5cccc6ccccc56)c4c3c2)cc1 ; c1ccc(N(c2ccc(-c3ccc4ccccc4c3)cc2)c2ccc3oc4c5ccccc5cc(-c5ccc6ccccc6c5)c4c3c2)cc1]

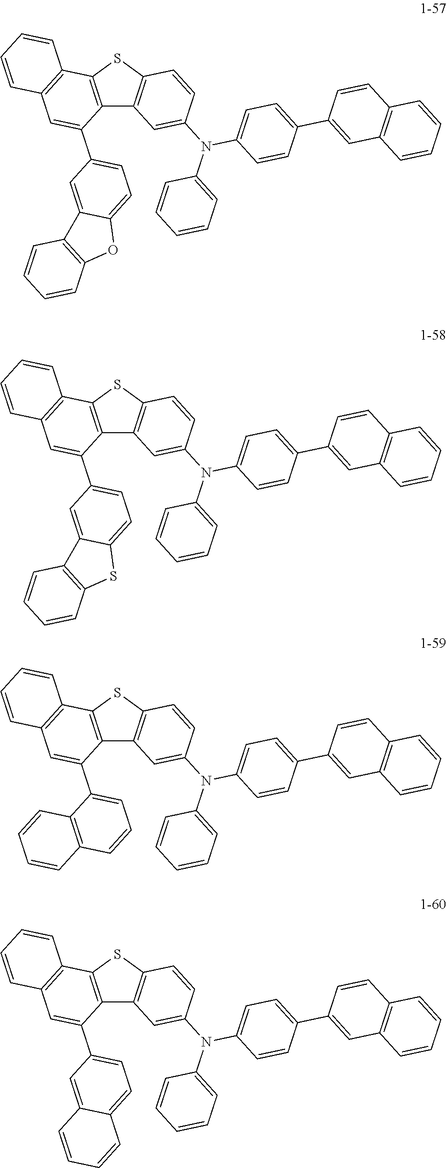 [SMILES: c1ccc(N(c2ccc(-c3ccc4ccccc4c3)cc2)c2ccc3sc4c5ccccc5cc(-c5ccc6oc7ccccc7c6c5)c4c3c2)cc1 ; c1ccc(N(c2ccc(-c3ccc4ccccc4c3)cc2)c2ccc3sc4c5ccccc5cc(-c5ccc6sc7ccccc7c6c5)c4c3c2)cc1 ; c1ccc(N(c2ccc(-c3ccc4ccccc4c3)cc2)c2ccc3sc4c5ccccc5cc(-c5cccc6ccccc56)c4c3c2)cc1 ; c1ccc(N(c2ccc(-c3ccc4ccccc4c3)cc2)c2ccc3sc4c5ccccc5cc(-c5ccc6ccccc6c5)c4c3c2)cc1]

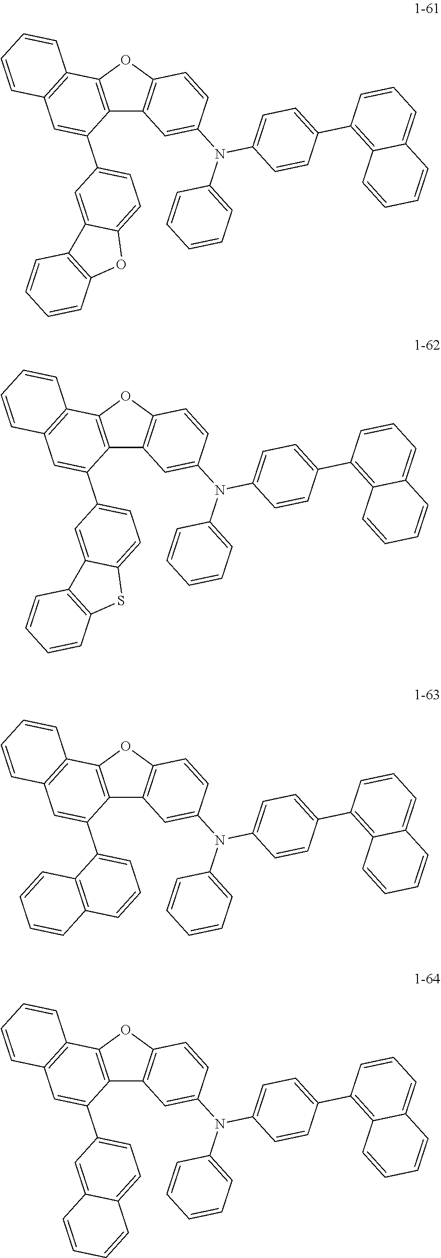 [SMILES: c1ccc(N(c2ccc(-c3cccc4ccccc34)cc2)c2ccc3oc4c5ccccc5cc(-c5ccc6oc7ccccc7c6c5)c4c3c2)cc1 ; c1ccc(N(c2ccc(-c3cccc4ccccc34)cc2)c2ccc3oc4c5ccccc5cc(-c5ccc6sc7ccccc7c6c5)c4c3c2)cc1 ; c1ccc(N(c2ccc(-c3cccc4ccccc34)cc2)c2ccc3oc4c5ccccc5cc(-c5cccc6ccccc56)c4c3c2)cc1 ; c1ccc(N(c2ccc(-c3cccc4ccccc34)cc2)c2ccc3oc4c5ccccc5cc(-c5ccc6ccccc6c5)c4c3c2)cc1]

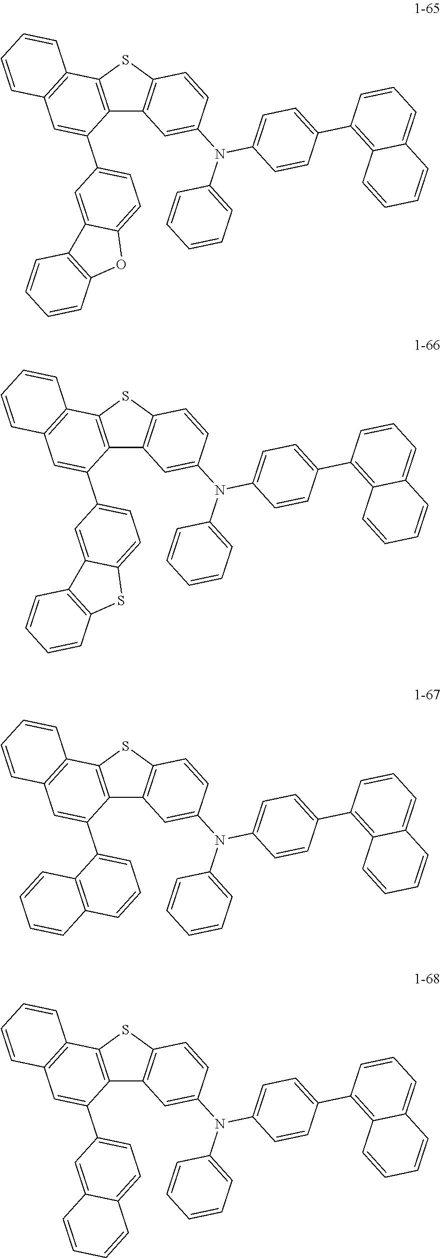 [SMILES: c1ccc(N(c2ccc(-c3cccc4ccccc34)cc2)c2ccc3sc4c5ccccc5cc(-c5ccc6oc7ccccc7c6c5)c4c3c2)cc1 ; c1ccc(N(c2ccc(-c3cccc4ccccc34)cc2)c2ccc3sc4c5ccccc5cc(-c5ccc6sc7ccccc7c6c5)c4c3c2)cc1 ; c1ccc(N(c2ccc(-c3cccc4ccccc34)cc2)c2ccc3sc4c5ccccc5cc(-c5cccc6ccccc56)c4c3c2)cc1 ; c1ccc(N(c2ccc(-c3cccc4ccccc34)cc2)c2ccc3sc4c5ccccc5cc(-c5ccc6ccccc6c5)c4c3c2)cc1]

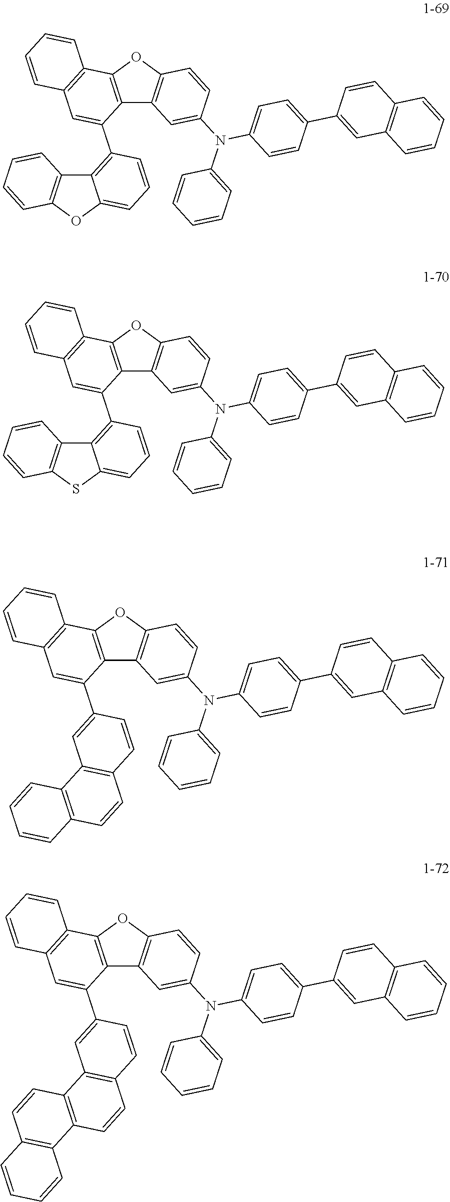 [SMILES: c1ccc(N(c2ccc(-c3ccc4ccccc4c3)cc2)c2ccc3oc4c5ccccc5cc(-c5cccc6oc7ccccc7c56)c4c3c2)cc1 ; c1ccc(N(c2ccc(-c3ccc4ccccc4c3)cc2)c2ccc3oc4c5ccccc5cc(-c5cccc6sc7ccccc7c56)c4c3c2)cc1 ; c1ccc(N(c2ccc(-c3ccc4ccccc4c3)cc2)c2ccc3oc4c5ccccc5cc(-c5ccc6ccc7ccccc7c6c5)c4c3c2)cc1 ; c1ccc(N(c2ccc(-c3ccc4ccccc4c3)cc2)c2ccc3oc4c5ccccc5cc(-c5ccc6ccc7c8ccccc8ccc7c6c5)c4c3c2)cc1]

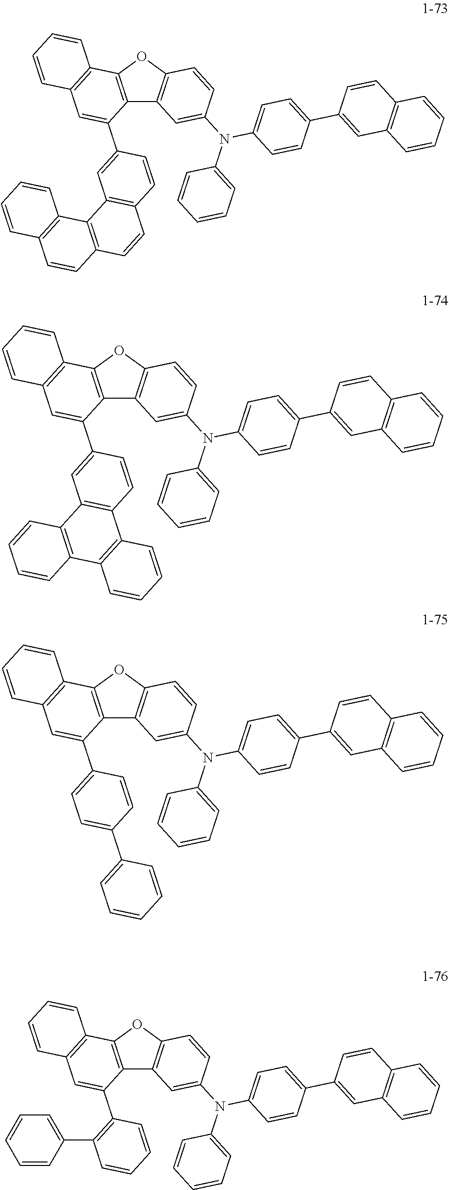 [SMILES: c1ccc(N(c2ccc(-c3ccc4ccccc4c3)cc2)c2ccc3oc4c5ccccc5cc(-c5ccc6ccc7ccc8ccccc8c7c6c5)c4c3c2)cc1 ; c1ccc(N(c2ccc(-c3ccc4ccccc4c3)cc2)c2ccc3oc4c5ccccc5cc(-c5ccc6c7ccccc7c7ccccc7c6c5)c4c3c2)cc1 ; c1ccc(-c2ccc(-c3cc4ccccc4c4oc5ccc(N(c6ccccc6)c6ccc(-c7ccc8ccccc8c7)cc6)cc5c34)cc2)cc1 ; c1ccc(-c2ccccc2-c2cc3ccccc3c3oc4ccc(N(c5ccccc5)c5ccc(-c6ccc7ccccc7c6)cc5)cc4c23)cc1]

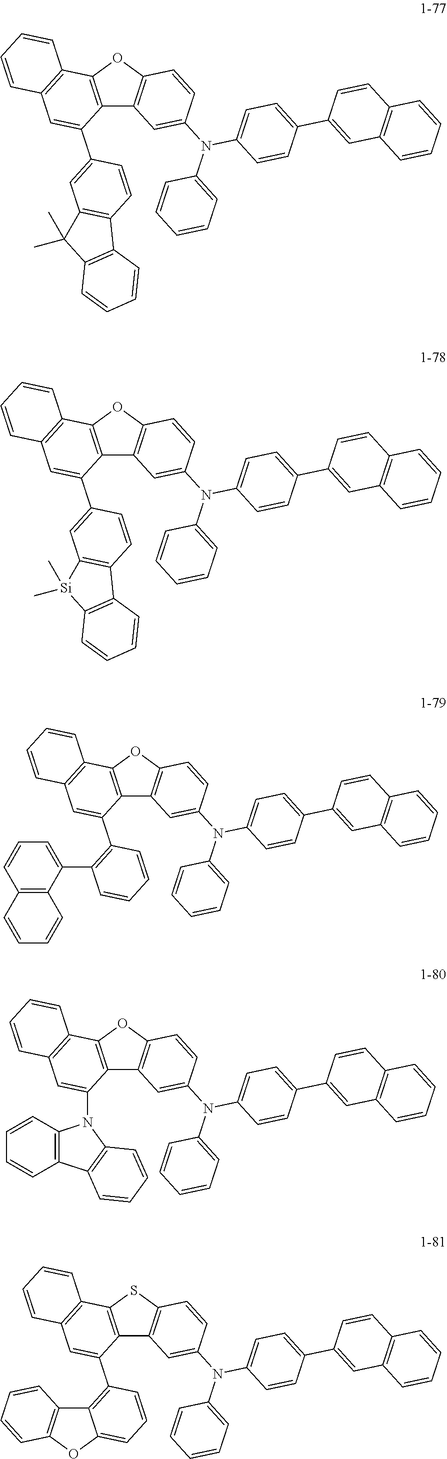 [SMILES: CC1(C)c2ccccc2-c2ccc(-c3cc4ccccc4c4oc5ccc(N(c6ccccc6)c6ccc(-c7ccc8ccccc8c7)cc6)cc5c34)cc21 ; C[Si]1(C)c2ccccc2-c2ccc(-c3cc4ccccc4c4oc5ccc(N(c6ccccc6)c6ccc(-c7ccc8ccccc8c7)cc6)cc5c34)cc21 ; c1ccc(N(c2ccc(-c3ccc4ccccc4c3)cc2)c2ccc3oc4c5ccccc5cc(-c5ccccc5-c5cccc6ccccc56)c4c3c2)cc1 ; c1ccc(N(c2ccc(-c3ccc4ccccc4c3)cc2)c2ccc3oc4c5ccccc5cc(-n5c6ccccc6c6ccccc65)c4c3c2)cc1 ; c1ccc(N(c2ccc(-c3ccc4ccccc4c3)cc2)c2ccc3sc4c5ccccc5cc(-c5cccc6oc7ccccc7c56)c4c3c2)cc1]

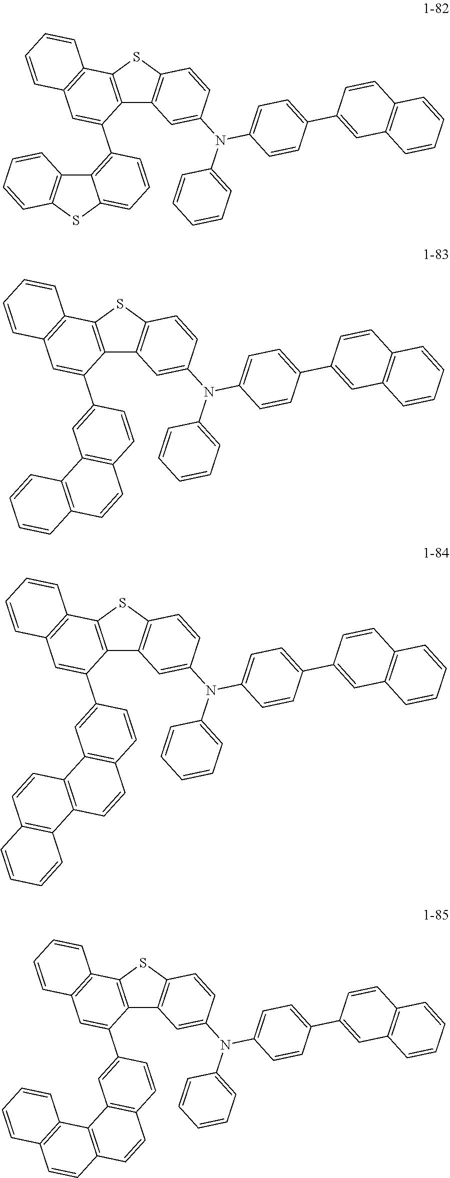 [SMILES: c1ccc(N(c2ccc(-c3ccc4ccccc4c3)cc2)c2ccc3sc4c5ccccc5cc(-c5cccc6sc7ccccc7c56)c4c3c2)cc1 ; c1ccc(N(c2ccc(-c3ccc4ccccc4c3)cc2)c2ccc3sc4c5ccccc5cc(-c5ccc6ccc7ccccc7c6c5)c4c3c2)cc1 ; c1ccc(N(c2ccc(-c3ccc4ccccc4c3)cc2)c2ccc3sc4c5ccccc5cc(-c5ccc6ccc7c8ccccc8ccc7c6c5)c4c3c2)cc1 ; c1ccc(N(c2ccc(-c3ccc4ccccc4c3)cc2)c2ccc3sc4c5ccccc5cc(-c5ccc6ccc7ccc8ccccc8c7c6c5)c4c3c2)cc1]

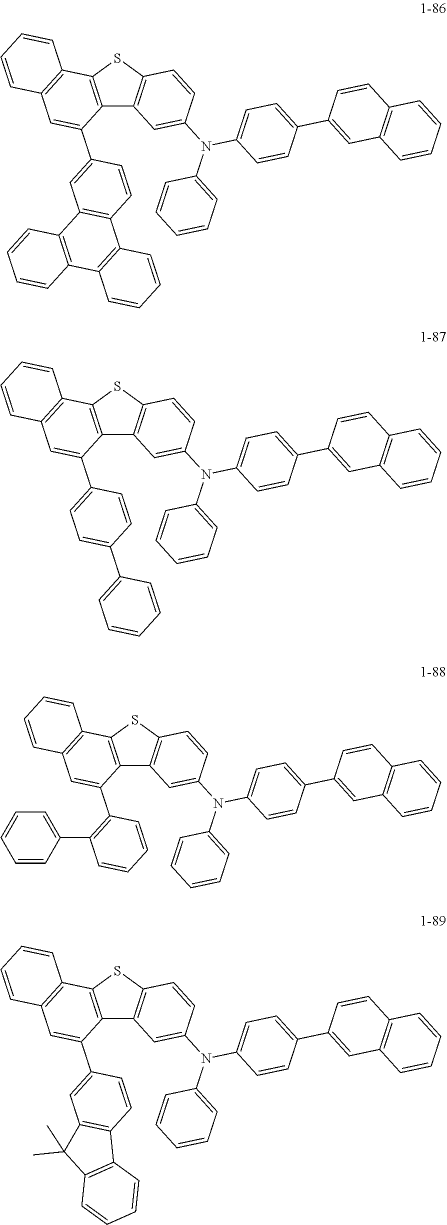 [SMILES: c1ccc(N(c2ccc(-c3ccc4ccccc4c3)cc2)c2ccc3sc4c5ccccc5cc(-c5ccc6c7ccccc7c7ccccc7c6c5)c4c3c2)cc1 ; c1ccc(-c2ccc(-c3cc4ccccc4c4sc5ccc(N(c6ccccc6)c6ccc(-c7ccc8ccccc8c7)cc6)cc5c34)cc2)cc1 ; c1ccc(-c2ccccc2-c2cc3ccccc3c3sc4ccc(N(c5ccccc5)c5ccc(-c6ccc7ccccc7c6)cc5)cc4c23)cc1 ; CC1(C)c2ccccc2-c2ccc(-c3cc4ccccc4c4sc5ccc(N(c6ccccc6)c6ccc(-c7ccc8ccccc8c7)cc6)cc5c34)cc21]

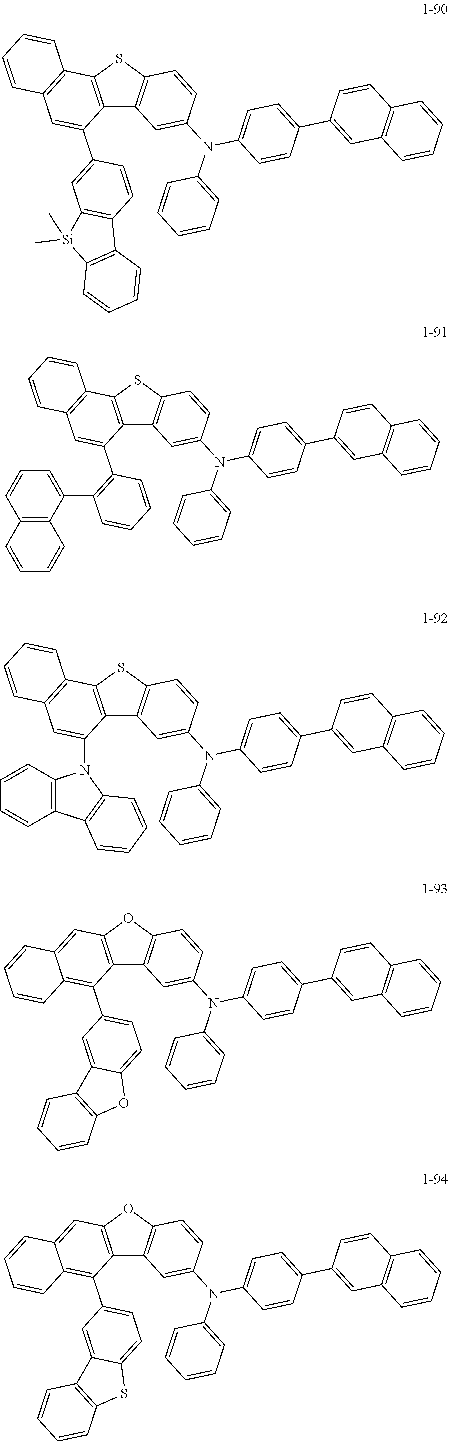 [SMILES: C[Si]1(C)c2ccccc2-c2ccc(-c3cc4ccccc4c4sc5ccc(N(c6ccccc6)c6ccc(-c7ccc8ccccc8c7)cc6)cc5c34)cc21 ; c1ccc(N(c2ccc(-c3ccc4ccccc4c3)cc2)c2ccc3sc4c5ccccc5cc(-c5ccccc5-c5cccc6ccccc56)c4c3c2)cc1 ; c1ccc(N(c2ccc(-c3ccc4ccccc4c3)cc2)c2ccc3sc4c5ccccc5cc(-n5c6ccccc6c6ccccc65)c4c3c2)cc1 ; c1ccc(N(c2ccc(-c3ccc4ccccc4c3)cc2)c2ccc3oc4cc5ccccc5c(-c5ccc6oc7ccccc7c6c5)c4c3c2)cc1 ; c1ccc(N(c2ccc(-c3ccc4ccccc4c3)cc2)c2ccc3oc4cc5ccccc5c(-c5ccc6sc7ccccc7c6c5)c4c3c2)cc1]

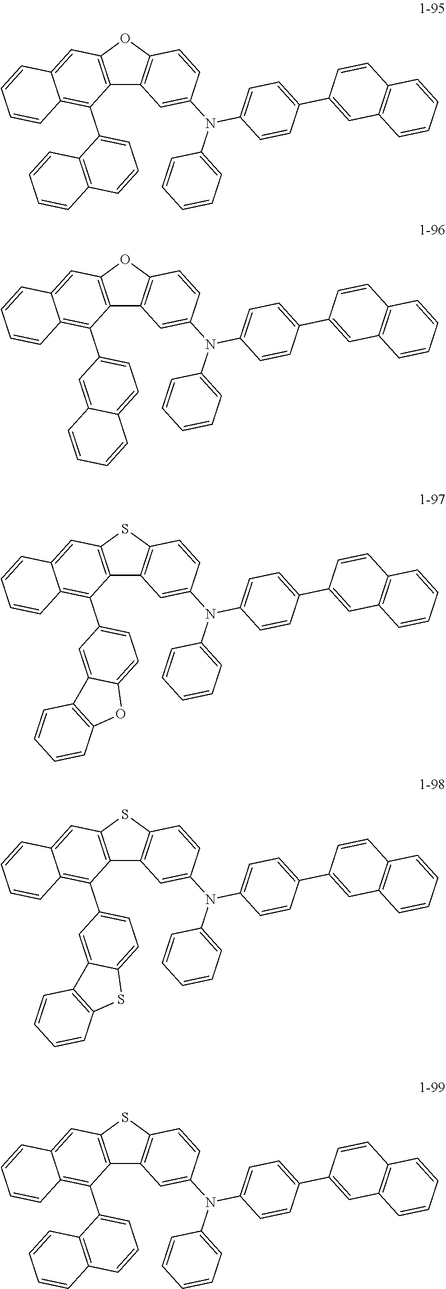 [SMILES: c1ccc(N(c2ccc(-c3ccc4ccccc4c3)cc2)c2ccc3oc4cc5ccccc5c(-c5cccc6ccccc56)c4c3c2)cc1 ; c1ccc(N(c2ccc(-c3ccc4ccccc4c3)cc2)c2ccc3oc4cc5ccccc5c(-c5ccc6ccccc6c5)c4c3c2)cc1 ; c1ccc(N(c2ccc(-c3ccc4ccccc4c3)cc2)c2ccc3sc4cc5ccccc5c(-c5ccc6oc7ccccc7c6c5)c4c3c2)cc1 ; c1ccc(N(c2ccc(-c3ccc4ccccc4c3)cc2)c2ccc3sc4cc5ccccc5c(-c5ccc6sc7ccccc7c6c5)c4c3c2)cc1 ; c1ccc(N(c2ccc(-c3ccc4ccccc4c3)cc2)c2ccc3sc4cc5ccccc5c(-c5cccc6ccccc56)c4c3c2)cc1]

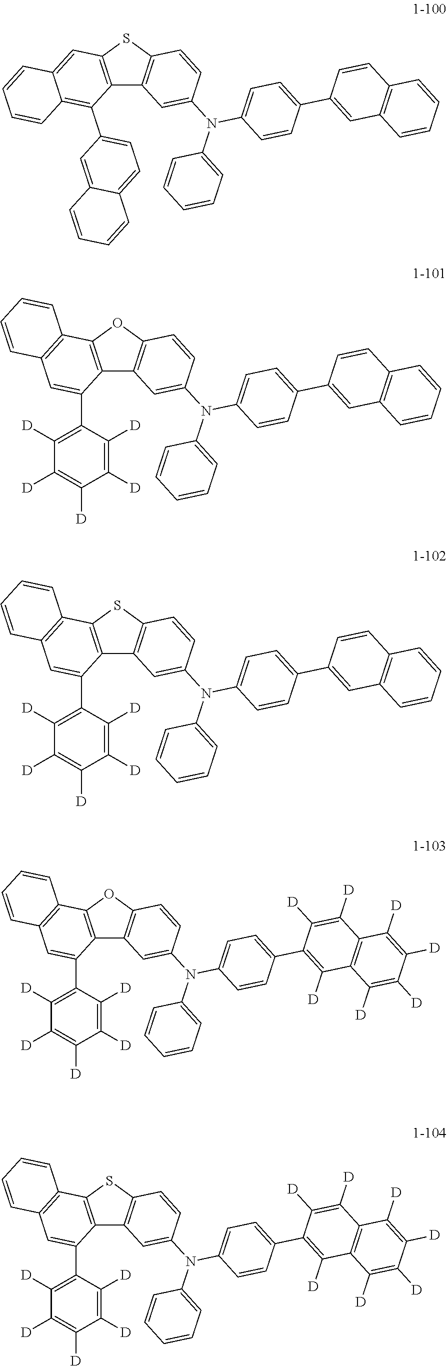

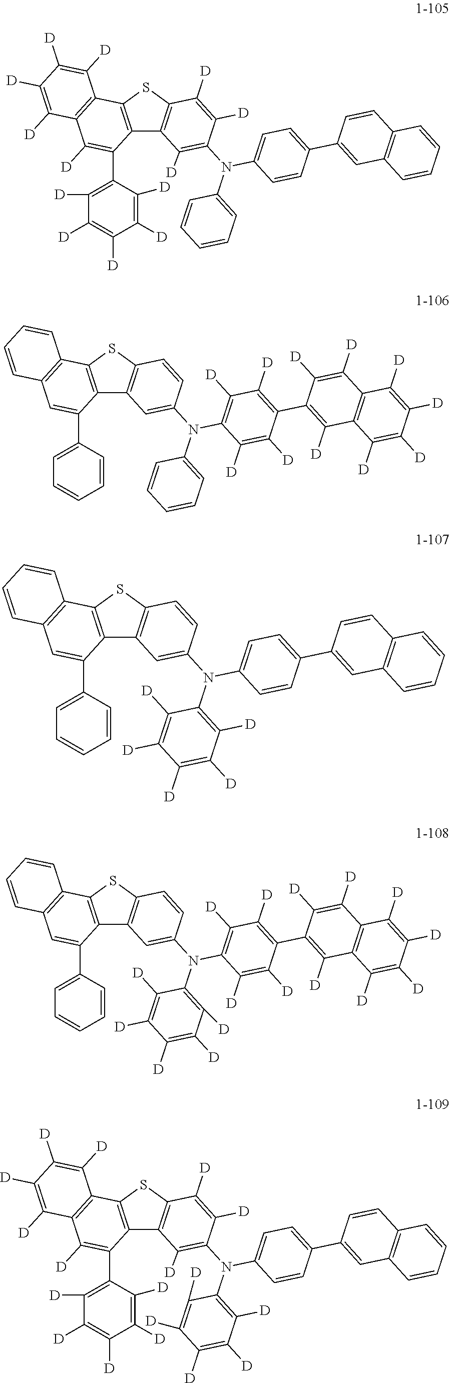

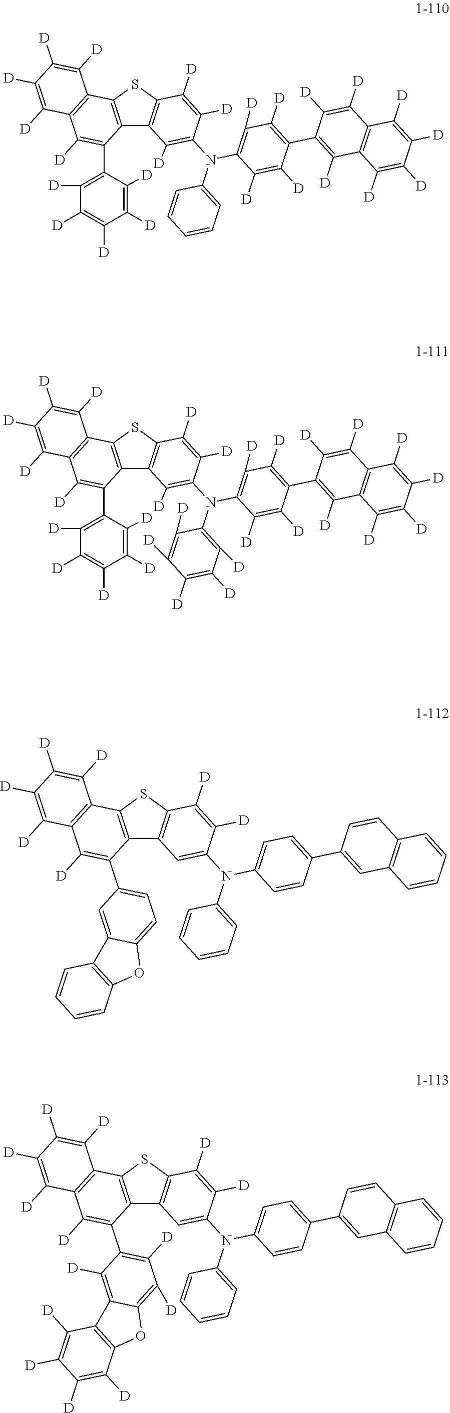

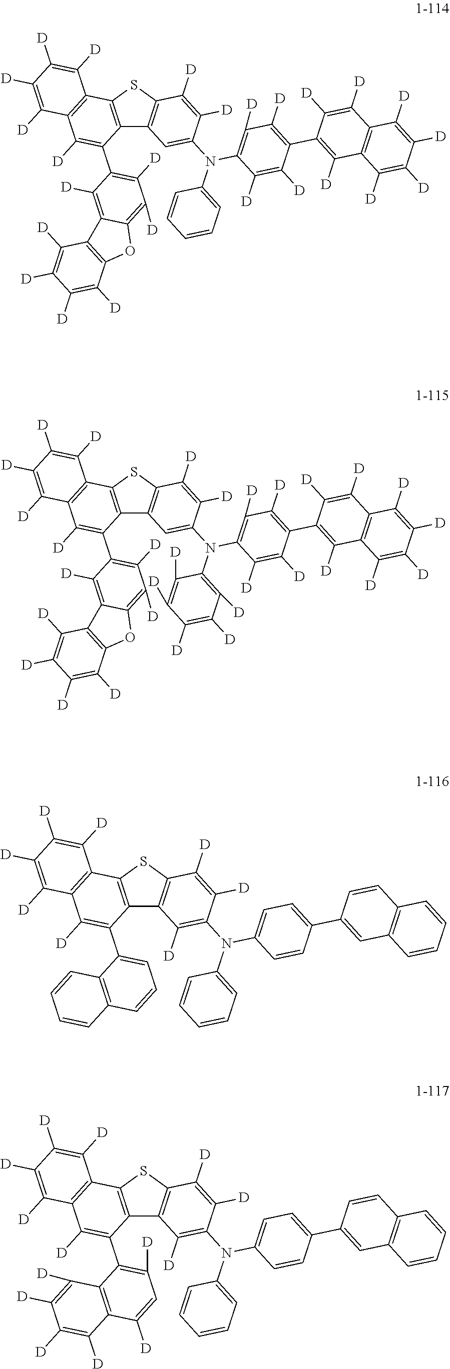

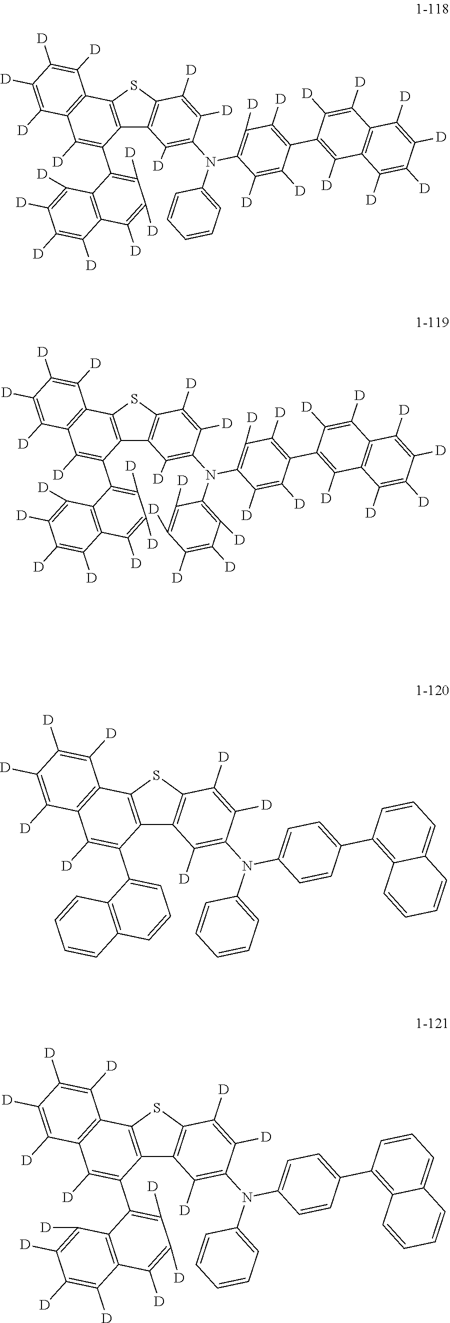

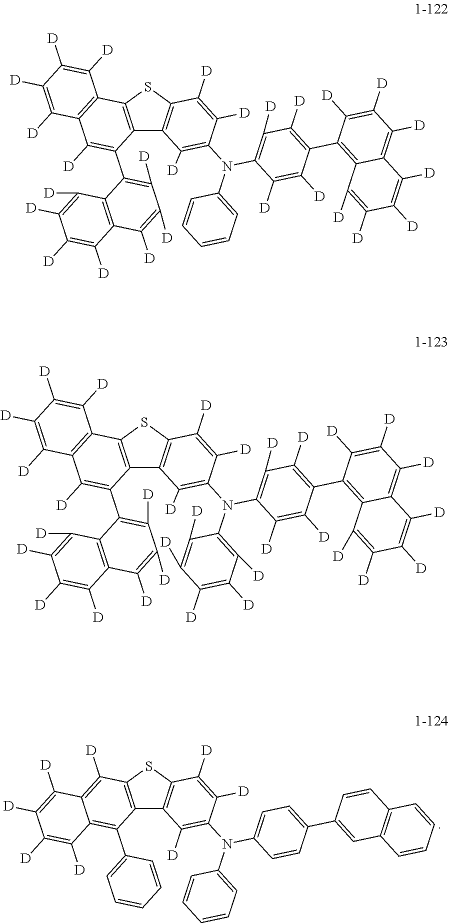

8. A composition for an organic optoelectronic device, the composition comprising:
a first compound; and
a second compound,
wherein:
the first compound is the compound for an organic optoelectronic device as claimed in claim 1, and
the second compound is represented by Chemical Formula 2: 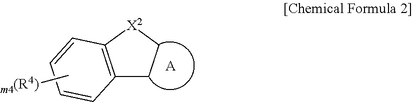
in Chemical Formula 2,
X² is O, S, N-L^{a}-R^{a}, CR^{b}R^{c}, or SiR^{d}R^{e},
L^{a} is a single bond or a substituted or unsubstituted C6 to C12 arylene group,
R^{a}, R^{b}, R^{c}, R^{d}, R^{e}, and R⁴ are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
m4 is an integer 1 to 4, and
A is a ring of Group III, 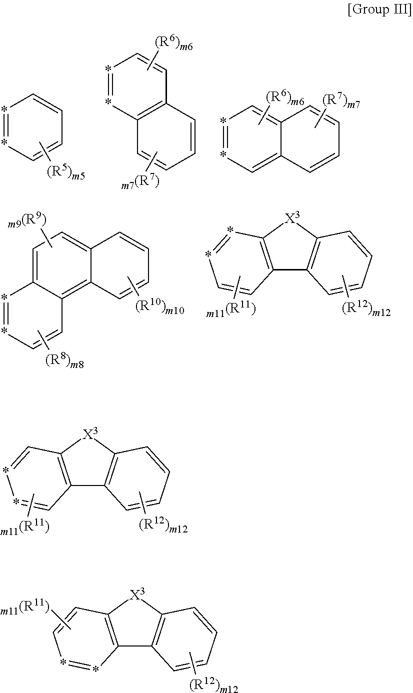
in Group III,
* is a linking carbon,
X³ is O or S,
R⁵ to R¹² are each independently hydrogen, deuterium, a substituted or unsubstituted C1 to C30 alkyl group, a substituted or unsubstituted C6 to C30 aryl group, or a substituted or unsubstituted C2 to C30 heterocyclic group,
m5, m7, m10, and m12 are each independently an integer of 1 to 4,
m6, m8, m9, and m11 are each independently 1 or 2, and
at least one of Ra and R⁴ to R¹² is a group represented by Chemical Formula a, 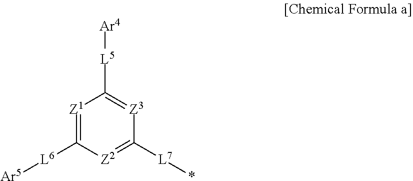
in Chemical Formula a,
Z¹ to Z³ are each independently N or CR^{f},
at least two of Z¹ to Z³ are N,
R^{f} is hydrogen, deuterium, a substituted or unsubstituted C1 to C10 alkyl group, or a substituted or unsubstituted C6 to C20 aryl group,
L⁵ to L⁷ are each independently a single bond or a substituted or unsubstituted C6 to C30 arylene group,
Ar⁴ and Ar⁵ are each independently a substituted or unsubstituted C6 to C30 aryl group or a substituted or unsubstituted C2 to C30 heterocyclic group, and
* is a linking point.

9. The composition as claimed in claim 8, wherein:
Chemical Formula 2 is represented by one of Chemical Formula 2-I to Chemical Formula 2-X, 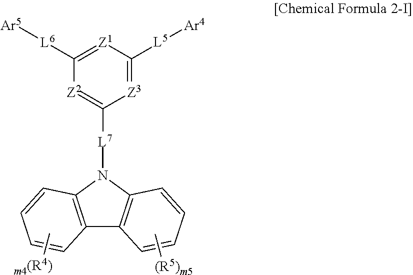
in Chemical Formula 2-I, Z¹ to Z³, R⁴, R⁵, L⁵ to L⁷, Ar⁴, Ar⁵, m4, and m5 are defined the same as those of Chemical Formula 2; 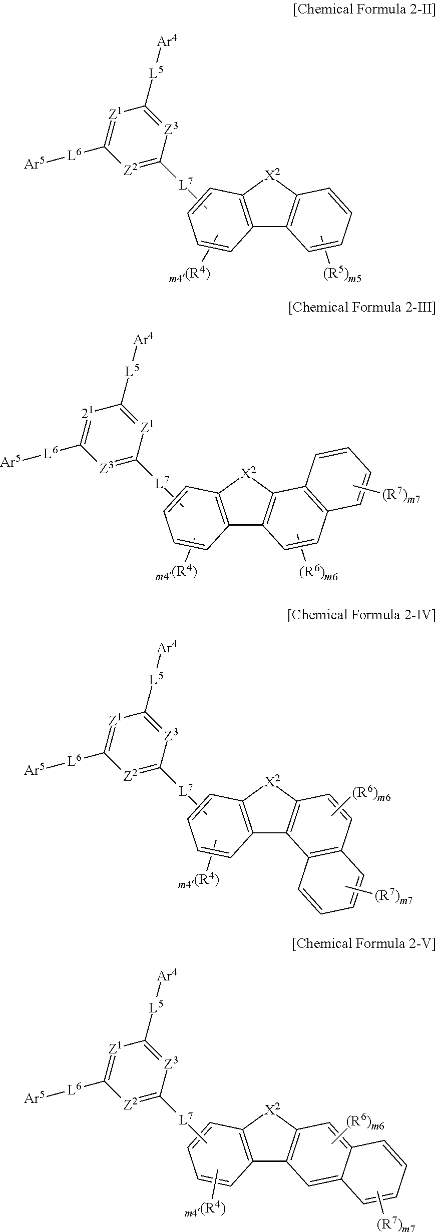
in Chemical Formula 2-II to Chemical Formula 2-V,
X², Z³ to Z³, R⁴ to R⁷, L⁵ to L⁷, Ar⁴, Ar⁵, and m5 to m7 are defined the same as those of Chemical Formula 2, and
m4′ is an integer of 1 to 3; 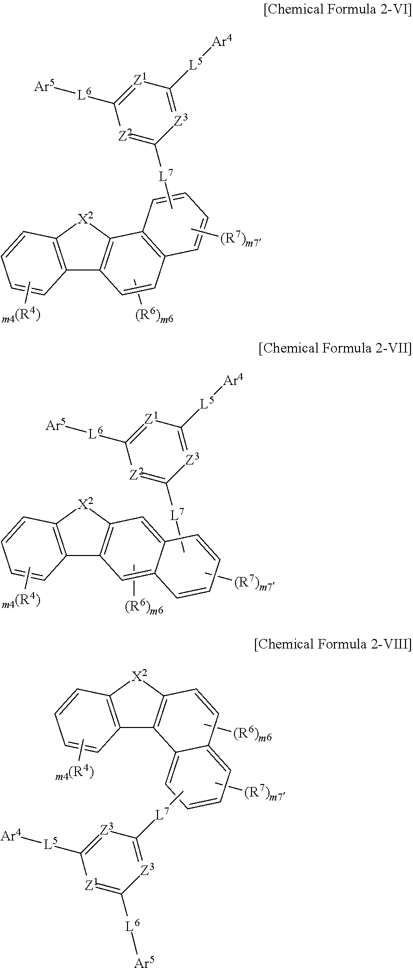
in Chemical Formula 2-VI to Chemical Formula 2-VIII,
X², Z¹ to Z³, R⁴, R⁶, R⁷, L⁵ to L⁷, Ar⁴, Ar⁵, m4, and m6 are defined the same as those of Chemical Formula 2, and
m7′ is an integer of 1 to 3; 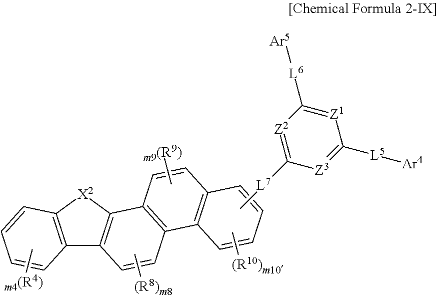
in Chemical Formula 2-IX,
X², Z¹ to Z³, R⁴, R⁸ to R¹⁰, L⁵ to L⁷, Ar⁴, Ar⁵, m4, m8, and m9 are defined the same as those of Chemical Formula 2, and
m10′ is an integer 1 to 3; 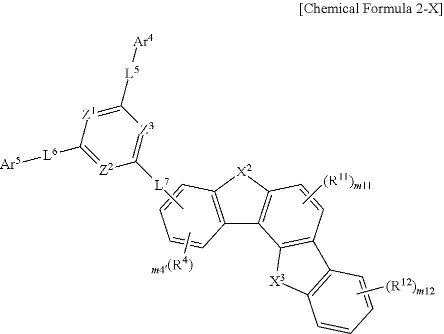
in Chemical Formula 2-X,
X², X³, Z¹ to Z³, R⁴, R¹¹, R¹², L⁵ to L⁷, Ar⁴, Ar⁵, m11, and m12 are defined the same as those of Chemical Formula 2, and
m4′ is an integer of 1 to 3.

10. The composition as claimed in claim 9, wherein the second compound is represented by Chemical Formula 2-II, Chemical Formula 2-III, or Chemical Formula 2-VI.

11. The composition as claimed in claim 9, wherein:
the second compound is represented by Chemical Formula 2-II-3, Chemical Formula 2-III-1, Chemical Formula 2-VI-1, or Chemical Formula 2-VI-3, 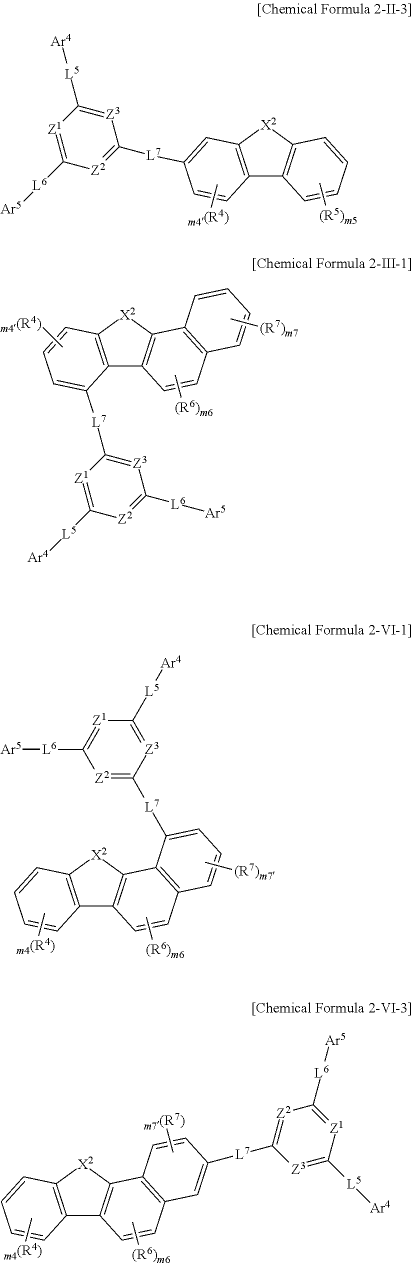
in Chemical Formula 2-II-3, Chemical Formula 2-III-1, Chemical Formula 2-VI-1, and Chemical Formula 2-VI-3, X², Z¹ to Z³, R⁴ to R⁷, L⁵ to L⁷, Ar⁴, Ar⁵, m4 to m7, m4′, and m7′ are defined the same as those of Chemical Formula 2.

12. The composition as claimed in claim 8, wherein Ar⁴ and Ar⁵ are each independently a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylene group, a substituted or unsubstituted carbazolyl group, a substituted or unsubstituted dibenzofuranyl group, a substituted or unsubstituted dibenzothiophenyl group, or a substituted or unsubstituted dibenzosilolyl group.

13. The composition as claimed in claim 8, wherein the second compound is a compound of Group 2:

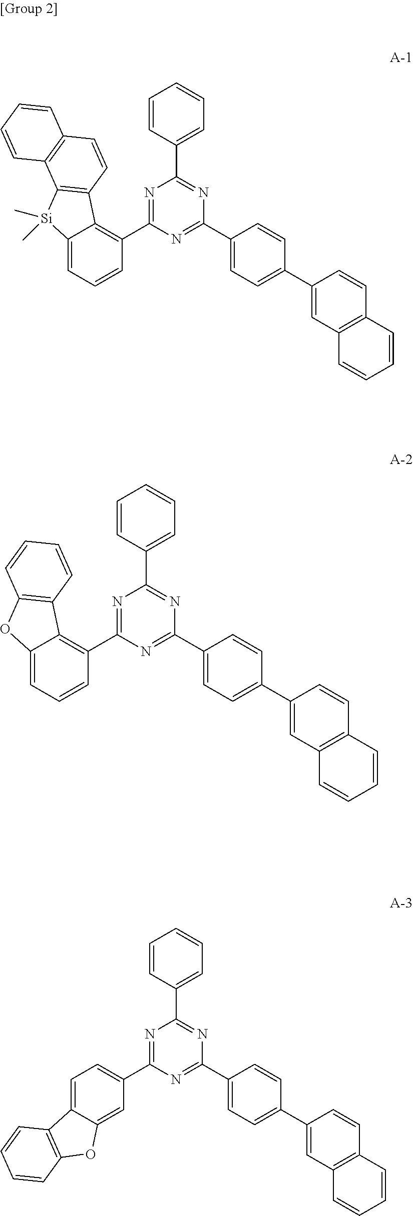 [SMILES: C[Si]1(C)c2cccc(-c3nc(-c4ccccc4)nc(-c4ccc(-c5ccc6ccccc6c5)cc4)n3)c2-c2ccc3ccccc3c21 ; c1ccc(-c2nc(-c3ccc(-c4ccc5ccccc5c4)cc3)nc(-c3cccc4oc5ccccc5c34)n2)cc1 ; c1ccc(-c2nc(-c3ccc(-c4ccc5ccccc5c4)cc3)nc(-c3ccc4c(c3)oc3ccccc34)n2)cc1]

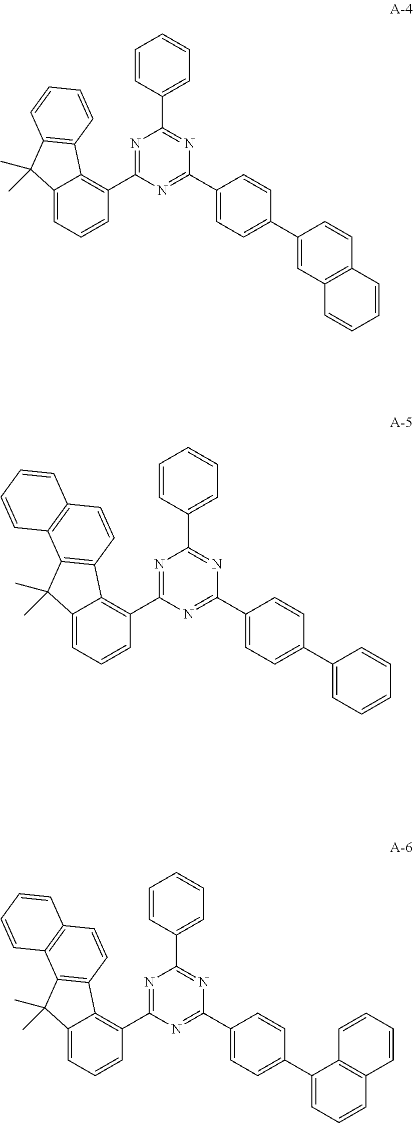 [SMILES: CC1(C)c2ccccc2-c2c(-c3nc(-c4ccccc4)nc(-c4ccc(-c5ccc6ccccc6c5)cc4)n3)cccc21 ; CC1(C)c2cccc(-c3nc(-c4ccccc4)nc(-c4ccc(-c5ccccc5)cc4)n3)c2-c2ccc3ccccc3c21 ; CC1(C)c2cccc(-c3nc(-c4ccccc4)nc(-c4ccc(-c5cccc6ccccc56)cc4)n3)c2-c2ccc3ccccc3c21]

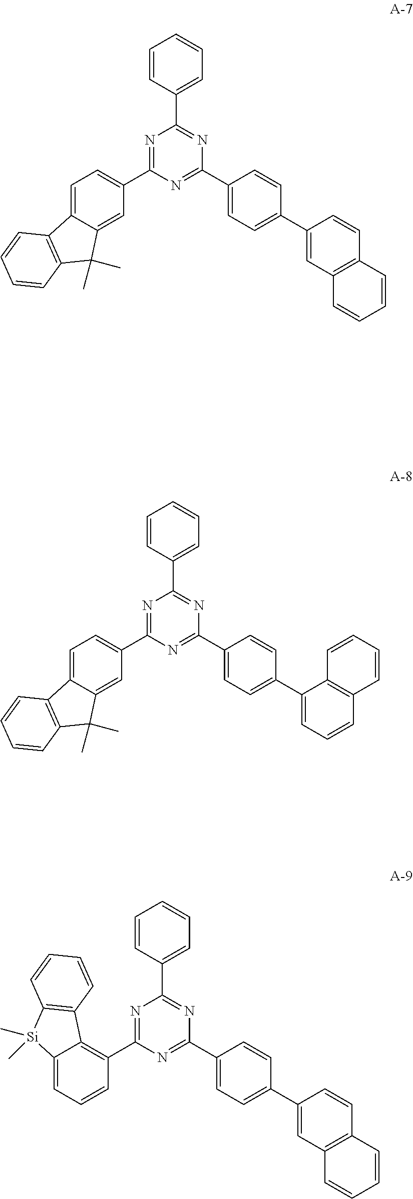 [SMILES: CC1(C)c2ccccc2-c2ccc(-c3nc(-c4ccccc4)nc(-c4ccc(-c5ccc6ccccc6c5)cc4)n3)cc21 ; CC1(C)c2ccccc2-c2ccc(-c3nc(-c4ccccc4)nc(-c4ccc(-c5cccc6ccccc56)cc4)n3)cc21 ; C[Si]1(C)c2ccccc2-c2c(-c3nc(-c4ccccc4)nc(-c4ccc(-c5ccc6ccccc6c5)cc4)n3)cccc21]

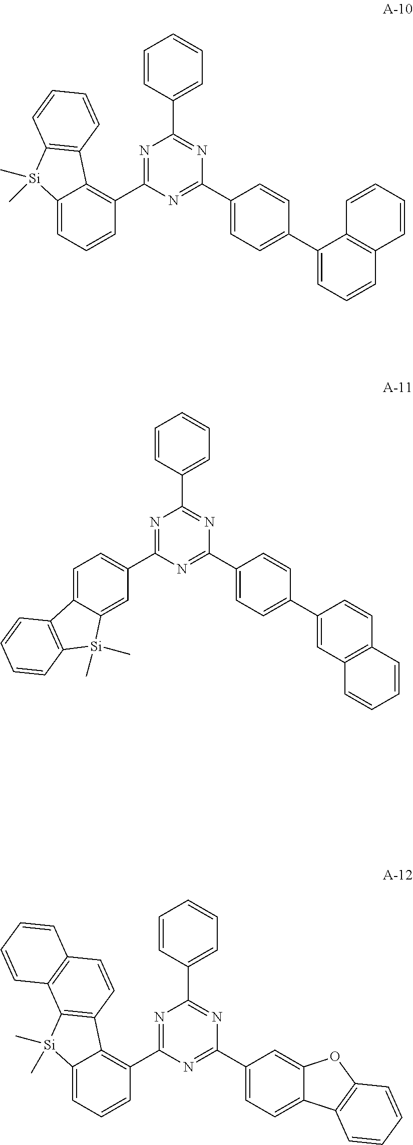 [SMILES: C[Si]1(C)c2ccccc2-c2c(-c3nc(-c4ccccc4)nc(-c4ccc(-c5cccc6ccccc56)cc4)n3)cccc21 ; C[Si]1(C)c2ccccc2-c2ccc(-c3nc(-c4ccccc4)nc(-c4ccc(-c5ccc6ccccc6c5)cc4)n3)cc21 ; C[Si]1(C)c2cccc(-c3nc(-c4ccccc4)nc(-c4ccc5c(c4)oc4ccccc45)n3)c2-c2ccc3ccccc3c21]

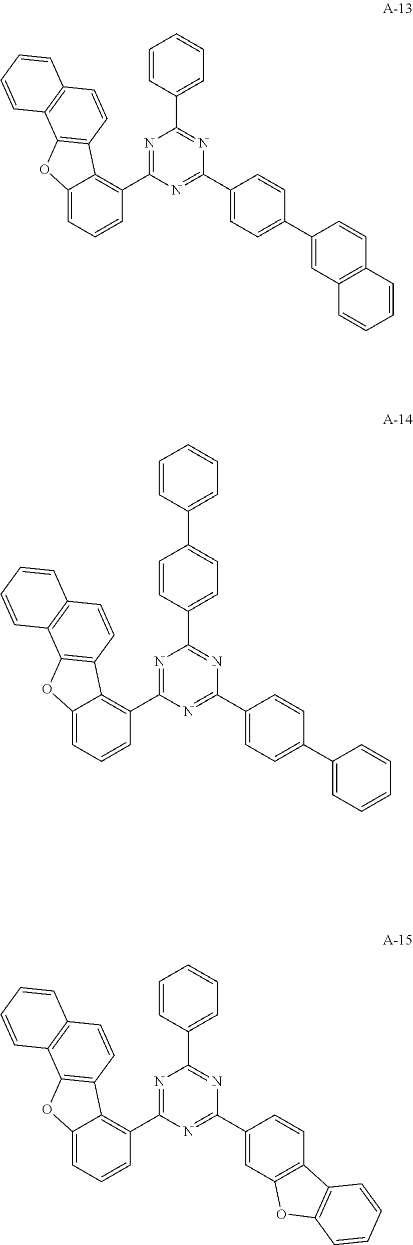 [SMILES: c1ccc(-c2nc(-c3ccc(-c4ccc5ccccc5c4)cc3)nc(-c3cccc4oc5c6ccccc6ccc5c34)n2)cc1 ; c1ccc(-c2ccc(-c3nc(-c4ccc(-c5ccccc5)cc4)nc(-c4cccc5oc6c7ccccc7ccc6c45)n3)cc2)cc1 ; c1ccc(-c2nc(-c3ccc4c(c3)oc3ccccc34)nc(-c3cccc4oc5c6ccccc6ccc5c34)n2)cc1]

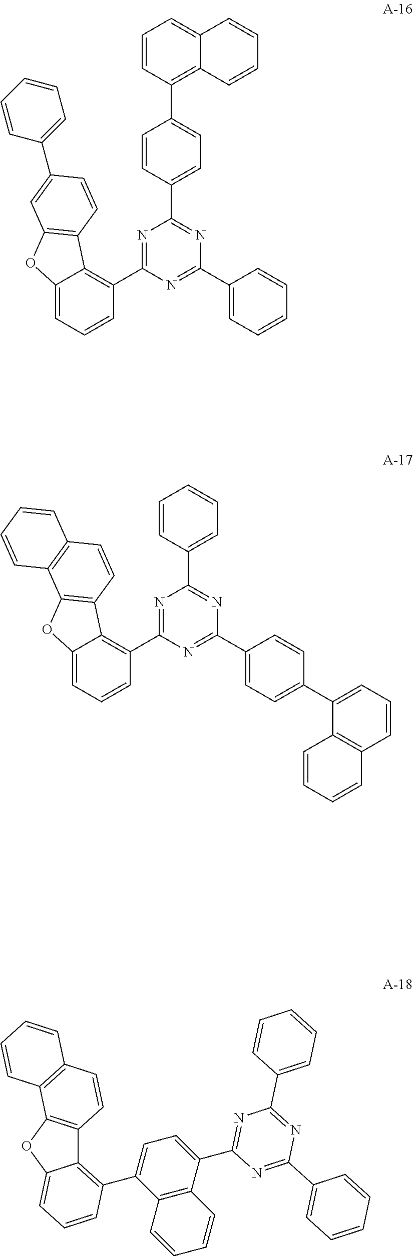 [SMILES: c1ccc(-c2ccc3c(c2)oc2cccc(-c4nc(-c5ccccc5)nc(-c5ccc(-c6cccc7ccccc67)cc5)n4)c23)cc1 ; c1ccc(-c2nc(-c3ccc(-c4cccc5ccccc45)cc3)nc(-c3cccc4oc5c6ccccc6ccc5c34)n2)cc1 ; c1ccc(-c2nc(-c3ccccc3)nc(-c3ccc(-c4cccc5oc6c7ccccc7ccc6c45)c4ccccc34)n2)cc1]

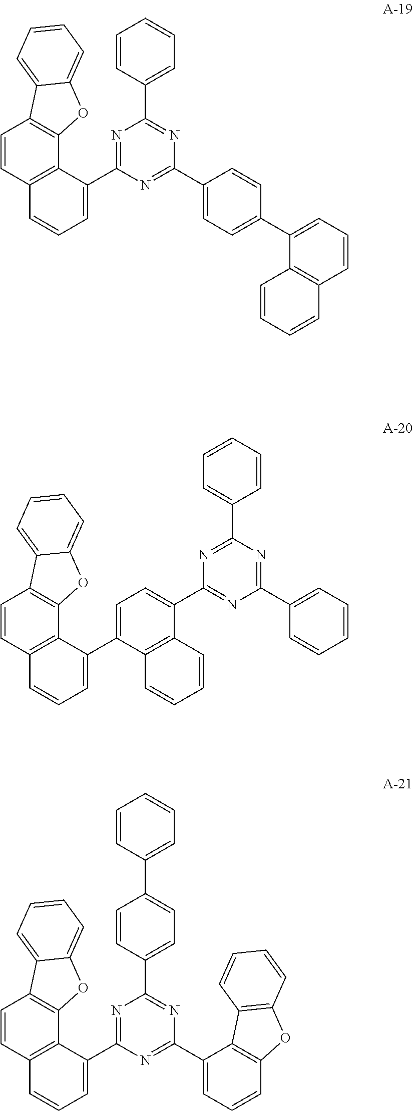 [SMILES: c1ccc(-c2nc(-c3ccc(-c4cccc5ccccc45)cc3)nc(-c3cccc4ccc5c6ccccc6oc5c34)n2)cc1 ; c1ccc(-c2nc(-c3ccccc3)nc(-c3ccc(-c4cccc5ccc6c7ccccc7oc6c45)c4ccccc34)n2)cc1 ; c1ccc(-c2ccc(-c3nc(-c4cccc5oc6ccccc6c45)nc(-c4cccc5ccc6c7ccccc7oc6c45)n3)cc2)cc1]

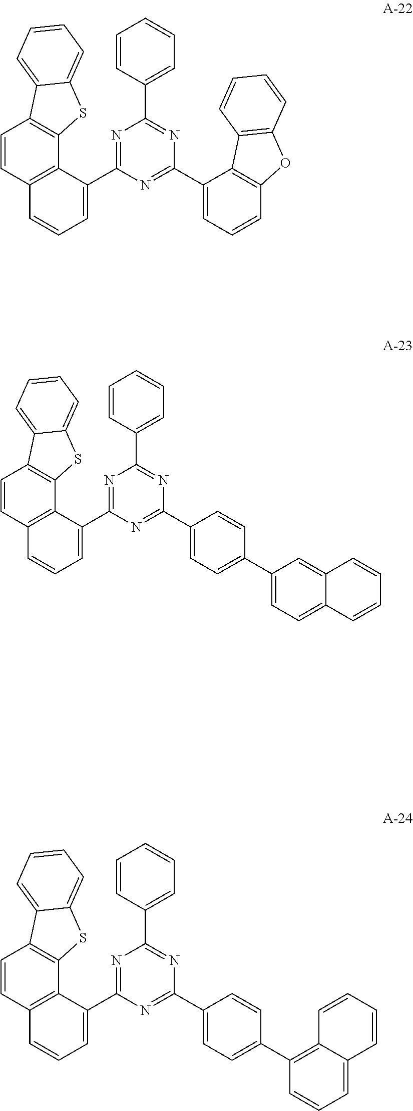 [SMILES: c1ccc(-c2nc(-c3cccc4oc5ccccc5c34)nc(-c3cccc4ccc5c6ccccc6sc5c34)n2)cc1 ; c1ccc(-c2nc(-c3ccc(-c4ccc5ccccc5c4)cc3)nc(-c3cccc4ccc5c6ccccc6sc5c34)n2)cc1 ; c1ccc(-c2nc(-c3ccc(-c4cccc5ccccc45)cc3)nc(-c3cccc4ccc5c6ccccc6sc5c34)n2)cc1]

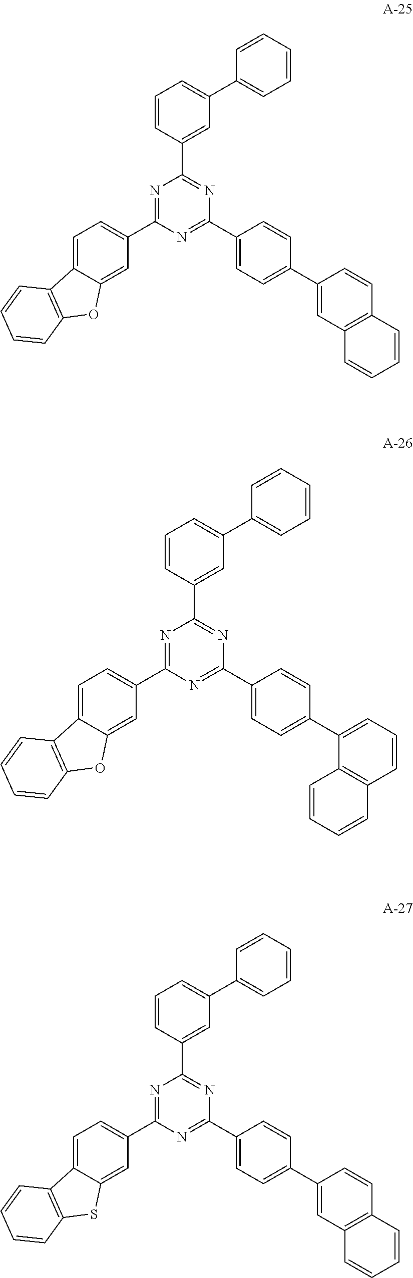 [SMILES: c1ccc(-c2cccc(-c3nc(-c4ccc(-c5ccc6ccccc6c5)cc4)nc(-c4ccc5c(c4)oc4ccccc45)n3)c2)cc1 ; c1ccc(-c2cccc(-c3nc(-c4ccc(-c5cccc6ccccc56)cc4)nc(-c4ccc5c(c4)oc4ccccc45)n3)c2)cc1 ; c1ccc(-c2cccc(-c3nc(-c4ccc(-c5ccc6ccccc6c5)cc4)nc(-c4ccc5c(c4)sc4ccccc45)n3)c2)cc1]

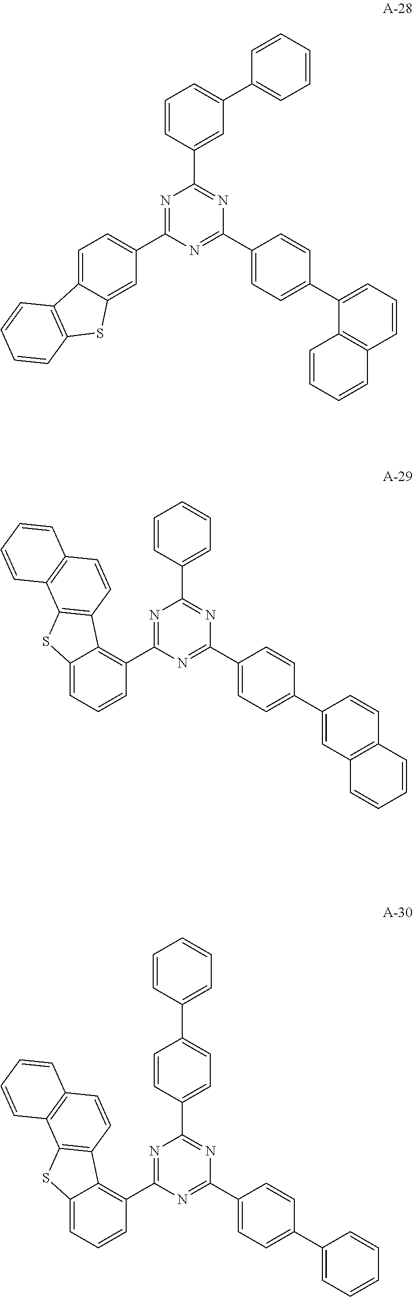 [SMILES: c1ccc(-c2cccc(-c3nc(-c4ccc(-c5cccc6ccccc56)cc4)nc(-c4ccc5c(c4)sc4ccccc45)n3)c2)cc1 ; c1ccc(-c2nc(-c3ccc(-c4ccc5ccccc5c4)cc3)nc(-c3cccc4sc5c6ccccc6ccc5c34)n2)cc1 ; c1ccc(-c2ccc(-c3nc(-c4ccc(-c5ccccc5)cc4)nc(-c4cccc5sc6c7ccccc7ccc6c45)n3)cc2)cc1]

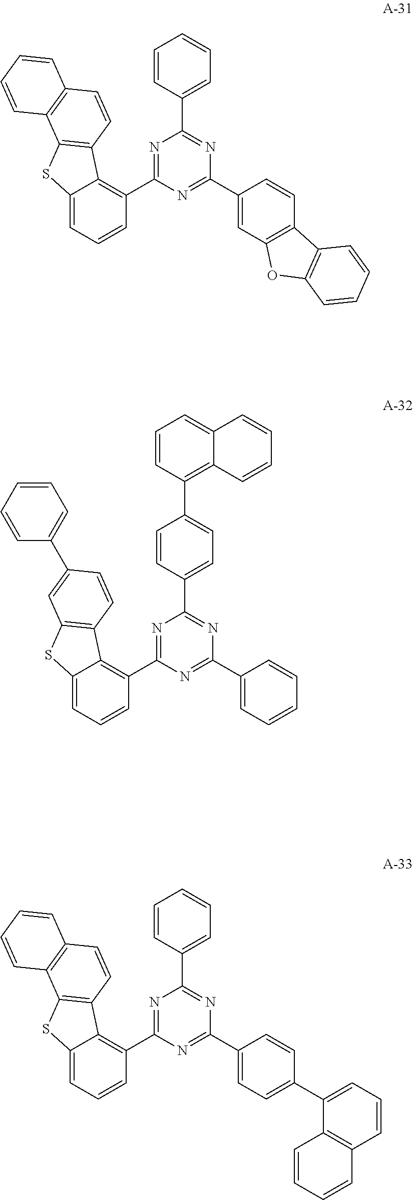 [SMILES: c1ccc(-c2nc(-c3ccc4c(c3)oc3ccccc34)nc(-c3cccc4sc5c6ccccc6ccc5c34)n2)cc1 ; c1ccc(-c2ccc3c(c2)sc2cccc(-c4nc(-c5ccccc5)nc(-c5ccc(-c6cccc7ccccc67)cc5)n4)c23)cc1 ; c1ccc(-c2nc(-c3ccc(-c4cccc5ccccc45)cc3)nc(-c3cccc4sc5c6ccccc6ccc5c34)n2)cc1]

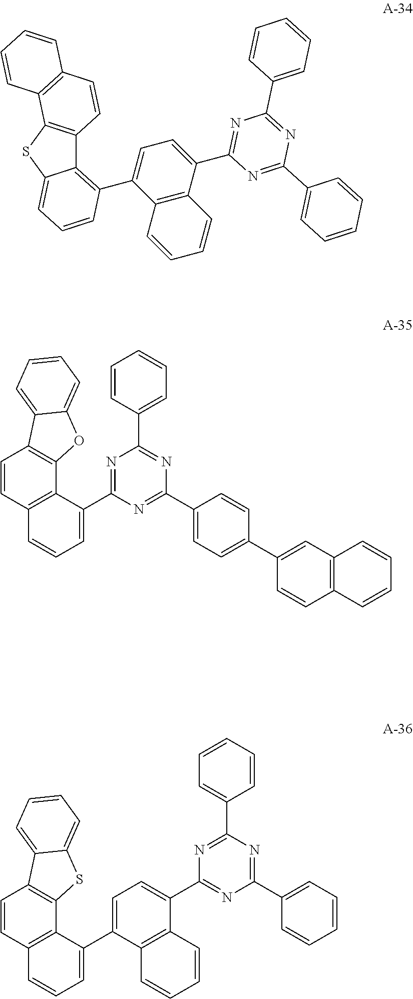 [SMILES: c1ccc(-c2nc(-c3ccccc3)nc(-c3ccc(-c4cccc5sc6c7ccccc7ccc6c45)c4ccccc34)n2)cc1 ; c1ccc(-c2nc(-c3ccc(-c4ccc5ccccc5c4)cc3)nc(-c3cccc4ccc5c6ccccc6oc5c34)n2)cc1 ; c1ccc(-c2nc(-c3ccccc3)nc(-c3ccc(-c4cccc5ccc6c7ccccc7sc6c45)c4ccccc34)n2)cc1]

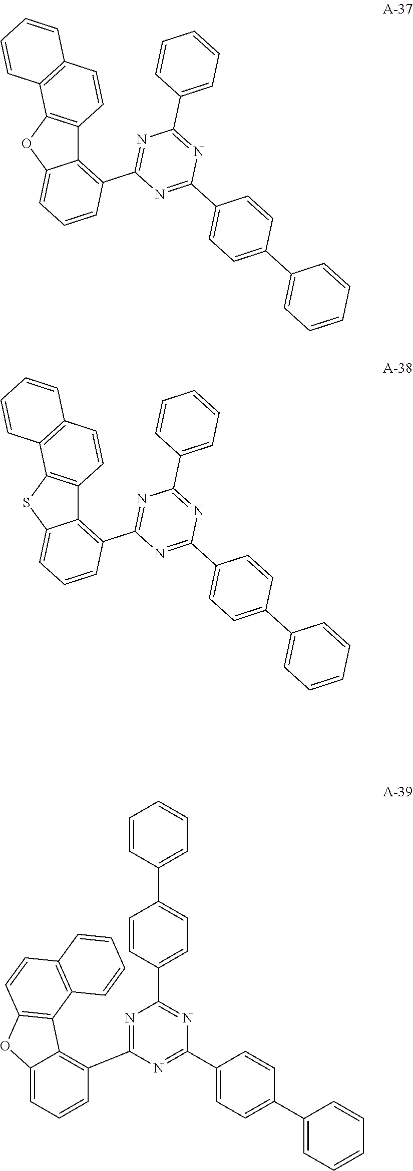 [SMILES: c1ccc(-c2ccc(-c3nc(-c4ccccc4)nc(-c4cccc5oc6c7ccccc7ccc6c45)n3)cc2)cc1 ; c1ccc(-c2ccc(-c3nc(-c4ccccc4)nc(-c4cccc5sc6c7ccccc7ccc6c45)n3)cc2)cc1 ; c1ccc(-c2ccc(-c3nc(-c4ccc(-c5ccccc5)cc4)nc(-c4cccc5oc6ccc7ccccc7c6c45)n3)cc2)cc1]

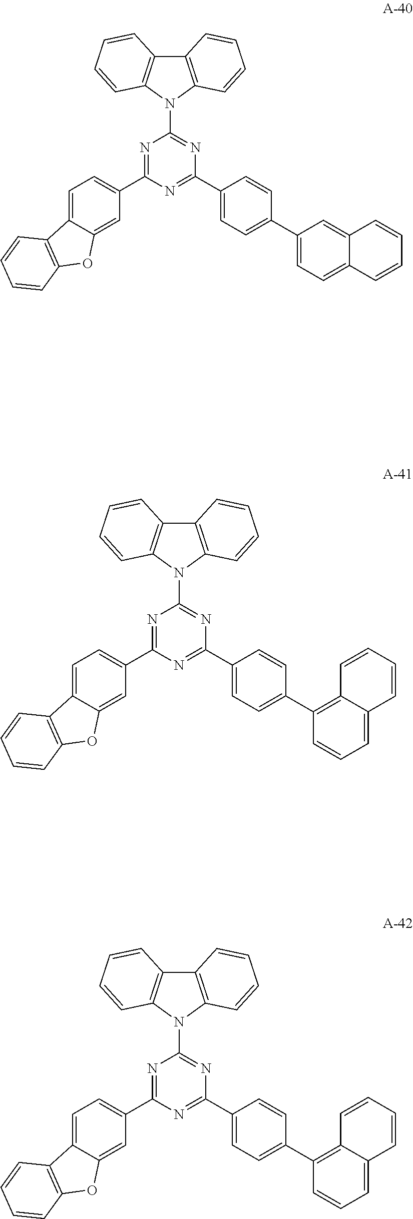 [SMILES: c1ccc2cc(-c3ccc(-c4nc(-c5ccc6c(c5)oc5ccccc56)nc(-n5c6ccccc6c6ccccc65)n4)cc3)ccc2c1 ; c1ccc2c(-c3ccc(-c4nc(-c5ccc6c(c5)oc5ccccc56)nc(-n5c6ccccc6c6ccccc65)n4)cc3)cccc2c1 ; c1ccc2c(-c3ccc(-c4nc(-c5ccc6c(c5)oc5ccccc56)nc(-n5c6ccccc6c6ccccc65)n4)cc3)cccc2c1]

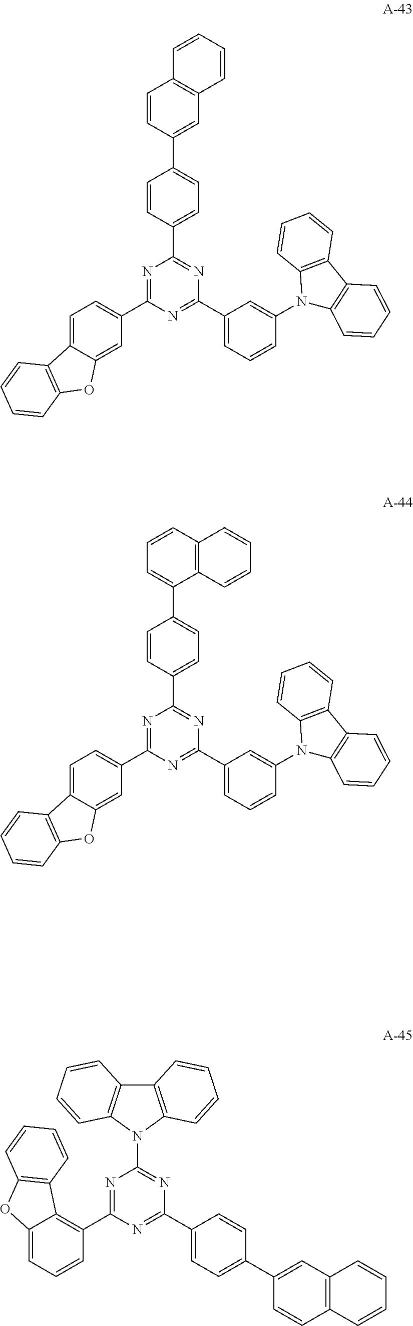 [SMILES: c1cc(-c2nc(-c3ccc(-c4ccc5ccccc5c4)cc3)nc(-c3ccc4c(c3)oc3ccccc34)n2)cc(-n2c3ccccc3c3ccccc32)c1 ; c1cc(-c2nc(-c3ccc(-c4cccc5ccccc45)cc3)nc(-c3ccc4c(c3)oc3ccccc34)n2)cc(-n2c3ccccc3c3ccccc32)c1 ; c1ccc2cc(-c3ccc(-c4nc(-c5cccc6oc7ccccc7c56)nc(-n5c6ccccc6c6ccccc65)n4)cc3)ccc2c1]

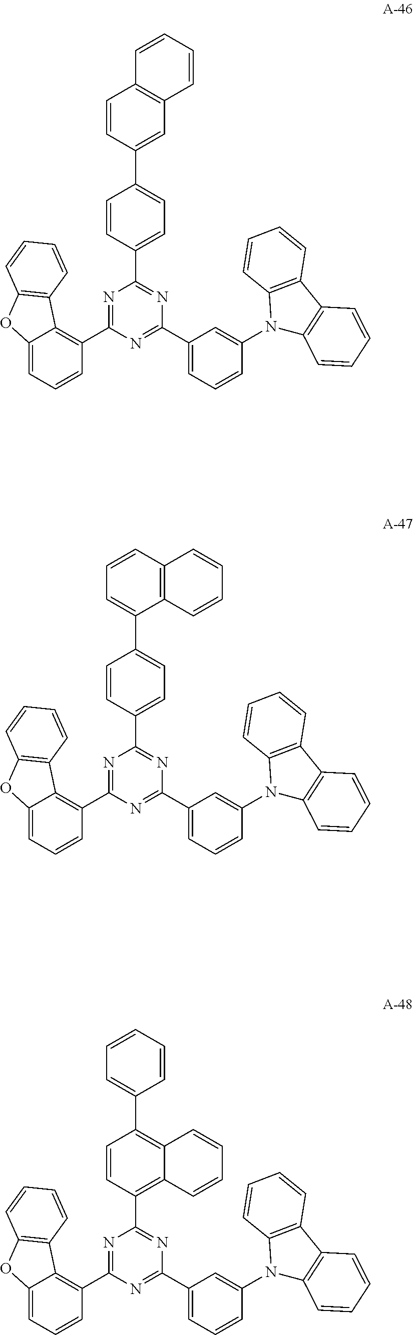 [SMILES: c1cc(-c2nc(-c3ccc(-c4ccc5ccccc5c4)cc3)nc(-c3cccc4oc5ccccc5c34)n2)cc(-n2c3ccccc3c3ccccc32)c1 ; c1cc(-c2nc(-c3ccc(-c4cccc5ccccc45)cc3)nc(-c3cccc4oc5ccccc5c34)n2)cc(-n2c3ccccc3c3ccccc32)c1 ; c1ccc(-c2ccc(-c3nc(-c4cccc(-n5c6ccccc6c6ccccc65)c4)nc(-c4cccc5oc6ccccc6c45)n3)c3ccccc23)cc1]

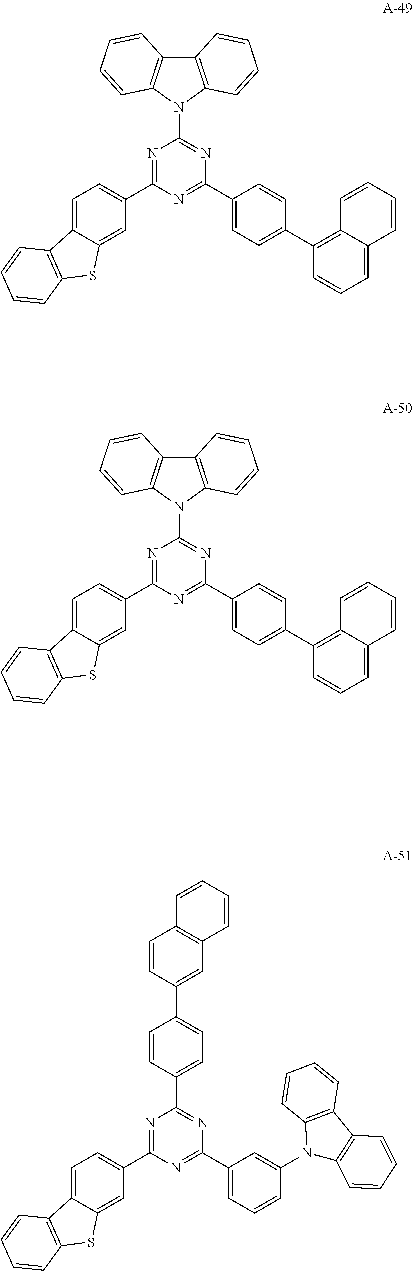 [SMILES: c1ccc2c(-c3ccc(-c4nc(-c5ccc6c(c5)sc5ccccc56)nc(-n5c6ccccc6c6ccccc65)n4)cc3)cccc2c1 ; c1ccc2c(-c3ccc(-c4nc(-c5ccc6c(c5)sc5ccccc56)nc(-n5c6ccccc6c6ccccc65)n4)cc3)cccc2c1 ; c1cc(-c2nc(-c3ccc(-c4ccc5ccccc5c4)cc3)nc(-c3ccc4c(c3)sc3ccccc34)n2)cc(-n2c3ccccc3c3ccccc32)c1]

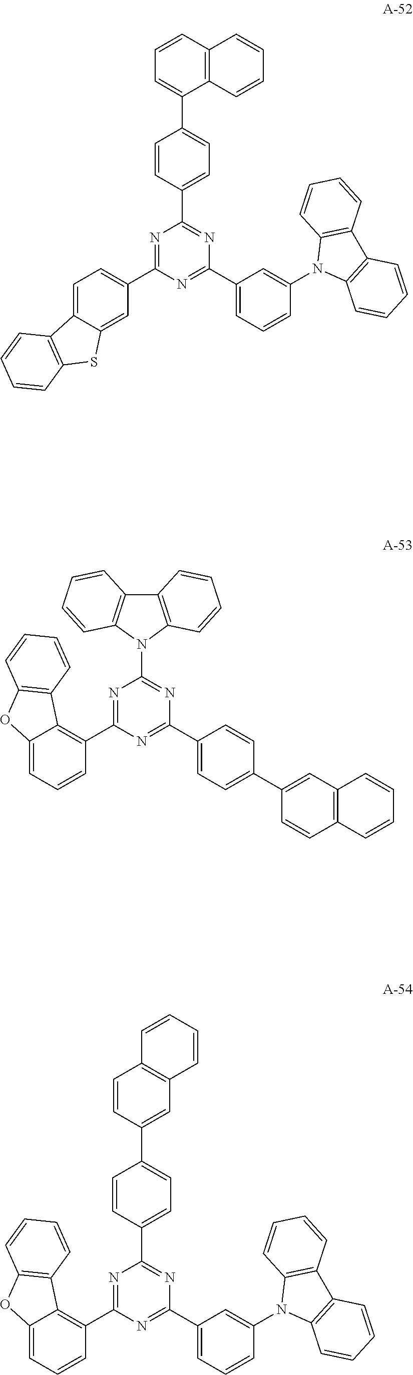 [SMILES: c1cc(-c2nc(-c3ccc(-c4cccc5ccccc45)cc3)nc(-c3ccc4c(c3)sc3ccccc34)n2)cc(-n2c3ccccc3c3ccccc32)c1 ; c1ccc2cc(-c3ccc(-c4nc(-c5cccc6oc7ccccc7c56)nc(-n5c6ccccc6c6ccccc65)n4)cc3)ccc2c1 ; c1cc(-c2nc(-c3ccc(-c4ccc5ccccc5c4)cc3)nc(-c3cccc4oc5ccccc5c34)n2)cc(-n2c3ccccc3c3ccccc32)c1]

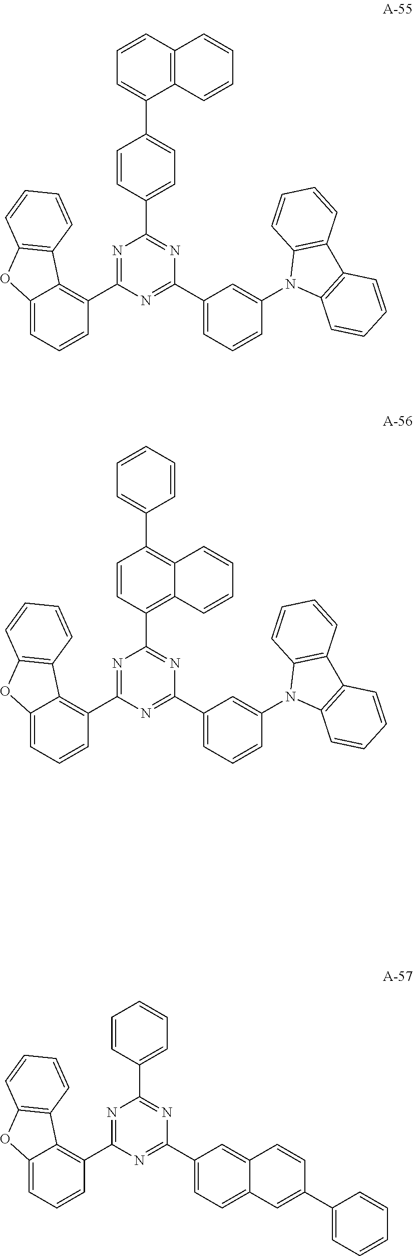 [SMILES: c1cc(-c2nc(-c3ccc(-c4cccc5ccccc45)cc3)nc(-c3cccc4oc5ccccc5c34)n2)cc(-n2c3ccccc3c3ccccc32)c1 ; c1ccc(-c2ccc(-c3nc(-c4cccc(-n5c6ccccc6c6ccccc65)c4)nc(-c4cccc5oc6ccccc6c45)n3)c3ccccc23)cc1 ; c1ccc(-c2ccc3cc(-c4nc(-c5ccccc5)nc(-c5cccc6oc7ccccc7c56)n4)ccc3c2)cc1]

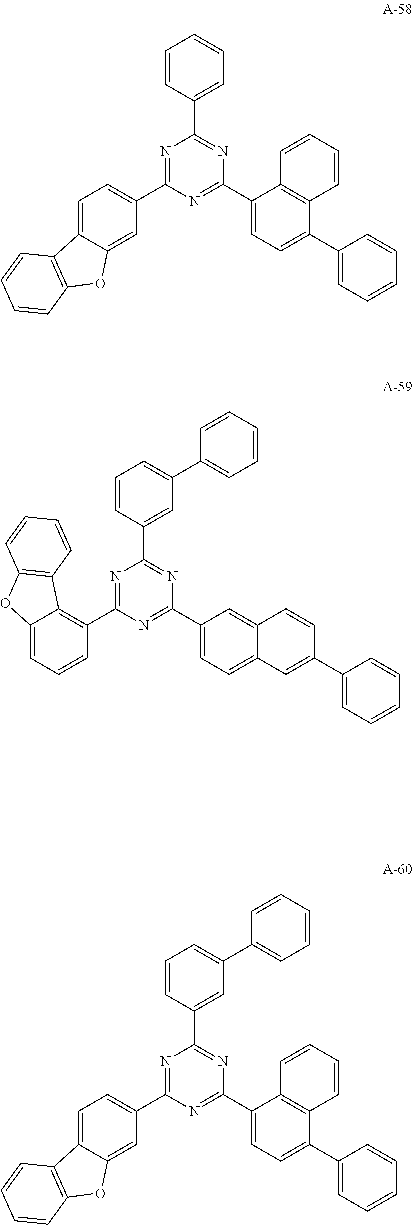 [SMILES: c1ccc(-c2nc(-c3ccc4c(c3)oc3ccccc34)nc(-c3ccc(-c4ccccc4)c4ccccc34)n2)cc1 ; c1ccc(-c2cccc(-c3nc(-c4ccc5cc(-c6ccccc6)ccc5c4)nc(-c4cccc5oc6ccccc6c45)n3)c2)cc1 ; c1ccc(-c2cccc(-c3nc(-c4ccc5c(c4)oc4ccccc45)nc(-c4ccc(-c5ccccc5)c5ccccc45)n3)c2)cc1]

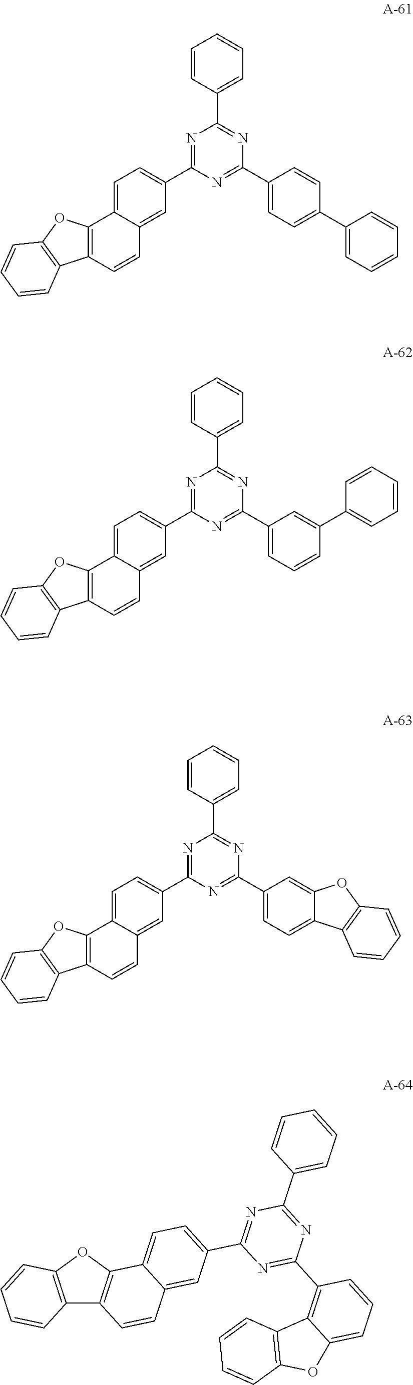 [SMILES: c1ccc(-c2ccc(-c3nc(-c4ccccc4)nc(-c4ccc5c(ccc6c7ccccc7oc56)c4)n3)cc2)cc1 ; c1ccc(-c2cccc(-c3nc(-c4ccccc4)nc(-c4ccc5c(ccc6c7ccccc7oc56)c4)n3)c2)cc1 ; c1ccc(-c2nc(-c3ccc4c(ccc5c6ccccc6oc45)c3)nc(-c3ccc4c(c3)oc3ccccc34)n2)cc1 ; c1ccc(-c2nc(-c3ccc4c(ccc5c6ccccc6oc45)c3)nc(-c3cccc4oc5ccccc5c34)n2)cc1]

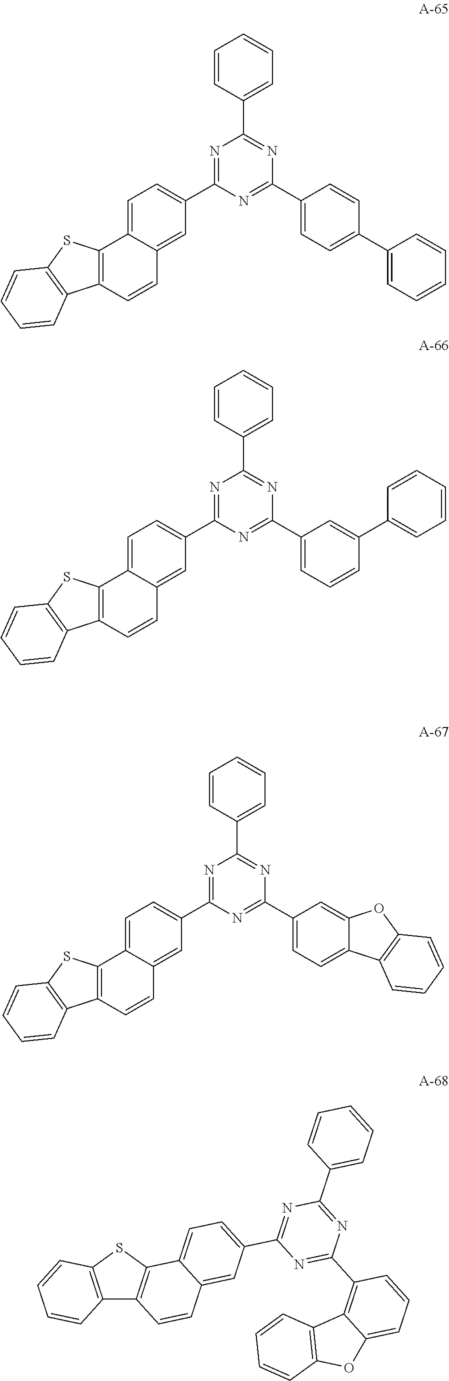 [SMILES: c1ccc(-c2ccc(-c3nc(-c4ccccc4)nc(-c4ccc5c(ccc6c7ccccc7sc56)c4)n3)cc2)cc1 ; c1ccc(-c2cccc(-c3nc(-c4ccccc4)nc(-c4ccc5c(ccc6c7ccccc7sc56)c4)n3)c2)cc1 ; c1ccc(-c2nc(-c3ccc4c(ccc5c6ccccc6sc45)c3)nc(-c3ccc4c(c3)oc3ccccc34)n2)cc1 ; c1ccc(-c2nc(-c3ccc4c(ccc5c6ccccc6sc45)c3)nc(-c3cccc4oc5ccccc5c34)n2)cc1]

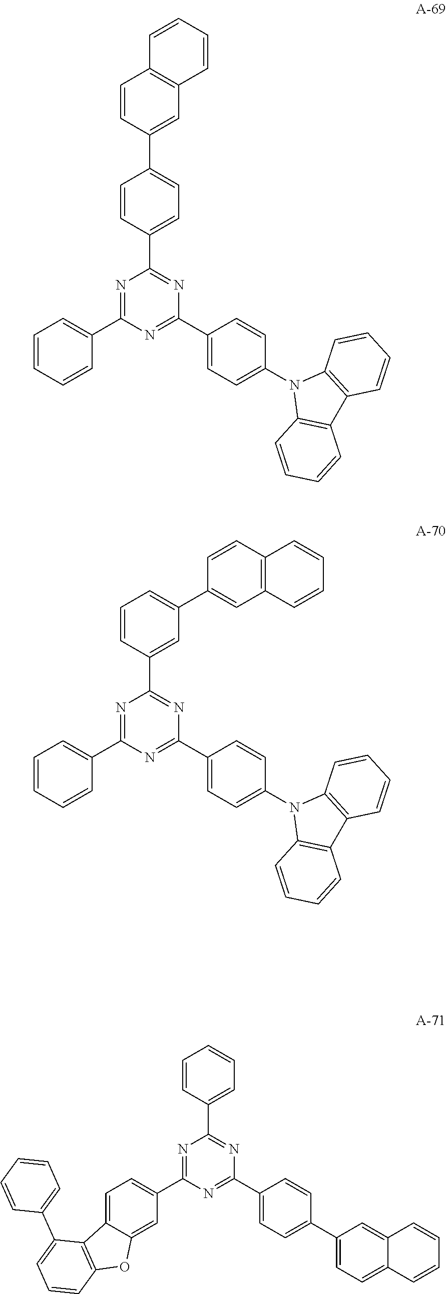 [SMILES: c1ccc(-c2nc(-c3ccc(-c4ccc5ccccc5c4)cc3)nc(-c3ccc(-n4c5ccccc5c5ccccc54)cc3)n2)cc1 ; c1ccc(-c2nc(-c3ccc(-n4c5ccccc5c5ccccc54)cc3)nc(-c3cccc(-c4ccc5ccccc5c4)c3)n2)cc1 ; c1ccc(-c2nc(-c3ccc(-c4ccc5ccccc5c4)cc3)nc(-c3ccc4c(c3)oc3cccc(-c5ccccc5)c34)n2)cc1]

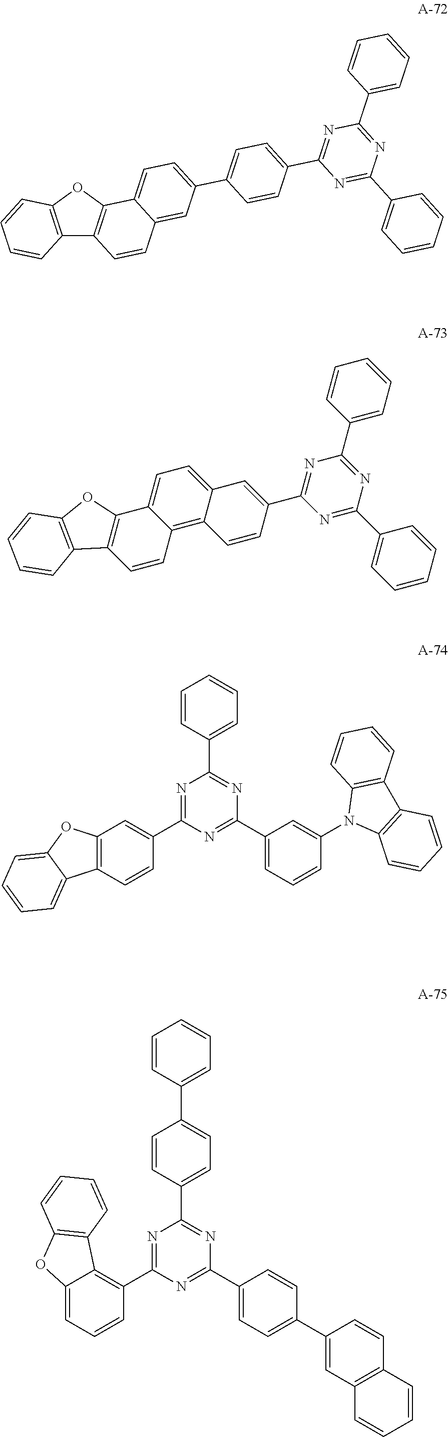 [SMILES: c1ccc(-c2nc(-c3ccccc3)nc(-c3ccc(-c4ccc5c(ccc6c7ccccc7oc56)c4)cc3)n2)cc1 ; c1ccc(-c2nc(-c3ccccc3)nc(-c3ccc4c(ccc5c4ccc4c6ccccc6oc45)c3)n2)cc1 ; c1ccc(-c2nc(-c3cccc(-n4c5ccccc5c5ccccc54)c3)nc(-c3ccc4c(c3)oc3ccccc34)n2)cc1 ; c1ccc(-c2ccc(-c3nc(-c4ccc(-c5ccc6ccccc6c5)cc4)nc(-c4cccc5oc6ccccc6c45)n3)cc2)cc1]

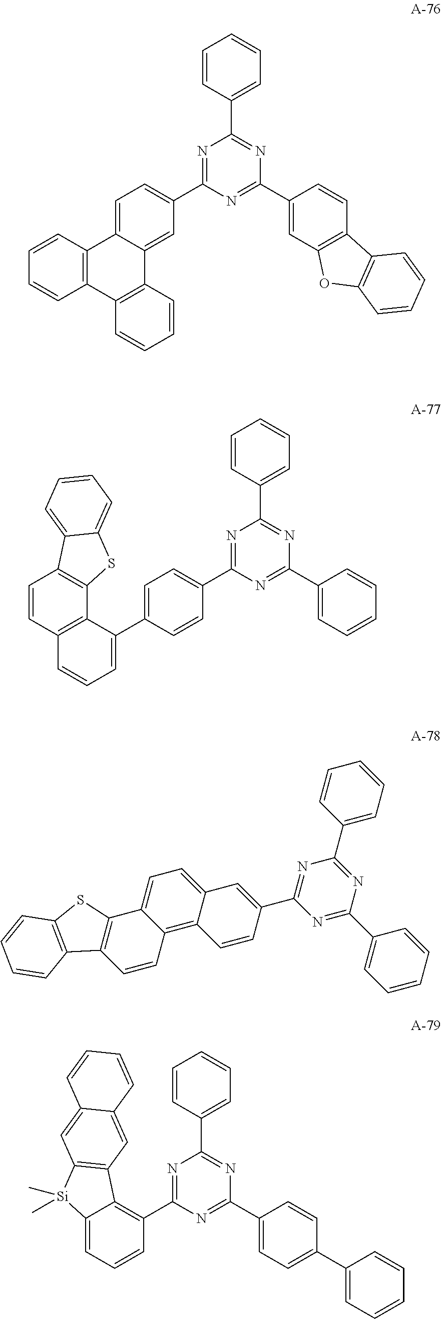 [SMILES: c1ccc(-c2nc(-c3ccc4c(c3)oc3ccccc34)nc(-c3ccc4c5ccccc5c5ccccc5c4c3)n2)cc1 ; c1ccc(-c2nc(-c3ccccc3)nc(-c3ccc(-c4cccc5ccc6c7ccccc7sc6c45)cc3)n2)cc1 ; c1ccc(-c2nc(-c3ccccc3)nc(-c3ccc4c(ccc5c4ccc4c6ccccc6sc45)c3)n2)cc1 ; C[Si]1(C)c2cc3ccccc3cc2-c2c(-c3nc(-c4ccccc4)nc(-c4ccc(-c5ccccc5)cc4)n3)cccc21]

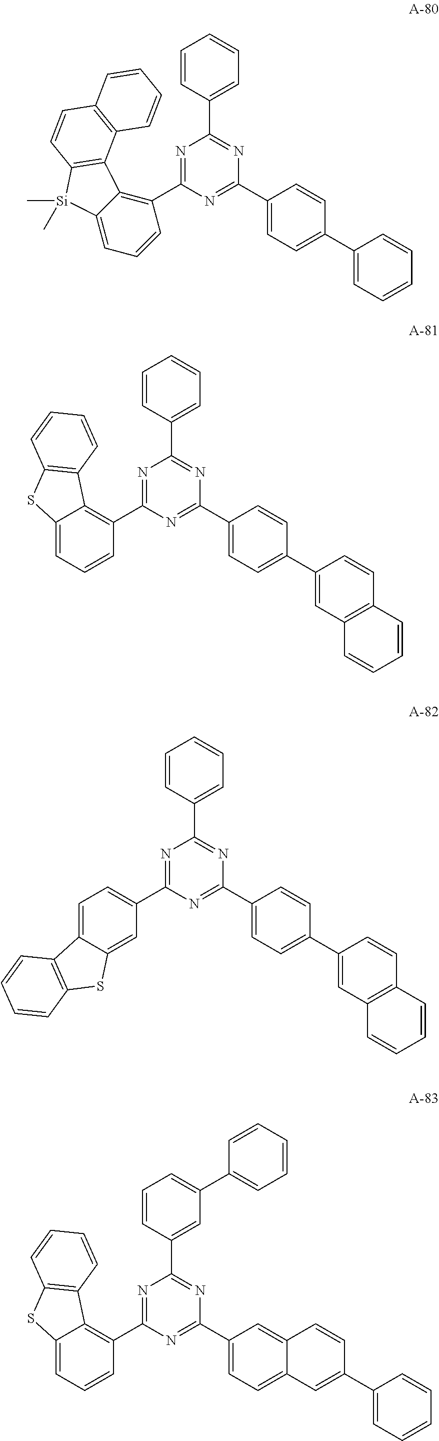 [SMILES: C[Si]1(C)c2cccc(-c3nc(-c4ccccc4)nc(-c4ccc(-c5ccccc5)cc4)n3)c2-c2c1ccc1ccccc21 ; c1ccc(-c2nc(-c3ccc(-c4ccc5ccccc5c4)cc3)nc(-c3cccc4sc5ccccc5c34)n2)cc1 ; c1ccc(-c2nc(-c3ccc(-c4ccc5ccccc5c4)cc3)nc(-c3ccc4c(c3)sc3ccccc34)n2)cc1 ; c1ccc(-c2cccc(-c3nc(-c4ccc5cc(-c6ccccc6)ccc5c4)nc(-c4cccc5sc6ccccc6c45)n3)c2)cc1]

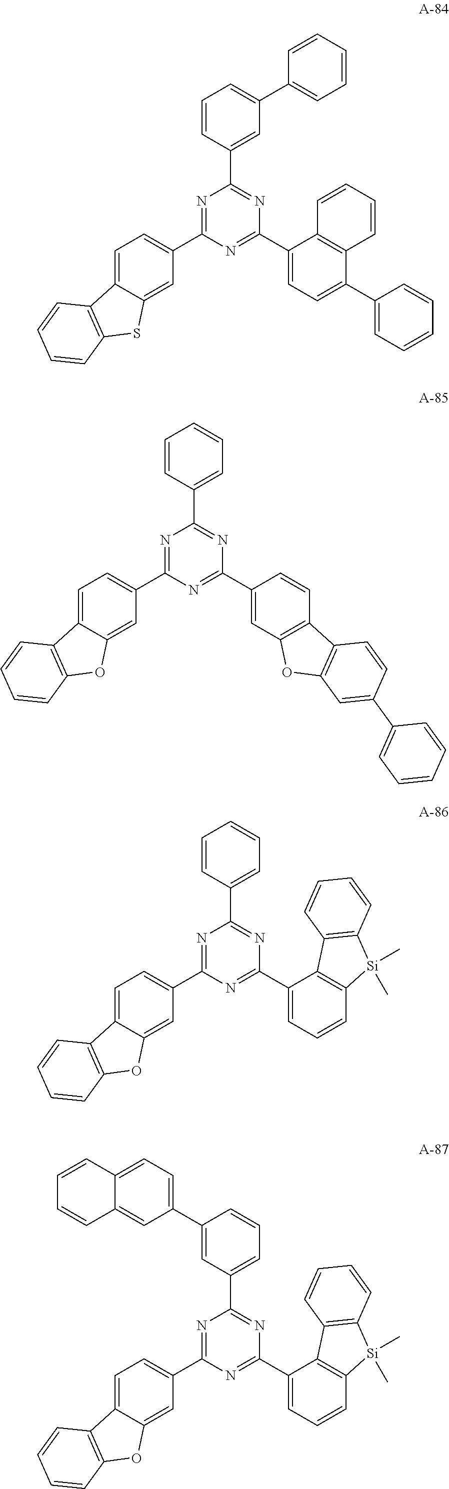 [SMILES: c1ccc(-c2cccc(-c3nc(-c4ccc5c(c4)sc4ccccc45)nc(-c4ccc(-c5ccccc5)c5ccccc45)n3)c2)cc1 ; c1ccc(-c2ccc3c(c2)oc2cc(-c4nc(-c5ccccc5)nc(-c5ccc6c(c5)oc5ccccc56)n4)ccc23)cc1 ; C[Si]1(C)c2ccccc2-c2c(-c3nc(-c4ccccc4)nc(-c4ccc5c(c4)oc4ccccc45)n3)cccc21 ; C[Si]1(C)c2ccccc2-c2c(-c3nc(-c4cccc(-c5ccc6ccccc6c5)c4)nc(-c4ccc5c(c4)oc4ccccc45)n3)cccc21]

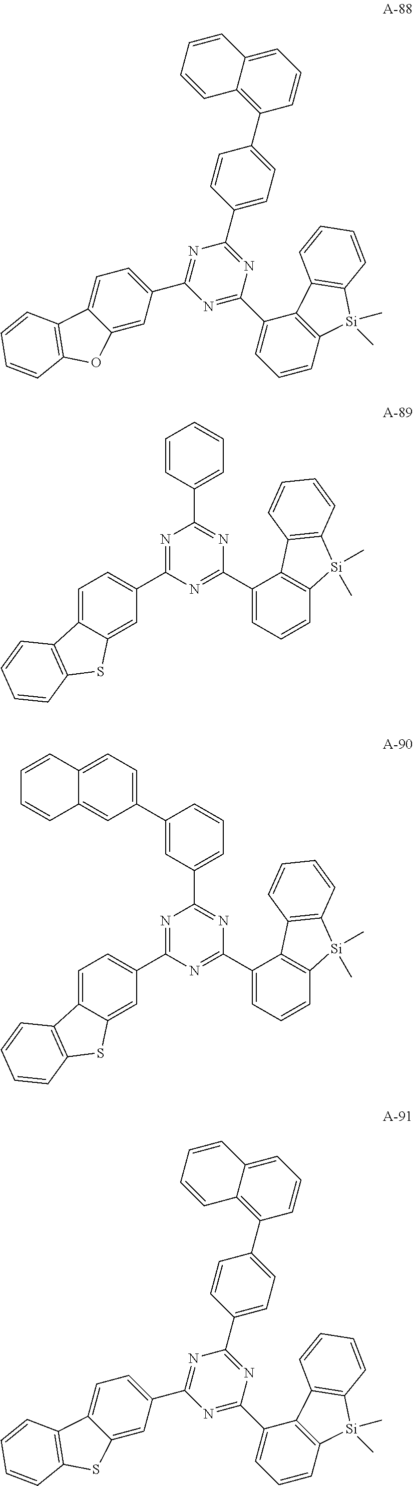 [SMILES: C[Si]1(C)c2ccccc2-c2c(-c3nc(-c4ccc(-c5cccc6ccccc56)cc4)nc(-c4ccc5c(c4)oc4ccccc45)n3)cccc21 ; C[Si]1(C)c2ccccc2-c2c(-c3nc(-c4ccccc4)nc(-c4ccc5c(c4)sc4ccccc45)n3)cccc21 ; C[Si]1(C)c2ccccc2-c2c(-c3nc(-c4cccc(-c5ccc6ccccc6c5)c4)nc(-c4ccc5c(c4)sc4ccccc45)n3)cccc21 ; C[Si]1(C)c2ccccc2-c2c(-c3nc(-c4ccc(-c5cccc6ccccc56)cc4)nc(-c4ccc5c(c4)sc4ccccc45)n3)cccc21]

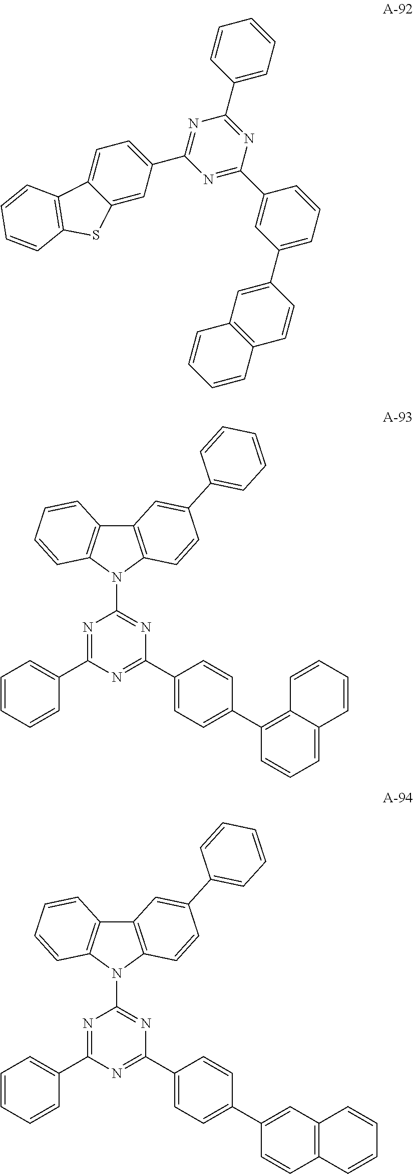

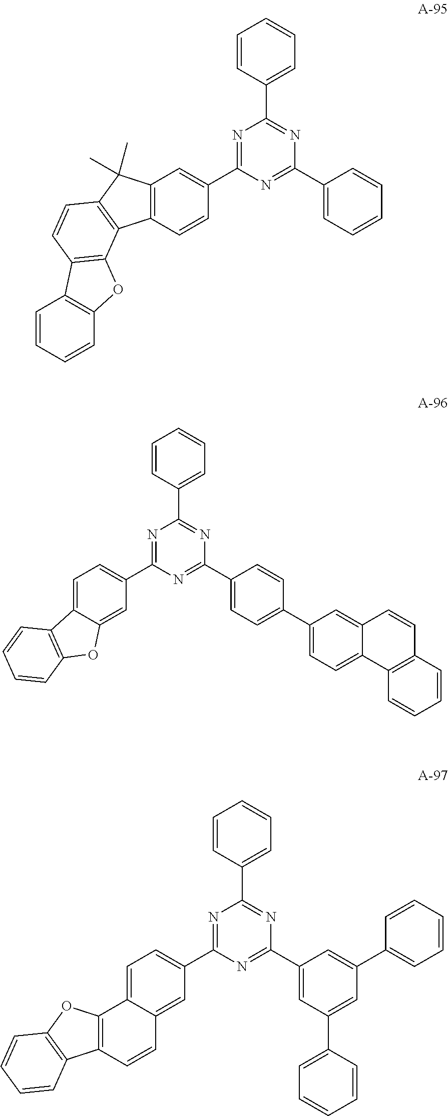 [SMILES: CC1(C)c2cc(-c3nc(-c4ccccc4)nc(-c4ccccc4)n3)ccc2-c2c1ccc1c2oc2ccccc21 ; c1ccc(-c2nc(-c3ccc(-c4ccc5c(ccc6ccccc65)c4)cc3)nc(-c3ccc4c(c3)oc3ccccc34)n2)cc1 ; c1ccc(-c2cc(-c3ccccc3)cc(-c3nc(-c4ccccc4)nc(-c4ccc5c(ccc6c7ccccc7oc56)c4)n3)c2)cc1]

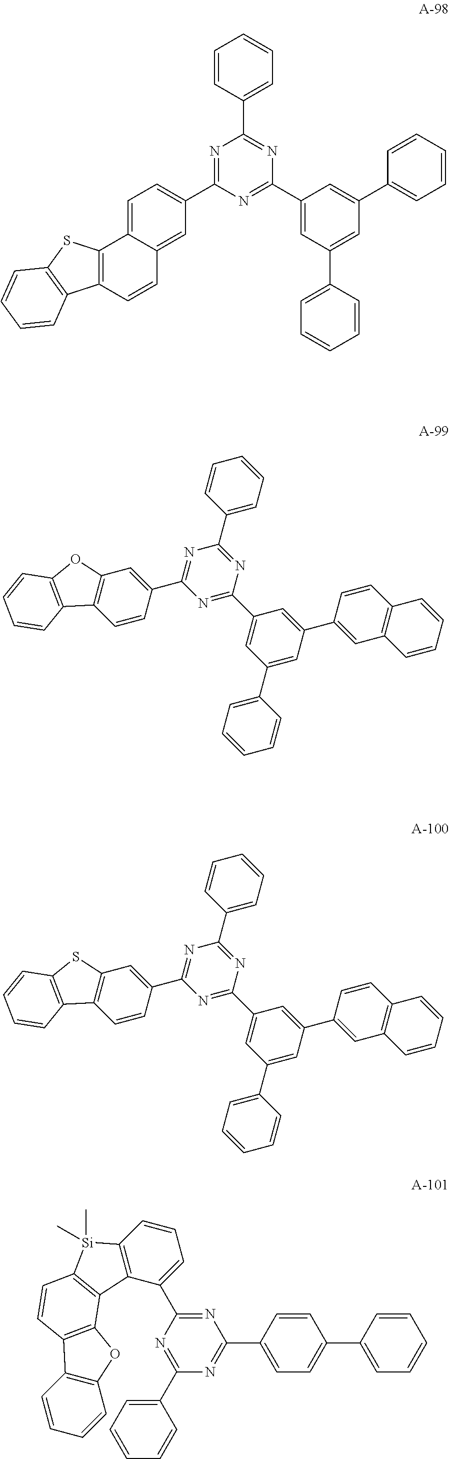 [SMILES: c1ccc(-c2cc(-c3ccccc3)cc(-c3nc(-c4ccccc4)nc(-c4ccc5c(ccc6c7ccccc7sc56)c4)n3)c2)cc1 ; c1ccc(-c2cc(-c3ccc4ccccc4c3)cc(-c3nc(-c4ccccc4)nc(-c4ccc5c(c4)oc4ccccc45)n3)c2)cc1 ; c1ccc(-c2cc(-c3ccc4ccccc4c3)cc(-c3nc(-c4ccccc4)nc(-c4ccc5c(c4)sc4ccccc45)n3)c2)cc1 ; C[Si]1(C)c2cccc(-c3nc(-c4ccccc4)nc(-c4ccc(-c5ccccc5)cc4)n3)c2-c2c1ccc1c2oc2ccccc21]

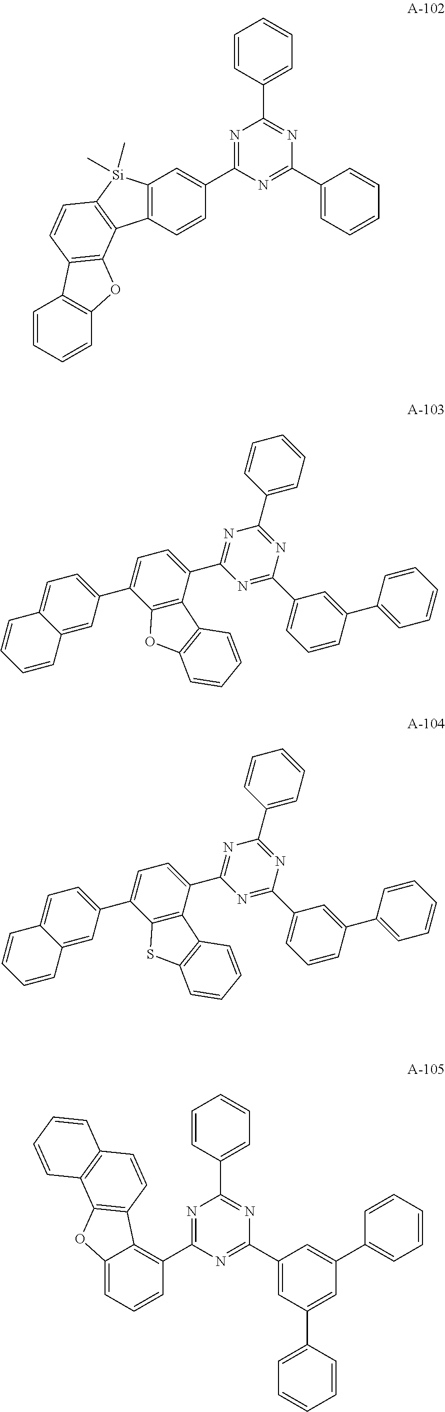 [SMILES: C[Si]1(C)c2cc(-c3nc(-c4ccccc4)nc(-c4ccccc4)n3)ccc2-c2c1ccc1c2oc2ccccc21 ; c1ccc(-c2cccc(-c3nc(-c4ccccc4)nc(-c4ccc(-c5ccc6ccccc6c5)c5oc6ccccc6c45)n3)c2)cc1 ; c1ccc(-c2cccc(-c3nc(-c4ccccc4)nc(-c4ccc(-c5ccc6ccccc6c5)c5sc6ccccc6c45)n3)c2)cc1 ; c1ccc(-c2cc(-c3ccccc3)cc(-c3nc(-c4ccccc4)nc(-c4cccc5oc6c7ccccc7ccc6c45)n3)c2)cc1]

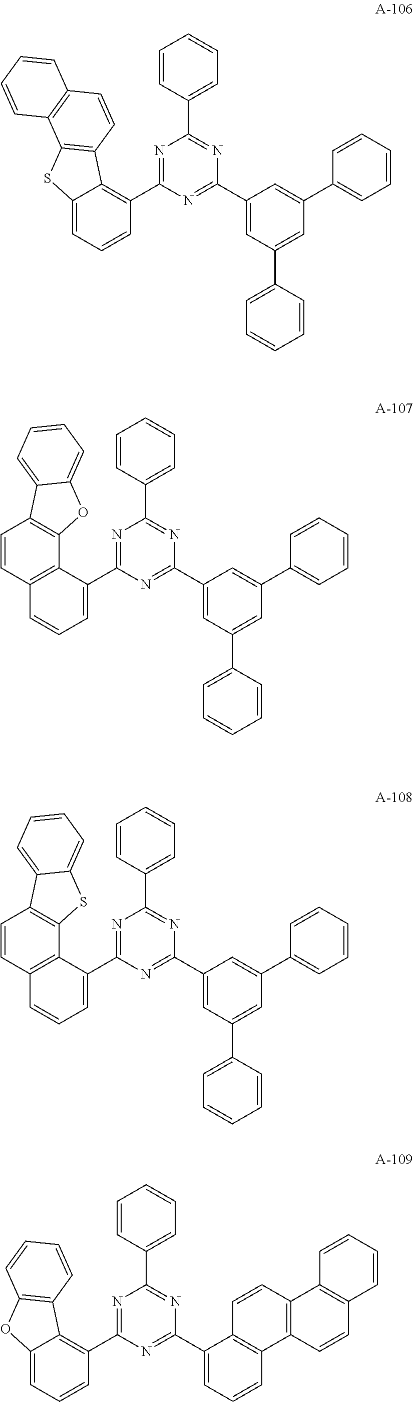 [SMILES: c1ccc(-c2cc(-c3ccccc3)cc(-c3nc(-c4ccccc4)nc(-c4cccc5sc6c7ccccc7ccc6c45)n3)c2)cc1 ; c1ccc(-c2cc(-c3ccccc3)cc(-c3nc(-c4ccccc4)nc(-c4cccc5ccc6c7ccccc7oc6c45)n3)c2)cc1 ; c1ccc(-c2cc(-c3ccccc3)cc(-c3nc(-c4ccccc4)nc(-c4cccc5ccc6c7ccccc7sc6c45)n3)c2)cc1 ; c1ccc(-c2nc(-c3cccc4c3ccc3c5ccccc5ccc43)nc(-c3cccc4oc5ccccc5c34)n2)cc1]

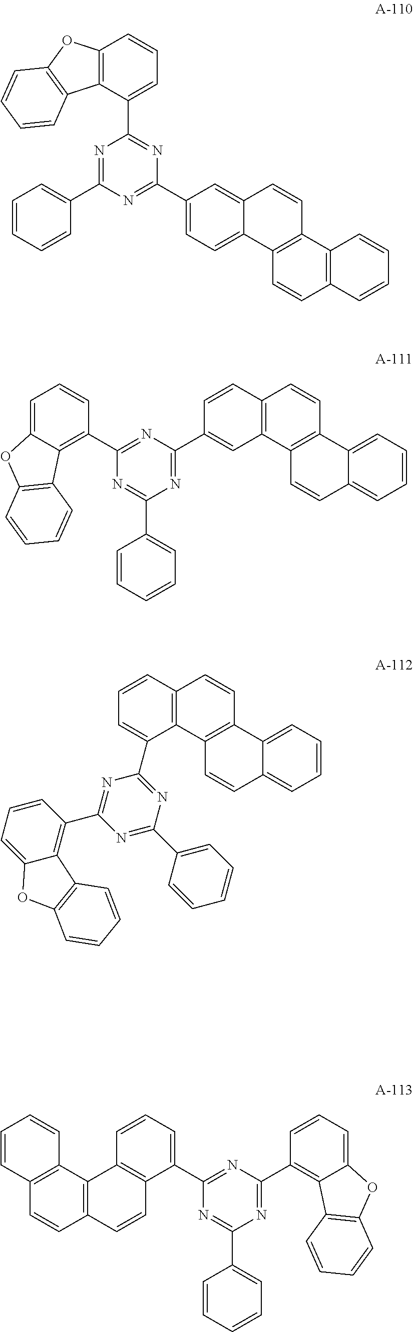 [SMILES: c1ccc(-c2nc(-c3ccc4c(ccc5c6ccccc6ccc45)c3)nc(-c3cccc4oc5ccccc5c34)n2)cc1 ; c1ccc(-c2nc(-c3ccc4ccc5c6ccccc6ccc5c4c3)nc(-c3cccc4oc5ccccc5c34)n2)cc1 ; c1ccc(-c2nc(-c3cccc4ccc5c6ccccc6ccc5c34)nc(-c3cccc4oc5ccccc5c34)n2)cc1 ; c1ccc(-c2nc(-c3cccc4c3ccc3ccc5ccccc5c34)nc(-c3cccc4oc5ccccc5c34)n2)cc1]

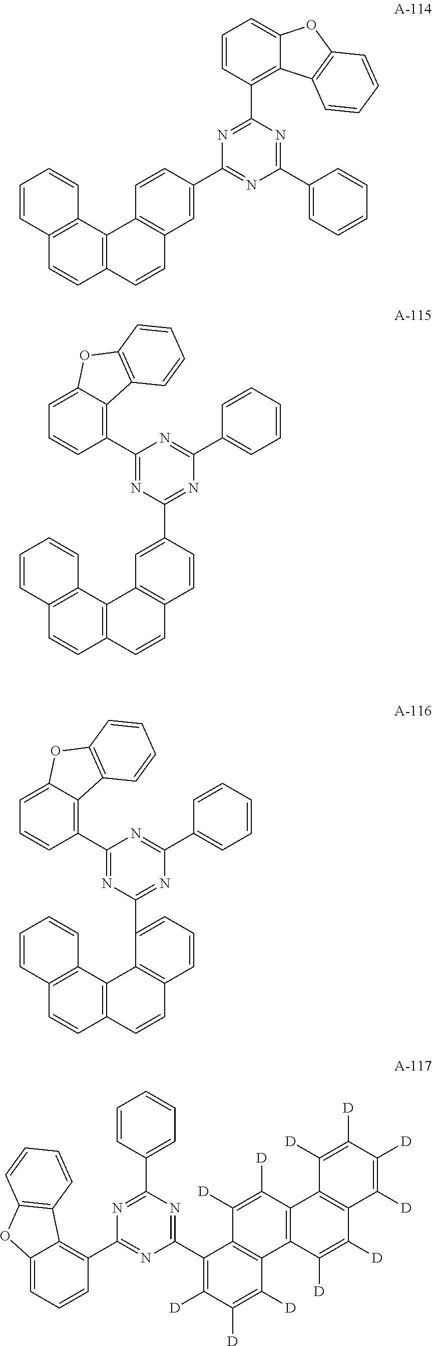

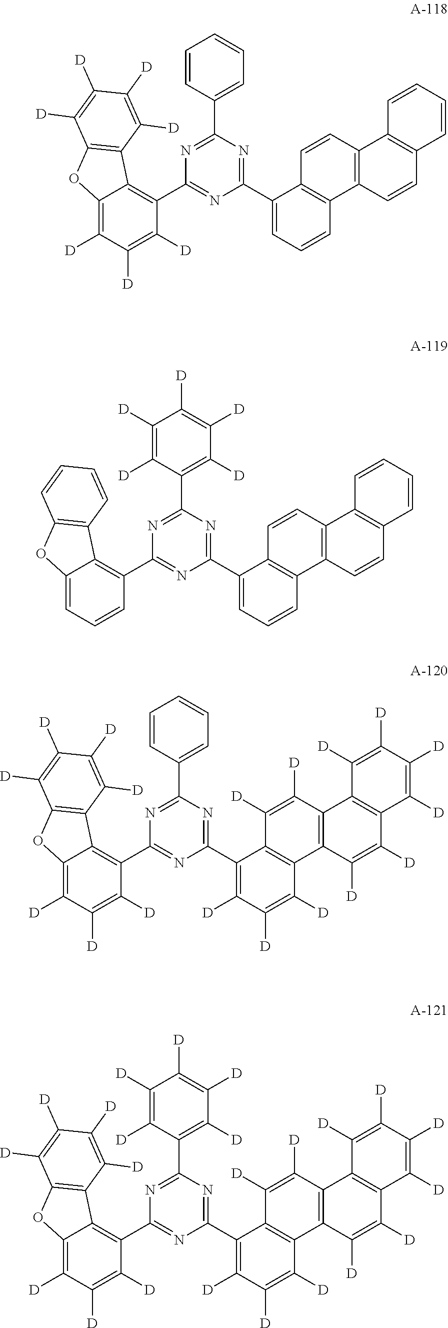

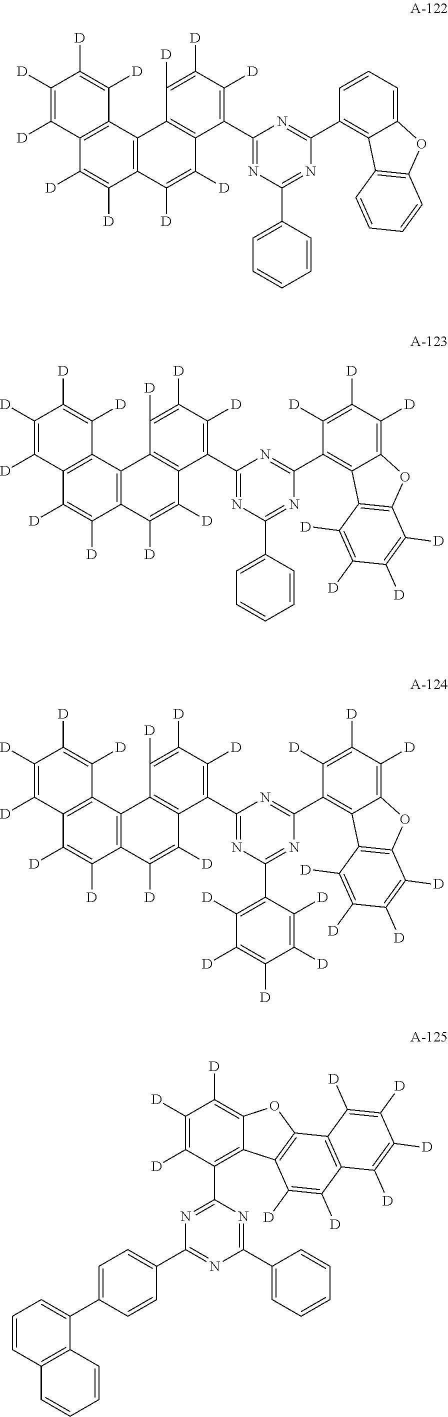

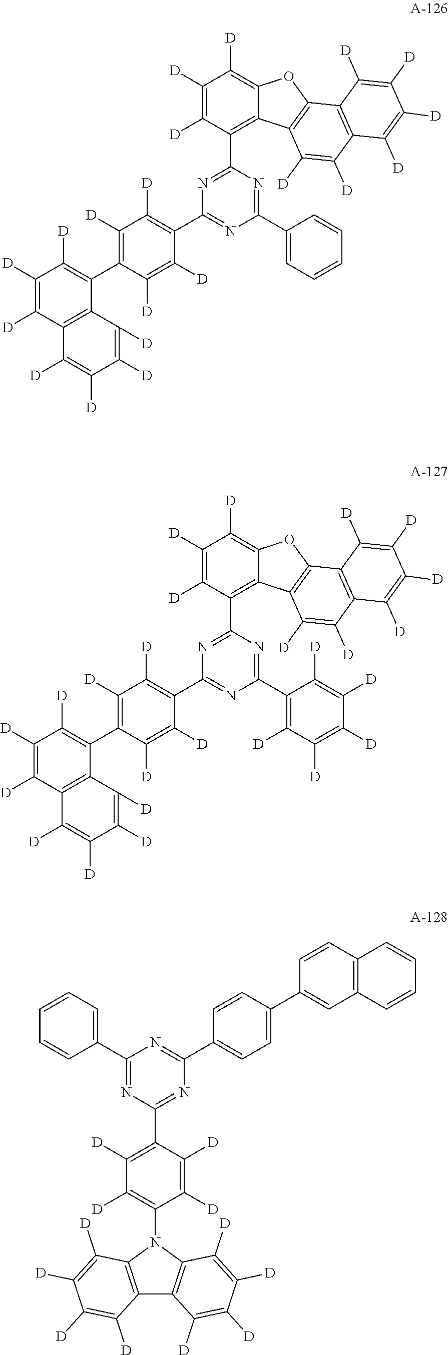

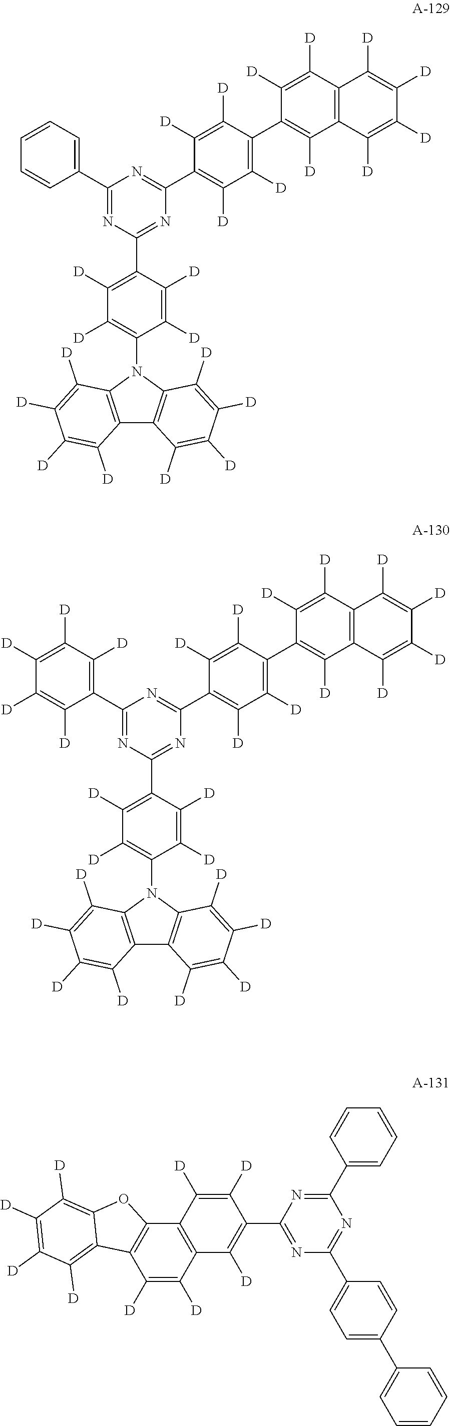

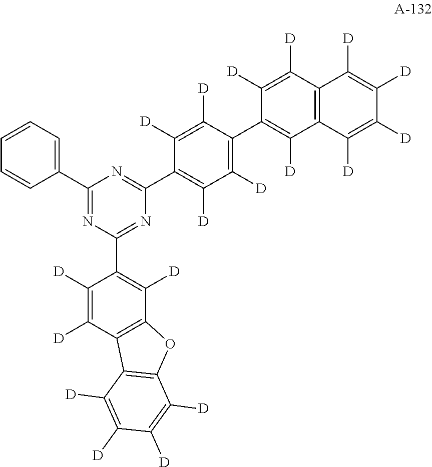 [SMILES: [2H]c1c([2H])c(-c2c([2H])c([2H])c3c([2H])c([2H])c([2H])c([2H])c3c2[2H])c([2H])c([2H])c1-c1nc(-c2ccccc2)nc(-c2c([2H])c([2H])c3c(oc4c([2H])c([2H])c([2H])c([2H])c43)c2[2H])n1]

14. An organic optoelectronic device, comprising:
an anode and a cathode facing each other, and
at least one organic layer between the anode and the cathode,
wherein the at least one organic layer includes the compound for an organic optoelectronic device as claimed in claim 1.

15. The organic optoelectronic device as claimed in claim 14, wherein:
the at least one organic layer includes a light emitting layer, and
the light emitting layer includes the compound for an organic optoelectronic device.

16. A display device comprising the organic optoelectronic device as claimed in claim 14.

17. An organic optoelectronic device, comprising:
an anode and a cathode facing each other,
at least one organic layer between the anode and the cathode,
wherein the at least one organic layer includes the composition for an organic optoelectronic device as claimed in claim 8.

18. The organic optoelectronic device as claimed in claim 17, wherein:
the at least one organic layer includes a light emitting layer, and
the light emitting layer includes the composition for an organic optoelectronic device.

19. A display device comprising the organic optoelectronic device as claimed in claim 17.
